(19) United States
(12) Patent Application Publication
(10) Pub. No.: US 2014/0350087 A9
(43) Pub. Date: Nov. 27, 2014

(54) Oncovector Nucleic Acid Molecules and Methods of Use

(71) Applicant: THE REGENTS OF THE UNIVERSITY OF CALIFORNIA, (US)

(72) Inventors: Gregory I. Frost, Del Mar, CA (US); Mark Roman, San Diego, CA (US); Frank McCormick, San Francisco, CA (US); Tanja Meyer Tamgüney, San Francisco, CA (US)

(73) Assignees: Halozyme, Inc., San Diego, CA (US); THE REGENTS OF THE UNIVERSITY OF CALIFORNIA, Oakland, CA (US)

(21) Appl. No.: 13/801,940

(22) Filed: Mar. 13, 2013

Related U.S. Application Data
(60) Provisional application No. 61/617,514, filed on Mar. 29, 2012.
(60) Provisional application No. 61/614,334, filed on Mar. 22, 2012.

(51) Int. Cl. A61K 48/00; A61K 31/513; C12N 15/85

(52) U.S. Cl. CPC A61K 48/0058; C12N 15/85; A61K 31/513
USPC 514/44 R; 435/320.1

(57) ABSTRACT

Provided herein are non-viral nucleic acid vectors, including non-viral oncovectors, that are autonomously replicating plasmids (ARPs). The non-viral nucleic acid vectors exhibit fusogenic activity and can exhibit other anti-tumor or cytotoxic activities. Also provided herein are methods and uses of the non-viral nucleic acid vectors for treating cancer.

### RELATED APPLICATIONS

Benefit of priority is claimed to U.S. Provisional Application Ser. No. 61/614,334, filed Mar. 22, 2012, entitled “Oncovector Nucleic Acid Molecules and Methods of Use,” and to U.S. Provisional Application Ser. No. 61/617,514, filed Mar. 29, 2012, entitled “Oncovector Nucleic Acid Molecules and Methods of Use.” The subject matter of each of the above-noted applications is incorporated by reference in its entirety.

This application is related to International PCT Patent Application No. (Attorney Docket No. UCSF-434WO), filed the same day herewith, entitled “Oncovector Nucleic Acid Molecules and Methods of Use,” which claims priority to U.S. Provisional Application Ser. Nos. 61/614,334 and 61/617,514.

The subject matter of each of the above-noted related application is incorporated by reference in its entirety.

### INCORPORATION BY REFERENCE OF SEQUENCE LISTING PROVIDED AS A TEXT FILE

A Sequence Listing is provided herewith as a text file, “UCSF-434_Seq_Listing” created on Mar. 12, 2013 and having a size of 1,964 KB. The contents of the text file are incorporated by reference herein in their entirety.

### FIELD OF THE INVENTION

Provided herein are non-viral nucleic acid vectors, including non-viral oncovectors, that are autonomously replicating plasmids (ARPs). The non-viral nucleic acid vectors exhibit fusogenic activity and can exhibit other anti-tumor or cytotoxic activities. Also provided herein are methods and uses of the non-viral nucleic acid vectors for treating cancer.

### BACKGROUND

The desired goal of cancer therapy is to kill cancer cells preferentially, without having a deleterious effect on normal cells. Several methods have been used in an attempt to reach this goal, including surgery, radiation therapy, chemotherapy and therapies with viral oncolytic vectors. Each of these has its limitations. Because of the limited effectiveness of the available therapies, there remains a need to develop alternative strategies for treating cancers and other diseases. Accordingly, it is among the objects herein to provide such alternative therapeutics and methods of treating cancer.

### SUMMARY

Provided herein are non-viral nucleic acid vector constructs that contain: a) an origin of replication; b) a first open reading frame coding for a fusogenic protein; and c) a second open reading frame coding for a replication initiator protein that activates the origin of replication for episomal replication of the vector in a cell in which it is expressed. The non-viral nucleic acid vectors include non-viral oncovectors. Any of the non-viral nucleic acid vectors provided herein can contain at least one promoter that is operatively linked to control expression of a first and/or second open reading frame. In some case, the non-viral nucleic acid vectors contain at least two promoters. The first and second promoter are the same or different.

In some examples of the non-viral nucleic acid vectors, including non-viral oncovectors, provided herein the first open reading frame and second open reading frame are separated by an internal ribosome entry site (IRES); and the first and second open reading frame are expressed under control of the same promoter. For example, the nucleic acid can contain in reading frame order 5′ to 3′: a promoter operatively linked to control expression of the first and second open reading frame, a first open reading frame coding for a fusogenic protein, an IRES, a second open reading frame coding for the replication initiator and an origin of replication. In some instances, the nucleic acid contains in reading frame order 5′ to 3′: a promoter operatively linked to control expression of the first and second open reading frame, a second open reading frame coding for the replication initiator, an IRES, a first open reading frame coding for a fusogenic protein and an origin of replication.

In other examples, the non-viral nucleic acid vectors, including non-viral oncovectors, provided herein contain a first promoter that is operatively linked to control expression of the first open reading frame coding for the fusogenic protein; and a second promoter that is operatively linked to control expression of the second open reading frame coding for the replication initiator. For example, the nucleic acid can contain in consecutive order: a first promoter operatively linked to control expression of the first open reading frame, a first open reading frame coding for a fusogenic protein, a second promoter that is operatively linked to control expression of the second open reading frame, a second open reading frame coding for a replication initiator and an origin of replication. In some instances, the nucleic acid contains in consecutive order: a second promoter that is operatively linked to control expression of the second open reading frame, a second open reading frame coding for a replication initiator, a first promoter operatively linked to control expression of the first open reading frame, a first open reading frame coding for a fusogenic protein.

In examples herein, the non-viral nucleic acid vectors, including non-viral oncovectors, provided herein, contain a non-viral nucleic acid vector, containing: a) a first nucleic acid molecule containing an origin of replication and a first open reading frame coding for a fusogenic protein; and b) a second nucleic acid molecule containing a second open reading frame coding for a replication initiator that activates the origin of replication in the first nucleic acid molecule when the first and second nucleic acid molecule are delivered into the same host cell for episomal replication of the first nucleic acid molecule. In such examples, the first and second nucleic acid molecule are part of the same molecule or are separate nucleic acid molecules. The first and second nucleic acid molecules can each contain at least one promoter that is operatively linked to control expression of the first and second open reading frame. The promoter can be the same or different.

In examples provided herein, the non-viral nucleic acid vectors, including non-viral oncovectors, contain an origin of replication and replication initiator selected from among: a) an SV40 origin and an SV40 T antigen; b) a BKV origin and BKV large T antigen; c) a BKV origin and SV40 T antigen; d) an EBV origin and Epstein Barr virus Nuclear Antigen (EBNA); and e) a JC Virus origin (see, e.g., Frisque (1983) J. Virol. 46:170) and a JC Virus large T antigen (see, e.g., Sock et al. (1993) Virol. 197:537).

In particular, the origin of replication is an SV40 origin and the replication initiator is an SV40 T antigen. For example, the SV40 origin contains an SV40 large T antigen core binding site set forth in SEQ ID NO:123, or a variant thereof having the formula set forth in SEQ ID NO:124 that exhibits at least 80% sequence identity to SEQ ID NO:123. For example, the sequence exhibits at least 85%, 86%, 87%, 88%, 89%, 90%, 91%, 92%, 93%, 94%, 95%, 96%, 97%, 98% or 99% sequence identity to SEQ ID NO:123. In some examples, the SV40 origin of replication contains a variant SV40 T antigen core binding site set forth in any of SEQ ID NOS: 125-189. In other examples, the origin of replication is a modified SV40 origin that is modified to remove upstream enhancers. The origin of replication also can be modified to remove CpG motifs and/or is human codon-optimized. Exemplary of such an origin of replication is an SV40 origin of replication that has the sequence set forth in SEQ ID NO:113, 114, 115, or 116 or that has a sequence that exhibits at least 80% sequence identity to any of SEQ ID NOS: 113, 114, 115 or 116. For example, in some embodiments, the sequence exhibits at least 85%, 86%, 87%, 88%, 89%, 90%, 91%, 92%, 93%, 94%, 95%, 96%, 97%, 98% or 99% sequence identity to any of SEQ ID NOS: 113, 114, 115, or 116.

In examples where the replication initiator is an SV40 T antigen, the second open reading frame can code for a replication initiator that is an SV40 large T antigen. The replication initiator, for example, SV40 T antigen, can be one that is modified to remove CpG motifs and/or is human-codon optimized. For example, the SV40 T antigen has the sequence set forth in any of SEQ ID NOS: 561, 562 or 563, degenerates thereof or a sequence that exhibits at least 80% sequence identity to any of SEQ ID NOS: 561, 562 or 563 or degenerates thereof. For example, the encoded SV40 large T antigen has the sequence of amino acids set forth in SEQ ID NO:564, or a variant thereof that exhibits at least 80% sequence identity to SEQ ID NO:564. For example, in some embodiments, the sequence exhibits at least 85%, 86%, 87%, 88%, 89%, 90%, 91%, 92%, 93%, 94%, 95%, 96%, 97%, 98% or 99% sequence identity to SEQ ID NO:564.

In particular examples of the non-viral nucleic acid vectors, including oncovectors, provided herein, the replication initiator is one that encodes an SV40 large T antigen that is modified to reduce its cellular transforming activity. For example, the encoded SV40 large T antigen is modified to reduce or eliminate binding to p53, HSP70 or Rb. Exemplary of encoded modified SV40 large T antigen are any that contain at least one amino acid replacement at an amino acid residue selected from among L17, G18, L19, E20, R21, S22, A23, W24, G25, N26, I27, P28, L29, M30, R31, K32, L103, C105, E107, E108, S112, S189, N366, D367, L368, L369, D370, D402, T434, L435, A436, A437, A438, L439, L440, E441, L442, C443, G444, P453, V585, D604, S677 or S679 corresponding to positions set forth in SEQ ID NO:564. For example, the encoded modified SV40 large T antigen can contain an amino acid replacement selected from among L19F, P28S, L103P, C105A, E107K, E107L, E108L, S112N, S189N, D402R, D402E, P453S, V585R, D604R, S677A and S679A. Exemplary of such amino acid replacements include, but are not limited to, E107L/E108L; E107L/E108L/D402R; E107L/E108L/P453S; E107L/E108L/V585R; E107L/E108L/D604R; L19F/E107L/E108L/D402R; L19F/E107L/E108L/P453S; L19F/E107L/E108L/V585R; L19F/E107L/E108L/D604R; P28S/E107L/E108L/D402R; P28S/E107L/E108L/P453S; P28S/E107L/E108L/V585R; P28S/E107L/E108L/V604R; L19F/P28S/L103P/C105A/E107L/E108L/V585R; L19F/P28S/L103P/C105A/E107L/E108L/D604R; L103P/C105A; L103P/E107K; C105A/E107K; C105A/D402E; C105A/V585R; E107K/V585R; E107K/D402E; L103P/D402E and L103P/V585R. For example, in examples of non-viral nucleic acid vectors provided herein, including oncovectors provided herein, the second open reading frame codes for a replication initiator protein that is an SV40 large T antigen containing the sequence of amino acids set forth in any of SEQ ID NOS: 565-604, or a sequence of amino acids that exhibits at least 80% sequence identity to any of SEQ ID NOS: 565-604. For example, the sequence exhibits at least 85%, 86%, 87%, 88%, 89%, 90%, 91%, 92%, 93%, 94%, 95%, 96%, 97%, 98% or 99% sequence identity to any of SEQ ID NOS: 565-604.

Included among the non-viral nucleic acid vectors provided herein, including non-viral oncovectors, are those in which the encoded fusogenic protein is a fusogenic protein that is a viral or eukaryotic fusogenic protein. For example, the fusogenic protein can be a VSV-G (Vesicular stomatitis virus G protein), MV (Measles virus) F protein, SIV (Simian immunodeficiency virus) F protein, HIV (Human immunodeficiency virus) 1+2 F protein, MuLV (Murine leukemia virus) F protein, Chicken LV Env Protein, SER virus F protein, NDV (Newcastle disease virus) F protein, GALV (Gibbon ape leukemia virus) F protein, SV5 (Simian virus 5) F protein, PPRV-F protein, Mumps F protein, Sendai virus F protein, Human parainfluenza virus types 1 (HPIV 1) F protein, HPIV 2 F protein, HPIV 3 F protein, CDV (Canine distemper virus) F protein, R′Pest F protein, SV41 (Simian virus 41) F protein, HRSV (Human respiratory syncytial virus) F protein, Human endogenous retroviral-3 (HERV-3), Reovirus FAST proteins, Avian Reovirus p10, Avian Reovirus p10 (S1133 variant V68I), Reptilian Reovirus p14, Baboon Reovirus p15, Eukaryotic Membrane Fusion Proteins, EFF-1, AFF-1, Tetraspanin Proteins, Yeast G Protein, Syncytin 1, Syncytin 2, Syntaxin (SNARE) or SNAP25 (SNARE), Synaptobrevin (SNARE) and variants thereof that exhibit fusogenic activity. In particular examples, the fusogenic protein is selected from among Reptilian Reovirus p14, Baboon Reovirus p15, Avian Reovirus p10, VSV-G fusion protein, SER virus F protein, SV5F, NDV F, Mumps F, Measles F or variants thereof that exhibit fusogenic activity. For example, the fusogenic protein is one that has the sequence of amino acids set forth in any of SEQ ID NO: 38, 39, 40, 41, 42, 43, 44 or 53 or a sequence of amino acids that exhibits at least 80% sequence identity to any of SEQ ID NOS: 38, 39, 40, 41, 42, 43, 44 or 53. For example, the sequence exhibits at least 85%, 86%, 87%, 88%, 89%, 90%, 91%, 92%, 93%, 94%, 95%, 96%, 97%, 98% or 99% sequence identity to any of SEQ ID NOS: 38, 39, 40, 41, 42, 43, 44 or 53. For example, the non-viral nucleic acid vectors provided herein contain an open reading frame coding for a fusogenic protein having the sequence of nucleotides set forth in SEQ ID NO: 6, 8, 10, 12, 14, 15, 17 or 27 or a sequence that exhibits at least 80% sequence identity to any of SEQ ID NOS: 6, 8, 10, 12, 14, 15, 17 or 27. For example, the sequence exhibits at least 85%, 86%, 87%, 88%, 89%, 90%, 91%, 92%, 93%, 94%, 95%, 96%, 97%, 98% or 99% sequence identity to any of SEQ ID NOS: 6, 8, 10, 12, 14, 15, 17 or 27.

In examples of non-viral nucleic acid vectors provided herein, the open reading frame coding for a bystander protein is a fusogenic protein that is modified to remove CpG motifs or is humanized. For example, the open reading frame codes for a fusogenic protein having the sequence of nucleotides set forth in SEQ ID NO:7, 9, 11, 13, 16, 18 or a sequence that exhibits at least 80% sequence identity to any of SEQ ID NOS:7, 9, 11, 13, 16 or 18. For example, in some embodiments, the sequence exhibits at least 85%, 86%, 87%, 88%, 89%, 90%, 91%, 92%, 93%, 94%, 95%, 96%, 97%, 98% or 99% sequence identity to any of SEQ ID NOS:7, 9, 11, 13, 16, 18.

In additional examples herein of non-viral nucleic acid vectors, the encoded fusogenic protein is a viral fusogenic F protein that contains an F1 subunit, wherein the F1 subunit has a modification in the N-terminal fusogenic peptide to increase the fusogenic activity of the encoded fusogenic protein. For example, the modification is an amino acid replacement (substitution), insertion or deletion. In examples where the modification is an amino acid replacement, the amino acid replacement is replacement of at least one Glycine residue with an Alanine.

In particular examples of the non-viral nucleic acid vectors provided herein, including oncovectors, the encoded fusogenic protein is a modified SV5F protein that has an amino acid replacement in the F1 subunit at an amino acid residue selected from among 105, 109 and 115 corresponding to positions set forth in SEQ ID NO:44. For example, the encoded modified SV5F fusogenic protein has an amino acid replacement selected from among G105A, G109A and G114A. Exemplary of such encoded modified SV5F fusogenic protein include those that contain an amino acid replacement selected from among G105A/G109A, G105A/G114A, G109A/G114A and G105A/G109A/G114A. For example, the fusogenic protein contains the sequence of amino acids set forth in any of SEQ ID NO: 45-51 or a sequence of amino acids that exhibits at least 80% sequence identity to any of SEQ ID NOS: 45-51. For example, in some embodiments, the sequence exhibits at least 85%, 86%, 87%, 88%, 89%, 90%, 91%, 92%, 93%, 94%, 95%, 96%, 97%, 98% or 99% sequence identity to any of SEQ ID NOS: 45-51. In examples herein of the non-viral nucleic acid vectors, the open reading frame coding for a fusogenic protein has the sequence of nucleotides set forth in any of SEQ ID NOS:19-25 or a sequence that exhibits at least 80% sequence identity to any of SEQ ID NOS: 19-25. For example, in some embodiments, the sequence exhibits at least 85%, 86%, 87%, 88%, 89%, 90%, 91%, 92%, 93%, 94%, 95%, 96%, 97%, 98% or 99% sequence identity to any of SEQ ID NOS: 19-25.

In other examples herein of non-viral nucleic acid vectors provided herein, including oncovectors, the encoded fusogenic protein is a modified Ser virus F protein. Exemplary of such an encoded fusogenic protein are those that have at least one amino acid replacement selected from among L539A, L548A, L548V and L548G corresponding to positions set forth in SEQ ID NO:53.

In the examples of non-viral nucleic acid vectors provided herein, including oncovectors, the promoter is a constitutive promoter, a tissue-specific promoter or a cell-specific promoter. For example, the promoter is a CMV promoter. In other examples, the promoter is a cell-specific promoter that is an endothelial nitric oxide synthase (eNOS) promoter; a vascular endothelial growth factor (VEGF) receptor (flk1) promoter; an insulin promoter; a promoter of gonadotropin-releasing hormone receptor gene; a matrix metalloproteinase 9 promoter; a promoter of parathyroid hormone receptor; or a dopamine beta-hydroxylase promoter. In additional examples, the promoter is a tumor-specific promoter. In such examples, for example, episomal replication occurs specifically in a tumor cell and not in a normal cell. For example, the tumor-specific promoter is a cell-cycle dependent promoter, such as an E2F responsive promoter, for example, an E2F responsive promoter that is a TATA-less promoter. In particular examples, the promoter is cycA, cdc2, cdc25, B-myb, E2F-1, p107, HsOrc1, or adenoE1A. In examples, the cell-cycle dependent promoter contains a CAT (CCAAT; SEQ ID NO:509) motif, for example, the promoter is a cdc25, cyclin B1, cyclin B2, Cdc2, topoisomerase IIa or E2F-1. In particular examples, the promoter is an E2F-1 promoter containing the sequence of nucleotides set forth as nucleotides 37 to 303 of SEQ ID NO:506, as nucleotides 1194 to 1460 of SEQ ID NO: 483 or nucleotides set forth in SEQ ID NO:534 or 535 or a variant sequence thereof that exhibits at least 80% sequence identity thereto. For example, the sequence exhibits at least 85%, 86%, 87%, 88%, 89%, 90%, 91%, 92%, 93%, 94%, 95%, 96%, 97%, 98% or 99% sequence identity to the sequence of nucleotides set forth as nucleotides 37 to 303 of SEQ ID NO:506, as nucleotides 1194 to 1460 of SEQ ID NO: 483 or nucleotides set forth in SEQ ID NO:534 or 535.

In examples of non-viral nucleic acid vectors provided herein, including non-viral oncovectors, the promoter can be modified by nucleotide changes, truncations, deletions, or insertions. For example, the promoter can be modified to remove CpG motifs. In other examples, the promoter is modified by deletion or truncation of nucleotides to reduce the promoter strength, to enhance the promoter strength, to reduce expression levels of the encoded protein or to increase expression levels of the encoded protein. For example, the promoter can be modified by addition or insertion of an enhancer element. The enhancer element can be an SP-1, CAT box or cycle genes homology region (CHR) element. In particular examples, the promoter is an E2F-1 promoter containing the sequence of nucleotides set forth in any of SEQ ID NOS:536-541 or a variant sequence thereof that exhibits at least 80% sequence identity thereto. For example, in some embodiments, the sequence exhibits at least 85%, 86%, 87%, 88%, 89%, 90%, 91%, 92%, 93%, 94%, 95%, 96%, 97%, 98% or 99% sequence identity to any of SEQ ID NOS:536-541.

In examples of any of the non-viral nucleic acid vectors provided herein, including non-viral oncovectors, the vector can further contain an open reading frame coding for a second bystander product other than the encoded fusogenic protein. For example, the vector can include an open reading frame coding for a bystander protein that is a prodrug modifying protein. The encoded prodrug modifying protein is herpes simplex 1 thymidine kinase gene (HSV-TK), cytosine deaminase (CD) or cytochrome p450. In such examples, the encoded prodrug modifying protein has the sequence of amino acids set forth in SEQ ID NO:501 or SEQ ID NO:502 or a variant sequence thereof that exhibits at least 80% sequence identity to SEQ ID NO:501 or 502. In other examples, the open reading frame codes for a prodrug modifying protein having the sequence of nucleotides set forth in SEQ ID NO: 498 or 500 or a variant sequence thereof that exhibits at least 80% sequence identity to SEQ ID NO: 498 or 500. For example, in some embodiments, the sequence exhibits at least 85%, 86%, 87%, 88%, 89%, 90%, 91%, 92%, 93%, 94%, 95%, 96%, 97%, 98% or 99% sequence identity to SEQ ID NO:498 or 500. In any of the examples of a prodrug modifying protein, the open reading frame coding for a bystander protein is modified to remove CpG motifs and/or is humanized. For example, the open reading frame coding for a prodrug modifying protein can have the sequence of nucleotides set forth in SEQ ID NO:499 or a variant sequence thereof that exhibits at least 80% sequence identity to SEQ ID NO:499. For example, in some embodiments, the sequence exhibits at least 85%, 86%, 87%, 88%, 89%, 90%, 91%, 92%, 93%, 94%, 95%, 96%, 97%, 98% or 99% sequence identity to SEQ ID NO:499. In examples of a nonviral vector provided herein containing a further open reading frame coding for a bystander product other than the encoded fusogenic protein, the nucleic acid molecule contains at least one promoter that is operatively linked to control expression of the first open reading frame, second open reading frame and/or the open reading frame coding for a second bystander product.

In examples of any of the non-viral nucleic acid vectors provided herein, including non-viral oncovectors, the vector can further contain an open reading frame coding for an adjunct therapy protein. The adjunct therapy protein can be a protein that induces apoptosis, a toxin, a prodrug modifying protein, a protein that interferes with a signal transduction cascade involved with cellular survival or proliferation, an immunomodulatory protein and an angiogenesis inhibitor. For example, the encoded adjunct therapy protein can be a cytokine or a chemokine. In examples of a nonviral vector provided herein containing a further open reading frame coding for a adjunct therapy protein, the nucleic acid molecule contains at least one promoter that is operatively linked to control expression of the first open reading frame, second open reading frame and/or the open reading frame coding for an adjunct therapy protein.

In examples of non-viral nucleic acid vectors provided herein, including non-viral nucleic acid vectors, the vector can contain an open reading frame coding for a reporter protein. The reporter protein can be a detectable protein, a protein capable of detection or a selectable marker. For example, the reporter protein can be chloramphenicol acetyl transferase (CAT), β-galactosidase, luciferase, alkaline phosphatase, a fluorescent protein, and horse radish peroxidase, an antibiotic resistance marker. In particular examples, the reporter protein is a green fluorescent protein (GFP), red fluorescent protein (RFP) luciferase or mKate. In examples of a nonviral vector provided herein containing a further open reading frame coding for a reporter protein, the nucleic acid molecule contains at least one promoter that is operatively linked to control expression of the first open reading frame, second open reading frame and/or the open reading frame coding for a reporter protein.

In any of the examples of a non-viral nucleic acid vector provided herein, the entire nucleic acid is modified to remove CpG motifs and/or is humanized. In other examples herein, the first and/or second open reading frame, or any of the further open reading frames, is operatively linked to one or more regulatory elements to control expression of the gene. For example, the regulatory element is a polyadenylation signal or an internal promoter.

In particular examples of non-viral nucleic acid vectors provided herein, including non-viral oncovectors, the vector contains: a) a promoter that controls expression of the first and second open-reading frame; b) a first open reading frame coding for a fusogenic protein or variant thereof that exhibits fusogenic activity; c) an IRES; d) a second open reading frame coding for a replication initiator or variant thereof that is capable of initiating episomal replication; and e) an origin of replication or variant thereof that effects replication. In such an example, the first open reading frame is positioned before the second open reading frame in the nucleic acid molecule. In other examples, the second open reading frame is positioned before the first open reading frame in the nucleic acid molecule.

Provided herein is a non-viral nucleic acid vector that has the sequence of nucleotides set forth in SEQ ID NO: 647, 649, 651, 653, 655, 657, 659-663, 693, 700-705, 722 and 727 or a sequence of nucleotides that exhibits at least 80% sequence identity to any of SEQ ID NO: 647, 649, 651, 653, 655, 657, 659-663, 693, 700-705, 722 and 727. For example, in some embodiments, the sequence of nucleotides exhibits at least 85%, 86%, 87%, 88%, 89%, 90%, 91%, 92%, 93%, 94%, 95%, 96%, 97%, 98% or 99% sequence identity to any of SEQ ID NO: 647, 649, 651, 653, 655, 657, 659-663, 693, 700-705, 722 and 727. Also provided herein is a non-viral nucleic acid vector that has the sequence of nucleotides set forth in SEQ ID NO:664 or 724, or a sequence of nucleotides that exhibits at least 80% sequence identity to SEQ ID NO:664 or 724. For example, in some embodiments, the sequence of nucleotides exhibits at least 85%, 86%, 87%, 88%, 89%, 90%, 91%, 92%, 93%, 94%, 95%, 96%, 97%, 98% or 99% sequence identity to SEQ ID NO:664 or 724.

In any of the examples provided herein, the non-viral nucleic acid vector is provided as a naked DNA.

Provided herein is a nanoparticle, containing any of the non-viral nucleic acid vectors provided herein, including any of the non-viral nucleic acid vectors. The nanoparticles provided herein include those that are based on polyethylenimine (PEI) polymers, polypropylenimine dendrimers PPIG3 polymers, B-amino-ester polymers, liposome formulations, or sugar molecules such as cyclodextrin polymers. For example, the nanoparticle is a liposome formulation. Also provided herein is a liposome containing any of the non-viral nucleic acid vectors provided herein, including any of the non-viral nucleic acid vectors. Any of the nanoparticles or liposomes provided herein can be conjugated to a protein that targets a tumor. For example, a protein that targets a tumor can be transferrin, an arginine-glycine-aspartate (RGD) peptide, an αvβ3 binding targeting peptide, folate or an antibody targeting a protein expressed or overexpressed on the surface of a tumor cell.

Provided herein is a combination containing any of the non-viral nucleic acid vectors provided herein, including non-viral oncovectors, or a nanoparticle or liposome thereof, and a hyaluronidase protein.

Provided herein is a method of treating cancer by administering any one of the non-viral nucleic acid vectors provided herein, nanoparticles or liposomes containing such vectors, or combination containing any of the vectors to a subject that has cancer. The cancer can be a sarcoma, mesothelioma, carcinoid, melanoma, neuroblastoma, retinoblastoma, osteosarcoma, or cancers of the lung, colon, esophagus, ovary, pancreas, skin, stomach, head and neck, bladder, prostate, liver, brain, adrenal gland, breast, endometrium, kidney, thyroid, parathyroid, cervix, bone, eye or hematological system. The methods provided herein can further include treating the subject by a targeted therapy, chemotherapy, radiotherapy, immunotherapy, hormonal therapy, cryotherapy or surgery.

Provided herein is a pharmaceutical composition for use in treating a cancer containing any of the non-viral nucleic acid vectors provided herein, nanoparticles or liposomes containing any of the non-viral nucleic acid vectors. Also provided herein is a pharmaceutical composition containing any of the combinations provided here for use in treating cancer. The compositions provided herein can be formulated as a medicament for treating cancer.

### BRIEF DESCRIPTION OF THE FIGURES

FIGS. 1A-F depict the fusogenic properties of the oncovectors described herein, including the tumor-specific replication and expression of fusogenic proteins encoded thereon, to selectively elicit oncolysis.

FIGS. 2A-H depict the evolution of an exemplary oncovector construct using schematic BspHI-linearized vector maps. Highlighted features are presented in boxes, which can be interchanged following digestion and ligation at the indicated restriction sites. FIG. 2A depicts features of the starting vector, pIRES2-EGFP (SEQ ID NO: 1). FIG. 2B depicts a modified starting vector wherein the gene encoding EGFP is replaced with a CpG-free, human codon-optimized gene encoding EGFP (e.g., pIRES2-zGFP; SEQ ID NO: 694). FIG. 2C depicts the modified vector presented in FIG. 2B with a nucleotide sequence encoding SV40 TAg, inserted between the NheI and BamHI restriction sites, for use as a vector to test autonomous replication (e.g., pC-T-I-zGFP; SEQ ID NO: 697). FIG. 2D depicts the modified test vector presented in FIG. 2C with a nucleotide sequence encoding a fusogenic protein, inserted between the NheI and BamHI restriction sites, for use as a vector to test fusogenic activity (e.g., pC-zGALV-IzG; SEQ ID NO: 713). FIG. 2E depicts the modified test vector presented in FIG. 2C with a cell cycle-dependent promoter (CCD) replacing the CMV promoter between the AseI and NheI restriction sites for use as a vector to test cell type selectivity of autonomous replication (e.g., pCMV/EF1-zGFP-I-T-BB3; SEQ ID NO: 666). FIG. 2F depicts the modified test vector presented in FIG. 2E with a nucleotide sequence encoding a fusogenic protein inserted between the NheI and BamHI restriction sites for use as a vector to test cell type-specific fusogenic activity. FIG. 2G depicts an oncovector derived from the test vector presented in FIG. 2F, wherein the nucleotide sequence for the fusogenic protein exhibiting the best fusogenic activity is present between the NheI and BamHI restriction sites (first position) and the nucleotide sequence encoding the SV40 TAg protein conferring the best replicative activity is inserted between the BstXI and Not I restriction sites (second position). FIG. 2H depicts a combinatorial oncovector derived from the vector presented in FIG. 2G, wherein a nucleotide sequence corresponding to an adjunct therapy gene, such as a prodrug modifying enzyme (e.g. cytosine deaminase), is inserted in a location isolated from the bicistronic sequence, for example between the Pf1FI and Bg1II restriction sites (third position).

FIGS. 3A-L depict plasmid maps for exemplary backbone and intermediate constructs and experimental vectors. FIG. 3A depicts Intermediate Vector 1 (SEQ ID NO: 2). FIG. 3B depicts Intermediate Vector 2 (SEQ ID NO: 3). FIG. 3C depicts Intermediate Vector 3 (SEQ ID NO: 4). FIG. 3D depicts Intermediate Vector 4 (SEQ ID NO: 5). FIG. 3E depicts an exemplary test vector derived from Intermediate Vector 4 with mammalian cell replication and expression capabilities. FIG. 3F depicts an exemplary test vector derived from Intermediate Vector 4 with tumor cell-specific replication and expression capabilities. FIG. 3G depicts an exemplary test vector (pCzGFP-I-T-BB3; SEQ ID NO: 607) derived from BB3 backbone. FIG. 3H depicts an exemplary replication-deficient test vector (pCzGFP-I-T-dSV; SEQ ID NO: 608) derived from BB3 backbone. FIG. 3I depicts an exemplary test vector (pCzGFP-I-T-BB4; SEQ ID NO: 719) derived from BB4 backbone. FIG. 3J depicts an exemplary replication-deficient test vector (pCzGFP-I-T-dSV4-1; SEQ ID NO: 720) derived from BB4 backbone. FIG. 3K depicts an exemplary test vector (pCzGFP-I-T-BB5; SEQ ID NO: 726) derived from BB5 backbone. FIG. 3L depicts an exemplary test vector, expressing an exemplary fusogenic protein (pCzGALV-I-T-BB3; SEQ ID NO: 653) derived from BB3 backbone.

FIGS. 4A-F illustrate the process of overlap extension polymerase chain reaction to construct an exemplary gene.

### DETAILED DESCRIPTION

### Outline

A. Definitions

B. Overview of the Oncovector System

C. Components of the Oncovector System and Resulting Oncovectors
1 Replication Unit
   a. SV40 Origin and Mutants Thereof
   b. SV40 T Antigen and Mutants Thereof
2. Therapeutic Genes
   a. Gene Encoding a Fusogenic Protein
      i. Exemplary Viral Fusogenic F Proteins and Variants
   b. Gene encoding a Prodrug Converting Enzyme
3. Promoter
   a. Cell-cycle Dependent Promoters
4. Other Elements
   a. Regulatory Elements
      i. IRES
      ii. Polyadenylation Signal
   b. Reporter Genes
   c. Adjunct Therapy Proteins
      i. Suicide Genes
      ii. Immunomodulatory Proteins
      iii. Angiogenesis Inhibitors
5. Modification of Components
6. Exemplary Oncovector Constructs

D. Methods of Designing Oncovector Constructs
1. Backbone Constructs
2. Experimental Test Vector Backbones
   a. Replication Competent Vector
   b. Fusogenic Competent Vector
   c. Tissue or Cell Specificity/Selectivity Competent Vector
3. Integration of Constructs to Generate an Oncovector

E. Methods of Producing Oncovector Constructs
1. Synthetic Genes and Peptides
2. Methods of Cloning and Isolating Component Genes
3. Methods of Generating and Cloning Constructs

F. Assays to Assess or Monitor Activities of Oncovector Constructs
1. Replication Assays
   a. Incorporation of detectable nucleoside and/or nucleotide analogs
   b. Real-time polymerase chain reaction (qPCR)
   c. Southern Blot Analysis
   d. DpnI digestion
   e. Binding of SV-T to SV40 origin of replication
2. Cell Fusion Assays
   a. Fluorescence dequenching
   b. Dye Transfer
   c. Content mixing
   d. Syncytium formation
3. Assays for Transformation of Normal Cells to Cancerous Cells
   a. Immortalization
   b. Growth in low serum
   c. Saturation density
   d. Focus formation
   e. Overcoming growth-inhibition of tumor suppressors
   f. Activation of cyclin A
   g. Anchorage independence
   h. Formation of tumors in animals
   i. Binding of SV-T to Tumor Suppressor Proteins
4. Expression Assays
5. Immunogenicity Assays
6. Animal Models

G. Preparation, Formulation and Administration of Oncovector Constructs and Oncovector Construct Compositions
Exemplary Delivery Methods

H. Exemplary Methods of Treatment
1. Cancers
   a. Lung Cancer
   b. Colorectal Cancer
   c. Bladder Cancer
   d. Ovarian Cancer
   e. Skin Cancer
   f. Prostate Cancer
   g. Breast Cancer
2. Selection of the Components of an Oncovector Construct for Treatment
3. Combination Therapies

I. Examples

### A. DEFINITIONS

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, self-replication refers to a plasmid which contains all components to allow for its own amplification (i.e. an origin and a gene expressing a replication initiator).

As used herein, episomal or extrachromosomal replication refers to amplification or replication of plasmid sequences without prior integration of these plasmid sequences into the mammalian genome, i.e. without integration into a chromosome.

As used herein, “autonomous replication” with reference to a nucleic acid molecule, such as an autonomously replicating plasmid (ARP), refers to a nucleic acid molecule or plasmid that is capable of self-replication that is episomal or extrachromosomal.

As used herein, nucleic acid molecule refers to single-stranded and/or double-stranded polynucleotides, such as deoxyribonucleic acid (DNA) and ribonucleic acid (RNA), as well as analogs or derivatives of either RNA or DNA. Also included in the term “nucleic acid” are analogs of nucleic acids such as peptide nucleic acid (PNA), phosphorothioate DNA, and other such analogs and derivatives. Nucleic acids can encode gene products, such as, for example, polypeptides, regulatory RNAs, microRNAs, siRNAs and functional RNAs. Hence, nucleic acid molecule is meant to include all types and sizes of DNA molecules including siRNA, aptamers, ribozymes, cDNA, plasmids and DNA including modified nucleotides and nucleotide analogs.

As used herein, a construct refers to a piece of circular double-stranded DNA, such as a vector or plasmid.

As used herein, “plasmid” or “vector” is used interchangeably and is meant a circular DNA vector. Plasmids contain an origin of replication that allows many copies of the plasmid to be produced in a bacterial or eukaryotic cell without integration of the plasmid into the host cell DNA.

As used herein, a non-viral nucleic acid vector refers to a nucleic acid molecule that contains an origin of replication and other elements for replication of the nucleic acid (i.e. it is capable of autonomous replication), which can be of viral origin, but does not include all of the requisite elements that result in a viral particle, such as elements for viral replication, packaging and/or expression. Such elements include, but are not limited to, one or more of the nucleic acid molecules encoding a capsid protein or coat protein, a packaging signal, an early promoter and regulators of late viral gene expression. Hence, for example, a non-viral nucleic acid vector is not packaged as a viral vector particle.

As used herein, an “oncovector” is a non-viral nucleic acid vector that contains an element or elements such that the vector preferentially replicates in tumors but not in normal tissue. Hence, the oncovector is an autonomously replicating plasmid (ARP) in tumor cells.

As used herein, the term “gene” refers to any and all discrete coding regions of a host genome, or regions that code for a functional RNA only (e.g., tRNA, rRNA, regulatory RNAs such as ribozymes etc) as well as associated non-coding regions and optionally regulatory regions. In certain embodiments, the term “gene” includes within its scope the open reading frame encoding specific polypeptides, introns, and adjacent 5′ and 3′ non-coding nucleotide sequences involved in the regulation of expression. In this regard, the gene can further contain control signals such as promoters, enhancers, termination and/or polyadenylation signals that are naturally associated with a given gene, or heterologous control signals. The gene sequences can be cDNA or genomic DNA or a fragment thereof. The gene can be introduced into an appropriate vector for extrachromosomal maintenance or for integration into the host.

As used herein, “open reading frame” (ORF) refers to a DNA sequence starting with a start codon and ending with a stop codon, and therefore signaling a coding sequence that is translated into a functional product RNA or polypeptide. Hence, an ORF is synonymous with coding sequence.

As used herein, a “DNA transcription unit” or “transcription unit” refers to nucleic acid molecule encoding a protein that contains not only the open reading frame (ORF) that is directly translated into the protein (the coding sequence), but also can include regulatory sequences that direct and regulate the synthesis of the protein. The regulatory sequences before (upstream from) the coding sequence is called the five prime (5′) untranslated region (5′UTR) and the sequence following (downstream from) the coding sequence is called the three prime (3′) untranslated region (3′UTR). For example, the 3′ untranslated region can include a polyadenylation site.

As used herein, an “expression cassette” refers to one or more genes and the sequences controlling their expression. Typically, an expression cassette includes a promoter sequence, an open reading frame and a 3′ untranslated region that, in eukaryotes, usually contains a polyadenylation signal.

As used herein, “replication competent” with reference to a plasmid means that a nucleic acid molecule or plasmid contains the minimal components required for autonomous replication. For purposes herein, a nucleic acid molecule is replication competent if it minimally contains an origin of replication that can be initiated upon binding of a cognate or compatible replication initiator. Generally, a nucleic acid molecule is replication competent if it contains a complete replication unit containing both the origin of replication and an open reading frame coding for expression of a cognate or compatible replication initiator.

As used herein, “non-replicating” or “replication-deficient” with reference to a nucleic acid molecule or plasmid refers to a nucleic acid molecule that is not capable of autonomous replication. For example, a non-replicating nucleic acid molecule is one that does not contain an origin of replication.

As used herein, a “replication unit” refers to the portions of a DNA molecule or molecule(s) that are capable of conferring independent replication of one of the molecules. For example, a replication unit confers extrachromosomal or episomal replication of a DNA molecule. The replication unit can be on the same DNA molecule or on separate DNA molecules. For purposes herein, a replication unit is generally derived from a virus system. A replication unit typically minimally contains an origin of replication and a compatible or cognate replication initiator to activate the origin.

As used herein, an origin of replication (origin) refers to a particular sequence of DNA that is required for replication to begin and at which DNA replication is initiated on a plasmid, virus or chromosome. For purposes herein, an origin of replication includes any origin, and typically any viral origin such as any polyomavirus origin, that can drive episomal replication in eukaryotic cells, such as mammalian cells or human cells. Exemplary of origins include, but are not limited to, origins from SV40, BKV, JC virus, lymphotropic papovavirus, and simian agent 12. An origin of replication also includes any sequence variant that exhibits a difference in its nucleotide sequence (e.g. due to nucleotide substitution or insertion, truncation or deletion or addition of nucleotides), but that is still capable of initiating replication of DNA in a eukaryotic cell. For example, an origin of replication includes any containing 2, 3, 4, 5, 6, 7, 8, 9, 10 or more binding sites for a compatible or cognate replication initiator.

As used herein an SV40 origin of replication refers to an origin of replication derived from the SV40 double-stranded DNA virus, which belongs to the Polyomaviridae family. An SV40 origin of replication contains four binding sites for its cognate replication initiator, SV40 large T antigen, arranged in a palindromic pattern containing two GAGGC motifs and two CTCCG antisense motifs (SEQ ID NO:123). Reference to an SV40 origin of replication also includes any sequence variant that exhibits a difference in its nucleotide sequence (e.g. due to nucleotide substitution or insertion, truncation or deletion or addition of nucleotides), but that is still capable of initiating replication of DNA in a eukaryotic cell in the presence of a replication initiator. For example, an SV40 origin of replication variants includes any that exhibits at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of nucleotides set forth in SEQ ID NO:123, and is capable of initiating replication of DNA in a eukaryotic cell in the presence of a replication initiator. For example, an SV40 origin of replication includes any containing 2, 3, 4, 5, 6, 7, 8, 9, 10 or more binding sites for a compatible or cognate replication initiator (see e.g. SEQ ID NO:37, 79, 113, or 124).

As used herein, a replication initiator refers to an encoded protein that can bind to a site or region of the origin of replication to initiate DNA replication. Typically, DNA replication is initiated upon binding of the initiator and separating of the two strands of DNA to expose single-stranded DNA, e.g. due to a helicase activity of the replication initiator or other recruited protein. A replication initiator is generally compatible with and can bind to the origin of replication. Exemplary of replication initiators are any that are virally-derived, such as from a polyomavirus. For example, a replication initiator includes, but is not limited to, large T antigen for SV40 (SV40 TAg), polyoma and BKV, and EBNA for EBV. Reference to a variant of a replication initiator refers to any encoded sequence variant that exhibits a difference in its amino acid sequence (e.g. due to amino acid substitution or insertion, truncation or deletion or additions), but that is still capable of binding to an origin of replication to initiate replication of DNA in a eukaryotic cell.

As used herein, SV40 large T Antigen (SV40 T Ag) refers to a replication initiator derived from the SV40 double-stranded DNA virus, and which can bind to the SV40 origin of replication. The SV40 TAg has the sequence of amino acids set forth in SEQ ID NO:564 and is encoded by a sequence of nucleotides set forth in SEQ ID NO:561. Reference to a variant of an SV40 large T antigen (or encoding nucleic acid molecule) refers to any that exhibits a difference in its sequence (e.g. due to nucleotide or amino acid substitutions or insertions, truncation or deletions or additions), but that is still capable of binding to (or encodes a protein that is still capable of binding to) an SV40 origin or replication or other compatible origin of replication to initiate replication of DNA in a eukaryotic cell. For example, an SV40 TAg variants includes any that exhibits at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of nucleotides set forth in SEQ ID NO:561 or degenerate codons thereof, and is capable of encoding a protein that binds an SV40 origin or replication or other compatible origin of replication to initiate replication of DNA in a eukaryotic cell. For example, an exemplary SV40 TAg replication initiator is encoded by the sequence of nucleotides set forth in SEQ ID NO:562 or 563. An SV40 TAg variant also includes any that exhibits at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of amino acids set forth in SEQ ID NO:564, and is capable of binding an SV40 origin or replication or other compatible origin of replication to initiate replication of DNA in a eukaryotic cell. For example, an exemplary SV40 TAg variant replication initiator has the sequence of amino acids set forth in SEQ ID NO:565-604.

As used herein, binding with reference to binding of a replication initiator (e.g. SV40 TAg) and origin of replication (e.g. SV40 origin) refers to specific binding to the origin of replication. For example, specific binding can be determined using an immunoprecipitation assay and analysis by electrophoresis and autoradiography (see e.g. Cole et al. (1986), J. Virol. 57(2):539-546; Scheller et al. (1982) Cell, 29:375-383). For example, nuclear extracts from cells expressing SV40 TAg (e.g., cells genetically modified with an expression construct that encodes SV40 TAg, such that SV40 TAg is expressed in the cells) can be obtained and incubated with radiolabeled SV40 DNA fragments (e.g. for 1 hour at 4° C.) Anti-SV40 Tag antibody or tumor antiserum can be added (e.g. for an additional 30 minutes). The material can be precipitated, immune complexes isolated by centrifugation and bound DNA dissociated therefrom and analyzed by electrophoresis. Such binding assays are well-known to one of skill in the art.

As used herein, the term “accumulate” refers to building up of plasmid or gene product expressed from the plasmid (after replication).

As used herein, “compatible” with reference to a replication initiator and origin refers to those pairs of origin/initiator that are able to support replication.

As used herein, “cognate” with reference to a replication initiator and origin refers to those pairs of origin/initiator that are derived from the same virus.

As used herein, “promoter” refers to a DNA region that controls initiation and rate of transcription. It can contain genetic elements capable of binding regulatory proteins and other molecules, such as RNA polymerase and other transcription factors. Promoter sequences are commonly, but not always, found in the 5′ non-coding region of genes. A promoter can be functional in a variety of tissue types and in several different species, or its function can be restricted to a particular species and/or a particular tissue or cell type. Further, a promoter can be constitutively active, or it can be selectively activated by certain substances (e.g., a tissue-specific factor), under certain conditions (e.g., tumor cell), or during certain developmental stages of the organism (e.g., active in fetus, silent in adult).

As used herein, “tissue-specific” or “cell-specific” promoter refers to a promoter that is capable of driving transcription of a gene in a particular tissue (e.g., lung, liver, breast, or others) or cell (e.g., leukocyte, myocyte, tumor cell, or others) while remaining largely “silent” or expressed at relatively low levels in other tissue or cell types. A tissue-specific or cell-specific promoter can be selective for any tissue or cell-type in a subject. Such promoters are known to one of skill in the art and are described herein. Exemplary of tissue-specific or cell-specific promoters are tumor-specific promoters. It is understood, however, that tissue-specific or cell-specific promoters can have a detectable amount of “background” or “base” activity in those tissues or cells where they are silent. Generally, the promoter is active to a greater degree in a predetermined target cell or tissue as compared to other cells or tissues. For example, the promoter has about or 2, 3, 4, 5, 6, 7, 8, 9, 10, 20, 30, 40, 50, 60, 70, 80, 90, 100, 200, 300, 400, 500, 600, 700, 800, 900 or more activity, i.e. ability to express a nucleic acid sequence operatively linked thereto, in a predetermined tissue or cell than in other tissue or cell types. Thus, a tissue-specific or cell-specific promoter that exhibits some low level activity, e.g., at or about 10% or less in another cell type is still considered to be a tissue-specific or cell-specific promoter if its activity is greater than the activity in a predetermined tissue or cell.

As used herein, a “tumor cell” or “cancer cell” refers to cells that divides and reproduces abnormally because growth and division is not regulated or controlled, i.e. cells that are susceptible to uncontrolled growth. A tumor cell can be a benign or malignant cell. Typically, the tumor cell is a malignant cell that can spread to other parts of the body, a process known as metastasis.

As used herein, a “tumor-specific” promoter is a promoter that is capable of driving transcription of a gene in a tumor cell, while remaining largely “silent” or expressed at relatively low levels in other tissue or cell types, such as for example, in normal cells. For example, a tumor-specific promoter has about 2-fold or 3, 4, 5, 6, 7, 8, 9, 10, 20, 30, 40, 50, 60, 70, 80, 90, 100, 200, 300, 400, 500, 600, 700, 800, or 900-fold or greater than 900-fold, higher activity, i.e. ability to express a nucleic acid sequence operatively linked thereto, in a tumor cell than in a normal cell.

As used herein, the phrases “operatively positioned,” “operatively linked,” “under control,” and “under transcriptional control” mean that a promoter is in a correct functional location and orientation in relation to a nucleic acid sequence to control transcriptional initiation and expression of that sequence.

As used herein, “endogenous” with respect to a promoter refers to a promoter that is naturally associated with a gene or sequence, as may be obtained by isolating a portion of the 5′ non-coding sequences located upstream of the coding segment or exon.

As used herein, “heterologous” with respect to a promoter refers to a promoter that is not normally associated with a nucleic acid sequence in its natural environment.

As used herein, “multicistronic” refers to a transcript with the potential to code for more than one final product.

As used herein, “bicistronic” refers to a transcript with the potential to code for two final products.

As used herein, an “internal ribosome entry site” (IRES) refers to a nucleotide sequence that allows for translation initiation in the middle of a messenger RNA (mRNA) sequence as part of protein synthesis.

As used herein, reference to an IRES-based vector, such as an IRES-based bicistronic vector, refers to a vector that permits the coordinated co-expression of two or more genes using the same promoter in a single nucleic acid molecule vector.

As used herein, a “therapeutic gene” is a gene that encodes a therapeutic product or is capable of producing a therapeutic effect. The product can be nucleic acid, such as a regulatory sequence or gene, or can encode a protein that has a therapeutic activity or effect.

As used herein, “activity” refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, a “bystander gene” refers to a gene that when expressed produces a protein that causes a bystander effect on adjacent tumor cells. A bystander gene can induce toxicity in the cells in which they are expressed and is also capable of inducing cytotoxicity in neighboring cells. Bystander genes include genes that, when expressed, induce cytotoxicity in targeted and neighboring cells by fusion or drug toxicity. Exemplary bystander genes include fusogenic genes and pro-drug converting enzymes.

As used herein, “bystander effect” with reference to tumor therapy refers to secondary effects on adjacent tumor cells and tissues triggered by treatment of a primary target tumor cell with a therapeutic agent. With reference to tumor therapy, the bystander effect can be of known or unknown origin and can be evoked by some forms of gene therapy in which a treatment kills more tumor cells than can be accounted for by the number of cells actually expressing an expressed tumor therapy gene. Exemplary bystander effects are caused by bystander genes that induce toxicity to targeted cells and neighboring cells via fusion or drug toxicity. For example, expression of the prodrug modifying gene HSV-TK is associated with bystander effects, since HSV-TK cells sensitive to ganciclovir (GSV) can be toxic to nearby tumor cells resistant to GSV (Freeman et al. (1993) Cancer Research, 53:5274-5283). Also, bystander effects also are achieved by bystander genes that produce fusogenic proteins.

As used herein, a “fusogenic” protein refers to a protein that effects cell-cell fusion. A fusogenic protein is generally a protein that is normally expressed by a virion to fuse with cell membranes. For purposes herein, the fusogenic protein is encoded by a gene that is synthetically or recombinantly generated based on sequences of known virion fusion proteins. Exemplary fusogenic proteins are described herein and include, for example, influenza fusion peptide for release of the viral genome, HIV gp41 fusion peptide that is responsible for clustering of helper T-cells via cell to cell fusion and the GALV fusogenic protein from Gibbon Ape Leukemia Virus that causes cell to cell fusion and syncytia formation. Exemplary fusogenic proteins include any that have a sequence of amino acids set forth in any of SEQ ID NOS: 38-44, 52, 53-58 or any that are encoded by a sequence of nucleotides set forth in any of SEQ ID NOS: 6, 8, 10, 12, 14, 15, 17, 26-35, or degenerates thereof. Reference to a variant of a fusogenic protein (or encoding nucleic acid molecule) refers to any that exhibits a difference in its sequence (e.g. due to nucleotide or amino acid substitutions or insertions, truncation or deletions or additions), and exhibits or retains fusogenic activity (or encodes a protein that exhibits or retains fusogenic activity). For example, a fusogenic protein variant includes any that exhibits at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of nucleotides set forth in any of SEQ ID NO: 6, 8, 10, 12, 14, 15, 17, 26-35 or degenerate codons thereof, and exhibits fusogenic activity. For example, an exemplary fusogenic protein that is modified is encoded by a sequence of nucleotides set forth in SEQ ID NO:7, 9, 11, 13, 16, 18 or 36 or degenerate codons thereof. Fusogenic protein variants also include any that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of amino acids set forth in SEQ ID NO: 38-44, 52, 53-58, and exhibits or retains fusogenic activity.

As used herein, “fusogenic activity” refers to any protein that when expressed from a cell facilitates fusion between and among neighboring cells. Fusogenic activity can be assessed or determined using cell fusion assays that are well-known to one of skill in the art. For example, exemplary of assays to assess cell fusion include, but are not limited to, visual assays for syncytium formation, qualitative or quantitative detection of syncytia (Corcoran et al. (2006), J. Biol. Chem. 281(42):31778-31789; Dupressoir et al. (2005), Proc. Natl. Acad. Sci. USA 102(3):725-730), a fluorescence dequenching assay (Bagai et al. (1996), J. Cell Biol. 135(1):73-84; Danieli et al. (1996), J. Cell Biol. 133(3):559-569), a dye transfer assay, a content mixing assay whereby aqueous contents of two different cell populations validate fusion (e.g. a cell contains a lacZ gene under the control of the T7 promoter and another cell contains bacteriophage T7 RNA polymerase). Exemplary of such assays are described herein.

As used herein, reference to an “adjunct tumor therapy gene” refers to a gene that when expressed in a tumor cell can result in therapeutic properties or activities, thereby reducing, preventing or ameliorating tumors or cancers. For example, an adjunct tumor therapy gene is one that can augment the recognition and subsequent elimination of tumor cells by effector cells or that can render a tumor cell susceptible to toxic actions of a drug. Exemplary of adjunct tumor therapy genes include, for example, cytokines, chemokines, or suicide genes.

As used herein, the term “suicide gene” refers to a gene that encodes a polypeptide that causes a cell that produces that polypeptide to die. Suicide genes include, but are not limited to, genes that induce apoptosis, toxins, prodrug modifying gene and genes that encode polypeptides that interfere with a signal transduction cascade involved with cellular survival or proliferation.

As used herein, a “prodrug modifying gene” or “prodrug modifying element” or gene encoding a “pro-drug converting enzyme,” or variations thereof refer to a suicide gene that encodes a polypeptide that converts a prodrug to a toxic compound. Exemplary of such a suicide prodrug modifying gene is herpes simplex 1 thymidine kinase gene (HSV-TK), which converts ganciclovir to a toxic nucleotide analog. Another exemplary prodrug converting enzyme is cytosine deaminase (CD) that converts non-toxic 5-fluorocytosine to 5-flurouracil, a potent chemotherapy compound. Exemplary prodrug converting enzymes include any that have the sequence of amino acids set forth in any of SEQ ID NOs: 501 or 502 or any that are encoded by a sequence of nucleotides set forth in SEQ ID NO: 498 or 500, or degenerates thereof. Reference to a variant of a prodrug converting enzyme (or encoding nucleic acid molecule) refers to any that exhibits a difference in its sequence (e.g. due to nucleotide or amino acid substitutions or insertions, truncation or deletions or additions), and exhibits or retains cytotoxic activity (or encodes a protein that exhibits or retains cytotoxic activity). For example, a prodrug converting enzyme variant includes any that exhibits at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of nucleotides set forth in any of SEQ ID NO: 498 or 500 or degenerate codons thereof, and exhibits cytotoxic activity. For example, an exemplary prodrug converting enzyme variant is encoded by a sequence of nucleotides set forth in SEQ ID NO:499, or degenerate codons thereof. Prodrug converting enzyme variants also include any that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to the sequence of amino acids set forth in SEQ ID NO: 501 or 502, and exhibits or retains cytotoxic activity.

As used herein, cytotoxic activity or cytotoxicity with reference to a prodrug converting enzyme or other toxin refers to the quality or property of being toxic to cells such that cells undergo necrosis or lysis, a decrease in cell viability, a decrease in cell growth and/or apoptosis. Assays to assess or measure cytotoxicity are well known to one of skill in the art and include, but are not limited to, assays that measure cell membrane integrity using a vital dye that is normally excluded from healthy cells (e.g. trypan blue or propidium iodide), an MTT (3-(4,5-dimethylthiazol-2-yl)-2,5-diphenyltetrazolium bromide) or MTS (3-(4,5-dimethylthiazol-2-yl)-5-(3-carboxymethoxyphenyl)-2-(4-sulfophenyl)-2H-tetrazolium) assay, the sulforhodamine B (SRB) assay, WST (water-soluble tetrazolium salt) assay or a clonogenic assay.

As used herein, “genetic therapy” or “gene therapy” involves the transfer of a nucleic acid molecule, such as heterologous DNA to certain cells, target cells, of a mammal, particularly a human, with a disorder or condition for which such therapy is sought. The DNA is introduced into the selected target cells in a manner such that the heterologous DNA is expressed and a therapeutic product encoded thereby is produced. Alternatively, the heterologous DNA can in some manner mediate expression of DNA that encodes the therapeutic product, it can encode a product, such as a peptide or RNA that in some manner mediates, directly or indirectly, expression of a therapeutic product. Genetic therapy also can be used to deliver nucleic acid encoding a gene product to replace a defective gene or supplement a gene product produced by the mammal or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product can be modified prior to introduction into the cells of the afflicted host in order to enhance or otherwise alter the product or expression thereof.

As used herein, a detectable label or detectable moiety or reporter protein refers to an atom, molecule or composition, wherein the presence of the atom, molecule or composition can be directly or indirectly measured or otherwise capable of detection. Detectable labels, moieties or reporters can be used included in any of the constructs herein. Detectable labels, moieties or reporters include, for example, chemiluminescent moieties, bioluminescent moieties, fluorescent moieties, radionuclides, and metals. For example, detectable labels, moieties or reporters include, for example, luciferase, green fluorescent protein, red fluorescent protein, colloidal gold, iron, gadolinium, and gallium-67. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition.

As used herein, operably or operatively linked when referring to nucleic acid arranged with regulatory and effector sequences of nucleotides, such as promoters, enhancers, transcriptional and translational stop sites, and other signal sequences refers to the relationship between such nucleic acid, such as DNA, and such sequences of nucleotides so that they function in concert for their intended purposes, e.g., transcription initiates in the promoter and proceeds through the coding segment to the terminator. For example, operative linkage of nucleic acid to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

The term “naked” polynucleotide, DNA or RNA, refers to sequences that are free from any delivery vehicles, complexes or agents that act to assist, promote, or facilitate entry into the cell, including viral particles, liposome formulations, lipofectin or precipitating agents.

As used herein, a “targeting molecule” or “targeting ligand” refers to any protein, polypeptide, or portion thereof that binds to a cell surface molecule, including, but not limited to, proteins, carbohydrates, lipids or other such moiety. Targeting ligands include, but are not limited to growth factors, cytokines, adhesion molecules, neuropeptides, protein hormones and single-chain antibodies (scFv).

As used herein, a nanoparticle refers to a colloidal particle for delivery of a molecule that is microscopic in size, e.g., has an average particle size of between about 1 and 1000 nanometers (nm), such as 1 and 100 nm, and that behaves as a whole unit in terms of transport and properties. Nanoparticles include monolithic nanoparticles (nanospheres) in which the molecule is absorbed, dissolved or dispersed throughout the matrix and nanocapsules in which the molecule is confined to an aqueous or oily core surrounded by a shell-like wall. Alternatively, the molecule can be covalently attached to the surface or into the matrix. Nanoparticles include, for example, liposomes, dendrimers, polymeric micelles, nanocapsules, nanospheres and solid lipid nanoparticles. Generally, nanoparticles are made from biocompatible and biodegradable materials such as natural or synthetic polymers (e.g. gelatin, albumin, polylactides, polyalkylcyanoacrylates) or solid lipids. Nanoparticles include those that contain a targeting molecule attached to the outside.

As used herein, production by recombinant means by using recombinant DNA methods means the use of the well known methods of molecular biology for expressing proteins encoded by cloned DNA.

As used herein, modification or variant is in reference to modification of a sequence of amino acids of a polypeptide or a sequence of nucleotides in a nucleic acid molecule and includes deletions, insertions, and replacements of amino acids and nucleotides, respectively. Modifications also can include post-translational modifications or other changes to the molecule that can occur due to conjugation or linkage, directly or indirectly, to another moiety. Methods of modifying a polypeptide are routine to those of skill in the art, such as by using recombinant DNA methodologies.

As used herein, “CpG motif” refers to nucleotides contains a cytosine “C” followed by a guanine “G”. When these CpG motifs are unmethylated, they can act as immunostimulants based on their recognition by immune cell receptors, such as Toll-like Receptors. Reference to “removal of CpG motifs” means that the C and/or G nucleotides are modified to remove the motif.

As used herein, “humanized” with respect to a nucleic acid molecule means that the nucleic acid molecule has a sequence or a portion of a sequence that resembles or closely resembles a human sequence or the molecule is otherwise made to be more functional in a human cell. For example, codons can be optimized for human usage based on known codon usage in humans in order to enhance the effectiveness of expression of the nucleic acid in human cells, e.g. to achieve faster translation rates and high accuracy.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | proline |
| K | Lys | Lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | asparagine |
| B | Asx | Asn and/or Asp |
| C | Cys | Cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1) and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refer to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, an isokinetic mixture is one in which the molar ratios of amino acids has been adjusted based on their reported reaction rates (see, e.g., Ostresh et al., (1994) Biopolymers 34:1681).

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such conservative amino acid substitutions can be made in accordance with those set forth in TABLE 2 as follows:

TABLE 2

| | Exemplary conservative |
|---|---|
| Original residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Typically, sequence identity is determined by global alignment across the full-length of both compared sequences. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g. terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequence, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Local alignment also can be used when the sequences being compared are substantially the same length.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species, have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. Exemplary of species variants provided herein are primates, such as, but not limited to, human, chimpanzee, macaque, cynomolgus monkey, gibbon, orangutan, or marmoset. Generally, species variants have 70%, 75%. 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly, where sequence identity is greater than 80%.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, leukemia, lymphoma, pancreatic cancer, lung cancer, ovarian cancer, breast cancer, cervical cancer, bladder cancer, prostate cancer, glioma tumors, adenocarcinomas, liver cancer and skin cancer. Exemplary cancers in humans include a bladder tumor, breast tumor, prostate tumor, basal cell carcinoma, biliary tract cancer, bladder cancer, bone cancer, brain and CNS cancer (e.g., glioma tumor), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, treatment of a subject that has a neoplastic disease, such as a cancer including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, a tumor, also known as a neoplasm, is an abnormal mass of tissue that results when cells proliferate at an abnormally high rate. Tumors may show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and blastic tumors.

As used herein, proliferative disorders include any disorders involving abnormal proliferation of cells (i.e. cells proliferate more rapidly compared to normal tissue growth), such as, but not limited to, neoplastic diseases.

As used herein, a “tumor cell” is any cell that is part of a tumor. Typically, the viruses provided herein preferentially infect tumor cells in a subject compared to normal cells.

As used herein, a “metastatic cell” is a cell that has the potential for metastasis. Metastatic cells have the ability to metastasize from a first tumor in a subject and can colonize tissue at a different site in the subject to form a second tumor at the site.

As used herein, “tumorigenic cell,” is a cell that, when introduced into a suitable site in a subject, can form a tumor. The cell can be non-metastatic or metastatic.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject in need of a therapeutic agent.

As used herein, a patient refers to a human subject.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional reagents and instructions for use of the combination or elements thereof. Kits optionally include instructions for use.

As used herein, the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect.

As used herein, “disease or disorder” refers to a pathological condition in an organism resulting from cause or condition including, but not limited to, infections, acquired conditions, genetic conditions, and characterized by identifiable symptoms.

As used herein, “treating” a subject with a disease or condition means that the subject's symptoms are partially or totally alleviated, or remain static following treatment. Hence treatment encompasses prophylaxis, therapy and/or cure. Prophylaxis refers to prevention of a potential disease and/or a prevention of worsening of symptoms or progression of a disease.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

As used herein, a multiple dosage formulation refers to a formulation for use in repeat administrations.

As used herein, an “article of manufacture” is a product that is made and sold. As used throughout this application, the term is intended to encompass delivery agents, such as non-viral nucleic acid vectors, contained in articles of packaging.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

For clarity of disclosure, and not by way of limitation, the detailed description is divided into the subsections that follow.

### B. OVERVIEW OF THE ONCOVECTOR SYSTEM

Provided herein are non-viral oncolytic DNA vector (oncovector) nucleic acid molecules that exhibit replicative and other bystander effects, such as oncovector activities. Existing viral and non-viral gene therapies have been explored for the treatment of solid tumors. For example, oncolytic viruses have been developed that specifically target tumor cells. Oncolytic viruses, however, are live viruses and can be limited by efficiency of viral infection or by the requirement for helper virus or producer cell line. Also, they can be pathogenic to humans or can be highly immunogenic (Chernajovsky et al. (2006) BMJ 332: 170-172). Hence, these therapies are limited due to the immune responses generated to viral vectors and the relatively low efficiency of delivery of non-viral vectors. For example, viral vectors are associated with poor delivery characteristics that cannot be offset by higher doses, since these vectors can be toxic at high concentrations. Also, repeated dosing is also an issue, since viral-based vectors are highly antigenic.

The oncovector system and constructs provided herein are modeled after oncolytic viral therapy, but they overcome these limitations because they are non-viral, yet they autonomously replicate in cells. Although the self-replication and runaway amplification of plasmid DNA can cause cell death by itself, the constructs also exhibit bystander effects on other adjacent cells. The constructs provided exhibit replicative and oncolytic properties, including bystander effects, based on the expression of a first gene product that permits the plasmid to accumulate or be reproduced or replicate in cells and a second gene that results in expression of a bystander product that not only results in killing of the targeted cell but also killing of adjacent cells in a specific and efficient matter. For example, the bystander gene can result in expression of a fusogenic peptide or protein that causes cell-cell fusion. The expression of fusogenic peptides can cause multinucleated syncytia formation and thereby result in spreading to neighboring cells. Other bystander genes, such as pro-drug modifying enzymes, also are contemplated as described elsewhere herein. The constructs also can contain other adjunct therapy genes that exhibit therapeutic activity, such as cytokines, chemokines or other bystander genes. The oncovectors can be engineered to selectively transform disease cells, such as tumor or cancer cells. Hence, the oncovectors can be amplified exclusively in cancer cells, and express proteins that kill targeted and/or adjoining cancer cells effectively and specifically. Thus, also provided herein are methods and uses of treating tumor and cancer cells using the provided oncovector nucleic acid molecules.

For the replicative activity, the nucleic acid constructs provided herein contain genes required for replication minimally containing an origin of replication (origin). Replication of the nucleic acid molecules can be mediated by non-viral (e.g., bacterial components) or viral mechanisms, including retrovirus systems and DNA-based virus systems. Generally, the constructs are episomally expressed and replicate extrachromosomally in host cells such that they are autonomously replicating plasmids (ARPs). Hence, the constructs provided herein typically use a DNA-based virus mechanism of replication, for example, mechanisms derived from polyomaviruses. Such systems permit episomal replication of the nucleic acid molecule and produce a high episomal copy number of expressed genes. In such systems, initiation of replication from the origin requires expression of a compatible or cognate replication initiator protein, which activates the origin. Hence, the origin and the replication initiator make up a replication unit, both of which are required for replication to occur. The replication initiator can be expressed by the host cell, or can be expressed from the same or different construct as the origin is located on. Where the replication initiator is contained on a separate construct, the nucleic acid molecule containing the origin and the nucleic acid molecule containing the replication origin must be delivered into the same cell for plasmid replication to occur. Where the replication initiator is contained on the same nucleic acid molecule as the origin, the nucleic acid molecule is capable of self-replication.

The concurrent use of a replication initiator (e.g. SV40 TAg or TAg) with its cognate origin of replication (e.g. SV40 ori) or variations thereof results in the replication of the plasmid containing the origin of replication. The results of plasmid replication are an increase in the plasmid copy number, an increase in expression of genes expressed by the plasmid, and an increase in the duration of gene expression. The use of an oncovector system is beneficial for gene replacement therapy and cancer therapeutics because of the increase in gene expression and duration of expression that enhances the production of therapeutic proteins.

The oncovector nucleic acid molecules provided herein also contain a second gene that is a therapeutic gene, and in particular a therapeutic gene with anti-tumorigenic activity. For example, the therapeutic gene can be a bystander gene. It is found herein that although some cell viability can be affected by replicative activity alone, to eradicate all the cells in a tumor, a bystander effect is required. Bystander genes can act to spread the killing effect from one targeted cell to several neighboring cells. Exemplary bystander genes in the nucleic acid constructs provided herein can include pro-drug modifying enzymes or fusogenic genes. For example, expression of cytosine deaminase, an enzyme that converts non-toxic 5-Fluorocytosine to the anti-cancer agent 5-fluorouracil (5-FU), can generate sufficient local 5-FU to kill adjoining tumor cells without systemic side effects (Mullen, 1992).

Bystander genes also include genes that express a fusogenic protein, which causes the cell expressing it to fuse with neighboring cells. Hence, in particular provided herein are oncovector nucleic acid molecules that contain a second gene that is a fusogenic gene. The fusogenic activity induces the formation of multinucleated cells that cannot support cell division, thereby killing the cells and rendering the nucleic acid molecule lytic. For example, upon fusion of cells the multinucleated mass of cells will eventually undergo apoptosis and die. Hence, the expression of a fusogenic gene will result in tumor cell-cell fusion and syncytial formation. Once tumor cells form syncytia they are no longer able to divide normally, ultimately resulting in cell death. Also, cell fragments produced from the mass of cells can be phagocytosed by antigen presenting cells (APCs), which can then induce an adaptive immune response against the tumor cells.

The replication component and oncolytic bystander component can be expressed under any constitutive promoter. Typically, the constructs are designed such that the origin is operative and initiates replication in a specific and selective manner so that the construct accumulates in a predetermined cell or tissue, such as, for example, a disease-specific cell or tumor cell. Accumulation of the constructs in specific cells or tissues can be maintained by regulation of the origin directly or indirectly by cellular components, expression of a gene(s) on a separate episomal or non-episomal nucleic acid, or by expression of a gene(s) contained on the same nucleic acid molecule as the ori.

In one example, expression of the cognate replication initiator gene is regulated, thereby indirectly regulating initiation of replication by the origin. For example, the replication initiator can be expressed under the control of a tissue-specific, cell-specific, or cell-cycle dependent promoter so that transcription only will occur where the promoter is active. In cells or tissues where the replication initiator is expressed, replication of the nucleic acid can proceed by binding of the replication initiator to the cognate origin. Hence, the oncovector constructs and system can be expressed in a tissue or cell-specific manner so that the expressed therapeutic specifically targets diseased or tumor cells.

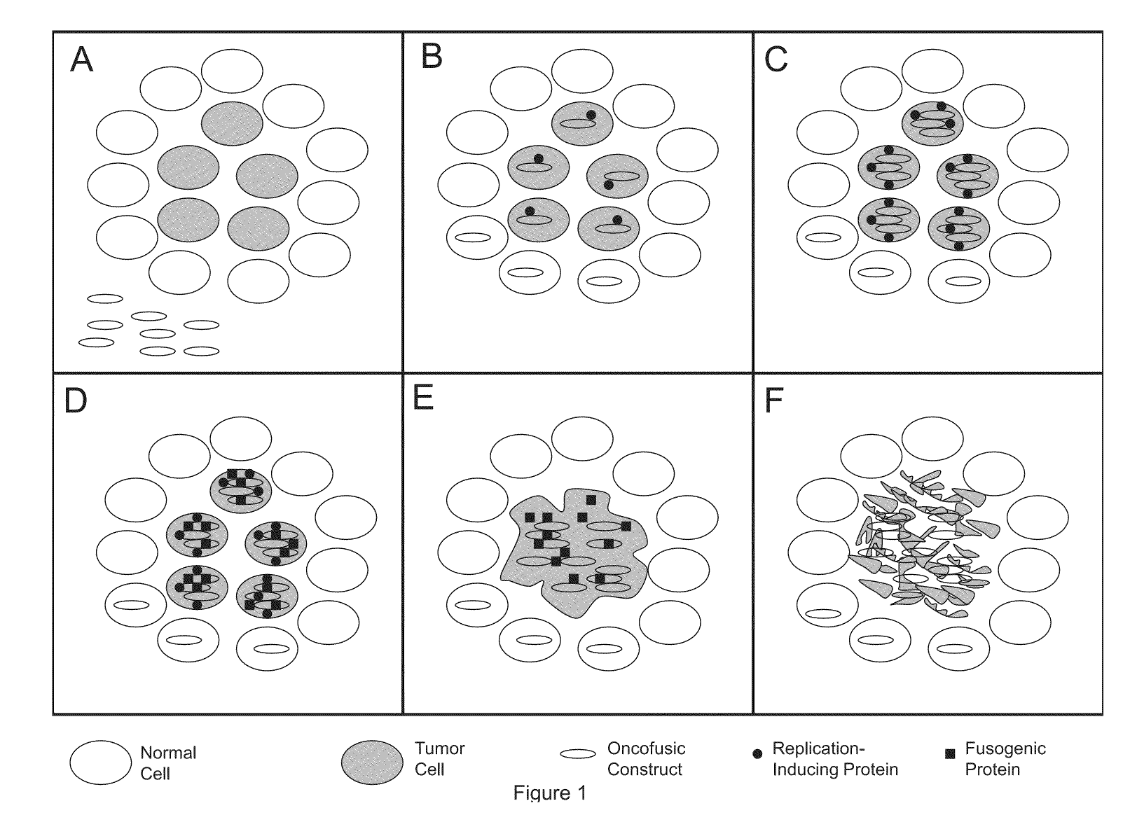

Exemplary of oncovector constructs provided herein are those that accumulate in tumor cells by virtue of cellular deficiency in or mutant for tumor suppressor genes, such as, for example, p53 or retinoblastoma (Rb). Hence, tumor cells that are transformed and that contain a mutated or inactivated p53 or Rb gene can be selectively targeted by the nucleic acid constructs herein. In normal cells containing active tumor suppressor genes, the oncovector nucleic acid molecules are designed to inhibit gene expression of the nucleic acid molecule so that expression of the therapeutic gene in normal cells does not occur. For example, a cell-cycle dependent promoter that is regulated by tumor suppressor genes can be included in the constructs. Hence, in the case of a nucleic acid construct under the control of a cell-cycle dependent protein, such as for example, E2F1, that is regulated by tumor suppressor genes such as p53 and Rb, the construct should accumulate in tumor cells that do not express these genes, and thereby express the fusogenic protein selectively in tumor cells. On the other hand, when such tumor cells come into contact with normal cells, the expression of the tumor suppressor genes in the normal cells should terminate expression of the co-expressed bystander gene, such as a bystander gene encoding a fusogenic protein. This is exemplified in FIG. 1, which shows that upon contact with a non-tumor cell fusion should cease (see FIG. 1). Thus, the constructs can replicate in tumor cells and spread to neighboring tumor cells only.

Upon delivery, replication and expression of the oncovector nucleic acid molecules encoding therapeutic genes, such as fusogenic genes and other genes, results in self-amplification and propagation of anti-cancer activity that can extend beyond the initially targeted cancer cells. For example, the oncovector nucleic acids provided herein can exhibit therapeutic effects upon delivery of the nucleic acid molecules to even a small subset of tumor cells within a tumor site, for example, less than 20%, less than 15%, less than 10% or less than 5% of tumor cells within a tumor site. Hence, the oncovectors nucleic acid molecules provided herein can be used treat cancers and tumors. For example, the oncovector nucleic acid molecules can be used to specifically target cancer cells. In other examples, the oncovector nucleic acid molecules can also treat cancer by effects on non-targeted cancer cells by killing adjoining cancer cells by bystander effects of expressed genes, such as fusogenic genes or other toxic genes. The oncovector nucleic acid molecules can be formulated to facilitate systemic administration.

### C. COMPONENTS OF THE ONCOVECTOR SYSTEM AND RESULTING ONCOVECTORS

Provided herein are oncovector nucleic constructs and systems that are capable of autonomously replicating in cells and that support the expression of cancer or tumor therapeutic proteins that can result in killing of targeted cells and other adjacent cells via bystander effects. Hence, the constructs provided herein are designed to self-replicate. The minimum components of an oncovector system provided herein are a replication initiator and a cognate origin of replication and an oncotherapeutic bystander gene that supports cancer or tumor therapy. The oncotherapeutic bystander gene can be any protein that has a known anti-tumorigenic property or activity and is associated with bystander effects on tumor cells. In some examples, multiple therapeutic genes can be expressed as different transcription units. The replication initiator can be expressed from the same nucleic acid construct as its origin of replication or on a separate nucleic acid construct, or it can be expressed from a stably expressed cell line.

For example, provided herein is an oncovector nucleic acid molecule that contains at least one origin of replication (Ori), at least one replication initiator capable of recognizing the at least one origin of replication, at least one oncotherapeutic bystander gene, and at least one promoter to drive expression of the at least one replication initiator and/or at least one oncotherapeutic bystander gene. In the constructs provided herein, the promoter can be a universal or constitutive promoter, or a tissue-specific, cell-specific or cell-cycle dependent promoter. Generally, the constructs provided herein contain one or more promoters that permit the accumulation of the construct in a desired cell or tissue, such as a tumor cell. The components therein can be in any order.

In the oncovector construct systems provided herein, nucleic acid molecules containing at least two open reading frames (ORFs) are provided where one ORF codes for a replication initiator and the other codes for a oncotherapeutic bystander protein. The ORFs can be on the same or different nucleic acid molecule. The ORF also can be under the control of the same or different promoter. In some examples, the ORF can be included as a complete transcription unit for the coded protein.

For example, provided herein is a nucleic acid molecule that contains at least two ORFs where one ORF codes for a replication initiator and the other codes for a oncotherapeutic bystander protein. Hence, typically, the oncotherapeutic bystander gene is expressed from the same nucleic acid construct as the replication initiator. For example, the nucleic acid construct is multicistronic, such as bicistronic. The oncotherapeutic bystander gene can be expressed from the same or different promoter as the replication initiator. In one example, the genes are expressed from different promoters as different expression cassettes. The promoter in each expression cassette can be the same or different. For example, the oncotherapeutic gene of interest is located as a first transcription unit in a first expression cassette and the replication initiator is in a second transcription unit in a second expression cassette. In other examples, the replication initiator is in the first expression cassette and the oncotherapeutic gene of interest is in the second expression cassette.

In particular examples, provided herein is a nucleic acid molecule where the components are positioned on the nucleic acid in a consecutive order to include: A) a first promoter that controls expression of the first gene containing a first ORF; B) a first ORF coding for a therapeutic protein, for example, a bystander protein; C) a second promoter that controls expression of the second gene containing a second ORF; D) a second ORF coding for a replication initiator; and E) an origin of replication. The first and second ORF can be in reverse order. For example, also provided herein is a nucleic acid molecule where the components are positioned on the nucleic acid in a consecutive order to include: A) a first promoter that controls expression of the first gene containing a first ORF; B) a first ORF coding for a replication initiator; C) a second promoter that controls expression of the second gene containing a second ORF; D) a second ORF coding for a therapeutic protein, for example, a bystander protein; and E) an origin of replication. The first and second promoters can be the same or different.

In another examples, the nucleic acid construct is an IRES-based vector and contains an internal ribosome binding site (IRES) separating the genes of interest such that the replication initiator and the oncotherapeutic bystander gene are expressed under the control of the same promoter. Thus, provided herein is a nucleic acid molecule whereby each ORF is separated by an internal ribosomal entry site (IRES) and is under the control of the same promoter.

In particular examples, provided herein is a nucleic acid molecule where the components are positioned on the nucleic acid in a consecutive order to include: A) a first promoter that controls expression of the genes; B) a first ORF coding for a therapeutic protein, such as a bystander protein; C) an IRES separating the genes of interest; D) a second ORF coding for a replication initiator; and E) an origin of replication. The first and second ORF can be in reverse order. Hence, also provided herein is a nucleic acid molecule where the components are position on the nucleic acid in a consecutive order to include: A) a first promoter that controls expression of the genes; B) a first ORF coding for a replication initiator; C) an IRES separating the genes of interest; D) a second ORF coding for a therapeutic protein, for example, a bystander protein; and E) an origin of replication.

The constructs also can contain reporter genes or other adjunct tumor therapies such as cytokines, chemokines or suicide proteins. For example, reporter genes can be included to facilitate detection of the construct in vitro or in vivo. Reporter genes include, but are not limited to green fluorescent protein (GFP), red fluorescent protein (RFP), and luciferase (Luc). Any one or more of these components can be added to an oncovector construct provided herein so long as the oncovector construct exhibits replication and oncolytic activities. In addition, the constructs provided herein are designed such that they do not exhibit transforming activities. In some examples, the further reporter gene and/or adjunct tumor therapy gene can be expressed as a separate expression cassette from one or both of the replication initiator or the therapeutic gene, for example, bystander gene. In such examples, the reporter gene and/or adjunct tumor therapy gene can be controlled by a promoter that is the same or different from the other promoters in the construct. In other examples, the further reporter gene and/or adjunct tumor therapy gene is co-expressed with one or both of the replication initiator or the bystander gene by the inclusion of an IRES or a further IRES to permit expression from the same promoter.

In addition, the nucleic acid molecules also can contain other regulatory elements, for example, any that control or modulate replication of the nucleic acid molecule or expression of the genes contained therein. Other elements include, but are not limited to, introns, untranslated regions, non-coding regions, polyadenylation signals, antibiotic resistance genes, IRES, other regulatory regions, and others. In particular examples, an internal promoter can be included in a transcription unit to control the expression of the second gene. This can be advantageous in IRES-based vectors where a first and second gene are co-expressed, but the expression of the second gene is not as strong as the first gene. This is exemplified, for example, in the constructs set forth in SEQ ID NOS: 727 and 728, whereby a Rous sarcoma virus (RSV) internal promoter has been included to control expression of the second gene. One skilled in the art will be able to determine the specific necessary elements for each specific oncovector nucleic acid molecule and application.

The constructs provided herein can be in linear or circular form. Typically, for polyoma-based DNA replication the constructs are in circular form. The constructs can be artificially synthesized or can be provided in the backbone of a plasmid or vector. The constructs also can be optimized for human codon usage and/or can be modified to remove CpG motifs to make them less immunogenic. In some examples, the constructs can be delivered as naked DNA. In other examples, the constructs are delivered as nanoparticles, such as in the form of liposomes or wrapped up in DNA condensing agents.

A description of the component parts of the oncovector system is provided below. It is within the level of one of skill in the art to generate and design oncovector systems as described herein. Exemplary oncovector construct systems are provided.

1. Replication Unit

The nucleic acid molecules provided herein are characterized by their ability to replicate extrachromosomally, thereby permitting the episomal expression of hundreds to thousands of copies. Episomal expression systems have principally been developed from several DNA viruses, typically polyomaviruses and herpesviruses, including bovine papilloma virus (BPV) (Sarver, et al., 1981, Mol. Cell. Biol, 1:486-496; Dimaio, et al., 1982, Proc. Natl. Acad. Sci, U.S.A., 97:4030-4034), SV40 (Tsui, et al., 1982, Cell, 30:49914), Epstein-Barr virus (EBV) (Yates, et al., 1985, Nature, 313:812-815; Margolskee, et al., 1988, Mol. Cell. Biol., 8:2837-2847; Belt et al., 1989, Gene, 84:407-417; Chittenden et al., 1989, J. Virol., 63:3016-3025), and BK virus (BKV) (Milansesi, et al., 1984, Mol. Cell. Biol., 4:1551-1560). Episomal replication relies on a viral origin of DNA replication and a virally encoded replication initiator that activates the viral origin and allows the episome to replicate in the host cell. The latter includes the large T antigen for SV40 (SV40 TAg), polyoma and BKV, and EBNA for EBV.

The replication initiator proteins recognize origin-specific sequences, melt the duplex DNA, and act as helicases exposing single-stranded DNA (ssDNA) for replication using host-encoded polymerases and other host DNA replication machinery (Meinke et al. (2006) J. Virol., 80:4304-4312). Generally, the viral origins contain multiple initiator binding sites containing short sequences of 5 or 6 base pairs organized as pairs of inverted repeats. Thus, a replication initiator can bind each origin at multiple sites. For example, the SV40 origin contains four GAGGC (SEQ ID NO:122) binding sites, termed P1 through P4, which supports binding of up to 12 molecules of SV40-TAg on the origin (Meinke et al. (2006) J. Virol. 80:4304-4312).

Accordingly, nucleic acid molecules provided herein contain a polyomavirus origin of DNA replication. Exemplary of origins include, but are not limited to, origins from SV40, BKV, JC virus, lymphotropic papovavirus, and simian agent 12. Any polyomavirus origin of replication that can be shown to drive episomal replication in cells, in particular human cells, is suitable for the nucleic acid molecule constructs provided herein.

DNA replication initiated at these loci is sensitive to control by the replication initiator (e.g., large T antigen or EBNA) of the same virus, and to a similar or lesser extent by large T antigen of other polyomaviruses. For example, the BKV origin drives episomal replication with either BKV large T antigen (BK-T) or SV40 TAg (see e.g., U.S. Pat. No. 6,339,065). Hence, in the presence of a compatible replication initiator, the polyomavirus origin will drive episomal replication. The origin/replication initiator combination should be tested to determine whether they drive replication of the episome. Exemplary of such a test for replication competency is described in Section F and involves transfecting or electroporating a population of cells with a nucleic acid molecule that expresses the large T antigen, or mutant thereof, with a vector containing the proposed origin of replication and then monitor the transfected cells for synthesis of episomal DNA, for example, by DpnI digest followed by quantitive polymerase chain reaction (PCR) or Southern Blot. Alternatively, a single nucleic acid molecule containing both the origin and replication initiator can be transfected to assess self-replication.

In addition to supporting replication, replication initiator proteins also can lead to transformation. For example, both SV40 TAg and BK-T, which are highly homologous, are tumorigenic and can bind to and thereby inactivate wild-type p53 and retinoblastoma (Rb) tumor suppressor genes products (Shin et al., 1975, Proc. Natl. Acad. Sci. USA, 72:4435-4439; Christian, et al., 1987, Cancer Res., 47:6066-6073; Michalovitz, et al., 1987, J. Virol., 61:2648-2654; Hanahan, et al., 1989, Science, 246:1265-1275; DeCaprio, et al., 1988, Cell, 54:275-283; Chen et al., 1990, J. Virol., 64:3350-3357; Chen et al., 1992, Oncogene, 7:1167-1175; Dyson et al., 1990, J. Virol., 64:1353-1356). Therefore, oncovector nucleic acid molecules containing a wild-type replication initiator can confer tumorigenic properties, making such nucleic acid molecules unsuitable for therapeutic purposes. Accordingly, mutations can be made to replication initiator genes to uncouple replication and transformation so that the replication initiators are not tumorigenic. Such mutations are known to one of skill in the art (see e.g., U.S. Pat. No. 6,339,065) and/or can be designed and tested using standard molecular biology techniques known to one of skill in the art. The mutants should be designed to be replication-competent and transformation-negative such that they induce DNA replication, but do not transform the host cell.

Exemplary assays to test for replication include, for example, Southern Blot analysis of Hirt supernatant or total cellular DNA extracted from transient episomal transfectants. Transforming activity of the replication initiator, or mutant thereof, can be tested directly (see e.g., Nakshatri, et al. (1988) J. Virol., 62:4613-21), or cells transfected with an expression vector expressing the replication initiator, or mutant thereof, can be tested for soft agar cloning activity or growth in nude or SCID mice. Alternatively, mutants can be selected based on negative binding studies with wild-type p53 and wild-type Rb. For example, one suitable assay measures binding by generating in vitro translated mutant replication initiator protein and mixing it with wild-type p53 or Rb (e.g., in vitro translated or baculovirus produced) before immunoprecipitation with antisera to p53 or Rb, respectively, to immunoprecipitate these proteins and any replication initiator complexed to them. Western blots to the immunoprecipitate can be developed with antisera to the replication initiator (e.g. large T antigen), which will detect mutant replication initiator that are positive for binding (see e.g., U.S. Pat. No. 6,339,065). In other examples, Rb and p53 wildtype cells can be transfected with plasmids encoding large T antigen mutants and cell lysates can be subject to immunoprecipitation to assess binding of T antigen and mutants thereof to p53 and Rb.

Thus, the nucleic acid molecules provided herein typically contain a polyomavirus origin that is compatible with a replication initiator. In one example, the nucleic acid molecule contains the polyomavirus origin and replication is initiated by a compatible replication initiator expressed by the host cell. In another example, the nucleic acid molecule contains the polyomavirus origin and replication is initiated by a compatible replication initiator, or mutant thereof, encoded on a separate nucleic acid molecule. In an additional example, the nucleic acid molecule is capable of autonomous replication and therefore contains a polyomavirus origin of DNA replication and a compatible replication initiator or mutant thereof, along with the other components of the vector as described herein such as a promoter that drives the expression of the replication initiator and/or a second therapeutic gene, for example, a bystander gene. Typically, in the above examples, the promoter is a promoter that drives expression of the replication initiator, and hence replication, in a tissue-specific or cell specific manner, for example, in a tumor-specific manner. Also, in any of the above examples, the replication initiator can be mutated such that it confers replication but not transformation of cells. The origin also can contain mutations to contain one or more pairs of binding sites for the cognate replication initiator. Exemplary of an origin is the origin of SV40 or mutants thereof and exemplary of a replication initiator is the SV40 TAg, or mutants thereof.

a. SV40 Origin and Mutants Thereof

Exemplary constructs provided herein contain an SV40 origin (ori). The core SV40 ori contains 4 binding sites for the SV40 TAg arranged in a palindromic pattern such that two of the GAGGC (SEQ ID NO:122) motifs are followed by two in the antisense orientation, CTCCG. SEQ ID NO:123 sets forth the SV40 core recognition sequence. SV40 ori can be described as a formula whereby the binding sites are defined as GAGGC and CTCCG, and the flanking regions as “N” (A, C, T or G; see e.g. SEQ ID NO:37, 79 or 124). The SV40 origin of replication, including the early promoter and origin of replication is set forth in SEQ ID NO:113.

In some examples, provided herein are nucleic acid molecule constructs containing an SV40 ori having variations of the core SV40 ori containing one or more pairs of binding sites for SV40 TAg. For example, the SV40 ori can be modified to contain 2, 3, 4, 5, 6, 7, 9, 10 or more binding sites for SV40 TAg. Exemplary of such variants are set forth in SEQ ID NOS: 123 or 124, whereby N can be A, C, T, G. Further variants also are provided herein whereby the N or flanking region can be any nucleotide. Exemplary of such variants are any set forth in SEQ ID NOS: 125-189. It is understood that any of the above sequences, and sequences adapted therefrom to contain further binding sites, can be included in the constructs provided herein.

In some examples, replication activity can be increased by modifying the SV40 origin of replication. For example, elimination of upstream enhancers of the SV40 promoter/enhancer can increase replication activity of the modified SV40 ori by reducing transcription from the SV40 promoter. For example, the sequence of an SV40 promoter containing a 5′ enhancer of SV40 promoter as set forth in SEQ ID NO:114 or 115 can be reduced to that set forth in SEQ ID NO:116 (in addition to Pac1 restriction sites added for ease of identification).

b. SV40 T Antigen and Mutants Thereof

Replication from the SV40 ori is initiated in the presence of the cognate SV40 TAg, or a mutant thereof, expressed from the host cell, or expressed from the same or separate nucleic acid molecule construct. Typically, the nucleic acid molecule is an autonomous replicating plasmid (ARP) and is self-replicating because the SV40 TAg, or mutant thereof, is contained on the same construct as the origin.

The SV40 TAg is a well characterized protein. The SV40 TAg has the amino acid sequence set forth in SEQ ID NO:564 (UniProt No. P03070), and is encoded by a sequence of nucleotides set forth in SEQ ID NO:561. It is a multidomain protein that contains an N-terminal J domain (corresponding to amino acids 1-82 of SEQ ID NO:564), a central origin binding domain (corresponding to amino acids 131-259 of SEQ ID NO:564), and a C-terminal helicase domain (corresponding to amino acids 251-627 of SEQ ID NO:564) (Gai et al., (2004) Cell, 119:47-60). The J domain is dispensable for DNA replication. For replicative activity, the origin binding domain recognizes the SV40 origin, to allow assembly of the SV40 TAg as a double hexamer necessary for distorting and melting of the double-stranded origin DNA via the helicase domain. For example, SV40 TAg multimers bind to 4 motifs of GAGGCG (two forwards and two in reverse orientation) within the core SV40 TAg binding domain (see e.g. SEQ ID NO:123) of the SV40 origin to initiate replication.

Nucleic acid constructs provided herein can contain an SV40 TAg replication initiator that has the nucleotide sequence set forth in SEQ ID NO:561. The encoding SV40 TAg replication initiator also can be modified to be CpG free or to be human codon-optimized. For example, an exemplary SV40 TAg replication initiator that has been modified to be CpG free is set forth in SEQ ID NO:562. An exemplary SV40 TAg replication initiator that has been human codon optimized is set forth in SEQ ID NO:563.

SV40 TAg also exhibits transforming activities. This ability is manifested by the binding of SV40 TAg to one or more tumor suppressors including, but not limited to, p53, Rb and HSP70 (Zalvide et al (1998) Mol. Cell. Biol., 18:1408-1415; Stubdal H et al. (1996) J. Virol., 70:2781-2788; Thompson D et al. (1990) Virology, 178:15-34; Sullivan C S et al. (2002) Microbol Mol Biol Rev. 66:179-202; Ludlow J W et al. (1990) Cell, 60:387-396; Tack et al. (1989) J. Virol., 63:3362-7; Pipas J M et al. (2001) Semin. Cancer Biol., 11:23-30). For example, SV40 TAg can disrupt the inhibitory complex formed between Rb and E2F (discussed in detail below), and this mechanism is important for SV40 TAg-mediated transformation (Zalvide et al (1998) Mol. Cell. Biol., 18:1408-1415). SV40 TAg also is capable of causing transformation of normal cells (Bennoun M et al., (1998) Oncogene, 17:1253-9; Ahuja D et al. (2005) Oncogene, 24:7729-45; Srinivasan A et al. (1989) J. Virol., 63:5459-5463). This activity has been localized to various parts of the protein structure by mutagenesis studies and studies of naturally occurring viral variants.

Typically, SV40 TAg contained in nucleic acid molecules provided herein contain mutations to encode a replication initiator that functions to uncouple transforming activity from replication activity. The resulting mutant SV40 TAg nucleic acid molecules can encode an SV40 TAg containing one or more amino acid replacements compared to the SV40 TAg set forth in SEQ ID NO:564. Generally, such mutants encode a mutant SV40 T Ag that exhibits decreased transforming activity, such as assessed by decreased binding to one or more of p53, Rb and/or HSP70. Decreased transforming activity is about or is less than 80%, 70%, 60%, 50%, 40%, 30%, 20%, 10%, 9%, 8%, 7%, 6%, 5%, 4%, 3%, 2%, 1%, 0.5% or less the transforming activity of wild-type SV40 T Ag, such as is set forth in SEQ ID NO:564. Further, such mutants generally retain at least or about at least 30%, 40%, 50%, 60%, 70%, 80%, 90%, 95% or more of the replication activity (i.e. helicase activity) of wild-type SV40 T Ag, such as is set forth in SEQ ID NO:564. Mutations in a nucleic acid molecule that encodes SV40 TAg amino acid replacements are known to one of skill in the art, or can be readily identified by routine molecular biology techniques. Assays to test for transforming and replication activities of SV-T also are well known to one of skill in the art and exemplary assays are described herein in Section F.

For example, amino acid replacement of SV40 TAg that uncouple p53 binding with helicase activity are known and include mutations D604R and V585R corresponding to amino acid positions set forth in SEQ ID NO:564 (Lilystron W. et al. (2006) Genes and Dev., 20:2373-2382). Other amino acid replacements are also known and characterized. Exemplary of such positions for replacement include, but are not limited to, L17, G18, L19, E20, R21, S22, A23, W24, G25, N26, I27, P28, L29, M30, R31, K32, L103, C105, E107, E108, S112, S189, N366, D367, L368, L369, D370, D402, T434, L435, A436, A437, A438, L439, L440, E441, L442, C443, G444, P453, V585, D604, S677 or S679 corresponding to positions set forth in SEQ ID NO:564. The substitution can be chosen from among any of the other 19 amino acids at that position, so long as SV40 TAg functions are not destroyed. Exemplary amino acid replacement include those that uncouple one or more of HSP70 binding, Rb family binding, and/or p53 binding. Exemplary of such amino acid replacements are set forth in Table 3 below. The sequence identifier number (SEQ ID NO) also is indicated.

TABLE 3
Transformation Mutants

| Amino Acid | SEQ ID | Amino Acid | SEQ ID |
|---|---|---|---|
| replacement | NO | Replacement | NO |
| L19F | 565 | E107L | 566 |
| E107K | 567 | E108L | 568 |
| D402R | 569 | D402E | 570 |
| P453S | 571 | V585R | 572 |
| D604R | 573 | P28S | 574 |
| L103P | 575 | C105A | 576 |
| S112N | 577 | S189N | 578 |
| S677A | | S679A | |

Typically, because different regions of the protein bind to different tumor suppressor genes, combination mutants are provided herein to uncouple replication and transformation induced by SV40 TAg. Such combination mutants are designed to reduce binding to tumor suppressor proteins, for example, p53, Rb and HSP70, yet sill allow SV40 TAg to function as an inducer of replication. The combination mutants can be tested to identify those that bind to and subvert the actions of tumor suppressors in normal cells, and therefore such mutant SV40 TAg provided in the nucleic acid molecules herein are designed so that cellular transformation does not occur in normal cell types. Exemplary of such combination mutants provided herein are set forth in Table 4.

TABLE 4
SV-T Combination Mutants

| | Mutational Effect | | | |
|---|---|---|---|---|
| | HSP70 | Rb Family | P53 | SEQ ID |
| Mutant | binding | Binding | Binding | NO: |
| E107L/E108L | | E107L; E108L | | 579 |
| E107L/E108L/D402R | | E107L; E108L | D402R | 589 |
| E107L/E108L/P453S | | E107L; E108L | P453S | 590 |
| E107L/E108L/V585R | | E107L; E108L | V585R | 591 |
| E107L/E108L/D604R | | E107L; E108L | D604R | 592 |
| L19F/E107L/E108L/D402R | L19F | E107L; E108L | D402R | 593 |
| L19F/E107L/E108L/P453S | L19F | E107L/E108L | P453S | 594 |
| L19F/E107L/E108L/V585R | L19F | E107L/E108L | V585R | 595 |
| L19F/E107L/E108L/D604R | L19F | E107L/E108L | D604R | 596 |
| P28S/E107L/E108L/D402R | P28S | E107L/E108L | D402R | 597 |
| P28S/E107L/E108L/P453S | P28S | E107L/E108L | P453S | 598 |
| P28S/E107L/E108L/V585R | P28S | E107L/E108L | V585R | 599 |
| P28S/E107L/E108L/V604R | P28S | E107L/E108L | V604R | 600 |
| L19F/P28S/L103P/C105A/ | L19F; | L103P; C105A; | V585R | 601 |
| E107L/E108L/V585R | P28S | E107L; E108L | | |
| L19F/P28S/L103P/C105A/ | L19F; | L103P; C105A; | D604R | 602 |
| E107L/E108L/D604R | P28S | E107L; E108L | | |
| L103P/C105A | | | | 580 |
| L103P/E107K | | | | 581 |
| C105A/E107K | | | | 582 |
| C105A/D402E | | | | 583 |
| C105A/V585R | | | | 584 |
| E107K/V585R | | | | 585 |
| E107K/D402E | | | | 586 |
| L103P/D402E | | | | 587 |
| L103P/V585R | | | | 588 |

In other examples, a variant SV40 TAg can include one that encodes an SV40 TAg that contain amino acid insertions or deletions, for example, compared to an SV40 TAg set forth in SEQ ID NO:564 (and encoded by an SV40 TAg set forth in SEQ ID NO:561). Exemplary of such variants are deletion mutations, for example, a nucleic acid molecule that encodes an SV40 TAg deletion mutant set forth in SEQ ID NO:603 or 604.

Corresponding nucleotide mutations in an SV40 TAg, such in an SV40 TAg set forth in SEQ ID NO:561, can be made by standard molecular biology techniques, which are routine to one of skill in the art. For example, wild-type SV40 TAg can be mutated by site-directed mutagenesis, such as by using a QuikChange site-directed mutagenesis kit (Strategene). Briefly, mutagenesis can be performed by designing a pair of oligonucleotides containing the mutations, hybridizing these oligonucleotides to the wild-type sequence, followed by re-synthesis of the gene using PCR. DpnI digestion of the PCR reaction result can eliminate the methylated template DNA. Subsequent transformation of newly PCR-synthesized plasmid into bacteria allows for amplification of the plasmid including the sequence with the desired mutation. After sequence verification, the mutated gene can be subcloned into a backbone or other vector as described herein.

2. Therapeutic Genes

The oncovector constructs provided herein contain a therapeutic gene that supports cancer or tumor therapy. For example, the therapeutic gene can be a bystander gene. The therapeutic gene can be any that encodes a protein that has a known anti-tumorigenic property or activity, for example, one that encodes a protein that is associated with bystander effects on tumor cells. For example, the bystander gene, when expressed, can facilitate cell death of a cell in which it is expressed (the targeted cell) as well as in adjacent or nearby neighboring cells. For example, cell death can occur due to toxic effects caused by expression of the bystander gene, or via apoptosis, such as due to syncytia formation. Exemplary bystander genes are genes that encode fusogenic proteins or genes for pro-drug modifying enzymes.

Typically, the effects on neighboring cells are specific to tumor cells, and not to normal cells. For example, the bystander gene is one that produces a toxic compound that exhibits toxicity to tumor cells, but not to normal cells. In another example, a promoter can be included in the constructs herein such that the bystander genes are expressed in a tissue or cell-specific manner. For example, exemplary oncovector constructs provided herein include a tumor-specific promoter such that the bystander gene is only capable of being expressed in tumor cells, including in neighboring tumor cells. This is exemplified in FIG. 1, which exemplifies the expression of a fusogenic protein as an exemplary expressed bystander gene. Here, the bystander gene is not capable of being expressed in normal cells, and thus only induces fusion and multinucleation, and subsequent apoptosis and cell death, of tumor cells.

Exemplary bystander genes that can be included in the constructs are described below. These include, for example, bystander genes that encode fusogenic proteins or pro-drug modifying enzymes. This description below is exemplary only and is not meant to limit the particular therapeutic gene, for example bystander gene, that can be included in the constructs provided herein.

a. Gene Encoding a Fusogenic Protein

Constructs provided herein contain a fusogenic gene, which is a gene encoding a protein that causes fusion of two membranes. Protein-mediated membrane fusion is a key step in cellular processes such as exocytosis, protein trafficking, fertilization, and enveloped virus infection. Most fusogenic proteins known in the art are viral glycoproteins used to infect host cells, while some eukaryotic fusogenic proteins also are known (see Table 5). Some fusogenic proteins must be expressed from within both adjacent cells (e.g., eukaryotic SNARE proteins), while other fusogenic proteins require other proteins to function. Generally, fusogenic genes used in the constructs provided herein are any that function without the need for other proteins. Some of these “stand alone” fusogenic genes encode proteins that function as a result of mutations, which eliminates the need for additional proteins. Hence, nucleic acid molecules in the constructs provided herein also include those that encode modified fusogenic proteins due to amino acid substitutions, insertions and/or deletions to increase their fusogenic activity.

The groups of fusogenic proteins provided herein achieve membrane fusion by various mechanisms. For viral fusion proteins, there are three classes of fusion proteins: Class I viral fusion proteins have a prominent alpha-helical coiled region that forms a 6 helical bundle structure (forms a pore); class II viral fusion proteins have an alpha structure that is different from class II; and class III viral fusion proteins have combined features of class I and class II. All classes of viral fusion proteins are associated with similar conformational changes to achieve fusion. In general, in response to a trigger, they insert the hydrophobic fusion peptide or loops to attach to the target membrane, and then a fold-back occurs bringing the membranes together. For example, viral F and G proteins sometimes, but not always, work together where the G protein binds to a target cell receptor and the viral F protein undergoes a conformational change. In response to a trigger, F proteins insert the hydrophobic fusion peptide (or fusogenic peptide, described below) into the target membrane followed by a fold-back mechanism which brings the two membranes together. In contrast, reovirus FAST proteins are nonstructural, transmembrane proteins that induce membrane fusion by a different mechanism of action. Because of their low molecular masses (ranging from 10 kDa to 15 kDa) and lack of typical fusion protein motifs, FAST proteins most likely induce membrane fusion through a mechanism that is different from the mechanism described above for F proteins. Eukaryotic SNARE proteins form a four-bundle structure of β-helical coils. Membrane fusion occurs via a zippering mechanism, and proteins must be present on both opposing membranes.

Generally, for viruses a number of genes can be involved in virus-cell or cell-cell fusion. For example, for vaccinia virus (VACV), 8 genes are involved: A16, A21, A28, H2, L5 and J5. Genes A16, G9 and J5 also appear to be distantly related. For Herpes, at least 3-4 genes are involved in fusion: gH, gL, gQ1 and gQ2. For retroviruses and paramyxoviruses/paraninfluenza viruses, 2 genes typically are involved but instances of single genes taking over the function of both has been seen.

Fusogenic proteins encoded by genes contemplated for use in the oncovector constructs provided herein can be derived from viral or eukaryotic fusion proteins. Exemplary genes encode viral fusogenic proteins that include, but are not limited to, viral glycoproteins (F and G proteins, such as SV5F and VSV-G) and reovirus FAST proteins (e.g., Avian Reovirus p10, Reptilian Reovirus p14, and Baboon Reovirus p15). Eukaryotic fusogenic proteins include, but are not limited to, FF proteins (e.g., EFF-1, AFF-1), tetraspanin proteins, and SNARE proteins (e.g., Syntaxin, SNAP25, Synaptobrevin). Fusogenic proteins also include fusogenic peptides that can be activated by tumor specific proteases (see e.g. Walker et al. (1994) Protein Engineering, 7:91-7; Abi-Habib et al. (2006) Mol. Cancer. Ther., 5:2556-62). Fusogenic proteins also include tumor specific protease activated toxins (see e.g. Tcherniuk et al. (2005) Mol. Ther., 11:196-204).

Exemplary of genes for use in the constructs herein include any that encode a fusogenic protein set forth in Table 5. The Tables sets forth exemplary DNA sequences. The fusogenic genes can be modified for use in the constructs herein, such as to remove CpG motifs or for human codon optimization. Also, as described elsewhere herein, the sequences can be designed to contain terminal restriction site sequences for purposes of cloning into vectors.

TABLE 5

| | | SEQ ID |
|---|---|---|
| | SEQ ID | NO. |
| | NO. | (amino |
| Fusogenic Protein | (nucleotide) | acid) |
| Glycoproteins of Enveloped Viruses (F and G Proteins) | | |
| VSV-G (Vesicular stomatitis | 6 | 38 |
| virus G protein) | 7 | |
| | (CpG free) | |
| | 70 or 71 | |
| | (terminal | |
| | restriction sites) | |
| MV (Measles virus) F | | |
| SIV (Simian immunodeficiency | | |
| virus) F | | |
| HIV (Human immunodeficiency | | |
| virus) 1 + 2 F | | |
| MuLV (Murine leukemia virus) F | | |
| Chicken LV Env Protein | 26 | 52 |
| (NM_001099360) | 91 | |
| | (terminal | |
| | restriction sites) | |
| SER virus F | 27 | 53 |
| | 92 | |
| | (terminal | |
| | restriction sites) | |
| NDV (Newcastle disease virus) F | | |
| GALV (Gibbon ape leukemia | 15 | 43 |
| virus) F | | |
| | 16 | |
| | (CpG free) | |
| | 80 or 81 | |
| | (terminal | |
| | restriction sites) | |
| SV5 (Simian virus 5) F | 17 | 44 |
| | 18 | |
| | (CpG free) | |
| | 82 or 83 | |
| | (terminal | |
| | restriction sites) | |
| PPRV-F | 28, 29 | 54 |
| | 93 or 94 | |
| | terminal | |
| | restriction sites) | |
| Mumps F | | |
| Sendai virus F | | |
| HPIV 1, 2 and 3 (Human | | |
| parainfluenza virus types 1, | | |
| 2, and 3) F | | |
| CDV (Canine distemper virus) F | | |
| R'Pest F | | |
| SV41 (Simian virus 41) F | | |
| HRSV (Human respiratory syncytial | | |
| virus) F | | |
| Human endogenous retroviral-3 | 35 | 58 |
| (HERV-3) | 36 | |
| | (CPG free) | |
| | 100 or 101 | |
| | (terminal | |
| | restriction sites) | |
| Reovirus FAST proteins | | |
| Avian Reovirus p10 | 8 | 39 |
| | 9 | |
| | (CpG free) | |
| | 72 or 73 | |
| | (terminal | |
| | restriction sites) | |
| Avian Reovirus p10 (S1133 | 10 | 40 |
| variant V68I) | 11 | |
| | (CpG free) | |
| | 74, 75 | |
| Reptilian Reovirus p14 | 12 | 41 |
| | 13 | |
| | (CpG free) | |
| | 76 or 77 | |
| | (terminal | |
| | restriction sites) | |
| Baboon Reovirus p15 | 14 | 42 |
| | 78 | |
| | (terminal | |
| | restriction sites) | |
| Eukaryotic Membrane Fusion | | |
| Proteins | | |
| EFF-1 | | |
| AFF-1 | | |
| Tetraspanin Proteins | 30 | 55 |
| | 95 | |
| | (terminal | |
| | restriction sites) | |
| Yeast G Protein | | |
| Syncytin 1 | 31, 32 | 56 |
| | 96 or 97 | |
| | (terminal | |
| | restriction sites) | |
| Syncytin 2 | 33, 34 | 57 |
| | 98, 99 | |
| | (terminal | |
| | restriction sites) | |
| Syntaxin (SNARE) | | |
| SNAP25 (SNARE) | | |
| Synaptobrevin (SNARE) | | |

i. Exemplary Viral Fusogenic F Proteins and Variants

Viral fusogenic F proteins contemplated for use in the oncovector constructs provided herein include, but are not limited to, Paramyxo/Parainfluenza F proteins such as, for example, SER virus F protein, SV5F, NDV F, Mumps F, Measles F, and variants and/or portions thereof that exhibit fusogenic activity. In viral fusogenic F proteins, the F protein is synthesized as an inactive precursor, F0, that is posttranslationally cleaved by a host protease into two disulfide-linked subunits called F1 and F2. The cleavage of F is required for virus-cell and cell-cell membrane fusion and also for viral infection. A well-conserved hydrophobic domain (fusogenic peptide) at the amino terminus of F1 is exposed by the cleavage and is involved in the fusion event. The F2 subunit contains that TM region. Three heptad repeat (HR) domains are found in the F1 ectodomain. HR1 is immediately adjacent to the carboxyl terminus of the fusion peptide, while HR2 is close to the transmembrane (TM) domain; HR3 is located between the HR1 and HR2 domains. The F1 subunits alpha-helical hydrophobic fusion peptide is inserted into the membrane, and an activation causes the F protein to ratchet the membranes together (fold-back). In some viruses, the hemagglutinin-neuraminidase protein (HN) is the attachment protein, which is often required to mediate the fusion of the F gene. Hence, mutations can be included that encode a fusogenic protein that alleviate the necessity for the HN protein. Also, in some viruses, the F2 subunit often contains an inhibitory F2 COOH-terminal R peptide that prevents cytotoxicity of the infected cell. The cytoplasmic tail (CT) domain of viral F proteins also has been shown to play a regulatory role in membrane fusion.

Viral fusion proteins can vary in one or more of the above mechanisms. For example, exemplary of a fusogenic gene for use in the constructs herein are nucleic acid molecules that contain the gene that encodes SV5F or variants thereof. The gene that encodes SV5F corresponds to nucleotides 4530-6118 of the nucleotide sequence set forth in SEQ ID NO:490 (GenBank Accession No. NC_{—}006430). A sequence of the gene is set forth in SEQ ID NO:17 and encodes an amino acid sequence set forth in SEQ ID NO:44. Genes that encode SV5F also can include modified forms thereof. For example, a CpG modified sequence encoding SV5F is set forth in SEQ ID NO:18 and 82. Other modified forms are described below. The SV5F fusion protein is fusogenic, and deletion of the CT ablates the fusogenic activity. SV5F is active at neutral pH. Strains of SV5F also have differing requirements for HN for activity. For example, encoded SV5F from strain W3A (SEQ ID NO:44) exhibits fusogenic activity without coexpression of HN, whereas SV5F from strain WR requires coexpression of HN for fusion activity. SV5F strains W3A and WR differ by three amino acid residues corresponding to positions 22, 443, and 516 of SEQ ID NO:44 (the W3A SV5F protein contains residues P22, S443, and V516 whereas the WR SV5F protein contains L22, P443, and A516).

In a further example, exemplary of a fusogenic gene for use in the constructs herein are nucleic acid molecules that contain a gene that encodes Reptilian Reovirus p14 (RRVp14), and Baboon Reovirus p15 (BRVp15). The gene that encodes Reptilian Reovirus p14 corresponds to nucleotides 25-402 of the nucleotide sequence set forth in SEQ ID NO:548 (GenBank Accession No. DD038189). A sequence of the gene is set forth in SEQ ID NO:12 or 13 and encodes an amino acid sequence set forth in SEQ ID NO:41.

In a further example, exemplary of a fusogenic gene for use in the constructs herein are nucleic acid molecules that contain a gene that encodes Baboon Reovirus p15 (BRVp15). The gene that encodes Baboon Reovirus p15 corresponds to nucleotides 25-447 of the nucleotide sequence set forth in SEQ ID NO:489 (GenBank Accession No. AF406787). A sequence of the gene is set forth in SEQ ID NO:14 and encodes an amino acid sequence set forth in SEQ ID NO:42.

In one example, exemplary of a fusogenic gene for use in the constructs herein are nucleic acid molecules that contain a gene that encodes the Avian Reovirus p10 (ARVp10) fusogenic protein such as set forth in SEQ ID NO:8 (GenBank Accession No. AY395797) or 9 and encoding a fusion protein set forth in SEQ ID NO:39 or variants thereof. For example, a variant of ARVp10, derived from a natural mutation in strain ARV-S1133 (SEQ ID NO: 525; Genbank AF330703). The gene that encodes a variant of Avian Reovirus p10, derived from a natural mutation in strain ARV-S1133 resulting in a V68I amino acid substitution, corresponds to nucleotides 25-321 of the nucleotide sequence set forth in SEQ ID NO:525 (GenBank Accession No. AF330703). This mutant V68I has been observed to have a greater fusogenic behavior. A nucleic acid sequences encoding the ARV-S1133 variant is set forth in SEQ ID NO:10 or 11 (CpG free) and encodes a fusogenic protein set forth in SEQ ID NO:40.

In a further example, exemplary of a fusogenic gene for use in the constructs herein are nucleic acid molecules that contain a gene that encodes VSV-G fusion protein. The gene that encodes VSV-G corresponds to nucleotides 1420-2955 of the nucleotide sequence set forth in SEQ ID NO:524 (GenBank Accession No. AJ318514). A sequence of the gene is set forth in SEQ ID NO:6 or 7 and encodes an amino acid sequence set forth in SEQ ID NO:38.

In another example, exemplary of a fusogenic gene for use in the constructs herein are nucleic acid molecules that contain the gene that encodes a SER Virus fusion protein or variants thereof. A sequence of the gene is set forth in SEQ ID NO:27 and encodes an amino acid sequence set forth in SEQ ID NO:53. Further variants or modified forms are contemplated. The SER Virus is not fusogenic because it contains extra amino acids. A SER Virus lacking the cytoplasmic tail portion, however, is fusogenic. Mutants also can be generated that do not require HN. Also, it is active at neutral pH.

Also provided herein are oncovector constructs containing genes encoding mutant viral F proteins. The mutations in the F protein generally enhance or increase the fusogenic activity of the encoded viral protein. For example, the included genes can encode F proteins that can contain mutations in the N-terminal fusogenic peptide of the F1 subunit that is involved in penetrating the membrane. In another example, the included genes can encode F proteins that do not require the hemagglutinin-neuraminidase protein (HN) is the attachment protein, which is often required to mediate the fusion of the F gene. In a further example, genes can encode fusogenic proteins that have deletions of the cytoplasmic tail (CT) that enhance fusogenic activity in some F proteins.

For example, F proteins with mutations in the N-terminal fusogenic peptide portion of the F1 subunit can be used. This region is a twenty amino acid hydrophobic α-helix which penetrates the lipid membrane and anchors it. Exemplary twenty amino acid fusogenic F1 peptide sequences are set forth in SEQ ID NOs:59-69 for SV5F, HPIV2, SV41, MUMPS, MV, R PEST, CDV, HPIV1, HPIV3, NDV and Sendai F proteins, respectively (Horvath and Lamb, J Virol 66:2443-55 (1992)). Glycines, for example, are known to be disruptive to α-helixes. Thus, Gly to Ala substitutions, for example, can increase the fusogenic activity of F proteins by improving the α-helix structure (Bagai and Lamb, Virology 238:283-90 (1997); Russell et al., J Virol 78:13727-42 (2004)). Any fusogenic gene encoding a viral F protein with a Gly to Ala substitution or plurality of Gly to Ala substitutions in the fusogenic peptide can be used in the oncovector constructs provided herein. For example, a gene encoding an SV5F protein variants with one or more Gly to Ala substitutions in the fusogenic peptide portion of the F protein (corresponding to amino acids 103-122 of the amino acid sequence set forth in SEQ ID NO:44) can be used. In particular, genes encoding SV5F protein variants with Gly to Ala substitutions at positions 3, 7 and/or 12 of the twenty amino acid fusogenic peptide (corresponding to Gly to Ala substitutions at positions 105, 109 and 115 of the SV5F protein set forth in SEQ ID NO:44) and all possible combinations of these mutations, can be used. Exemplary SV5F protein variants (nucleic acid and encoding protein) are presented in Table 6.

TABLE 6
SV5F Fusogenic Peptide Variants

| | Nucleic Acid | | |
|---|---|---|---|
| | SEQ ID NO | | |
| | | terminal | Protein |
| | | restriction | SEQ ID |
| | | site | NO |
| WT | 17, 18 | 82, 83 | 44 |
| G105A | 19 | 84 | 45 |
| G109A | 20 | 85 | 46 |
| G114A | 21 | 86 | 47 |
| G105A/G109A | 22 | 87 | 48 |
| G105A/G114A | 23 | 88 | 49 |
| G109A/G114A | 24 | 89 | 50 |
| G105A/G109A/G114A | 25 | 90 | 51 |

As noted above, some fusogenic proteins require other proteins to function. For example, some F proteins require coexpression the Hemagglutinin-neuraminidase protein (HN) attachment protein to mediate membrane fusion activity. However, variations among viral strains and mutations known in the art within certain F proteins can alleviate the necessity for the HN protein. Nucleic acids encoding such mutant viral F proteins or strain variants can be used in the oncovector constructs provided herein. The encoded mutations can be within the fusogenic peptide or within the F2 subunit COOH-terminal region (R peptide). Examples of genes encoding fusogenic proteins containing mutations that negate the requirement for coexpression of the HN protein to induce membrane fusion include, but are not limited to, genes encoding an SER virus F mutant (L539A/L548A, L548V, L548G corresponding to positions set forth in SEQ ID NO:53); NDV mutant (L289A); MuLV (R peptide mutations); and GALV (R peptide mutations). The need for HN coexpression also varies among fusogenic proteins from different strains.

As noted, the cytoplasmic tail (CT) domain of viral F proteins also has been shown to play a regulatory role in membrane fusion. For example, F-protein CT truncations (−CT) in Newcastle disease virus (NDV) result in highly reduced fusogenic activity. CT truncations in SV5F proteins abolish fusogenic activity. CT truncations also can enhance fusogenic activity in some F proteins. For example, truncations or mutations in the CT domain of the MV, SIV, HIV 1 and 2, MuLV, and SER virus F proteins were found to enhance fusogenic activity.

b. Gene Encoding a Prodrug Converting Enzyme

Provided herein are oncovector constructs containing genes encoding prodrug modifying elements. A pro-drug is a compound that, on administration, must undergo chemical conversion by metabolic processes before becoming the pharmacologically active drug for which it is a prodrug. Pro-drug modifying elements carry out this conversion. For example, the herpes simplex 1 thymidine kinase gene (HSV-TK) which can covert the pro-drug ganciclovir (GCV) into a toxic metabolite, thereby killing the cell which expresses the TK gene. If the expression of HSV-TK is coupled to the differential expression of the tumor-specific promoter, such as those provided herein, this gene activity can result in selective cellular toxicity of the tumor cells. This strategy can be employed within the vector containing both the fusogenic gene and the TAg, or in a self-replicating vector where TAg is expressed along with HSV-TK.

Such prodrug converting enzymes include, but are not limited to the HSV-TK polypeptide, which converts ganciclovir to a toxic nucleotide analog. An exemplary sequence of HSV-TK is set forth in SEQ ID NO: 498 and encodes a protein set forth in SEQ ID NO:501. A codon-optimized and CpG free HSV1-TK gene is set forth in SEQ ID NO:499. In particular, provided herein are nucleic acid constructs that contain a synthetic TK transcription unit containing a cell cycle dependent promoter, an HSV1-TK gene that has been codon optimized and is CpG free, a synthetic pA sequence (see e.g. SEQ ID NO:497). The sequence set forth in SEQ ID NO:497 also contains restriction site sequence such that digestion with Pf1F1 and Bg12 can permit insertion into a backbone construction provided herein, and in particular the backbone intermediate 4 vector. It is understood, however, that the particular sequence can be adapted and modified for cloning into any desired plasmid or vector using standard recombinant DNA techniques.

Another exemplary pro-drug modifying enzyme is cytosine deaminase (CD), which converts the non-toxic nucleotide analog 5-fluorocytosine into a toxic analog, 5-fluorouracil (Yazawa et al., 2002). An exemplary gene sequence for CD is set forth in SEQ ID NO:500 and encodes a protein set forth in SEQ ID NO:502. Another exemplary prodrug-modifying enzyme is cytochrome p450, which converts certain aliphatic amine N-oxides into toxic metabolites.

3. Promoter

Constructs provided herein contain one or more promoters to drive expression of nucleic acid sequences contained therein. Typically, the promoter is operatively linked to one or more than one nucleic acid molecule. Where more than one promoter is used in the construct, the promoter can be the same or different. The promoter can be endogenous or heterologous to the gene or sequence.

For example, a construct can contain a first promoter operatively linked to a first nucleic acid sequence (e.g., replication initiator) and a second promoter operatively linked to a second nucleic acid sequence (e.g. fusogenic sequence). In some cases, a construct also can contain a third promoter operatively linked to a third nucleic acid sequence (e.g. an adjunct tumor therapeutic gene) and so on.

In other examples, constructs provided herein can contain a single promoter that drives the expression of one or more nucleic acid molecules. Such promoters are said to be multicistronic (bicistronic or tricistronic, see e.g., U.S. Pat. No. 6,060,273). In instances of multicistronic expression, it is contemplated that internal ribosome entry site (IRES) can be used, which aid in the initiation of translation internally.

A promoter used in the constructs provided herein can be functional in a variety of tissue or cell types and in several different species or organisms. Alternatively, its function is restricted to a particular species and/or a particular tissue or cell type. Further, a promoter can be constitutively active, or it can be selectively activated in certain cells or tissues, for example, due to the presence of a cell-type (e.g. tumor) or tissue-specific factor. Such promoters are known to one of skill in the art. Papadakis et al. (Current Gene Therapy (2004) 4:89-113) describes exemplary tissue and disease-specific promoters, including tumor-specific promoters.

Generally, promoters contained in the constructs provided herein are tissue-specific or cell-specific promoters. Such promoters include, but are not limited to, those that are active in heart, lung, esophagus, muscle, intestine, breast, prostate, stomach, bladder, liver, spleen, pancreas, kidney, neurons, myocytes, leukocytes, immortalized cells, neoplastic cells, tumor cells, cancer cells, duodenum, jejunum, ileum, cecum, colon, rectum, salivary glands, gall bladder, urinary bladder, trachea, larynx, pharynx, aorta, arteries capillaries, veins, thymus, mandibular lymph nodes, mesenteric lymph node, bone marrow, pituitary gland, thyroid gland, parathyroid glands, adrenal glands, brain, cerebrum, cerebellum, medulla, pons, spinal cord, sciatic nerve, skeletal muscle, smooth muscle, bone, testes, epididymides, prostate, seminal vesicles, penis, ovaries, uterus, mammary glands, vagina, skin, eyes or optic nerve.

Exemplary cell-specific promoters include, for example, endothelial nitric oxide synthase (eNOS) promoter expressed in endothelial cells (Guillot, P. V. et al. (199) J. Clin. Invest. 103:799-805); vascular endothelial growth factor (VEGF) receptor (flk1) promoter expressed in endothelial cells (Kappel et al. (1999) Blood, 93:4282-4292); insulin promoter expressed in beta cells of the pancreas (Ray et al., J. Surg. Res. (1999) 84:199-203); promoter of gonadotropin-releasing hormone receptor gene expressed in cells of the hypothalamus (Albarracin et al. (1999) Endocrinology, 140:2415-2421); matrix metalloproteinase 9 promoter expressed in osteoclasts and keratinocytes (Munant et al., (1999) J. Biol. Chem., 274:5588-5596); promoter of parathyroid hormone receptor expressed in bone cells (Amizuma et al. (1999) J. Clin. Invest., 103:373-381); and dopamine beta-hydroxylase promoter expressed in noradrenergic neurons (Yang et al., (1998) J. Neurochem. 71:1813-1826).

Cell-specific promoters also include tumor-specific promoters. Tumor-specific promoters include, for example, cell-cycle dependent promoters that are regulated by cell cycle genes. Typically, tumor cells have runaway cell cycle, and thus cell cycle-dependent gene promoters are highly active in tumor cells versus normal cells in which these promoters are repressed. For example, the tumor suppressor genes p53 and retinoblastoma (Rb) proteins are deleted or mutant in greater than 50% of human cancers, but not in normal cells. It is for this reason that elements of the cell-cycle dependent gene promoters are being utilized as differentially expressing promoters for expression in tumor cells and repression in normal cells. This phenomenon has been observed and/or utilized for various oncolytic viruses (Jounaidi et al. (2007) Curr Cancer Drug Targets, 7:285-301; Markert et al. (2000) Gene Ther., 7:867-74).

Other tumor-specific promoters are known to those of skill in the art (see e.g., Hardcastle J et al. (2007) Current Cancer Drug Targets, 7: 181-189. These include, but are not limited to, promoters that have been derived from genes that encode tyrosinase (allowing for targeting to melanoma; see e.g., Vile, R. G. and Hart, (1993) Cancer Res., 53:962-967; Vile, R. G. and Hart I. R., (1994) Ann. Oncol 5 (Suppl. 4):S59-S65; Hart, I. R. et al., (1994) Curr. Opin. Oncol., 6:221-225); c-erbB-2 oncogene (targeting to breast, pancreatic, gastric and ovarian cancers; see e.g., Hollywood D and Hust H (1993) EMBO J, 12:2369-2375); carcinoembryonic antigen (CEA) (targeting to lung and gastrointestinal malignancies, including colon, pancreatic and gastric cancer; see e.g., Thompson, J. A. et al. (1991) J. Clin. Lab. Anal., 5:344-366; Osaki T et al. (1994) Cancer Res., 54:5258-5261); DF3/MUC1 (targeting to breast cancer; see e.g., Abe M and Kufe D (1993) Proc. Natl. Acad. Sci. USA, 90:282-286); Manome Y et al. (1995) Gene Ther. 2:685, A051; Chen L et al. (1995) J. Clin. Invest., 96:2775-2782); prostate specific antigen (PSA) (targeting to prostate cancer; see e.g., Lundwall, A (1989) Biochem. Biophys. Res. Commun., 161:1151-1156); and alpha-fetoprotein (AFP) (targeting to hepatocellular carcinoma; see e.g., Arbuthnot P et al. (1995) Hepatology, 22:1788-1796; Ido et al. (1995) Cancer Res., 55:3105-3109); L-plastin (LP-P) (targeting to epithelial-derived tumors; see e.g., Chung et al. (1999) Cancer Gene Ther., 6:99-106); α-lactalbumin (ALA) (targeting to breast cancer; see e.g., Anderson et al. (2000) Cancer Gene Ther., 7:845-852); midkine (MK) (targeting to pancreatic cancer; see e.g., Yoshida et al. (2002) Anticancer Res., 22:117-120); cyclooxygenase-2 (COX-2) (targeting gastrointestinal cancer; see e.g., Yamamoto et al., (2001) Mol. Ther., 3:385-394; Wesseling et al. (2001) Cancer Gene Ther., 8:990-996); probasin (ARR2PB) (targeting prostate cancer; see e.g., Lowe et al. (2001) Gene Ther., 8:1363-1371; Rubinchik et al. (2002) Gene Ther., 8:247-253); hypoxic response elements (HRE) (not tissue specific; see e.g., Dachs et al. (2000) Eur. J. Cancer, 36:1649-1660; Ruan H et al. (2001) Curr Opin Invest Drugs, 2:839-843); hTERT (not tissue specific; see e.g., Gu et al. (2000) Cancer Res., 60:5359-5364; Koga et al. (2000) Hum. Gene Ther., 11:1397-1406; Lin et al. (2002) Cancer Res., 62:3620-3625; Majumdar et al. (2001) Gene Ther., 8:568-578; Fujiwara T et al. (2007) Current Cancer Drug Targets, 7: 191-201); promoters that target the angiogenic tumor vasculature, for example, flt-1, flk1/KDR, E-selectin, endoglin, ICAM-2, preproendothelin 1 (PPE-1) (see e.g., Jaggar et al. (1997) Hum. Gene Ther., 8:2239-2247; Walton et al. (1998) Anticancer Res., 18:1357-1360; Savontaus et al. (2002) Gene Ther., 9:972-979; Bauerschmitz et al. (2002) Cancer Res., 62:1271-1274; Velasco et al. (2001) Gene Ther., 8:897-904; Varda-Bloom et al. (2001)); prolactin (PRL) (targeting pituitary tumors; see e.g., Southgate et al. (2000) Endocrinology, 141:3493-3505); osteocalcin 2 (targeting osteosarcoma; see e.g., Barnett et al. (2002) Mol. Ther., 6:377-385); survivin (Van Houdt et al., (2006) J Neurosurg. 104(4):583-592); CXCR4 tumor-specific promoters (Ulasov I V et al. (2007) Cancer Biology and Therapy, 65:675-689; Zhu, Z B et al. (2006) J Thorac. Oncol., 1:701-711); and human papilloma virus 16 (Delgado-Enciso et al. (2007) J of Gene Medicine).

The level of expression of a gene under the control of a particular promoter can be modulated by manipulating the promoter region. For example, different domains within a promoter region can possess different gene-regulatory activities. For example, promoters typically bind one or more transcription factors that are able to regulate transcription. Thus, promoters can be modified to alter the configuration of regulatory binding regions, such as for transcription factors and/or can be made to have specific regions deleted. Such mutational and deletional analysis can be rationally or empirically performed and the resulting constructs tested by one of skill in the art. For example, the various modified promoter constructs can be tested in a construct whereby the modified promoter is operatively linked to a reporter gene, such as EGFP, which can be used to determine the activity of each promoter variant under different conditions. Application of such a mutational and deletional analysis enables the identification of promoter sequences containing desirable activities and thus identifying a particular promoter domain, including core promoter elements. This approach can be used to identify, for example, the smallest region capable of conferring tissue or cell specificity.

a) Cell-Cycle Dependent Promoters

Cell-cycle dependent promoters include those that are regulated by tumor suppressor proteins, such as RB family proteins or p53. Included among these are E2F responsive promoters. The E2F transcription factor (sometimes referred to as E2F protein or E2F) can regulate expression of numerous genes effecting cellular proliferation including proto-oncogenes and genes regulating cell cycle progression. For example, E2F is a binding target of retinoblastoma (RB) family of tumor suppressors including p107, p103 and pRb itself. In growth-arrested cells, the pRb family of proteins (pRB, p107 and p130) can mediate transcriptional repression in at least two ways: via direct repression domains and by recruiting the activity of the protein RBP1, which directly represses transcription and indirectly represses transcription by recruiting HDAC. In normal tissues, E2F responsive promoters are typically repressed by RB family/E2F such as pRb/E2F or p130/E2F. The ability of pRB to act as a growth suppresser is linked to this property (Sellers, W. R. et al. (1995) Proc. Natl. Acad. Sci. U. S. A. 92: 11544-11548). In dividing cells, pRB family members are no longer bound to E2F, thus allowing for transcriptional activation of promoters containing E2F binding sites.

E2F responsive promoters are characterized by the presence of E2F consensus sites, which can show homology with the CDE/CHR bipartite repressor element. For example, many E2F responsive promoters contain a GC-rich E2F binding motif (CDE) and a few nucleotides downstream a TGG/A motif, designated as CHR. The E2F consensus site can be either activating or repressing depending on the presence of a canonical TATA box (Tommasi et al. (2007) J Biol. Chem., 272:30483-30490). Typically, E2F responsive promoters contained in the oncovector constructs provided herein are TATA-less promoters, thereby repressing transcription in the presence of repressor proteins such as E2F/Rb or E2F/p130. Exemplary of such promoters include, but are not limited to, cycA (SEQ ID NO:519), cdc2 (SEQ ID NO:520), cdc25 (SEQ ID NO:521), B-myb (SEQ ID NO:522), E2F-1 (SEQ ID NO:506), p107 (SEQ ID NO:523), HsOrc1, adenoE1A. Promoters from the genes TK (SEQ ID NO:526), DNA pol alpha (SEQ ID NO:527), H2A (SEQ ID NO:528), and C-myc (SEQ ID NO:529) also can be utilized if the TATA box is deleted.

Another mechanism of cell-cycle dependent repression is via the presence of CAT boxes (CCAAT motifs; SEQ ID NO:509) to which nuclear factor Y (NF-Y) complexes bind. The p53 tumor suppressor protein then binds to these bound complexes resulting in gene repression. Thus, under normal conditions, NF-Y and p53 are co-resident on promoters containing CAT boxes, and lacking p53 binding sequences. This ultimately results in repression of transcription via acetylation of c-terminal lysines on p53, the recruitment of HDACs, de-acetylation of histones and release of PCAF and p300 from the promoters. Exemplary promoters containing CAT boxes regulated by NF-Y/p53 complexes include G2/M promoters such as, but not limited to, cdc25, cyclin B1, cyclin B2, Cdc2, topoisomerase IIα (Imbriano et al. (2005) Mol. Cell. Biol., 25:3737-3751). E2F-1 also contains CAT boxes, and is regulated by p53.

Exemplary of a cell-cycle dependent promoter used in the oncovector constructs provided herein is the E2F1 promoter, and variations thereof. E2F1 is regulated by both RB family members and p53 to mediate repression of transcription in normal cells, but not in tumor cells that are deficient or mutant in any one or more of the RB family (pRB, p130 or p107) or p53 genes. E2F1 is derived from the E2F gene (5′ DNA sequence set forth in SEQ ID NO:483; GenBank Accession No. S74230). The nucleotide sequence of the E2F1 promoter corresponds to nucleotides 1194 to 1460 in the sequence of nucleotides set forth in SEQ ID NO:483. A variant nucleic acid sequence for the EF2 5′UTR is set forth in SEQ ID NO:506; GenBank Accession No. 579170 (see also SEQ ID NO:534). The nucleotide sequence of the E2F1 promoter corresponds to nucleotides 37 to 303 in the sequence of nucleotides set forth in SEQ ID NO:506. The nucleotide sequence of the E2F1 promoter corresponding to nucleotides 1194 to 1460 in the sequence of nucleotides set forth in SEQ ID NO:483 contains a cysteine (C) nucleotide at position 1250 (corresponding to nucleotide position 57 in the E2F1 promoter), whereas the nucleotide sequence of the E2F1 promoter corresponding to nucleotides 37 to 303 in the sequence of nucleotides set forth in SEQ ID NO:506 contains a thymine (T) nucleotide at position 93 (corresponding to nucleotide position 57 in the E2F1 promoter). A further variation in the E2F1 promoter is set forth in SEQ ID NO:535, which contains a thymine (T) nucleotide at position 262 (corresponding to nucleotide position 256 in the E2F1 promoter). As depicted in Table 7, the E2F1 promoter is characterized by putative binding sites for MBF-1, Sp1 and NF-kB. The E2F1 promoter also includes two canonical CAAT boxes (CAT boxes) and two palindromic E2F-binding sites. The promoter does not contain a TATA motif nor an initiator element. Hence, repression of transcription from the E2F1 promoter is mediated in an ETF/Rb family and p53-dependent manner.

E2F1 promoters provided herein can be modified. For example, E2F promoters can be modified to remove CpG motifs, for example TCG can be removed. Exemplary E2F1 promoters with optimal central TLR9 motifs removed are set forth in SEQ ID NOs:536-537. As depicted in Table 7 below, the CpG motifs are removed in regions between the transcription factor binding sites. Thus, the CpG modified E2F1 promoter (E2F-279-TCG) retains the same pattern of transcription factor binding sites as the wild-type E2F1 (E2F-WT279). In another example, a chimeric b-myb E2F can be designed to reduce CG. This is exemplified in the E2F1 promoter designated E2F-Syn216 and set forth in SEQ ID NO:538.

In some embodiments, the E2F1 promoter can be modified to increase the number of enhancer elements and/or to modulate sites for p53- or Rb family-mediated repression. For example, extra enhancer elements, such as SP-1, can be added. In one example, such enhancer elements can be spaced in intervals of about or equal to 10 bp allowing the proteins that bind to the sequences to line up on the same side of the DNA, since one turn of the alpha helix is approximately 10 bps. In another example, extra CAT boxes can be designed to provide more locations for p53-induced repression. In other examples, extra CHR elements can be included to increase the amount of E2F-based repression.

Any of the above modifications can be combined in the creation of a modified E2F1 promoter. Exemplary of such an E2F1 promoter is designated E2F-Syn216 corresponding to nucleotides 7-210 of the nucleic acid sequence set forth in SEQ ID NO:538. The E2F-Syn216 promoter contains replacement of CAT boxes every 40 bp and the addition of extra SP-1 sites. In addition, in the E2F-Syn216 promoter, the first E2F site is replaced with the B-myb/CHR combination as noted above in order to reduce CpG motifs.

Further, E2F1 promoters, including modified E2F1 promoters, can be generated as truncation variants to titer responses down to the amount of desired activity. The deletion variants are designed to provide decreasing strengths to each promoter. For example, promoters can be too strong in some cells such that they have deleterious effects on normal cells ultimately affecting the level of expression therefrom. The truncated promoters provided herein can be used to titer the expression down (lower the base line expression) so as to protect normal cells from deleterious effects. Exemplary of such truncated promoters are set forth in Table 7.

TABLE 7

| | | SEQ ID |
|---|---|---|
| Promoter | Motifs | NO |
| E2F-WT279 | E2F-like boxes MBF1 SP1 SP1 SP1 CAT | 534 |
| | CAT E2F CHR E2F CHR NFKB SP1 | |
| E2F-279-TCG | E2F-like boxes MBF1 SP1 SP1 SP1 CAT | 536 |
| | CAT E2F CHR E2F CHR NFKB SP1 | |
| E2F-WT216 | MBF1 SP1 SP1 SP1 CAT CAT E2F CHR | |
| | E2F CHR NFKB SP1 | |
| E2F-Syn216 | CAT SP1 SP1 CAT SP1 SP1 CAT SP1 CAT | 538 |
| | B-myb E2F CHR E2F CHR NFKB CAT SP1 | |
| E2F-Syn141 | SP1 CAT SP1 CAT B-myb E2F CHR E2F | 539 |
| | CHR NFKB CAT SP1 | |
| E2F-Syn109 | SP1 CAT B-myb E2F CHR E2F CHR NFKB | 540 |
| | CAT SP1 | |
| E2F-Syn63 | E2F CHR NFKB CAT SP1 | 541 |

Generally, any cell-cycle dependent promoter provided herein, including any E2F1 promoter or modified E2F1 promoter, is responsive to Rb family- (pRb, p130 or p107) or p53-mediated repression. Thus, such promoters are active in cells not expressing or mutant in p53 or Rb, but are repressed in normal cells. One of skill in the art can test such promoters in in vitro or in vivo systems, described herein in Section F. For example, cell lines deficient in such tumor suppressor genes can be used to test the activity of the promoter, compared to normal cells containing tumor suppressor genes. The E2F-responsive promoter does not have to be the full-length or wild type promoter, but should have a tumor-selectivity of at least 3-fold, at least 5-fold, at least 10-fold, at least 20-fold, at least 30-fold, at least 50-fold, at least 100-fold or even at least 300-fold. Tumor-selectivity can be determined by a number of assays using known techniques, such as the techniques employed in WO 02/067861

4. Other Elements

a. Regulatory Elements

Any of the oncovector constructs provided herein can contain one or more regulatory elements. Typically, regulatory elements are nucleic acid sequences that regulate the expression of other nucleic acid sequences at the level of transcription and/or translation. Thus, regulatory elements include, without limitation, promoters, operators, enhancers, ribosome binding sites, transcription termination sequences (i.e., a polyadenylation signal), T2A and other elements known to one of skill in the art. In addition, regulatory elements can be, without limitation, synthetic DNA, genomic DNA, intron DNA, exon DNA, and naturally-occurring DNA as well as non-naturally-occurring DNA.

i. IRES

The oncovector constructs provided herein contain one or more genes under the control of a conditional promoter. In embodiments where the expression of two or more genes is driven by a single promoter, an internal ribosome entry site (IRES) element can be included between coding sequences. The function of an IRES element is to enable the translation of two or more genes from one mRNA molecule, thus creating a bi-cistronic, tri-cistronic or poly-cistronic transcriptional/translational unit. Typically, eukaryotic translation can only be initiated at the 5′ end of the mRNA molecule. IRES elements form three dimensional structures (through sequence hybridization) that can directly or indirectly bind the 40S ribosomal subunit in such a way that their initiator codons are located in ribosomal P-site, allowing initiation of translation of mRNAs without the need for 5′ cap recognition.

IRES elements for the oncovector constructs provided herein can be derived from various organisms. Generally, IRES elements will be selected from among viral IRES elements. IRES elements contemplated for use in the oncovector constructs provided herein include, but are not limited to, IRES elements from Poliovirus, Rhinovirus, Encephalomyocarditis virus, Foot-and-mouth disease virus, Hepatitis A virus, Hepatitis C virus, Classical swine fever virus, Bovine viral diarrhea virus, Moloney murine leukemia (MMLV), Human immunodeficiency virus, Plautia stali intestine virus, Rhopalosiphum padi virus, Cricket paralysis-like virus, Triatoma virus, and Kaposi's sarcoma-associated herpesvirus. An exemplary IRES element is derived from the Cricket paralysis-like virus (CPLV IRES; set forth in SEQ ID NO:103) or Encephalomyocarditis virus EMCV (set forth in SEQ ID NO:104).

IRES elements provided herein can be modified. For example, IRES elements can be modified to remove or reduce the number of CpG motifs. Typically, IRES elements will be CpG modified such that the nucleotide substitutions in the hybridization regions still maintain the proper match and thus do not disrupt the three dimensional structure necessary for IRES function. An exemplary CPLV IRES element with CpG motifs removed is set forth in SEQ ID NO:102.

ii. Polyadenylation Signal

Expressed gene elements in the oncovector constructs provided herein generally will have a polyadenylation (pA) signal attached to the 3′ end of the coding sequence of the gene. The pA signal also can be included as part of a bi-, tri- or poly-cistronic transcriptional/translational unit. Typically, the pA signal is placed at the 3′ terminal end of such units. Polyadenylation signals initiate transcription termination and direct the addition of approximately 200-250 adenosine residues to the 3′ end of the mRNA transcript. Polyadenylation of mRNA molecules enhances RNA stability and translation. For example, the polyadenosine (poly-A) tail protects the mRNA molecule from exonucleases and promotes export of the mRNA from the nucleus.

Polyadenylation occurs after transcription of DNA into RNA in the nucleus. After the polyadenylation signal has been transcribed, the mRNA chain is cleaved through the action of an endonuclease complex associated with RNA polymerase. The cleavage site is characterized by the presence of the base sequence AAUAAA (SEQ ID NO:197) near the cleavage site. After the mRNA has been cleaved, 50 to 250 adenosine residues are added to the free 3′ end at the cleavage site. This reaction is catalyzed by polyadenylate polymerase.

Polyadenylation signal elements contemplated for use herein can be any nucleotide sequence that functions as a pA signal in the manner described herein or any pA signal element known in the art (e.g., SV40 early and late pA). Exemplary pA signals are set forth in SEQ ID NOs:191-196. Polyadenylation signal elements provided herein can be modified. For example, pA elements can be modified to remove or reduce the number of CpG motifs. An exemplary pA element with CpG motifs removed is set forth in SEQ ID NO:194.

b. Reporter Genes

Any of the constructs provided herein also can contain one or more reporter genes, that allow for the selection, identification or detection of reporter constructs and expressed genes therefrom. The reporter gene can encode a reporter protein, including, but not limited to, chloramphenicol acetyl transferase (CAT), β-galactosidase (encoded by the lacZ gene), luciferase, alkaline phosphatase, fluorescent protein, such as a green fluorescent protein (GFP) or red fluorescent protein (RFP), and horse radish peroxidase. A construct provided herein also can be constructed to contain a gene encoding a product conditionally required for survival (e.g., an antibiotic resistance marker). For example, a construct can contain nucleic acid that encodes a polypeptide that confers resistance to a selection agent such as neomycin (also called G418), puromycin, or kanamycin. Exemplary reporter genes encode EGFP and are set forth in SEQ ID NO:543 or 545 and encoding a protein set forth in SEQ ID NO:544 or 546; luciferase set forth in SEQ ID NO:547; or mKate set forth in SEQ ID NO:549.

c. Adjunct Therapy Proteins

Provided herein are oncovector constructs containing additional transcription units. These units can contain a gene or plurality of genes which encode proteins that can serve as adjunct therapeutic factors. These additional transcription units can be under the control of a conditional promoter that is the same as or is different from the conditional promoter driving expression of the other elements provided herein. These additional transcription units also can be an independent transcriptional unit within the construct or can be part of a bi-, tri- or poly-cistronic transcription/translation unit. Generally these additional transcription units contain genes which encode proteins that promote the selective destruction of a target cell population which is the same population of cells targeted by the oncovector constructs. Exemplary elements of the additional transcription units include, but are not limited to, suicide genes such as prodrug modifying elements, cytotoxic protein, and apoptosis-inducing proteins; cytokines; chemokines; and angiogenesis inhibitors. These elements can be modified to remove or reduce CpG motifs and/or to optimize for human codon usage, as described herein.

For example, anticancer genes have been expressed from viral vectors and include prodrug-activating or “suicide” genes, cytokine genes (to enhance immune defense against the tumor), tumor toxic genes such as diphtheria toxin, anti-angiogenesis genes, tumor vaccination genes, tumor suppressor genes, radiosensitivity genes, antisense RNA and ribozymes (see e.g., U.S. Pat. No. 6,897,067). Hence, the nucleic acid molecule also can contain one or more gene, such as an anticancer transgene, including, but not limited to, a suicide gene, a prodrug, cytokine genes, for example to enhance immune defense against the tumor (Blankenstein, T et al. J. Exp. Med. 173:1047-1052 (1991); Colombo, M. P., et al., Cancer Metastasis Rev. 16:421-432 (1997); Colombo, M. P., et al., Immunol. Today 15:48-51 (1994)), tumor toxic genes, such as diphtheria toxin (Coll-Fresno, P. M., et al., Oncogene 14:243-247 (1997)), pseudomonas toxin, anti-angiogenesis genes, radiosensitivity genes, antisense RNA and ribozymes (Zaia, J. A., et al., Ann. N.Y. Acad. Sci. 660:95-106 (1992)).

i. Suicide Gene

Any oncovector construct provided herein also can contain one or more suicide genes. Such suicide genes, when expressed, encode a protein that causes cell death. Suicide genes include, but are not limited to a gene encoding a protein that induces apoptosis, a toxin, a prodrug modifying gene, or a gene encoding a polypeptide that interferes with a signal transduction cascade involved with cellular survival or proliferation. Any one or more of these genes can be contained in the oncovector constructs provided herein.

a) Cytotoxic Proteins

Provided herein are oncovector constructs containing genes encoding cytotoxic proteins. Such genes encode proteins that kill cells directly and include bacterial toxin genes, which are normally found in the genome of certain bacteria and encode polypeptides (i.e. bacterial toxins) that are toxic to eukaryotic cells. Bacterial toxins include but are not limited to diphtheria toxin.

b) Apoptosis-Inducing Proteins

Provided herein are oncovector constructs containing genes encoding apoptosis-inducing proteins, which can cause cell death directly, for example, by inducing apoptosis. Such a gene is referred to as an “apoptosis-inducing gene”, and includes, but is not limited to TNF-α. Some apoptosis-inducing proteins, such as cysteine proteases, play a key role in the initiation, regulation, and execution of cell death through their proteolytic activities. Such exemplary apoptosis-inducing proteins include, but are not limited to, vesicular stomatitis virus M, cysteine proteases, caspases and calpains.

c) Proteins that Interfere with Cellular Survival and Proliferation

Additionally, a suicide gene can encode a polypeptide that interferes with a signal transduction cascade involved with cellular survival or proliferation. Such cascades include, but are not limited to, the cascades mediated by the Flt1 and Flk1 receptor tyrosine kinases). Polypeptides that can interfere with Flt1 and/or Flk1 signal transduction include, but are not limited to, a soluble Flt1 receptor (s-Flt1) and an extracellular domain of the Elk-1 receptor (ex-Flk1).

ii. Immunomodulatory Proteins

Any oncovector construct provided herein can contain any one or more genes encoding immunomodulatory proteins. Exemplary of such proteins are cytokine and/or chemokine proteins. Cytokines and/or chemokines can be included in the oncovector constructs provided herein for their ability to potentiate the induction of a secondary immune response against the cells expressing these proteins. Exemplary cytokines include, for example, interferon, interleukins and tumor necrosis factor cytokines. Such cytokines include, but are not limited to, interleukin 1, interleukin 2 (see, e.g., U.S. Pat. No. 4,738,927 or 5,641,665); interleukin 4, interleukin 5, interleukin 7 (see e.g., U.S. Pat. No. 4,965,195 or 5,328,988); interleukin 12 (see e.g., U.S. Pat. No. 5,457,038); interleukin 18, tumor necrosis factor alpha (see e.g., U.S. Pat. No. 4,677,063 or 5,773,58); interferon gamma (see e.g., U.S. Pat. No. 4,727,138 or 4,762,79); interferon alpha, or GM-CSF (see e.g., U.S. Pat. No. 5,393,870 or 5,391,485). Exemplary chemokines include, but are not limited to, macrophage inflammatory proteins, including MIP-3, (See, Well, T. N. and Peitsch, M C. J. Leukoc. Biol vol 61 (5): pages 545-50, 1997) and MCP-3. Other immunomodulatory proteins include proteins that stimulate interactions with immune cells (B7, CD28, MHC class I, MHC class II, TAPs).

iii. Angiogenesis Inhibitors

The oncovector constructs provided herein can contain genes encoding angiogenesis inhibitors. Exemplary angiogenesis inhibitors include, but are not limited to, anti-angiogenic proteins including, but not limited to, METH-1, METH-2, TrpRS fragments, proliferin-related protein, prolactin fragment, PEDF, vasostatin, various fragments of extracellular matrix proteins and growth factor/cytokine inhibitors. Various fragments of extracellular matrix proteins include, but are not limited to, angiostatin, endostatin, kininostatin, fibrinogen-E fragment, thrombospondin, tumstatin, canstatin, and restin.

5. Modification of Components

The genes and non-coding regions of the oncovector constructs provided herein can be modified to be optimized for human usage and/or to be optimized for therapeutic use. For example, one of the problems with many therapeutic vaccines in humans is their stimulation of a host immune response against them (Ma X et al. (2002) Vaccine, 20:3263-71.) Thus, the constructs, or components of the constructs, provided herein can be optimized by modification to modulate the immunostimulatory response to the construct's composition.

It is known that different forms of nucleic acids can be potent inducers of immune responses, particularly foreign nucleic acid (Wattrang et al. (2005) Vet. Immunol. Immunopath., 107:265-279). The innate immune defense has evolved mechanisms to protect against invading microorganisms through the recognition of foreign patterns, such as carbohydrates and certain types of nucleotide sequences prevalent in microbial genomes. Among such sequences are unmethylated CpG motifs present in DNA, which are principally recognized by toll receptor 9 (TLR9), leading to the induction of inflammatory responses and the secretion of various cytokines that can be immunostimulatory (Raz et al. (1996) Proc. Natl. Acad. Sci. USA, 93:5141-5; Sato Y et al. (1996) Science, 273:352-4; Wattrang et al. (2005) Vet. Immunol. Immunopath., 107:265-279; Ma X et al. (2002) Vaccine, 20:3263-71; Stacey K J et al. (2003) J Immunol., 170:3614-20). Thus, the binding of TLR9 by bacterial or viral DNA, which is predominantly unmethylated as compared to mammalian DNA, results in the activation of proinflammatory cytokine production. The production of pro-inflammatory cytokines has a negative affect on gene expression. This may occur by activation of innate immune responses that disrupt normal cellular pathway, and can induce the methylation of exogenous DNA, which decreases transcription factor binding, to reduce gene expression. The production of pro-inflammatory cytokines also can potentiate the induction of a secondary immune response.

Particular DNA sequences containing the CG dinucleotide (CpG), often referred to as CpG motifs are recognized by pattern recognition receptors of the TLR9 family of innate immune response receptors. The optimal human motif contains a TCG sequence having the core sequence GTCGTT (SEQ ID NO:515; (Bauer V et al. (2001) Proc. Natl. Acad. Sci. USA, 98:9237-9242). The optimal mouse motifs contain the core sequences AACGTT (SEQ ID NO:516), GACGTT (SEQ ID NO:517), or AACGTC (SEQ ID NO:518), and also can induce some proinflammatory immune responses from human cells.

Any of the constructs provided herein, or components of the constructs provided herein, can be modified to remove or alter the CpG motifs. Such modification is known to one of skill in the art (see e.g., published U.S. Patent Appln. No. US 2004/0053870). In one example, CpG motifs of the construct, or any one or more components of the constructs, provided herein can be methylated to reduce the immunostimulatory response. Methylation of CpG motifs suppresses inflammation. In another example, the CpG motifs of the construct, or any one or more components of the construct, can be removed or altered to reduce the inflammatory response. In one embodiment, removal can be achieved by deleting or altering non-essential regions of a construct. In another embodiment, nucleic acid molecules encoding CpG motifs can be mutated, such as by site-directed mutagenesis, by altering the coding sequence of the nucleic acid molecule to remove the CpG motif. In such an embodiment, it is desired that the mutations are silent mutations such that the encoded amino acid sequence remains unchanged. For the use within human cells, the removal of the TCG motif is desired.

Thus, the entire construct provided herein can be re-designed to reduce the amount of CpG motifs available for the stimulation of TLR9 receptors. Any one or more components of the construct can be modified to remove or alter the CpG motifs including, but not limited to, the origin, the promoter, the replication initiator (e.g. SV-T), the antibiotic resistance, which aids in the selection and growth of the construct in E. Coli bacteria, the IRES, and others including any expressed genes. CpG modification is exemplified in Example 1.

Modification of the CpG motifs in the constructs, or any one or more components of the constructs, provided herein results in decreased inflammatory responses induced by the construct. Upon modification, the constructs can be tested to determine if they exhibit reduced immunostimulatory responses. Such assays are known to one of skill in the art, and include in vitro and in vivo assays. For example, any of the modified constructs, or any one or more components of the constructs, can be tested to determine if they exhibit a decreased induction of inflammatory cytokines compared to the unmodified construct. Induction of inflammatory cytokines can be tested in vitro using cell lines such as, but not limited to, THP-1 cells, RAW264.7, and J774A1 (Yasuda et al. (2004) Immunology, 111:282-290) or TLR9 transfected cells, or using primary cells such as macrophages, neutrophils or dendritic cells. Induction of inflammatory cytokines also can be tested following in vivo administration, such as from blood, serum or bronchoalveolar lavage fluid (see e.g., US 2004/0053870), depending on the route of administration. As discussed below, it is desired that any construct modified to remove CpG motifs will retain activity, such as for example, replication and fusogenic activities.

Besides modification of immunostimulatory elements, such as CpG motifs, the constructs, or components of the constructs provided herein, can be modified by optimization of the codons for expression in humans. Codon optimization involves balancing the percentages of codons selected with the published abundance of human transfer RNAs so that none is overloaded or limiting. This is necessary because most amino acids are encoded by more than one codon, and codon usage varies from organism to organism. Differences in codon usage between transfected genes and host cells can have effects on protein expression and immunogenicity of a vaccine construct. Table 8 below sets forth the Human codon usage frequency table. Thus, codons are chosen to select for those codons that are in balance with human usage frequency. The redundancy of the codons for amino acids is such that different codons code for one amino acid as depicted in Table 9 below. In selecting a codon for replacement, it is desired that the resulting mutation is a silent mutation such that the codon change does not affect the amino acid sequence. Generally, the last nucleotide of the codon can remain unchanged without affecting the amino acid sequence.

TABLE 8
Human Codon Usage Frequency

| | | | | | | | | | | | |
|---|---|---|---|---|---|---|---|---|---|---|---|
| TTT | 17.5 | (676381) | TCT | 15.1 | (585967) | TAT | 12.1 | (470083) | TGT | 10.5 | (407020) |
| TTC | 20.4 | (789374) | TCC | 17.7 | (684663) | TAC | 15.3 | (592163) | TGC | 12.6 | (487907) |
| TTA | 7.6 | (294684) | TCA | 12.2 | (471469) | TAA | 1.0 | ( 38222) | TGA | 1.5 | (59528) |
| TTG | 12.9 | (498920) | TCG | 4.4 | (171428) | TAG | 0.8 | ( 30104) | TGG | 13.2 | (510256) |
| CTT | 13.1 | (508151) | CCT | 17.5 | (676401) | CAT | 10.8 | (419726) | CGT | 4.6 | (176691) |
| CTC | 19.6 | (759527) | CCC | 19.8 | (767793) | CAC | 15.1 | (583620) | CGC | 10.5 | (405748) |
| CTA | 7.2 | (276799) | CCA | 16.9 | (653281) | CAA | 12.2 | (473648) | CGA | 6.2 | (239573) |
| CTG | 39.8 | (1539118) | CCG | 6.9 | (268884) | CAG | 34.2 | (1323614) | CGG | 11.5 | (443753) |
| ATT | 15.9 | (615699) | ACT | 13.1 | (506277) | AAT | 16.9 | (653566) | AGT | 12.1 | (469641) |
| ATC | 20.9 | (808306) | ACC | 18.9 | (732313) | AAC | 19.1 | (739007) | AGC | 19.5 | (753597) |
| ATA | 7.4 | (288118) | ACA | 15.0 | (580580) | AAA | 24.3 | (940312) | AGA | 12.1 | (466435) |
| ATG | 22.1 | (853648) | ACG | 6.1 | (234532) | AAG | 31.9 | (1236148) | AGG | 11.9 | (461676) |
| GTT | 11.0 | (426252) | GCT | 18.5 | (715079) | GAT | 21.8 | (842504) | GGT | 10.8 | (416131) |
| GTC | 14.5 | (562086) | GCC | 27.9 | (1079491) | GAC | 25.2 | (973377) | GGC | 22.3 | (862557) |
| GTA | 7.1 | (273515) | GCA | 15.9 | (614754) | GAA | 28.8 | (1116000) | GGA | 16.5 | (637120) |
| GTG | 28.2 | (1091853) | GCG | 7.4 | (286975) | GAG | 39.6 | (1532589) | GGG | 16.4 | (636457) |

TABLE 9
Codon Redundancy

| Amino Acid | Codon(s) |
|---|---|
| Aspartic Acid (Asp) | GAC; GAT |
| Lysine (Lys) | AAA; AAG |
| Valine (Val) | GTG; GTA; GTC; GTT |
| Leucine (Leu) | CTA; CTT; CTC; CTG; |
| | TTA; TTG |
| Asparagine (Asn) | AAC; AAT |
| Arginine (Arg) | CGA; CGT; CGC; CGG; |
| | AGA; AGG |
| Glutamic Acid (Glu) | GAA; GAG |
| Serine (Ser) | TCA; TCT; TCC; TTG; |
| | AGC; AGT |

For example, the codons TCT, TCC, TCA, TCG, AGT and AGT all code for Serine (note that T is the DNA equivalent to the U in RNA). From a human codon usage frequency as set forth in Table 8 above, the corresponding usage frequencies for these codons are 15.1, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal that you are optimizing for.

Generally, the strategy for optimizing a construct provided herein, or a component of a construct provided herein, is to optimize both human codon usage and also to optimize the immunostimulatory effect of the construct, such as by modifying CpG motifs. To do so requires consideration of several factors. First, any codon that contains a CpG motif is not used in optimization of the construct. For example, many Arginine codons contain CpG motifs and are not used at all. The remaining two Arginine codons then are balanced so that each is used approximately equally. This is exemplified with optimization of SV-T as described in Example 1. For example, the remaining two codons for Arginine were balanced in the SV-T gene at 48% and 51% each (AGA, AGG, respectively, shown in Table 15 in the usage Table). Second, in the sequence of a gene or non-coding region in a construct, any two codons placed next to one another could form a CpG motif. Thus, in assessing such sequences, it is desired that the choice of codon is made to avoid any formation of a CpG motif, even with an adjacent codon. Third, each of the above two requirements are primary considerations when selecting codons for human optimization. Thus, any codon that is selected to balance the codons based on human usage frequency must be made such that the replacing codons do not introduce codons containing CpG motifs, nor introduce CpG motifs with adjacent codons.

The final consideration in modifying a construct, or a component of a construct, provided herein, is to assess the modified sequences for introduced restriction sites. New restriction sites can be generated during the modification process. In one example, the newly modified sequence can be checked with a sequence analysis program in order to find newly generated restriction sites. If a restriction site has been introduced, silent mutations (nucleotide changes that do not change the amino acid sequence) can be introduced into the sequence to disrupt the unwanted restriction sites. Exemplary of such a sequence program is the DNA analysis program Gene Construction Kit® (Textco). This program can be used to design sequences, analyze CpG motifs, and to analyze restriction sites.

It is expected that modification of the constructs, or components of the constructs, to remove CpG motifs will often result in a shift in the codon usage frequency of the sequence. The objective for human optimization is then to attempt to balance the abundance of the codons while still adhering to the factors discussed above, in particular the first and second factors regarding removal of any CpG motifs. Because of the difficulty in following all of the above considerations simultaneously, the codon usage frequency is not always proportional to the human codon usage table. It is an objective, however, to get the codons as closely associated and balanced as possible.

Any one or more of a coding and/or non-coding region of a construct can be optimized as described herein, for example, to remove immunostimulatory elements, such as CpG motifs or to optimize for human usage. Exemplary of such coding and non-coding regions include, but are not limited to, coding genes such as the replication initiator, fusogenic component, or other genes encoding cytokines, chemokines, prodrugs, suicide genes and others, promoter, origin of replication, regulatory genes including the IRES, marker or selective genes such as genes encoding GFP or antibiotic resistance genes, and others.

Generally, if the region is truly a non-functional domain, the CpG can be removed and/or the sequence can be human optimized. Where the sequence is coding, or even for many non-coding sequences, it is necessary to rationally design and empirically test modifications to ensure that the resulting components or constructs function as contemplated. Such design and testing is within the skill of one in the art using assays as described herein and other assays known in the art. For example, despite the sequence being non-coding, many non-coding sequences are necessary for regulatory purposes, such as, but not limited to, replication, transactivation, stability of transcripts and others. Thus, it is necessary to ensure that modification of the CpG motif does not inhibit such functions of the non-coding regions. In one example, the SV40 origin set forth in SEQ ID NO:113 contains four closely spaced GAGGC (SEQ ID NO:122) motifs, two in one direction, and two inverted, that are necessary for binding of SV40 TAg. The SV40 recognition sequence for SV40 TAg is set forth in SEQ ID NO:123. This recognition sequence is the core binding domain for SV40 TAg and changing any nucleotides can negate all replicative activities associated with the SV40 TAg. It can be possible, however, to change the intervening nucleotides in order to reduce the CpG. For example, amino acid residues in the SV40 origin can be modified from GAGGCGGAGGCCGCCTCGGCCTC (SEQ ID NO:123) to GAGGCTGAGGCTGCCTCTGCCTC (SEQ ID NO:124 where the N's are T). The recognition motifs are underlined. The CG in SEQ ID NO:123 are in bold and the nucleotides proposed for modification in SEQ ID NO:124 are in italics. Thus, in this example, a modified origin can be tested for function in an assay to assess replication.

Typically, the constructs provided herein, or components of the constructs, including coding and non-coding regions, retain function or activity of the wild-type construct or component. The retained function or activity is about or is at least or about at least 30%, 40%, 50%, 60%, 70%, 80%, 90%, 95% or more of the function or activity of the wild-type construct or sequence not containing any modifications. For example, as discussed above, a modified and optimized origin of replication contained within a construct retains replicative activity. In another example, a modified and optimized replication initiator, such as for example, SV40 TAg also retains replicative ability. Where the SV40 TAg also is modified to uncouple transformation from replication, the SV40 TAg retains only replicative activity, but is deficient in its transformative activity. In an additional example, a modified and optimized E2F-1 promoter retains ability to induce gene transcription in a tumor-specific manner. One of skill in the art knows or can determine the function of the particular component or construct, and can empirically test such components following modification and optimization to identify those that retain function or activity. Generally, any construct provided herein containing modified and optimized non-coding and coding elements will retain replication and fusogenic activities. Where the construct is modified to remove CpG motifs it also will exhibit reduced immunostimulatory activity.

6. Exemplary Oncovector Constructs

In one example, provided herein is an autonomous replicating nucleic acid molecule where the components are positioned on the nucleic acid in a consecutive order to include: A) a first promoter that controls expression of the genes; B) a first ORF coding for a reporter gene; C) an IRES separating the genes of interest; D) a second ORF coding for a replication initiator or variant thereof; and E) an origin of replication. The first and second ORF can be in reverse order. Hence, also provided herein is a nucleic acid molecule where the components are positioned on the nucleic acid in a consecutive order to include: A) a first promoter that controls expression of the genes; B) a first ORF coding for a replication initiator or variant thereof; C) an IRES separating the genes of interest; D) a second ORF coding for a reporter gene; and E) an origin of replication. The reporter genes can encode any reporter gene that encodes a detectable protein or a protein capable of being detected. Exemplary reporter genes are described above, and include for example, RFP, GFP, mKate2, luciferase, or beta-galactosidase. The replication initiator can be any that is compatible with the origin of replication in order to induce autonomous replicative activity of the construct. Exemplary origin/replication initiatior combinations are described herein, and include, but are not limited to, an SV40 origin and an SV40 T antigen; a BKV origin and BKV large T antigen; a BKV origin and SV40 T antigen; and an EBV origin and Epstein Barr virus Nuclear Antigen (EBNA), or mutants or variants thereof. The promoter can be a constitutive promoter or a cell-type of tumor-specific promoter. For example, the promoter can be a CMV promoter. In another example, a promoter is a tumor-specific promoter, such as but not limited to, EF1 or EF2 or a variant thereof. Exemplary of such constructs are set forth in Table 10 and 10A:

TABLE 10

| | | | | | | | SEQ |
|---|---|---|---|---|---|---|---|
| Construct | | | | | polyA | SV40 | ID |
| name (s) | Promoter | 1^{st} gene | IRES | 2^{nd} gene | signal | ori^{a} | NO. |
| pCzGFP-I- | CMV | zGFP | + | TAg (E107L) | SV40 | o | 706 |
| T(E107L) | | | | | | | |
| pCzGFP-I- | CMV | zGFP | + | TAg | SV40 | o | 707 |
| T(E107L/D402R) | | | | (E107L/D402R) | | | |
| pCzGFP-I- | CMV | zGFP | + | TAg | SV40 | o | 708 |
| T(E107L/E108L) | | | | (E107L/E108L) | | | |
| pCzGFP-I- | CMV | zGFP | + | TAg | SV40 | o | 709 |
| T(E107L/E108L/ | | | | (E107L/E108L/ | | | |
| D402R) | | | | D402R) | | | |
| pCzGFP-I- | CMV | zGFP | + | TAg | SV40 | o | 710 |
| T(E107L/E108L/ | | | | (E107L/E108L/ | | | |
| D453S) | | | | D453S) | | | |
| pCzGFP-I- | CMV | zGFP | + | TAg | SV40 | o | 711 |
| T(E107L/E108L/ | | | | (E107L/E108L/ | | | |
| V585R) | | | | V585R) | | | |
| pCzGFP-I- | CMV | zGFP | + | TAg | SV40 | o | 712 |
| T(E107L/E108L/ | | | | (E107L/E108L/ | | | |
| D604R) | | | | D604R) | | | |
| pCMV-GFP-IRES- | CMV | zGFP | + | TAg | syn | + | 607 |
| LTAg-WT BB3 | | | | | | | |
| (pCzGFP-I-T-BB3) | | | | | | | |
| (pCzGFP-I-T(WT)- | | | | | | | |
| BB3) | | | | | | | |
| pCzGFP-I-nT-BB3 | CMV | zGFP | + | native TAg^{b} | syn | + | 609 |
| pC-mKate2-I-T- | CMV | mKate2 | + | TAg | syn | + | 611 |
| BB3 | | | | | | | |
| pC-Luc-I-T-BB3 | CMV | Luciferase | + | TAg | syn | + | 612 |
| pC-Bgal-I-T-BB3 | CMV | Beta- | + | TAg | syn | + | 614 |
| | | galactosidase | | | | | |
| pCzGFP-I- | CMV | zGFP | + | TAg (L19F) | syn | + | 616 |
| T(L19F)-BB3 | | | | | | | |
| pCzGFP-I- | CMV | zGFP | + | TAg (P28S) | syn | + | 617 |
| T(P28S)-BB3 | | | | | | | |
| pCzGFP-I- | CMV | zGFP | + | TAg (L103P) | syn | + | 618 |
| T(L103P)-BB3 | | | | | | | |
| pCzGFP-I- | CMV | zGFP | + | TAg (C105A) | syn | + | 619 |
| T(C105A)-BB3 | | | | | | | |
| pCzGFP-I- | CMV | zGFP | + | TAg (E107L) | syn | + | 620 |
| T(E107L)-BB3 | | | | | | | |
| pCzGFP-I- | CMV | zGFP | + | TAg (E107K) | syn | + | 621 |
| T(E107K)-BB3 | | | | | | | |
| pCzGFP-I- | CMV | zGFP | + | TAg (E108L) | syn | + | 622 |
| T(E108L)-BB3 | | | | | | | |
| pCzGFP-I- | CMV | zGFP | + | TAg (S112N) | syn | + | 623 |
| T(S112N)-BB3 | | | | | | | |
| pCzGFP-I- | CMV | zGFP | + | TAg (S189N) | syn | + | 624 |
| T(S189N)-BB3 | | | | | | | |
| pCzGFP-I- | CMV | zGFP | + | TAg (D402R) | syn | + | 625 |
| T(D402R)-BB3 | | | | | | | |
| pCzGFP-I- | CMV | zGFP | + | TAg (P453S) | syn | + | 626 |
| T(P453S)-BB3 | | | | | | | |
| pCzGFP-I- | CMV | zGFP | + | TAg (V585R) | syn | + | 627 |
| T(V585R)-BB3 | | | | | | | |
| pCzGFP-I- | CMV | zGFP | + | TAg (D604R) | syn | + | 628 |
| T(D604R)-BB3 | | | | | | | |
| pCzGFP-I- | CMV | zGFP | + | TAg | syn | + | 629 |
| T(L103P/C105A)- | | | | (L103P/C105A) | | | |
| BB3 | | | | | | | |
| pCzGFP-I- | CMV | zGFP | + | TAg | syn | + | 630 |
| T(L103P/E107L)- | | | | (L103P/E107L) | | | |
| BB3 | | | | | | | |
| pCzGFP-I- | CMV | zGFP | + | TAg | syn | + | 631 |
| T(L103P/E108L)- | | | | (L103P/E108L) | | | |
| BB3 | | | | | | | |
| pCzGFP-I- | CMV | zGFP | + | TAg | syn | + | 632 |
| T(C105A/E107L)- | | | | (C105A/E107L) | | | |
| BB3 | | | | | | | |
| pCzGFP-I- | CMV | zGFP | + | TAg | syn | + | 633 |
| T(C105A/E108L)- | | | | (C105A/E108L) | | | |
| BB3 | | | | | | | |
| pCzGFP-I- | CMV | zGFP | + | TAg | syn | + | 634 |
| T(E107L/E108L)- | | | | (E107L/E108L) | | | |
| BB3 | | | | | | | |
| pCzGFP-I- | CMV | zGFP | + | TAg | syn | + | 635 |
| T(C105A/E107L/ | | | | (C105A/E107L/ | | | |
| E108L)-BB3 | | | | E108L) | | | |
| pCzGFP-I- | CMV | zGFP | + | TAg | syn | + | 636 |
| T(L103P/E107L/ | | | | (L103P/E107L/ | | | |
| E108L)-BB3 | | | | E108L) | | | |
| pCzGFP-I- | CMV | zGFP | + | TAg | syn | + | 637 |
| T(L103P/C105A/ | | | | (L103P/CA105A/ | | | |
| E107L/E108L)- | | | | E107L/E108L) | | | |
| BB3 | | | | | | | |
| pCzG-I- | CMV | zGFP | + | TAg | syn | + | 638 |
| T(E107L/E108L/ | | | | (E107L/E108L/ | | | |
| S112N)-BB3 | | | | S112N) | | | |
| pCzG-I- | CMV | zGFP | + | TAg | syn | + | 639 |
| T(C105A/E107L/ | | | | (C105A/E107L/ | | | |
| E108L/S112N)- | | | | E108L/S112N) | | | |
| BB3 | | | | | | | |
| pCzG-I- | CMV | zGFP | + | TAg | syn | + | 640 |
| T(C105A/E108L/ | | | | (C105A/E108L/ | | | |
| S112N)-BB3 | | | | S112N) | | | |
| pCzG-I- | CMV | zGFP | + | TAg | syn | + | 641 |
| T(C105A/E107L/ | | | | (C105A/E107L/ | | | |
| S112N)-BB3 | | | | S112N) | | | |
| pCzG-I- | CMV | zGFP | + | TAg | syn | + | 642 |
| T(C105A/S112N)- | | | | (C105A/S112N) | | | |
| BB3 | | | | | | | |
| pCzG-I- | CMV | zGFP | + | TAg | syn | + | 643 |
| T(L103P/E107K) | | | | (L103P/E107K) | | | |
| pCzG-I- | CMV | zGFP | + | TAg | syn | + | 644 |
| T(L105A/E107K) | | | | (L105A/E107K) | | | |
| pCzGFP-I- | CMV | zGFP | + | TAg | syn | + | 645 |
| T(Δ366-370)- | | | | (Δ366-370) | | | |
| BB3 | | | | | | | |
| pCzGFP-I- | CMV | zGFP | + | TAg | syn | + | 646 |
| T (Δ434-444)- | | | | (Δ434-444) | | | |
| BB3 | | | | | | | |
| pCMV/EF1-zGFP- | CMV | zGFP | + | TAg | syn | + | 666 |
| I-T-BB3 | enhancers/ | | | | | | |
| | EF1 | | | | | | |
| | promoter | | | | | | |
| pCMV/EF2-zGFP- | CMV | zGFP | + | TAg | syn | + | 667 |
| I-T-BB3 | enhancers/ | | | | | | |
| | EF2 tata- | | | | | | |
| | less | | | | | | |
| | promoter | | | | | | |
| pCMV/EF2 (ss)- | CMV | zGFP | + | TAg | syn | + | 668 |
| zGFP-I-T-BB3 | enhancers/ | | | | | | |
| | EF2 tata- | | | | | | |
| | less | | | | | | |
| | promoter | | | | | | |
| | (redundant | | | | | | |
| | BamHI | | | | | | |
| | site | | | | | | |
| | removed) | | | | | | |
| pCMV/EF2-zGFP- | CMV | zGFP | + | TAg | syn | + | 669 |
| I-T (L103P)-BB3 | enhancers/ | | | (L103P) | | | |
| | EF2 tata- | | | | | | |
| | less | | | | | | |
| | promoter | | | | | | |
| pCMV/EF2-zGFP- | CMV | zGFP | + | TAg | syn | + | 670 |
| I-T (C105A)-BB3 | enhancers/ | | | (C103A) | | | |
| | EF2 tata- | | | | | | |
| | less | | | | | | |
| | promoter | | | | | | |
| pCMV/EF2-zGFP- | CMV | zGFP | + | TAg | syn | + | 671 |
| I-T (E107K)-BB3 | enhancers/ | | | (E107K) | | | |
| | EF2 tata- | | | | | | |
| | less | | | | | | |
| | promoter | | | | | | |
| pCMV/EF2-zGFP- | CMV | zGFP | + | TAg | syn | + | 672 |
| I-T | enhancers/ | | | (L103P/C105A) | | | |
| (L103P/C105A)- | EF2 tata- | | | | | | |
| BB3 | less | | | | | | |
| | promoter | | | | | | |
| pCMV/EF2-zGFP- | CMV | zGFP | + | TAg | syn | + | 673 |
| I-T | enhancers/ | | | (L103P/E107K) | | | |
| (L103P/E107K)- | EF2 tata- | | | | | | |
| BB3 | less | | | | | | |
| | promoter | | | | | | |
| pCMV/EF2-zGFP- | CMV | zGFP | + | TAg | syn | + | 674 |
| I-T | enhancers/ | | | (C105A/E107K) | | | |
| (C105A/E107K)- | EF2 tata- | | | | | | |
| BB3 | less | | | | | | |
| | promoter | | | | | | |
| pCMV/EF1/E2F- | CMV | zGFP | + | TAg | syn | + | 675 |
| zGFP-I-T-BBS | enhancers/ | | | | | | |
| | EF1 and | | | | | | |
| | tata-less | | | | | | |
| | E2F | | | | | | |
| | promoter | | | | | | |
| pCMV/int-zGFP-I- | CMV plus | zGFP | + | TAg | syn | + | 676 |
| T-BB3 | pCI intron | | | | | | |
| pC-SEAP-I-T-BB3 | CMV | SEAP | + | TAg | syn | + | 677 |
| pE2F1-zGFP-I-T- | E2F-1 | zGFP | + | TAg | syn | + | |
| BB3 | | | | | | | |
| pC-T-I-zGFP-BB3 | CMV | TAg | + | zGFP | syn | + | 689 |
| pC-T-I-Luc-BB3 | CMV | TAg | + | Luciferase | syn | + | 691 |

^{a}SV40 ori o: original, unmodified SV40 ori from pIRES2-EGFP (contains CpG)
^{b}native TAg is the unmodified SV40-T Ag gene sequence (not modified to remove CpG or optimized for human codon frequency)

TABLE 10A

| | | | | | | | | SEQ |
|---|---|---|---|---|---|---|---|---|
| Construct | | | Internal | | Internal | | SV40 | ID |
| name | Promoter | 1^{st} gene | Promoter | IRES | Promoter | 2^{nd} gene | ori | NO. |
| pC-Luc-RSV- | CMV | Luciferase | Reos | + | — | TAg | + | 728 |
| I-T-BB3 | | | sarcoma | | | | | |
| | | | virus | | | | | |
| | | | (RSV) | | | | | |
| pCzG-1I-T-BB3 | CMV | zGFP | — | 1I^{a} | — | TAg | + | 729 |
| pCzG-1I-T-dSV | CMV | zGFP | — | 1I^{a} | — | TAg | − | 688 |

^{a}IRES mutant 1I: ATGG at 3′ end mutated to ATCC to remove potentially redundant ATG start site for gene in the 2^{nd} position

In another example, provided herein is an autonomous replicating nucleic acid molecule where the components are positioned on the nucleic acid in a consecutive order to include: A) a first promoter that controls expression of the genes; B) a first ORF coding for a fusogenic protein; C) an IRES separating the genes of interest; D) a second ORF coding for a replication initiator or a variant thereof; and E) an origin of replication. The first and second ORF can be in reverse order. Hence, also provided herein is a nucleic acid molecule where the components are positioned on the nucleic acid in a consecutive order to include: A) a first promoter that controls expression of the genes; B) a first ORF coding for a replication initiator or a variant thereof; C) an IRES separating the genes of interest; D) a second ORF coding for a fusogenic protein; and E) an origin of replication. The nucleic acid encoding a fusogenic protein can include any fusogenic protein described above or known in the art. Exemplary of such proteins are ARVp10, RRVp14, BRV p15, GALV, SV5F, VSVG or any variants thereof. The replication initiator can be any that is compatible with the origin of replication in order to induce autonomous replicative activity of the construct. Exemplary origin/replication initiatior combinations are described herein, and include, but are not limited to, an SV40 origin and an SV40 T antigen; a BKV origin and BKV large T antigen; a BKV origin and SV40 T antigen; and an EBV origin and Epstein Barr virus Nuclear Antigen (EBNA), or mutants or variants thereof. The promoter can be a constitutive promoter or a cell-type of tumor-specific promoter. For example, the promoter can be CMV. Exemplary of such constructs are set forth in Table 11 and Table 12:

TABLE 11

| | | | | | | | SEQ |
|---|---|---|---|---|---|---|---|
| Construct | | | | | polyA | SV40 | ID |
| name (s) | Promoter | 1st gene | IRES | 2nd gene | signal | oria | NO. |
| pCzARVp10- | CMV | zARVp10 | + | TAg | syn | + | 647 |
| I-T-BB3 | | | | | | | |
| pCzRRVp14- | CMV | zRRVp14 | + | TAg | syn | + | 649 |
| I-T-BB3 | | | | | | | |
| pCzBRVp15-I-T-BB3 | CMV | zBRVp15 | + | TAg | syn | + | 651 |
| pCzGALV-I-T-BB3 | CMV | GALV | + | TAg | syn | + | 653 |
| pCzSV5F-I-T-BB3 | CMV | SV5F | + | TAg | syn | + | 655 |
| pCzVSVG-I-T-BB3 | CMV | VSVG | + | TAg | syn | + | 657 |
| pCzSV5F-I- | CMV | SV5F | + | TAg | syn | + | 659 |
| T(G105A)-BB3 | | (G105A) | | | | | |
| pCzSV5F-I- | CMV | SV5F | + | TAg | syn | + | 660 |
| T(G109A)-BB3 | | (G109A) | | | | | |
| pCzSV5F-I- | CMV | SV5F | + | TAg | syn | + | 661 |
| T(G114A)-BB3 | | (G114A) | | | | | |
| pCzSV5F-I- | CMV | SV5F | + | TAg | syn | + | 662 |
| T(G105A/G109A)- | | (G105A/G109A) | | | | | |
| BB3 | | | | | | | |
| pCzSV5F-I- | CMV | SV5F | + | TAg | syn | + | 663 |
| T(G105A/G109A/ | | (G105A/G109A/ | | | | | |
| G114A)-BB3 | | G114A) | | | | | |
| pC-T-I-zGALV-BB3 | CMV | TAg | + | zGALV | syn | + | 693 |
| pCzSV5F-I-T | CMV | zSV5F | + | TAg | SV40 | o | 700 |
| pCzVSVG-I-T | CMV | zVSVG | + | TAg | SV40 | o | 701 |
| pCz ARVp10-I-T | CMV | zARVp10 | + | TAg | SV40 | o | 702 |
| pCz RRVp14-I-T | CMV | zRRVp14 | + | TAg | SV40 | o | 703 |
| pCz BRVp15-I-T | CMV | zBRVp15 | + | TAg | SV40 | o | 704 |
| pCzGALV-I-T | CMV | zGALV | + | TAg | SV40 | o | 705 |
| pCzGALV-I-T-BB4 | CMV | zGALV | + | TAg | SV40 | + | 722 |

aSV40 ori o: original, unmodified SV40 ori from pIRES2-EGFP (contains CpG)

TABLE 12

| | | | | | | | | SEQ |
|---|---|---|---|---|---|---|---|---|
| Construct | | | Internal | | Internal | | SV40 | ID |
| name | Promoter | 1^{st} gene | Promoter | IRES | Promoter | 2^{nd} gene | ori | NO. |
| pC-T-I-RSV-GALV-BB3 | CMV | TAg | — | + | Reos | GALV | + | 727 |
| | | | | | sarcoma | | | |
| | | | | | virus | | | |
| | | | | | (RSV) | | | |

In a further example, provided herein is an autonomous replicating nucleic acid molecule where the components are positioned on the nucleic acid in a consecutive order to include: A) a first promoter that controls expression of the genes; B) a first ORF coding for a pro-drug modifying enzyme or variant thereof; C) an IRES separating the genes of interest; D) a second ORF coding for a replication initiator or a variant thereof; and E) an origin of replication. The first and second ORF can be in reverse order. Hence, also provided herein is a nucleic acid molecule where the components are position on the nucleic acid in a consecutive order to include: A) a first promoter that controls expression of the transgenes; B) a first ORF coding for a replication initiator or a variant thereof; C) an IRES separating the genes of interest; D) a second ORF coding for a pro-drug modifying enzyme; and E) an origin of replication. The nucleic acid encoding a prodrug-modifying enzyme can include any described above or known in the art. Exemplary of such proteins are HSV1-TK or CD or any variants thereof. The replication initiator can be any that is compatible with the origin of replication in order to induce autonomous replicative activity of the construct. Exemplary origin/replication initiatior combinations are described herein, and include, but are not limited to, an SV40 origin and an SV40 T antigen; a BKV origin and BKV large T antigen; a BKV origin and SV40 T antigen; and an EBV origin and Epstein Barr virus Nuclear Antigen (EBNA), or mutants or variants thereof. The promoter can be a constitutive promoter or a cell-type of tumor-specific promoter. For example, the promoter can be CMV. Exemplary of such constructs are set forth in Table 13:

TABLE 13

| | | | | | | | SEQ |
|---|---|---|---|---|---|---|---|
| Construct | | | | | polyA | SV40 | ID |
| name (s) | Promoter | 1st gene | IRES | 2nd gene | signal | oria | NO. |
| pC-zCDase-I-T-BB3 | CMV | zCDase | + | TAg | syn | + | 664 |
| pCzCDase-I-T-BB4 | CMV | zCDase | + | TAg | SV40 | + | 724 |

### D. METHODS OF DESIGNING ONCOVECTOR CONSTRUCTS

One of skill in the art can empirically design oncovector constructs, for example, by using any of the components described herein or other components known to one of skill in the art, and test them for replicative, oncotherapeutic, and bystander activities, such as by using assays described in Section E. The oncovector constructs can be designed from a series of intermediate or backbone constructs each containing one or more or all of the components of the construct, or modified forms thereof. Empirically designing and testing a series of intermediate constructs permits the individual assessment of each component of the construct on the replication, oncotherapeutic, and bystander activities of the construct and/or their interaction with other components in the construct. The final oncovector constructs can be integrated from the various intermediates.

Thus, in one example, each component can be designed into a separate construct and tested individually for the desired activity, and then the desired components can be integrated into a single construct. For example, the replication components (i.e. origin of replication and/or replication initiator), or modified forms thereof, can be incorporated into a single construct and tested for replication activity in any appropriate host cell, using for example, a reporter gene expressed from the construct as a readout of replication. Reporter gene expression also can be combined with quantitative PCR to determine plasmid replication. Such replication components also can be tested to ensure that they do not induce transformation, for example by confirming a lack of binding to tumor suppressor proteins such as Retinoblastoma (Rb) or p53 proteins. Likewise, the therapeutic gene, such as a bystander gene, or modified forms thereof, can be incorporated into a single construct under the control of a desired promoter and tested for oncotherapeutic (e.g., oncolytic, fusogenic, cytotoxic) and bystander activities of the gene within transfected host cells. In an additional example, the tissue or cell-specific promoter, or modified forms thereof, can be incorporated into a single construct and tested for induction of gene expression of a reporter gene operatively linked thereto in the appropriate cells, for example, tumor cells.

The experimental test constructs can be artificially synthesized as discussed below, and then tested. Once each individual component has been tested to identify those that exhibit the desired activity, the components can be combined into a single construct to integrate all activities. Those constructs that exhibit all desired activities are selected as oncovector constructs. Such a method permits a rational assessment of desired actives, which can occur in parallel, thereby providing an efficient means to test the activity of each individual component before integration.

A backbone vector also can be reconstructed and modified to facilitate the insertion and/or integration of all components into a common background containing fixed restriction sites. Thus, each of the components, or modified components, can be easily “swapped out” to permit the efficient manipulation of the components in the intermediate constructs. Each individual component can be recombinantly generated by standard molecular biology techniques to have the appropriate restriction sites. Also, as discussed below, each individual component can be artificially synthesized to contain the appropriate restriction sites. The artificially synthesized constructs, or the vector constructs, can be tested for activity in appropriate assays.

Alternatively, the oncovector constructs can be designed by creating all combinations of desired components into single constructs, and testing each construct individually to identify those that retain replication, anti-tumor activities and bystander effects. Thus, single constructs can be generated containing all possible permutations of each desired component, and each single construct can be tested to identify those constructs that exhibit a minimum of replication and fusogenic activities. The advantage of such a method is that it avoids any bias of any individual component, since all components of the constructs are integrated from the beginning.

Furthermore, multiple constructs can be designed and used such that combinations of desired components are incorporated, individually or in combination, into two or more backbone constructs, which are co-expressed and tested together to identify combinations of vectors that exhibit desired activities, including replication, oncotherapeutic, and bystander activities.

Exemplary methods to design and identify oncovector constructs involves a system of intermediate experimental constructs and backbone vectors such that the development of the final oncovector construct occurs in parts, which can be integrated later. Thus, in one example, a starting or initial backbone vector can be used to generate experimental intermediate test vectors to test replicative, oncotherapeutic, or bystander activities individually, or combinations thereof. The initial starting backbone vector also can be separately modified to optimize the plasmid backbone, thereby resulting in intermediate backbone vectors. Reconstruction of the plasmid backbone can be performed separately and/or in parallel to the construction of the experimental test vectors. Once optimized, the transcriptional unit can be removed from an experimental test vector and ligated into a designed backbone vector in order integrate the components into a single construct.

This is described with respect to the generation of a self-replicating construct containing an SV40 TAg, or mutant thereof, a cell cycle-dependent promoter (e.g., E2F), or mutant thereof, for accumulation of the construct specifically in tumor cells, and a therapeutic gene, or modified form thereof, such as one that encodes a bystander protein. One of skill in the art can adapt the method discussed below to design a construct containing other replication components, promoter elements such as cell-cycle dependent promoters, oncotherapeutic components and other desired components, such as genes for adjunct therapy, so long as the intermediate constructs exhibit the expected activity and the fully integrated final construct minimally contains replication, oncotherapeutic, and bystander activities.

1. Backbone Constructs

Backbone constructs can be generated that contain various regulatory elements and other elements necessary for gene expression. The vectors can be modified or reconstructed to remove unwanted segments (e.g. f1 single stranded ori), to add unique restriction sites, to add other transcription units of interest, and/or to reduce CpG content. The backbone constructs can be used to introduce replicative, fusogenic and/or promoter elements in order to generate intermediate experimental oncovector constructs for testing of each component individually or in combination. Adjunct therapy genes also can be incorporated into backbone constructs to enhance therapeutic activity of the constructs. Once the replicative, fusogenic, promoter, and/or other elements are identified, they can be integrated together to generate a final construct. Genes or nucleotide sequences of interest can be generated de novo by synthetic construction (e.g. overlapping PCR and/or oligonucleotide hybridization) or desired sequences can be removed from already existing sources such as commercially available vectors. Synthesized or harvested sequences can be further modified by site directed mutagenesis, PCR, or other methods known to those of skill in the art. Such modifications include, but are not limited to modifying a sequence to reduce antigenicity (e.g. reduce CpG motifs), modification of a coding sequence to optimize human codon usage, modification of a coding sequence to reduce or enhance a desired activity, and addition of restriction sequences for cloning purposes. Insertion of a desired gene or other nucleotide sequence into a backbone vector also can involve subcloning into other intermediate vectors, such as pCR-2.1-topoTA (SEQ ID NO: 470) prior to integration into the backbone vector.

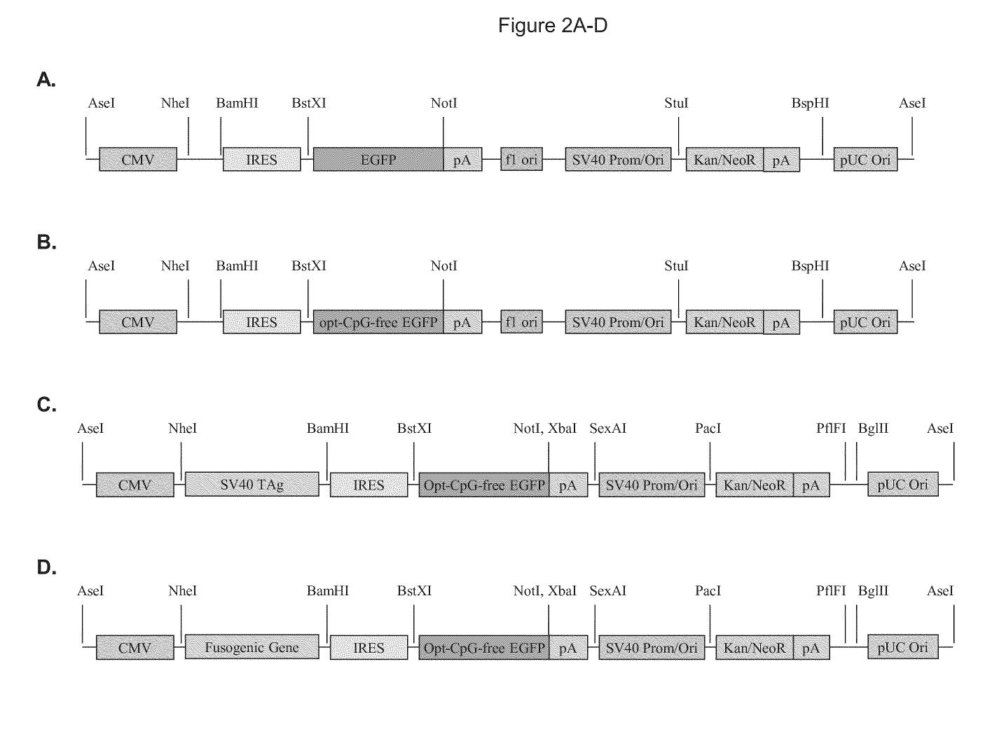

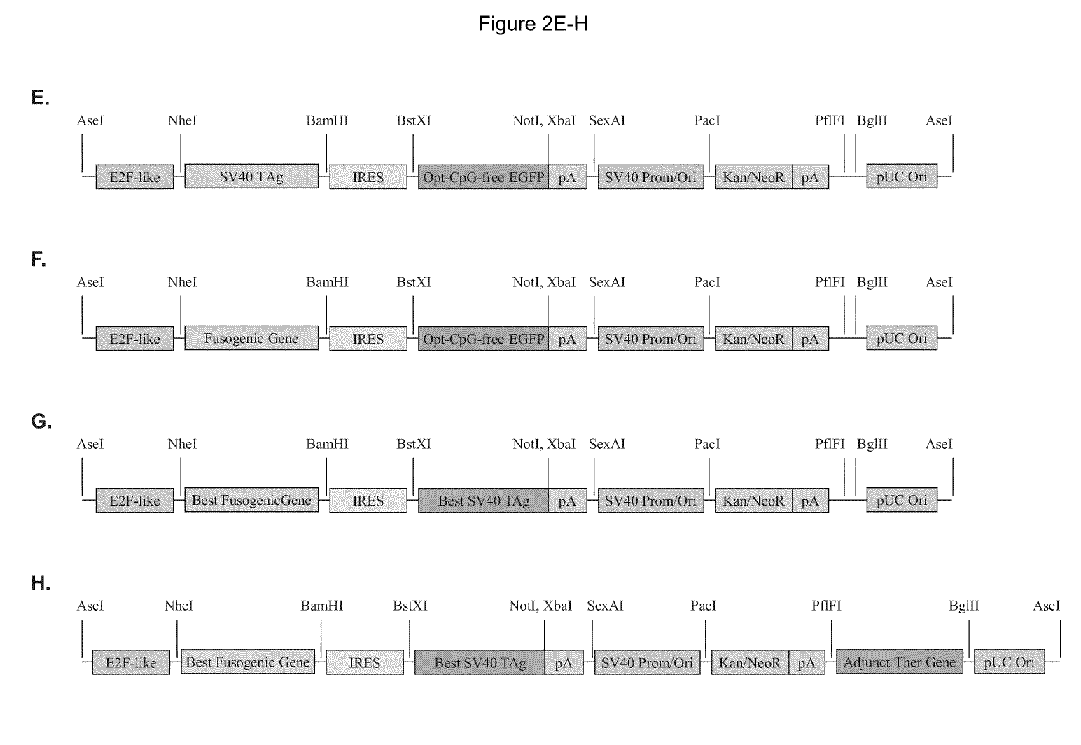

Exemplary of a backbone vector is pIRES2-EGFP (SEQ ID NO: 1). The pIRES2-EGFP contains a bacterial pUC origin of replication between restriction sites BspHI and AseI (SEQ ID NO: 118); a CMV eukaryotic promoter between restriction sites AseI and NheI (SEQ ID NO: 504); a multiple cloning site between restriction sites NheI and BamHI (SEQ ID NO: 112); an internal ribosome entry site (IRES) from Encephalomyocarditis virus (EMCV) (SEQ ID NO:104) between restriction sites BamHI and BstXI; an enhanced green fluorescent protein (EGFP) gene (SEQ ID NO:543, encoding a protein set forth in (SEQ ID NO: 544)) between restriction sites BstXI and NotI; an SV40 polyadenylation (pA) signal sequence (SEQ ID NO: 191); an f1 single-strand DNA origin (f1 ss ori; SEQ ID NO: 117); an SV40 early promoter and an SV40 origin of replication (SEQ ID NO: 113) between restriction sites NotI and StuI; a Kanamycin/Neomycin resistance gene (Kan/NeoR; SEQ ID NO:105, encoding a protein set forth in SEQ ID NO: 110); and an HSV thymidine kinase (TK) polyadenylation signal sequence (HSV-1TK-pA) between restriction sites StuI-BspHI (SEQ ID NO: 192). Features of the backbone vector pIRES2-EGFP are set forth in FIG. 2A.

The above backbone vector can be used as a starting vector for which backbone modifications can be made and incorporated. The objective in modifying the backbone is to remove unwanted segments (e.g., f1 ss ori); to introduce or relocate unique restriction sites to permit insertion and swapping of replicative, fusogenic, reporter, and/or other genes or sequences of interest; to add an adjunct therapy gene, such as a pro-drug modifying gene; and to reduce the CpG content of components of the backbone construct, such as of the pUC ori and the SV40 ori without destroying their functions. For example, the individual components can be modified to remove CpG motifs and, in cases where the modified component encodes a protein, to optimize for human codon usage. Components of the backbone construct also can be individually tested and optimized. Such experiments can be performed in parallel with the design and generation of the experimental vectors, including the self-replicative and fusogenic vectors, as described below. Desired modifications can be included in the final integrated vector.

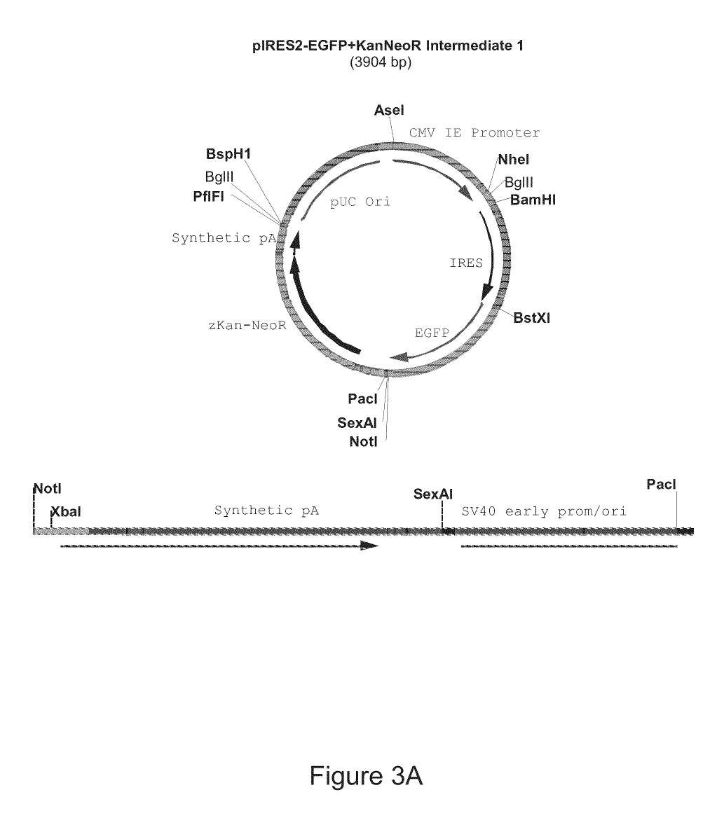

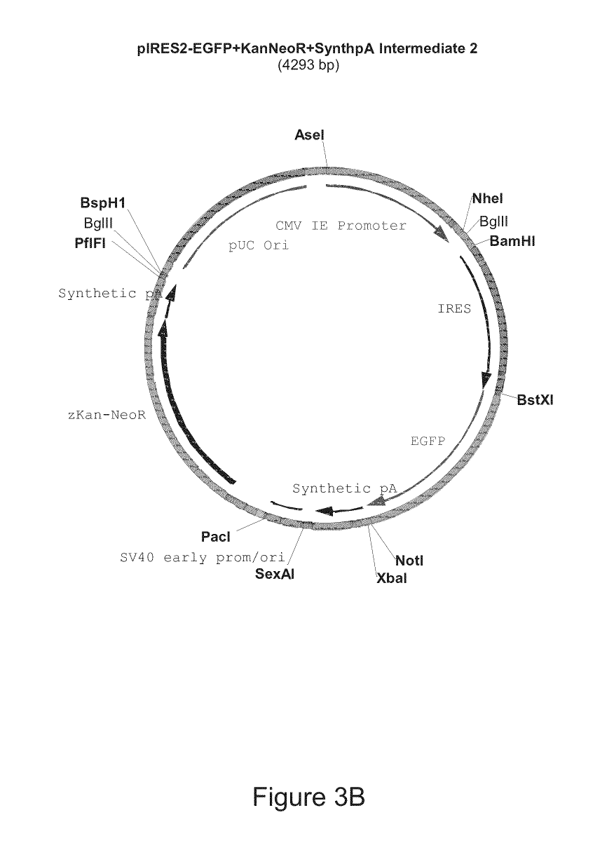

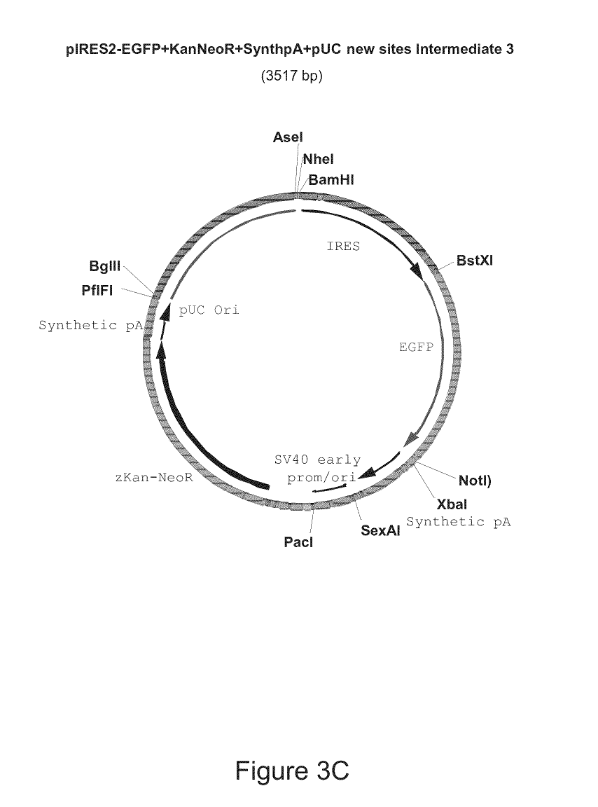

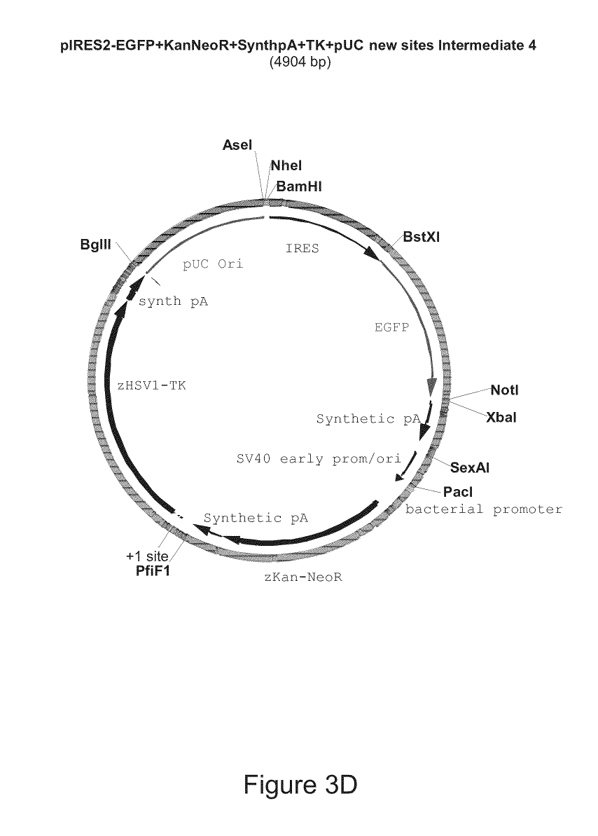

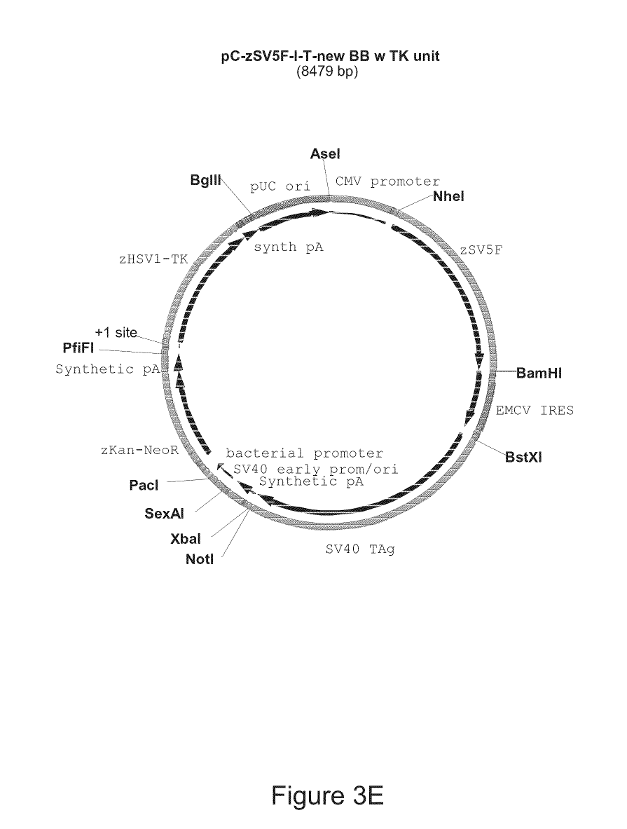

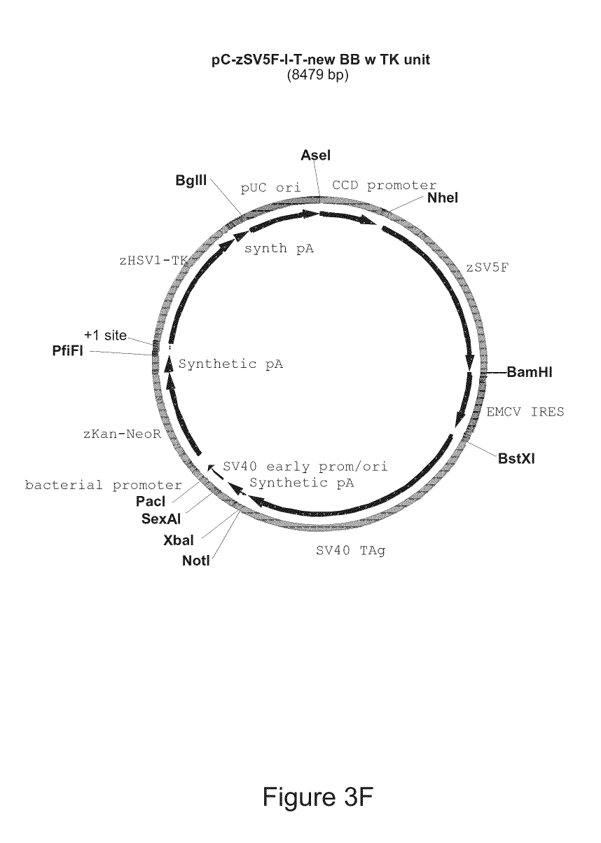

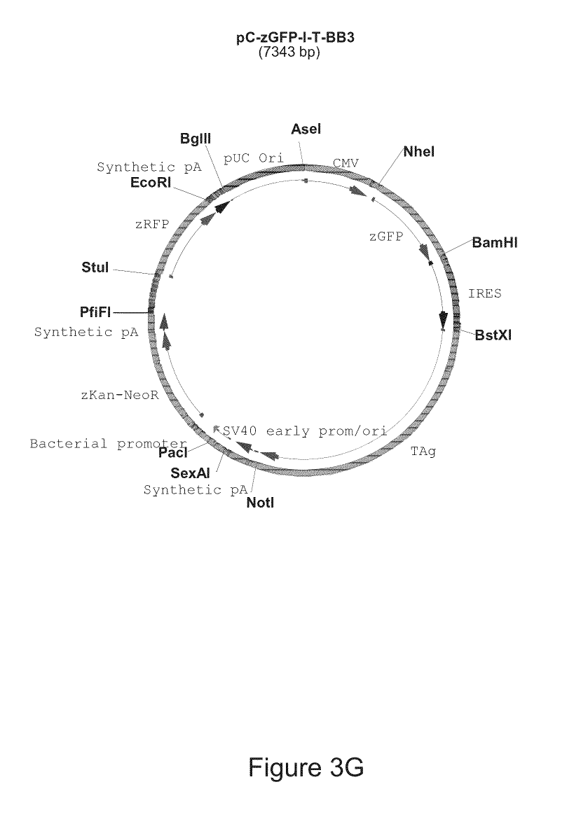

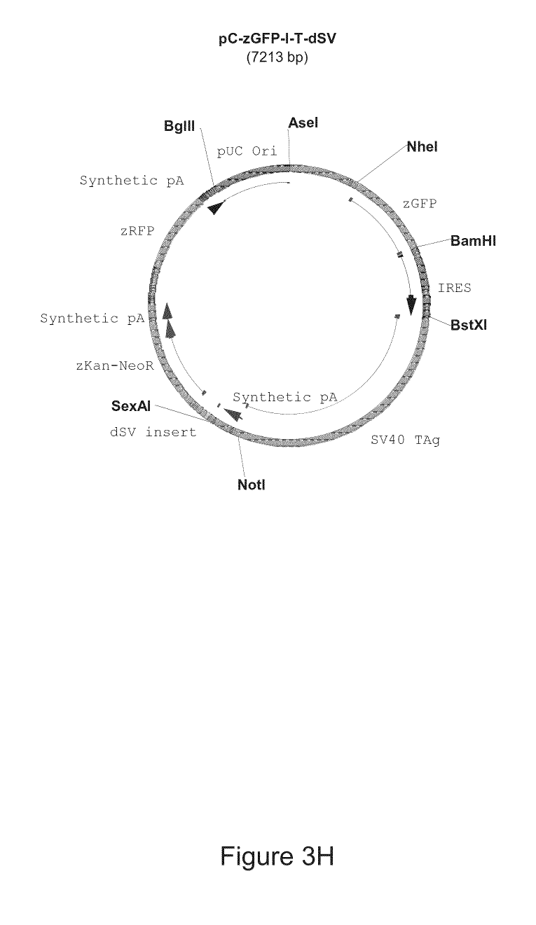

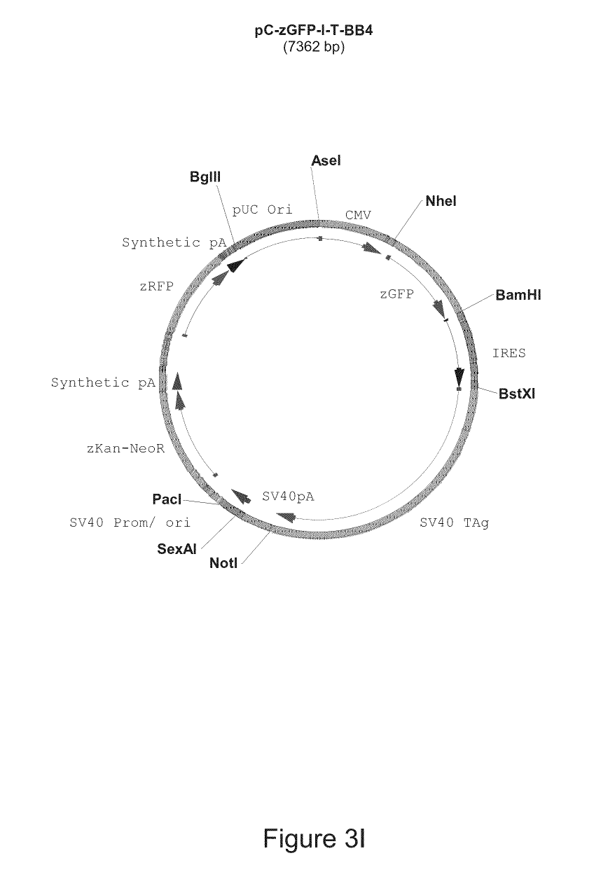

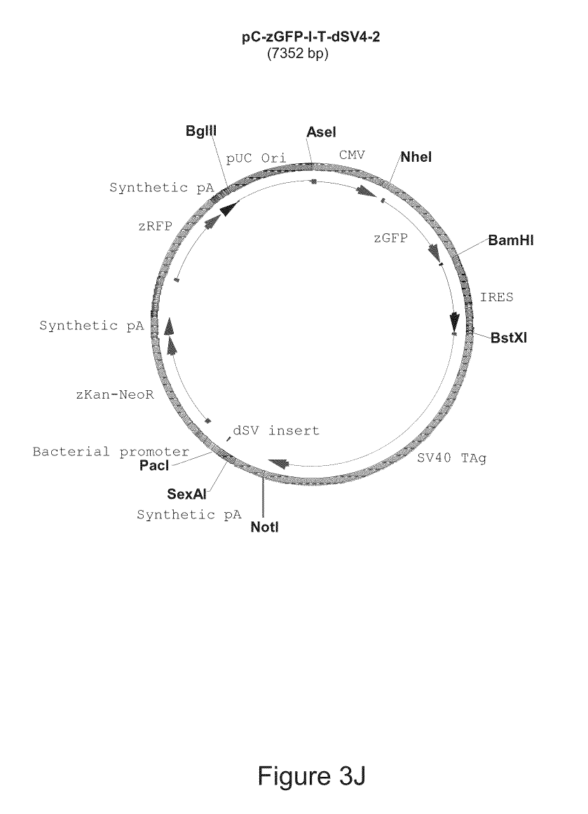

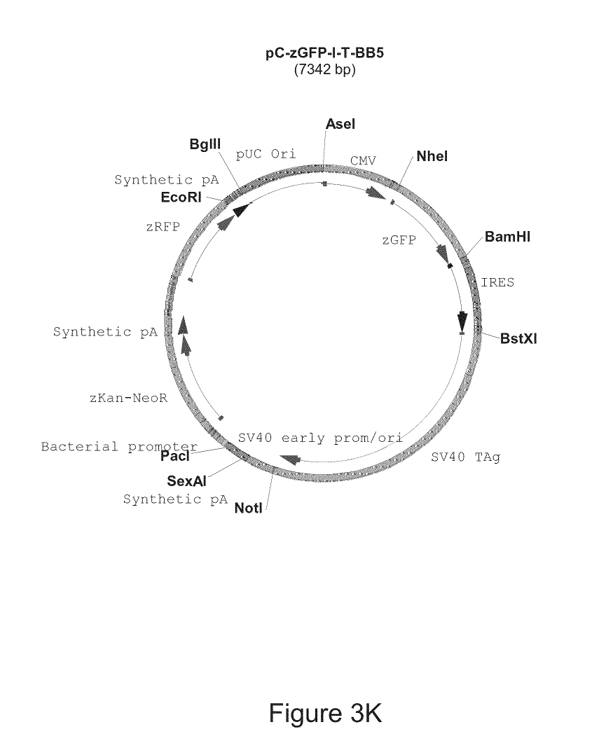

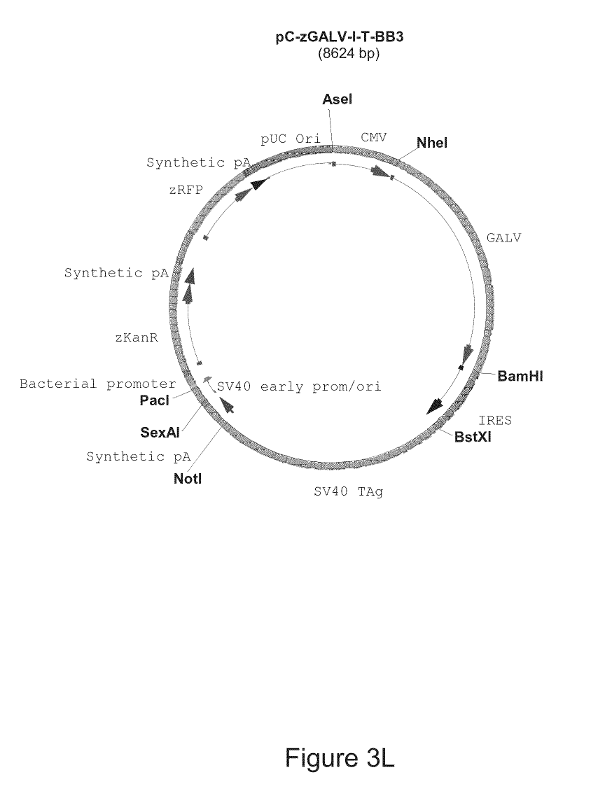

Example 5 herein details the generation of exemplary further intermediate backbone vector constructs that were generated from pIRES2-EGFP. For example, Intermediate 1 (SEQ ID NO: 2) is a backbone vector derived from pIRES2-EGFP that removes the SV40 prom/ori, introduces new restriction sites, and replaces the Kan/NeoR transcription unit with a human codon-optimized and CpG-free Kan/NeoR transcription unit (SEQ ID NO: 106), including a synthetic cell cycle-dependent promoter (SEQ ID NO: 107), a CpG-free, human codon-optimized Kan/NeoR gene (SEQ ID NOS: 108 and 109, encoding a Kan/NeoR protein set forth in SEQ ID NO: 110), and CpG modified pA signal for the Kan/NeoR gene (SEQ ID NO: 193), between NotI and BspH1 restriction sites. New SexA1, PacI, Pf1FI, and Bg1II restriction sites are also integrated into Intermediate 1 (see FIG. 3A).

Intermediate 2 (SEQ ID NO: 3) is a backbone vector derived from Intermediate 1 wherein a fragment, containing a CpG modified synthetic polyadenylation sequence and a CpG-modified SV40 promoter/ori separated by a SexAI restriction site, is incorporated between the newly introduced NotI and SexAI restriction sites (see FIG. 3B). Various modifications of the SV40 promoter/ori sequence in Intermediate 2 backbone can be functionally tested and optimized for replication in mammalian cells known to express SV40 TAg, e.g.

Intermediate vector 3 (SEQ ID NO:4) is constructed from backbone Intermediate 2 wherein the pUC ori, CMV promoter, and multiple cloning site, between the Pf1FI and BamHI restriction sites, are replaced with a synthetic fragment containing a CpG-modified pUC, flanked by Bg1II and AseI restriction sites, and an NheI site all contained between Pf1FI and BamHI restriction sites (see e.g. FIG. 3C). Various modifications of the pUC ori sequence in Intermediate 3 backbone can be functionally tested and optimized for replication in bacteria.

Backbone constructs also can be constructed to contain adjunct therapy genes, in addition to the fusogenic gene. It is understood that any additional adjunct therapy gene can be included in a construct herein, including but not limited to expression of a suicide gene, a pro-drug modifying enzyme, a cytotoxic protein, an apoptosis-inducing protein, proteins that interfere with cellular survival or proliferation, an immunomodulatory protein or an angiogenesis inhibitor. Exemplary of an adjunct therapy gene is the pro-drug modifying gene HSV-TK. A backbone construct, designated intermediate 4 backbone construct, containing this adjunct therapy gene is exemplified in FIG. 3D, which contains an additional transcription unit for the HSV-TK gene flanked by Pf1FI and Bg1II restriction sites (SEQ ID NO: 5). Hence, any additional transcription unit can be inserted into a construct between the Pf1FI and Bg1II restriction sites using this intermediate vector. For example, further backbone or intermediate constructs can be generated containing a reporter gene or other gene in that position. This is exemplified for the backbone construct designated BB3, which is exemplified in FIG. 3G, and contains a further reporter gene (e.g. red fluorescent protein) flanked by Pf1FI and Bg1II restriction sites (SEQ ID NO: 607).

Example 5 sets forth the generation of exemplary backbone constructs including Intermediate 1, Intermediate 2, Intermediate 3 and Intermediate 4 (see e.g., FIG. 3A-3D). The intermediate 3 and 4 backbone constructs are set forth in SEQ ID NOS: 4 and 5, respectively. Each of the intermediate backbone constructs, such as intermediate 3 and intermediate 4 backbone constructs, can be modified further to remove components and/or optimize sequences as desired. This is exemplified for the generation of further intermediate constructs designated BB3 (see e.g. SEQ ID NO:607 and FIG. 3G) and BB4 (see e.g. SEQ ID NO:719 and FIG. 3I) and BB5 (SEQ ID NO:726 and FIG. 3K). Any of the intermediate vectors also can be used to generate non-replicating vectors lacking the SV40 core origin (see e.g. SEQ ID NO: 608 and 721 and FIGS. 3H and 3J). In other examples, the Intermediate 3 backbone construct and the Intermediate 4 backbone construct, or other backbone or intermediate constructs described herein or derived from any described herein, can be used as recipient vectors for testing pUC origins that can be modified to reduce CpG motifs. Newly synthesized versions of pUC oris or any other test oris can be inserted into the unique Bg1II-Ase1 restrictions sites. The functional assay to test for the function of the pUC ori is the ability of the plasmid construct to replicate in E. coli bacteria.

Any one or more of the components can be inserted to replace any one of the above components in any of the above backbone constructions. For example, the initial backbone construct can be used to generate experimental test vectors to assess the expression, replication and/or fusogenic activities of one or more components alone or in combination. The series of experimental and backbone intermediate constructs can be generated in parallel. For example, one experimental vector set can be generated to assess the replication components, including those components that permit self-replication, and in particular, to select for an SV-T mutant that can induce replication without inducing transformation. Another experimental vector set can be generated to assess various fusogenic or other oncolytic genes, and variants thereof, for fusogenic or cytotoxic activity and/or bystander effects.

2. Experimental Test Vector Backbones

Experimental vectors can be generated whereby one or more components can be tested for replicative, fusogenic, oncolytic, cytotoxic, bystander, and/or other desired activity or activities. In order to test the replication and fusogenic activity of the above experimental intermediate vectors, a reporter gene can be used. Once activity is confirmed, the component parts can be integrated into one or more final vectors that exhibit replicative, fusogenic, oncolytic, cytotoxic, bystander, and/or other desired activity.

Exemplary of a reporter gene are any described in Section B above. For example, the reporter gene can be RFP or EGFP. For example, the EGFP already contained in the pIRES2-EGFP initial backbone vector above can be used. Alternatively, an EGFP gene can be synthesized, such as is described in Example 1, which is an EGFP gene that has been optimized for human codon usage, as well as removing CG dinucleotides (CpG motifs). The EGFP gene can be synthesized to contain flanking sequences which contain the BstXI restriction site sequence (CCANNNNNNTGG; SEQ ID NO: 554) and the NotI restriction site sequence (GCGGCCGC; SEQ ID NO: 556). The BstXI restriction site is an ambiguous one as the Ns in the site recognition formula can be any nucleotide (A, T, C, and G). For example, the Ns in the BstXI restriction site can be designed to be CAACCA, giving rise to the BstXI-recognized sequence CCACAACCATGG (SEQ ID NO: 605). This BstX1 sequence can be integrated as part of the coding sequence of the EGFP gene, where ACCATGG (corresponding to nucleotides 6-12 of the sequence set forth in SEQ ID NO: 605) becomes the Kozak sequence for efficient initiation of gene translation. The NotI restriction site does contain two CpG motifs. Although these are not optimum CpG motifs, they can be removed if desired from any final version of vectors by site directed mutagenesis. An exemplary sequence of a modified EGFP gene is set forth in SEQ ID NO: 545 and encoding a sequence of amino acids set forth in SEQ ID NO: 546.

The modified EGFP can be inserted into pIRES2-EGFP to replace the unmodified EGFP. For example, the pIRES2-EGFP vector can be digested with BstXI/NotI and the digested vector can be ligated together with the modified EGFP fragment using standard molecular biology techniques. Upon transformation and purification, the resultant vector can be sequenced. Exemplary primers for sequencing include a forward primer located within the IRES sequence having a sequence of 5′-GAGGTTAAAAAAACGTCTAGG-3′ (SEQ ID NO: 463; synthesized by Allele Biotechnology, San Diego, Calif.) and a reverse primer located within the SV40pA sequence having a sequence of 5′-TTTCAGGTTCAGGGGGAGGTG-3′ (SEQ ID NO:464; synthesized by Allele Biotechnology). Such an intermediate backbone vector is termed pIRES2-zGFP and a plasmid map and sequence are set forth in FIG. 2B and SEQ ID NO: 694, respectively. The backbone vector can be further modified by inserting genes to be tested as described below.

a. Replication Competent Vector

A first series of experimental intermediate vectors can be made to test the replication activity of a replication initiator protein. For example, a gene or a modified form of a gene for a replication initiator, including but not limited to the SV40 TAg, and other polyomaviruses, and Epstein-Barr virus nuclear antigen (EBNA) for Epstein-Barr virus (EBV) can be tested for replicative activity. In addition, a gene or modified form of a gene for a replication initiator also can be tested for transforming activity. Typically, as described in Section B, constructs herein are designed such that replicative and transforming activities are uncoupled so that the vector constructs are capable of replication but exhibit minimal to no transforming activities.

In one example, vectors containing the SV40 TAg gene or modified form thereof can be generated and tested for activity. For example, SV40 TAg gene sequence (SEQ ID NOS: 561; encoding the amino acid sequence set forth in SEQ ID NO: 564), or modified forms thereof such as any provided herein or known to one of skill in the art, can be inserted into the multiple cloning site of the pIRES2-zGFP vector, for example between the NheI and BamHI restriction sites. As discussed above, the SV40 TAg sequence can be optimized for human codon usage and/or can be modified to remove CpG motifs without (SEQ ID NOS: 562 or 563, both encoding the amino acid sequence set forth in SEQ ID NO: 564). In addition, other modifications, which result in modifications of the TAg protein, known in the art including any described herein (see e.g., any of SEQ ID NOS: 565-604), can be used. Thus, any SV40 TAg sequence including, but not limited to, SEQ ID NOS: 561-563 or nucleic acid sequences encoding proteins set forth in SEQ ID NOS: 564-604 can be inserted into the pIRES2-zEGFP vector or other backbone cassette. Any SV40 TAg gene sequence contemplated to be inserted can be artificially synthesized to contain flanking NheI (GCTAGC; SEQ ID NO: 555) and BamHI (GGATCC; SEQ ID NO: 551) restriction sites to permit cloning into the multiple cloning site in pIRES2-zGFP. The SV40 TAg sequence also can contain internal BstXI-NotI sites, so that it can be cut out of the vector and moved into the BstXI-NotI position in the final vector (i.e. replacing the EGFP sequence currently residing in the intermediate vector). Sequences of resulting experimental intermediate vectors can be confirmed using the forward primer sequence located in the CMV promoter having a sequence 5′-GTAGGCGTGTACGGTGGGAGG-3′ (SEQ ID NO: 462; Allele Biotechnology) and a reverse primer located in the IRES element having a sequence of 5′-CATATAGACAAACGCACACC-3′ (SEQ ID NO: 464; Allele Biotechnology). The features of the resulting vector are set forth in FIG. 2C. The resulting vector is designated pC-T-I-zGFP and has a sequence of nucleotides set forth in SEQ ID NO: 697.

Any of the vectors containing SV40 TAg or a modified form of SV40 TAg, including any derived from pC-T-I-zGFP, can be tested for replicative activity as described in Section E below. In addition, the vectors also can be tested for transforming activity to identify a mutant whose replicative and transforming activities are uncoupled. Such an analysis permits the identification of mutations of SV40 TAg that allow replication despite the mutations eliminating the binding to Rb, p53 or HSP70 protein (to minimize transforming activity). Initial tests can be performed using permissive (Rb−/− and/or p53−/−) tumor cells lines, with expression of the SV-T driven by the cytomegalovirus (CMV) promoter contained within these intermediate constructs. As discussed below, the plasmid copy number can be correlated to expression of a reporter gene, such as EGFP fluorescence. Plasmid copy number also can be determined by quantitative real-time polymerase chain reaction, also called qPCR. EGFP fluorescence can be determined by direct cell fluorescence, by average cellular fluorescence using flow cytometry, or by measuring the fluorescence of cell lysates. Control studies can be performed by placing an irrelevant gene, such as Dihydrofolate Reductase (DHFR), into a control vector in place of the SV40 TAg. The modified SV40 TAg capable of uncoupling replication and transformation also can be tested for replication abilities compared to the wild-type TAg. Candidate TAg mutations can be identified that retain autonomous vector replication but do not exhibit transforming activities.

b. Fusogenic Competent Vectors

The second series of experimental intermediate vectors can be made by inserting into the pIRES2-zGFP, or other experimental intermediate vector or backbone construct, a fusogenic gene. Fusogenic genes that can be tested include any set forth in Section B above. Viral fusogenic proteins include, but are not limited to, Simian Virus 5F (SV5F), Vesicular Stomatitis Virus G protein (VSVG), Gibbon Ape Leukemia Virus envelope protein (GALV), Avian Reovirus (ARV) p10, Reptilian Reovirus (RRV) p14, and Baboon Reovirus (BRV) p15, or modified forms thereof. To permit efficient insertion of the fusogenic gene into the pIRES2-zGFP vector or other backbone vector, the sequences can be synthesized to contain flanking restriction sites to allow insertion into any of the backbone vectors described herein. In examples herein, the sequences can be synthesized to contain flanking NheI and BamHI sites to allow insertion into the multiple cloning site of the vector.

For example, the SV5F can be introduced as the fusogenic gene. The wild type sequence of SV5F is set forth in SEQ ID NO: 18 and encodes a sequence of amino acids set forth in SEQ ID NO: 44. Nucleic acids encoding Gly to Ala substitutions of F proteins, such as modified SV5F provided herein, also can be inserted into the vector and tested for fusogenic activity (see SEQ ID NOS: 19-25). Exemplary of these are mutant SV5F sequences that have been artificially synthesized to contain flanking NheI and BamHI restriction sites such as set forth in any of SEQ ID NOS: 82-90 (each with a 5′ NheI sequence corresponding to nucleotides 1-6 and a 3′ BamHI sequence corresponding to nucleotides 1603 to 1608), and encoding a sequence of amino acids set forth in any of SEQ ID NOS: 44-51, respectively.

Other fusogenic genes (e.g., SEQ ID NOS: 6-18, 26-36), including modified genes designed to be human optimized and CpG-free and/or modified to have more fusogenic activity in “stand alone” form as discussed above, also can be artificially synthesized with flanking restriction sites (e.g., SEQ ID NOS: 70-78, 80-83, 91-101) for insertion into the pIRES2-zGFP vector. These include, but are not limited to, a wild type VSVG set forth in SEQ ID NO: 6 or a human codon optimized and CpG-free form (zVSVG) set forth in SEQ ID NO: 7, each encoding a sequence of amino acids set forth in SEQ ID NO: 38; a wild type AVRp10 set forth in SEQ ID NO: 8 or a human codon optimized, CpG free form (zAVRp10) set forth in SEQ ID NO: 9, each encoding a sequence of amino acids set forth in SEQ ID NO: 39; an AVRp10 S1133 variant set forth in SEQ ID NO:10 or a human codon optimized, CpG free form (zAVRp10(S1133)) set forth in SEQ ID NO:11, each encoding a sequence of amino acids set forth in SEQ ID NO: 40; and a wild type RRVp14 set forth in SEQ ID NO:12 or a human codon optimized, CpG free form (zRRVp14) set forth in SEQ ID NO:13, each encoding a sequence of amino acids set forth in SEQ ID NO:41.

For example, following ligation into the pIRES2-zGFP vector digested with NheI and BamHI, fusogenic intermediate vectors can be generated. The intermediate vectors can be named after the fusogenic gene contained therein. Exemplary of such vectors derived from pIRES2-zGFP include, for example, pCzARVp10-I-zGFP (SEQ ID NO: 715), pCzRRVp14-I-zGFP (SEQ ID NO: 716), pCzBRVp15-I-zGFP (SEQ ID NO: 717), pCzSV5F-I-zGFP (SEQ ID NO: 718), pCzVSVG-IzGFP (SEQ ID NO: 714), and pCzGALV-I-zGFP (SEQ ID NO: 713). The features of such vector constructs are set forth in FIG. 2D. Any of the experimental intermediate fusogenic vectors including any containing a fusogenic gene, or modified form thereof, can be tested for fusogenic activity as described in Section E below. For example, vectors containing the fusogenic genes or mutations thereof, can be transfected into cells, such as 293T cells, and examined for their ability to cause cell fusion. EGFP expression facilitates observation and evaluation of the formation of cellular syncytia as described in Section E below.

c. Tissue or Cell Specificity/Selectivity Competent Vectors

A third set of experimental intermediates vectors can be made by inserting into the pIRES2-zGFP or other intermediate or backbone construct a conditional promoter, such as a cell-cycle dependent promoter. Exemplary promoters, including cell cycle-dependent promoters, are set forth in Section B above. The promoter can be tested for its tissue-specific or cell-specific activity. Exemplary of promoters are cell cycle-dependent promoters, such as, for example, an E2F responsive promoter.

The cell-cycle promoter can be artificially synthesized to also contain flanking restriction sites to be easily inserted into a backbone or intermediate experimental vector provided herein. In particular examples, the cell cycle-dependent promoter is synthesized to containing a flanking AseI (ATTAAT; SEQ ID NO: 550) and NheI (GCTAGC; SEQ ID NO: 555) restriction sites, which permits insertion into the pC-T-I-zGFP vector or derivative thereof in place of the CMV promoter.

Exemplary of a cell cycle-dependent promoter is E2F1, which is turned off in the presence of tumor suppressor genes such as Rb family genes or p53, and thus is active in cells, such as tumor cells, that are deficient in these proteins. Exemplary E2F1 promoters are set forth in SEQ ID NO: 534 or SEQ ID NO: 535 (containing an A262T mutation), which each contain a 5′ AseI restriction site (corresponding to nucleotides 1-6) and a 3′ NheI restriction site (corresponding to nucleotides 274-279). E2F1 promoters that are modified by removing CpGmotifs are set forth in SEQ ID NOS: 536 and 537 (A262T mutant). Other truncated forms of the E2F1 promoter are set forth in any of SEQ ID NOS: 538-541. Each of the above sequences contains E2F1 promoters with a 5′ flanking AseI restriction sequence and a 3′ NheI restriction sequence. It is understood that similar promoter sequences can be generated or synthesized without flanking restriction sites or with any flanking restriction site sequence depending on the particular backbone vectors.

The activity of the conditional promoters contained in each of the above vectors can be tested in permissive (Rb−/− and/or p53−/−) tumor cells lines or normal cells such as is described herein, and promoter activity can be measured by plasmid copy number measured by qPCR and/or by EGFP fluorescence. Since E2F1 is a conditional promoter that is active in tumor cells, but not in normal cells, the resulting vectors can be tested to determine the replicative and fusogenic activities of the resulting vectors in cells deficient in, for example, p53 or Rb family members, as compared to normal cells, such as by using any of the assays described herein.

The best conditional promoter candidates can be identified from above, and can be integrated into experimental test vectors containing a replication component and/or an therapeutic component capable of bystander activity, such as any generated and tested in the subsections above. For example, a conditional promoter candidate can be subcloned into an intermediate series of vectors containing TAg or modified forms thereof. For example, the vector designated pC-T-I-zGFP (see FIG. 2C) can be digested with AseI and NheI to remove the CMV promoter, and any candidate promoter with the compatible restriction sites ligated therein. Thus, a series of vector combinations containing various permutations of E2F1 and SV40 TAg combined into one vector can be tested for cell cycle-specific replicative activity and/or transforming activity. The features of such resulting vectors are set forth in FIG. 2E.

In other examples, any conditional promoter candidate, such as any of the above E2F1 sequences, can be subcloned into an intermediate series of vectors containing an oncotherapeutic gene capable of bystander activity, or modified form thereof. For example, any vector with features set forth in FIG. 2D (e.g. pCzARVp10-I-zGFP, pCzRRVp14-I-zGFP, pCzBRVp15-I-zGFP, pCzSV5F-I-zGFP, pCzVSVG-I-zGFP and pCzGALV-I-zGFP) can be digested with AseI and NheI to remove the CMV promoter, and any candidate promoter with the compatible restriction sites ligated therein. Thus, a series of vector combinations containing various permutations of a candidate promoter, such as an E2F1 or modified form thereof, and an oncotherapeutic bystander gene combined into one vector can be tested for fusogenic activity. The features of the resulting vector are set forth in FIG. 2F.

3. Integration of Constructs to Generate an Oncovector

Oncovector constructs, including those capable of bystander activity (e.g. fusogenic activity), can be developed based on the analysis of the above experimental intermediates and backbone vectors and integration of each of the components. For example, any of the above experimental intermediate constructs can be generated and tested to identify any one or more of a replication initiator, oncotherapeutic bystander gene, or cell-cycle dependent promoter, or modified forms thereof, to use in the resulting oncovector constructs. Any resulting construct can be developed to contain any one or more desired components, and to also contain unique restriction sites. Such unique restriction sites permit the further optimization and testing of components by facilitating integration of other fusogenic genes or mutants thereof, or other replication initiators or mutants thereof, including mutants of SV40 TAg.

Accordingly, once the best conditions and gene variants, from the therapeutic bystander activity-capable intermediates and the replication intermediates (e.g., containing SV40 TAg or modified SV40 TAg), are identified, the two elements can be combined into one test vector. In addition, the optimal cell cycle-dependent promoter can be added into this final vector. Further, an adjunct therapy gene also can be included. The components can be separate transcription units or can be contained within a single transcription unit. Thus, constructs containing combinations of elements from any of the above experimental intermediates can be combined and tested for activity.

To generate a final integrated construct, the transcription unit of an experimental construct containing the promoter, replication initiator, and/or therapeutic bystander gene is integrated into a backbone construct, such as any of the exemplary intermediate 1-4 backbone constructs set forth above and in Example 5.

Restriction sites engineered into the backbone constructs facilitate the integration and subcloning steps. For example, the transcription units of backbone constructs 1-4 are flanked by AseI and NotI restriction sites. Thus, an AseI/NotI digested transcription unit of any of the experimental vectors (see FIG. 2, e.g., CMV-TAg-IRES-zGFP; cell cycle-dependent (CCD) promoter-TAg, IRES-zGFP; CMV-oncotherapeutic bystander gene-IRES-zGFP; CCD promoter-oncotherapeutic bystander gene-IRES-zGFP; or any combination of integrated units such as CMV-oncotherapeutic bystander gene-IRES-TAg; CCD promoter-therapeutic bystander gene-IRES-TAg) can be ligated into an intermediate backbone vector, such as Intermediate 3 or Intermediate 4 backbone construct, at the unique AseI-NotI cloning sites. For example, a transcription unit from an experimental intermediate construct containing a CMV promoter or a CCD promoter, human optimized and CpG-free SV5F fusogenic gene, an EMCV IRES, and a human optimized CpG-free TAg can be generated by digestion of an experimental intermediate construct with AseI/NotI. The recipient Intermediate 4 vector also can be digested with AseI/NotI for ligation of the digested fragment by standard procedures. Exemplary of such a resulting vector is set forth in FIG. 3E (containing a CMV promoter) and FIG. 3F (containing a CCD promoter). The resulting constructs can be tested for replication, oncotherapeutic, and bystander activities.

Table 14 below sets forth exemplary components of an oncovector construct, such as an oncovector construct depicted in FIG. 3F, including exemplary restriction sites that permit substitution of any one or more of the components. It is understood that the order of the components can be reversed or altered. For example, the replication initiator (e.g. SV40-TAg) need not be contained on the same nucleic acid molecule construct as the cognate origin of replication, but can be contained on a separate nucleic acid molecule for expression therefrom, or can be stably expressed from a recipient cell line. Also, the order of the therapeutic bystander gene and replication initiator can be reversed. In addition, in some examples, the therapeutic bystander gene, or replication gene, can be removed and replaced with a reporter gene, such as for example, a gene encoding a fluorescent protein (e.g. GFP, RFP, or mKate), Luciferase or beta-galactosidase. In further examples, the therapeutic bystander gene can be removed or replaced with another adjunct therapy gene. In some examples, the nucleic acid construct can contain a single transcription unit for replicative, therapeutic, and bystander activities. In other examples, the nucleic acid construct can additionally contain a transcription unit for adjunct gene therapy.

TABLE 14
Summary of Components in an Exemplary Oncovector Construct

| Restriction | |
|---|---|
| Sites | Component |
| AseI-NheI | Cell cycle-dependent promoter (e.g., E2F-like, |
| | telomerase-like, or modified forms thereof) |
| NheI-BamHI | Oncotherapeutic Bystander Gene (e.g., VSVG, |
| | SV5F, ARVp10, RRVp14, BRVp15, CDase, or |
| | modified forms thereof) |
| BamHI-BstXI | IRES (e.g., EMCV, CPLV) |
| BstXI-NotI | Replication initiator (e.g., TAg, including |
| | modified forms thereof such as human codon |
| | optimized and CpG fee, or a mutant thereof) |
| NotI-SexAI | Synthetic pA signal |
| SexAI-PacI | Origin (e.g., SV40 early promoter/ori, or |
| | modified form thereof) |
| PacI-PflFI | Bacterial promoter; antibiotic resistance |
| | gene (e.g., Kan/NeoR, or modified form |
| | thereof); synthetic pA |
| PflF1-BglII | Cell cycle-dependent promoter, or modified |
| | form thereof; adjunct therapy gene (e.g., |
| | HSV1-TK, CDase, cytokine, Chemokine, or |
| | modified form thereof); synthetic pA |
| BglII-Ase1 | Bacterial origin of replication (e.g., pUC |
| | origin or modified form thereof) |

The features of an exemplary final oncovector construct are set forth in FIG. 2G. In this example, candidate E2F-like promoter, candidate oncotherapeutic bystander gene and/or candidate SV40-TAg are identified from the generation and testing of the experimental vectors as described above and used to generate a final construct. It is understood that FIG. 2G is exemplary only and that a resulting oncovector construct can contain any cell type- or tumor-specific promoter of choice that exhibits cell type-specific promoter activity, any oncotherapeutic bystander gene that exhibits oncotherapeutic and bystander activities and/or any replication initiator that exhibits replicative activity but no or little transformation activity. Also, it is understood that the order of the components also can be varied. Typically, the resulting construct is bicistronic such that the replication initiator and the fusogenic gene are expressed under the same promoter, but this is not required. For example, the replication initiator and fusogenic gene can be expressed under different promoters that are the same or different. The construct also can be generated to contain a further adjunct therapy gene, reporter gene or other gene of interest, which is exemplified in FIG. 2H.

The constructs can be tested against a series of cell lines, both cancerous and normal, for oncotherapeutic, bystander, and replicative activities. These studies can be designed to determine which cell types are permissive to the self-replication, oncotherapeutic, and bystander actions of the construct.

Control constructs also can be generated. For example, replicative (e.g. designated BB3) and non-replicative (designated dSV) vector pairs can be generated that are identical except for their ability to support autonomous replication. As discussed above, exemplary oncovectors provided herein are autonomous replicating plasmids (ARPs) because they contain an SV40 TAg in combination with the SV40 ori region so that plasmid replication and amplification of the transgenes contained therein (e.g. a reporter or fusogenic gene) is achieved. Multimers of the TAg bind to GAGGC motifs (SEQ ID NO: 122) within the core of the SV40 ori. The wild type SV40 recognition sequence contains 4 TAg binding domains (two in forward and two in reverse orientation) within the core TAg binding domain (GAGGCGGAGGCCGCCTCGGCCTC; SEQ ID NO: 123) of the SV40 ori, which initiates DNA replication. A replication competent vector (e.g., BB3) includes the core TAg binding sites of the SV40 ori and all or some of a 5′ enhancer region. A replication incompetent vector (e.g., dSV) lacks the core TAg binding sites of the SV40 ori and some or all of the 5′ enhancer regions. Also, for the replication incompetent control vector, the nucleic acid molecule also can include a unique linker sequence added for recognition, for example for diagnostic purposes. Exemplary of such a linker sequence added for recognition is GGAGGGGAGGAGG (SEQ ID NO: 678). For example, the dSV ori sequence (including 5′ SexAI restriction site, reduced 5′ enhancer, linker and a 3′ PacI restriction site) can be reduced to the region ACCTGGTTAGGAGGGGAGGAGGATTAATAA (SEQ ID NO: 111). Hence, the replication incompetent plasmid designated dSV is typically 100 base pairs shorter than the BB3 SV40 ori.

### E. METHODS OF PRODUCING ONCOVECTOR CONSTRUCTS

Any suitable method for generating oncovector constructs can be used. Exemplary methods for generating nucleic acid molecules, including any of the constructs provided herein, are provided. Such methods include in vitro synthesis methods for nucleic acid molecules such as PCR, standard cloning methods, synthetic gene construction such as by using overlapping oligos, and in vitro ligation of isolated and/or synthesized nucleic acid fragments. In one example, nucleic acid molecules for any of the components of the constructs provided herein, can be isolated by cloning methods, including PCR or RNA and DNA isolated from primary cells or transfected cells. In another example, nucleic acid molecules for any of the components of the constructs provided herein can be artificially synthesized.

1. Synthetic Genes and Peptides

Nucleic acids molecules can be synthesized by methods known to one of skill in the art using synthetic gene synthesis. For example, individual oligonucleotides corresponding to fragments of a construct sequence of nucleotides are synthesized by standard automated methods and mixed together in an annealing or hybridization reaction. Thus, in some strategies, synthetic genes are assembled from a large number of short partially overlapping DNA oligonucleotides, generally about 100 nucleotides in length. Such oligonucleotides can be commercially obtained, such as from Integrated DNA Technologies (Coralville, Iowa). Adjacent overlapping oligonucleotides contain sequences from opposite strands of the desired gene and have complementary overlapping ends. These segments are allowed to anneal and then assembled into longer double-stranded DNA, for example, by ligation and/or polymerase extension reactions, either alone or in combination. Single nucleotide “nicks” in the duplex DNA are sealed using ligation, for example with bacteriophage T4 DNA ligase. Such strategies are variously referred to as “assembly PCR,” “splicing by overlap extension,” “polymerase chain assembly” and others. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to a nucleic acid molecule by gene synthesis methods, including, for example, linker sequences containing restriction endonuclease sites for the purpose of cloning the synthetic gene into a backbone construct vector. Furthermore, additional nucleotide sequences specifying functional DNA elements can be operatively linked to a nucleic acid molecule. Examples of such sequences include, but are not limited to, regulatory sequences such as promoter sequences or sequences that facilitate the purification and/or detection of an expressed polypeptide. For example, a fusion tag such as an epitope tag or fluorescent moiety can be fused or linked to a nucleic acid molecule.

Hence, such a strategy can be used to synthesize individual components of the constructs, which can be used in conventional cloning procedures. Thus, in one example, restriction endonuclease linker sequences are added to the 3′ and 5′ flanking ends of a synthesized gene. Such restriction sites then can be used to insert the synthetic gene into any of one of a variety of backbone construct vectors. For example, restriction sites can be introduced into synthesized genes to permit the insertion of the resulting gene fragment into the pIRES2-EGFP backbone construct, or any one or more of the experimental intermediate constructs provided herein, or into any one or more of the intermediate backbone constructs provided herein such as Intermediate 3 (set forth in SEQ ID NO: 4) or Intermediate 4 (set forth in SEQ ID NO: 5).

Synthetic gene synthesis techniques also can be used to generate a complete construct. For example, generating a 5-6-kb segment of DNA from synthetic oligonucleotides has become routine (see e.g., Smith et al. (2003) Proc. Natl. Acad. Sci., 100:11440-15445).

2. Methods of Cloning and Isolating Component Genes

Components of nucleic acid constructs provided herein, can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

For example, methods for amplification of nucleic acids can be used to isolate nucleic acid molecules for any one or more of the components provided herein. Such amplification methods include polymerase chain reaction (PCR) methods. A nucleic acid containing material can be used as a starting material from which a desired nucleic acid molecule can be isolated. For example, DNA and mRNA preparations, cell extracts, tissue extracts, fluid samples (e.g., blood, serum, saliva), samples from healthy and/or diseased subjects, or vectors or plasmids can be used in amplification methods. Nucleic acid libraries also can be used as a source of starting material. Primers can be designed to amplify a nucleic acid molecule. For example, primers can be designed based on the known sequence of one or more components. Thus, a nucleic acid sequence for one or more components of a construct provided herein can be PCR amplified using primers that hybridize to opposite strands and flank the region of interest in a target DNA. Cells or tissues or other sources known to express a target DNA molecule, or a vector containing a sequence for a target DNA molecule, can be used as a starting product for PCR amplification events. Nucleic acid molecules generated by amplification can be confirmed by sequencing.

PCR primers used in the PCR amplification also can be engineered to facilitate the operative linkage of nucleic acid sequences. For example, non-template complementary 5′ extensions can be added to primers to allow for a variety of post-amplification manipulations of the PCR product without significant effect of the amplification itself. For example, these 5′ extensions can include restriction sites, promoter sequences, restriction enzyme linker sequences, a protease cleavage site sequence or sequences for epitope tags.

3. Methods of Generating and Cloning Constructs

Constructs provided herein contain multiple components. For example, minimally a construct provided herein contains an origin of replication, a promoter and a fusogenic gene. Generally, such constructs also contain a replication initiator to enable self-replication. The constructs also can contain other components such as, but not limited to, a reporter gene, an antibiotic resistance gene, a gene encoding an adjunct therapeutic protein such as a prodrug, cytokine or chemokine and others.

Such constructs can be prepared using conventional techniques of enzyme cutting and ligation of fragments from desired sequences. For example, as described above, desired sequences can be synthesized by PCR with overlapping PCR oligonucleotides, isolated from the DNA of a parent cell which expresses the gene by appropriate restriction enzyme digestion, or obtained from a target source, such as a cell, tissue, vector, or other target source. In any of the above examples, the resulting fragment can be designed to contain 3′ and 5′ flanking restriction sites. Thus, in one example, constructs can be generated by successive rounds of ligating DNA target sequences, amplified by PCR, into a backbone construct at engineered recombinations sites. The PCR amplified product can be subcloned into a backbone construct for further recombinant manipulation of a sequence, for example, in order to create intermediate constructs or to generate a final integrated construct.

In one example, incorporation of restriction enzyme sites into a primer can facilitate subcloning of the amplification product into a backbone vector that contains a compatible restriction site, such as by providing sticky ends for ligation of a nucleic acid sequence. Subcloning of multiple PCR amplified products into a single vector can be used as a strategy to operatively link or fuse different nucleic acid sequences to generate the constructs provided herein. Other methods for subcloning of PCR products into vectors include blunt end cloning, TA cloning, ligation independent cloning, and in vivo cloning.

The creation of an effective restriction enzyme site into an artificially synthesized gene or into a primer to amplify a desired gene requires the digestion of the PCR fragment with a compatible restriction enzyme to expose sticky ends, or for some restriction enzyme sites, blunt ends, for subsequent subcloning. There are several factors to consider in engineering a restriction enzyme site so that it retains its compatibility for a restriction enzyme. First, the addition of 2-6 extra bases upstream of an engineered restriction site can greatly increase the efficiency of digestion of the amplification product. Other methods that can be used to improve digestion of a restriction enzyme site by a restriction enzyme include proteinase K treatment to remove any thermostable polymerase that can block the DNA, end-polishing with Klenow or T4 DNA polymerase, and/or the addition of spermidine. An alternative method for improving digestion efficiency of synthesized fragments or PCR products also can include concatamerization of the fragments after amplification. For example, this is achieved by first treating the cleaned up PCR product with T4 polynucleotide kinase (if the primers have not already been phosphorylated). The ends may already be blunt if a proofreading thermostable polymerase such as Pfu was used or the amplified PCR product can be treated with T4 DNA polymerase to polish the ends if a non-proofreading enzyme such as Taq is used. The PCR products can be ligated with T4 DNA ligase. This effectively moves the restriction enzyme site away from the end of the fragments and allows for efficient digestion.

Prior to subcloning of a PCR product containing exposed restriction enzyme sites into a backbone construct vector, it is sometimes necessary to resolve a digested PCR product from those that remain uncut. In such examples, the addition of fluorescent tags at the 5′ end of a primer can be added prior to PCR. This allows for identification of digested products since those that have been digested successfully will have lost the fluorescent label upon digestion.

In some instances, the use of artificially synthesized fragments or amplified PCR products containing restriction sites for subsequent subcloning into a vector for the generation of a construct can result in the incorporation of restriction enzyme linker sequences in the construct, and the resulting expressed proteins. Generally such linker sequences are short and do not impair the function of a polypeptide so long as the sequences are operatively linked.

### F. ASSAYS TO ASSESS OR MONITOR ACTIVITIES OF ONCOVECTOR CONSTRUCTS

The activities and properties of the oncovector constructs can be assessed in vitro and/or in vivo. Assays for such assessment are known to those of skill in the art and are known to correlate tested activities and results to therapeutic and in vivo activities. Exemplary in vitro and in vivo assays are provided herein to assess the biological activity of oncovector constructs. In addition, numerous assays for biological activities of oncovector constructs are known to one of skill in the art, and any assay known to assess the activity of an oncovector construct can be chosen depending on the specific activity and/or property of the oncovector construct to be tested. Exemplary activities and/or properties of the oncovector construct that can be assessed include replication, effect on cell fusion, cell transformation, and expression. For all assays, positive and negative control oncovector constructs can be subjected to the same procedures for comparison.

In vitro assays include any laboratory assay known to one of skill in the art, such as for example, cell-based assays including dye transfer, syncytium formation, and anchorage independence. For example, in vitro assays can be performed following transfection of the oncovector constructs into any desired cells. Methodologies of transfection are known to one of skill in the art and include, but are not limited to, calcium phosphate, electroporation, heat shock, magnetofection, and the use of cationic lipids such as Lipofectamine™, Fugene®, Lipofectin™, Optifect™ and others known to one of skill in the art. One of skill in the art can determine which cell type should be transfected and by which transfection method, based on empirical determination. Exemplary of cell lines include, but are not limited to 293T cells, COS cells, CHO cells, HeLa cells, HEK293, THP-1, A549, Caco-2, HT29, MCF-7, NIH-3T3, WI-38, SAOS-2, 293T/17, U-2OS, and HT-1080. Cells lines also can include cells deficient in tumor suppressor proteins such as p53−/− and/or Rb−/− cells. For example, the human colon cancer cell line HCT116 (p53+/+) can be used in any experiments described herein and compared to its derivative cell line HCTp53KO (p53−/−), which has both p53 alleles disrupted (Bunz et al. (1998) Science, 282:1497-501). Other p53 deficient cells include PC3 cells (p53−/−) and their stable p53 transfectant PC3-p53 counterpart (Hastak et al. (2005) 19: 789-791). Rb−/− cells also can be used such as SA0S-2 cells; BC5637 cells (Rb−/−), which can be compared to a pRB+ clone 5637-RB-5 (Schnier et al. (1996) Proc. Natl. Acad. Sci, 93:5941-5946), and the prostate cancer cell line DU145 (Mack et al. (1999) Clinical Cancer Research, 5:2596-2604).

In vivo assays include animal model assays as well as administration to humans. Animal models include disease models in which a biological activity can be observed and/or measured. Dose response curves of an oncovector construct in such assays can be used to assess modulation of biological activities and as well as to determine therapeutically effective amounts of an oncovector construct for administration.

Also, in any of the assays described below, the experiments can be performed in the presence of a stuffer plasmid. The goal of using a stuffer plasmid is to reduce the copy number of construct per cell. Thus, when the constructs replicate they can be optimized to exhibit a more robust copy number differential. Thus, in order not to overload the system and to test for true self-replication, replication assays, fusion assays and other assays can be performed by diluting any of the constructs provided herein with a neutral stuffer plasmid. Exemplary of a neutral stuffer plasmid is the backbone of a synthetic vector pCpG-SEAP (Invivogen), with the full transcription unit removed, including promoter, reporter gene and pA sequences) (SEQ ID NO: 482). The resulting 1950 base pair plasmid is devoid of CpG motifs and does not contain a pUC ori, and SV40 ori or a Kan/NeoR gene. Therefore, there should be no competition for transfactors and no promoter interference.

Exemplary assays are described below. The assays described below can be adapted for in vitro or in vivo analyses.

1. Replication Assays

The assays for replication described herein can be used to detect, measure, or quantify replication of the oncovector constructs provided herein. Replication of the nucleic acid molecules can be mediated by non-viral (e.g., bacterial components) or viral mechanisms, including retrovirus systems and DNA-based virus systems. Generally, the constructs are episomally expressed and replicate extrachromosomally in host cells.

Replication assays can include detection through fluorescence, spectrophotometric, radioactive, immunological, radioimmunological and hybridization methods. Replication assays include qualitative comparison of the replication levels of different oncovector constructs and quantitative detection of copy number. More than one replication assay can be used on the same or different samples. Replication assays can be validated by comparing the results to one or more different replication assay(s). If the oncovector constructs include a reporter gene, replication assays can be validated by assessing the relationship between replication levels and activity of the reporter gene. Any reporter gene known to one of skill in the art can be used, such as, for example, enhanced green fluorescent protein (EGFP).

Replication assays can be used to determine the effect of mutations of the oncovector constructs on replication. Such mutations can include, but are not limited to, removal of CpG motifs, codon optimization, mutation of the origin of replication, mutation of the fusogenic protein, mutation of the prodrug activating enzyme, and mutation of the promoter or promoters.

In one non-limiting example, the oncovector construct contains a replication initiator, and constructs that have different mutations in the replication initiator are assayed to identify replication initiator mutants that retain the ability to initiate replication. The replication initiator can be the large T antigen of a papovavirus. The replication initiator can be SV40 large T antigen. For example, the replication initiator is a mutant SV40 large T antigen, and oncovector constructs are assayed to identify mutations in SV40 that do not impair replication of the oncovector constructs, but limit transformation of normal cells into cancerous cells. For example, the replication initiator is a mutant SV40 large T antigen, where one or more mutations are selected from those listed in Table 3 or 4.

In one embodiment, the oncovector constructs replicate in a specific and selective manner so that the construct accumulates in a predetermined cell or tissue, such as, for example, a disease-specific cell or tumor cell. In another embodiment, the oncovector construct contains a replication initiator under control of a promoter selective for expression in a predetermined cell or tissue, such as, for example, a disease-specific cell or tumor cell. In a further embodiment, the diseased cells are cancer cells. In a further embodiment, the oncovector construct contains a replication initiator under control of a promoter selective for expression in cancerous cells.

a. Incorporation of Detectable Nucleoside and/or Nucleotide Analogs

Cells can be assayed for replication of the oncovector constructs by incorporation and detection of nucleoside and/or nucleotide analogs. Such analogs and methods are known in the art and include, for example, bromodeoxyuridine (BrdU), and immunocytochemistry, respectively. For example, cells containing SV-T can be labeled with BrdU for minutes, hours or days. During exposure to BrdU, the cells can be kept in the dark to minimize DNA damage. After labeling, cells are washed, fixed, washed again, and permeabilized with any suitable reagent such as, for example, Triton X-100. Subsequently, the cells are washed again, blocked, and incubated with primary antibodies to SV-T and primary antibodies to BrdU. The antibodies can be polyclonal serum or monoclonal antibodies. Benzon nuclease can be added to increase access of the antibodies to the DNA.

The primary antibodies for SV-T and BrdU are selected from different species so that labeled secondary antibodies allow independent detection of SV-T and BrdU. In one non-limiting example, the primary antibodies are rabbit polyclonal antiserum raised against SV-T and a murine monoclonal antibody against BrdU, and the cells are stained with fluorescein-coupled goat anti-rabbit antibody and Texas Red-coupled swine anti-mouse antibody. By comparing immunofluorescence, the fraction of cells positive for T-antigen that incorporate BrdU can be determined. Background levels of negative control cells without SV-T can be subtracted (Dickmanns et al., J. Virol. 68(9):5496-5508 (1994)).

b. Real-Time Polymerase Chain Reaction (qPCR)

Copy number of the oncovector constructs can be determined by real-time polymerase chain reaction (qPCR) (Shadrina et al. (2007), BMC Medical Genetics, 8:6; Wilhelm et al. (2003), Chembiochem 4(11):1120-1128; Ayra et al. (2005), Future Drugs 5(2):209-219; Lee et al. (2006), J. Microbiol. Methods 65:258-267). Whereas endpoint PCR can be semi-quantitative due to saturation in the final stages of amplification, qPCR can provide a wide dynamic range for linear quantitative detection. Furthermore, qPCR has high sensitivity that allows determination using low amounts or with low abundance of biological samples (Lee et al. (2006), J. Microbiol. Methods 65:258-267), although use is not limited to those instances.

Any qPCR assay known to one of skill in the art is contemplated. For example, various chemistries are available for qPCR, including DNA intercalating agents, hydrolysis probes, dual hydrolysis probes, molecular beacons, and scorpion probes, (Ayra et al. (2005), Future Drugs 5(2):209-219). In one example, qPCR can be used to monitor fluorescence levels during PCR. The method can use the dye SYBR Green, which fluoresces upon binding to double stranded DNA. Dilutions of pure DNA with known concentrations can establish a standard curve for comparison, to provide the initial template concentration (Lee et al. (2006), J. Microbiol. Methods 65:258-267.

c. Southern Blot Analysis

Replication of oncovector constructs can be assayed by Southern blot analysis (Ziegler et al. (2004), J. Virological Methods 122(1):123-127). One of skill in the art knows different detection methods that can be used for Southern blot and can choose an appropriate detection method depending on the particular circumstances of the assay. For example, probes for use in Southern blot analysis can be radioactive or have biotin tags for immunodetection.

d. DpnI Digestion

Extrachromosomal replication can be assayed by determining if Hirt supernatant DNA is partially resistant to digestion by DpnI (Peden et al. (1992), Virus Genes 6(2):107-118); Campbell et al. (1997), Genes & Dev. 11:1098-1110). Whereas plasmid DNA prepared in DNA adenine methylase positive bacteria are methylated at adenine nucleotides in the sequence GATC, mammalian cells lack this enzyme, and hence human DNA is resistant to digestion by DpnI. Therefore, Hirt DNA that is digested by DpnI does not indicate episomal replication. In contrast, Hirt DNA that is largely resistant to digestion by DpnI indicates extrachromosomal replication.

e. Binding of SV40 TAg to SV40 Origin of Replication

The wild-type and/or mutant SV-T can be assayed for binding to the SV40 origin of replication. In one non-limiting example, radiolabeled DNA containing the SV40 origin of replication is incubated with extracts of cells containing SV40 TAg or control cells. The reaction is immunoprecipitated, and analyzed by electrophoresis and autoradiography (Cole et al. (1986), J. Virol. 57(2):539-546).

2. Cell Fusion Assays

The oncovector nucleic acid molecules provided herein include a gene that expresses a fusogenic protein, which when expressed by a cell causes cell fusion with neighboring cells. Cell fusion induced by any of the oncovector constructs provided herein can be assayed by any method known to one of skill in the art, examples of which are described herein. The fusogenic genes contained within the constructs include any provided herein or any known to one of skill in the art, such as any wild-type fusogenic gene and fusogenic genes that contain one or more mutations, such as 1, 2, 3, 4, 5 or more mutations.

Cell fusion involves mixing of both the outer and inner leaflet membrane lipids as well as mixing of the aqueous contents of donor and recipient cells (Kemble et al. (1994), Cell 76:383-391). Therefore, methods to assay for cell fusion can include analysis of lipid mixing of cells, content mixing of cells, and a combination of lipid and content mixing of cells. Cell fusion assays can be used to qualitatively compare activity of different fusogenic proteins expressed from the oncovector constructs. Cell fusion assays can be used to quantitatively compare the kinetics of cell fusion. Any cell fusion assay known to one of skill in the art can be used to assay oncovector constructs for their cell fusion properties.

In some examples, cell populations can be selected such that one is smaller than the other to facilitate distinction between the two populations (Cheng et al. (2005), J. Virol. 79(3):1853-1860). For example, normal cells and tumor cell populations can be mixed in order to assay for the specific accumulation of the constructs in tumor cells and induction of tumor cell fusion. If necessary, the tumor cells versus normal cells can be labeled with different dyes in order to visualize selective fusion.

Further, fusion assays can be performed to determine any bystander effect, i.e. the ability of oncovector constructs to facilitate fusion of bystander cells that themselves do not express the fusogenic protein. The goal is to select an amount of oncovector construct that accumulates in the desired cell or tissue, such as a tumor cell, and to thereby selectively induce fusion of those cells, while at the same time not being leaky so as to induce fusion of neighboring cells. Accordingly, fusion assays can be designed to test for the bystander effect. In such examples, the construct can be diluted such that the number of transfected cells is small. The cells can then be assayed for fusion using any of the assays described below. Generally, in performing such experiments, the oncovector construct also expresses some other reporter or detectable gene so that it is possible to identify those cells that have been transfected.

In addition, assays can be performed to test for the specificity of fusion based on cells that have accumulated the construct, for example, due to the presence of a conditional promoter that drives gene transcription. In such experiments, normal cells and the cell for which the oncovector construct is designed to accumulate can be co-transfected with the oncovector construct in mixing experiments. For example, normal cells and tumor cells (deficient or absent in p53 or an Rb family member) can be mixed. The different cell types can be mixed at various ratios. Fusion of the cells can be assayed to determine the specificity of the fusogenic activity of the oncovector construct for the designated cell type as compared to normal cells. Differentiation of the cell types can be facilitated by differentially labeling the cells, for example, with cell surface dyes known to one of skill in the art.

The assays described herein are exemplary in nature and not meant to be limiting.

a. Fluorescence Dequenching

An exemplary assay used to measure lipid and/or content mixing is fluorescence dequenching (Bagai et al. (1996), J. Cell Biol. 135(1):73-84; Danieli et al. (1996), J. Cell Biol. 133(3):559-569). In this method, cells containing a fusogenic protein or a gene that encodes a fusogenic protein are allowed to fuse with smaller cells labeled with one or more fluorescent labels. The fluorescent labels can be membrane probes, aqueous probes, or both membrane and aqueous probes. Dilution of the fluorescent label, due to cell fusion, results in fluorescence dequenching. In an exemplary assay, the fluorescently labeled cells are erythrocytes. For example, the fluorescently labeled cells can be labeled with the lipid probe octadecyl rhodamine B (R18). Any method known to one of skill in the art can be used to measure, detect, or visualize fluorescence dequenching. For example, fluorescence dequenching can be measured using a spectrofluorometer or by microscopy, such as, for example, confocal microscopy. Measurements can be made using a spectrofluorometer to detect fluorescence changes as a result of fusion of R18-labeled erythrocytes with acceptor cells. To normalize the data, percentage fluorescence dequenching (% FDQ) can be calculated according to the equation % FDQ=100•(F−F₀/Fₜ−F₀), where F₀ and F are fluorescence intensities at time 0 and at a given time point, and Fₜ is the fluorescence intensity in the presence of 0.1% Triton X-100 and is defined as fluorescence at “infinite” dilution of the probe (Dutch et al. (1998), J. Virol. 72(10):7745-7753). In one suitable assay, kinetics of lipid mixing activity are calculated from initial rates of fluorescence dequenching as measured by the maximum slopes of the curves (see e.g., Bagai et al. (1997), Virology 238:283-290; Bagai et al., J. Virol. 67, 3312-3318 (1993)).

b. Dye Transfer

Cells can be labeled with one or more detectable probes and incubated with cells containing the fusogenic protein and/or fusogenic gene to be tested. Fusion is detected by monitoring the distribution of the detectable probes. Exemplary detectable probes include, for example, fluorescent dyes. One of skill in the art knows how to select suitable probes for detection as well as methods to detect the probes. For example, fluorescent probes can be detected by fluorescence microscopy and/or by confocal microscopy. Cells can be labeled at the cell membrane, the cell interior, or both the cell membrane and the cell interior. A lipophilic label can be used to label cell membranes, and an aqueous dye can be used to label the interior of cells. Cells can be labeled with different labels at the cell membrane and at the cell interior. Cells can be labeled with the same label at the cell membrane and at the cell interior.

In some exemplary assays, the labeled cells are red blood cells (RBCs). In some exemplary assays, cell membranes are labeled with the lipophilic probe dye octadecyl rhodamine B (R18) (Bagai and Lamb (1996) J. Cell Biol., 135:73-84; Bagai and Lamb (1995) J. Virol. 69:6712-6719). In one example of an assay, red blood cells are labeled with R18 and incubated with cells that express a fusogenic protein. Following incubation, confocal microscopy is used to detect dye transfer from the labeled RBCs to the cells that express the fusogenic protein. Measurements can be made at varying time points in order to determine kinetics of cell fusion by measuring the rate of dye transfer.

In one non-limiting example of an assay for cell fusion, the interior of the labeled cells is labeled by entrapment with an aqueous dye, such as, for example N-(7-nitrobenz-2-oxa-1,3 diazol-4-yl)aminoethanosulfonic acid-taurine (NBD-taurine) (Sarkar et al., J. Cell Biol. 109:113-122 (1989)). In another example, one cell population can be labeled with a content probe, such as, for example, calcein. Cell populations can be selected such that one is smaller than the other to facilitate distinction between the two populations (Cheng et al. (2005), J. Virol. 79(3):1853-1860)

c. Content Mixing

Assays for cell fusion include assays that are dependent on the mixing of the aqueous contents of two different cell populations (see e.g., Nussbaum et al. (1994), J. Virol. 68:5411-5422; Bagai et al. (1996), J. Cell Biol. 135(1):73-84; Bagai et al. (1995), J. Virol. 69(11):6712-6719; Earp et al. (2003), J. Virol. 77(5):3058-3066).

In one example, fusion of two distinct cell populations can activate a reporter gene by content mixing. In one suitable assay, the first cell population contains bacteriophage T7 RNA polymerase; the second cell population contains lacZ gene linked to the T7 promoter. Either or both of the cell populations can contain the fusogenic protein. Cell fusion can be analyzed by any method known to one of skill in the art (e.g. X-gal staining and visualization of cells or quantitative colorimetric assay (see e.g., Nussbaum et al. (1994), J. Virol. 68:5411-5422).

d. Syncytium Formation

Cell fusion can be assayed by visual qualitative or quantitative detection of syncytia (Corcoran et al. (2006), J. Biol. Chem. 281(42):31778-31789; Dupressoir et al. (2005), Proc. Natl. Acad. Sci. USA 102(3):725-730). In one example, two cell populations are labeled with dyes of different colors, such as, 5-(6)-(((4-chloromethyl)benzoyl)amino)-tetramethylrhodamine and 7-amino-4-chloromethylcoumarin. Fluorescence microscopy can be used to reveal the presence of syncytial foci containing nuclei of both colors, indicating cell fusion. The number of syncytial nuclei per field can be determined by counting random microscopic fields and the percent fusion can be calculated relative to a negative or positive control (Corcoran et al., J. Biol. Chem. 281(42):31778-31789 (2006)). A fusion index can be calculated as [(N−S)/T]×100, where N is the number of nuclei in the syncytia, S is the number of syncytia, and T is the total number of nuclei counted (Dupressoir et al. (2005), Proc. Natl. Acad. Sci. USA 102(3):725-730).

3. Assays for Transformation of Normal Cells to Cancerous Cells

The SV40 T antigen (SV-T) has binding sites for HSP70, the tumor suppressor retinoblastoma protein (Rb) (Zalvide et al. (1998), Mol. Cell. Biol. 18(3):1408-1415; Stubdal et al. (1996), J. Virol. 70(5):2781-2788; Thompson et al. (1990), Virology 178:15-34; Sullivan et al. (2002), Microbiol. Mol. Biol. 66(2):179-202; Ludlow et al. (1990), Cell 60:387-396), and the tumor suppressor protein p53 (Tack et al. (1989), J. Virol. 63(8):3362-3367; Pipas et al. (2001), Seminars Cancer Biol. 11:23-30. SV-T can cause transformation of normal cells to cancerous cells (Bennoun et al. (1998), Oncogene 17:1253-1259; Ahuja et al. (2005) Oncogene 24:7729-7745, Srinivasan et al. (1989), J. Virol. 63(12):5459-5463). In order to use the wild-type or mutant SV-T for use in the oncovectors, mutant SV-T proteins will be used that retain replication properties, but do not induce transformation.

Any method known to one of skill in the art to distinguish a normal cell from a transformed cell can be used to measure transformation of SV-T. For example methods can be used where cells are subjected to conditions in which transformed cells grow, but normal cells do not grow. The following list of methods to assay for transformation of normal cells to cancerous cells is exemplary and not meant to be limiting.

a. Immortalization

When passaged, normal primary cells eventually undergo growth arrest and irreversible senescence. In contrast, some types of transformed cells exhibit immortalization Immortalization assays can be used to test for transformation (Kierstead et al. (1993), J. Virol. 67(4):1817-1829). For example, mouse embryo fibroblasts or rat embryo fibroblasts expressing large T antigen are immortal and propagate in culture for an indefinite period. Some cell types have additional requirements to exhibit immortalization when transformed. For example, human fibroblasts expressing large T antigen can be propagated for extended periods in culture but eventually senesce. An active telomerase is required to escape senescence in these cells (Ahuja et al. (2005), Oncogene 24:7729-7745).

One of skill in the art can determine which types of cells exhibit immortalization when transformed and is able to select appropriate cells to use for immortalization assays. For example, primary cells are unlikely to have acquired cellular mutations that might yield false positives. In addition, cells that rapidly senesce in culture, if seeded at low cell density, do not divide enough times to form colonies. In that event, it is not necessary to use a dominant selectable marker to eliminate nontransfected cells, and a monolayer does not form in flasks, avoiding dual selection for immortalization and dense focus formation (Kierstead et al. (1993), J. Virol. 67(4):1817-1829).

b. Growth in Low Serum

Transformed cells can be identified by proliferation and/or survival in medium lacking sufficient serum for normal cells to proliferate and/or survive. For example, normal fibroblasts require serum-supplemented medium to proliferate and survive, while SV40-transformed cells can proliferate and survive in medium with little or no serum (Ahuja et al. (2005), Oncogene 24:7729-7745).

c. Saturation Density

Contact of fibroblasts with adjacent cells arrests cell growth so that growth of fibroblasts in a culture disk typically terminates in a monolayer of cells. Transformed cells can be identified by increased saturation density, which is the maximum number of cells per unit area of culture surface. For example, SV40-transformed cells do not arrest cell growth upon reaching a monolayer but rather reach higher or indefinite saturation densities (Ahuja et al. (2005), Oncogene 24:7729-7745).

d. Focus Formation

Unlike untransformed cells, transformed cells can proliferate on the surface of a growth arrested monolayer of untransformed cells. Cells can also contain an activated oncogene such as, for example, ras (Cavender et al. (1995), J. Virol. 69(2):923-934). In one example, cells can be mixed with an excess of untransformed cells and then maintained in culture dishes. Untransformed cells will growth arrest when reaching monolayer while transformed cells can form dense regions of multilayered cells, called foci, on the surface of the monolayer. In another embodiment, cells to be assayed can be plated on a preformed monolayer of normal cells (Ahuja et al., Oncogene 24:7729-7745 (2005)).

e. Overcoming Growth-Inhibition of Tumor Suppressors

Constructs can be assayed for transformation by detecting the ability of SV-T to overcome growth-inhibitory effects of tumor suppressors, such as, for example, Rb. (Beachy et al. (2002), J. Virol. 76(7):3145-3157; Hinds et al. (1992), Cell 70(6):993-1006). In some examples, the tumor suppressors can be overexpressed. For example, overexpression of Rb can cause cells to arrest growth and adopt morphological and biochemical properties of senescent cells (Beachy et al. (2002), J. Virol. 76(7):3145-3157). The cells stop dividing and adopt a large flat shape (Templeton et al. (1991), Proc. Natl. Acad. Sci. USA 88:3033-3037). In one example, Rb-deficient cells are transfected with a construct expressing Rb and a construct expressing wild-type or mutant SV-T. After incubation for minutes, hours, or days, the cultures are fixed and stained, and large flat cells counted.

f. Activation of Cyclin A

Cells can be assayed for transformation by examining the ability of SV-T to transactivate the cyclin A promoter, which is a requirement for cell cycle progression. Cells can be transfected with SV-T and a reporter under the control of a cyclin A promoter region. After transfection, cells can be assayed for luciferase activity.

g. Anchorage Independence

Cells can be assayed for transformation by observing growth and proliferation in the absence of contact with a culture vessel surface and components of serum that coat the surface. In culture, normal cells require contact to proliferate. In contrast, some transformed cells, such as SV40-transformed cells, proliferate in the absence of contact. In one exemplary assay, cells can be suspended in a slurry of agarose supplemented with medium and serum. Under these conditions, untransformed cells can remain viable for weeks but do not proliferate, while SV40-transformed cells proliferate and grow as multicellular spheres (Ahuja et al. (2005), Oncogene 24:7729-7745). In another example, since expression of wild-type SV-T will confer soft agar growth in a suitable recipient cell line, cell lines that do not clone in soft agar can be transfected with an oncovector nucleic acid or other vector containing the wild-type or mutant SV-T, and the cells phenotypically screened for the ability to grow in soft agar (see e.g., U.S. Pat. No. 6,339,065).

h. Formation of Tumors in Animals

Normal cells are nontumorigenic when injected into test animals such as immunocompromised mice. Cells transfected with an oncovector nucleic acid or other vector containing the wild-type or mutant SV-T can be screened for formation of tumors in test animals such as nude mice (see, e.g. U.S. Pat. No. 6,339,065; Ahuja et al. (2005), Oncogene 24:7729-7745).

i. Binding of SV-T to Tumor Suppressor Proteins

Binding assays can be used to identify SV-T mutants that do not bind tumor suppressor proteins. Modified SV-T binding and/or affinity for tumor suppressor proteins can be determined using assays well known in the art. Any binding assay known to one of skill in the art is contemplated. As one example, electrophoretic mobility-shift assays (EMSA) can be used to measure interaction between SV-T and tumor suppressor proteins. Furthermore, pull-down methods can be used to detect formation of a SV-T tumor suppressor protein complex. In one example, wild-type or mutant SV-T is expressed as a fusion with glutathione S-transferase GST. The SV-T/GST fusion is immobilized on glutathione resin, washed, and incubated with p53. The resin is washed again, and analyzed by electrophoresis to detect p53 (Dickmanns et al. (1994), J. Virol. 68(9):5496-5508). Western blot analysis and/or immunoprecipitation can also be used to detect binding of SV-T to tumor suppressor proteins, such as, for example p53. In one example, tumor suppressor proteins can be detected by radiolabeling or immunodetection with anti-p53 primary antibodies and a secondary antibody linked to a reporter (Kierstead et al. (1993), J. Virol. 67(4):1817-1829).

The crystal structure of SV-T in complex with p53 shows that SV-T occupies the p53 DNA-binding surface and likely interferes with formation of a functional p53 tetramer (Lilyestrom et al. (2006), Genes Dev. 20:2373-2382). Therefore, transformation assays can include measuring SV-T dependent inhibition of tumor suppressor protein binding to target sequence. For example, the effects of SV-T on p53 binding to target sequences such as, for example, the human ribosomal gene cluster RGC sequence, can be measured by immunobinding, methylation interference (Kern et al. (1991), Oncogene 6:131-136), EMSA, and DNase I footprinting reactions (Bargonetti et al. (1992), Genes & Dev. 6:1886-1898). Since disrupting SV-T binding to p53 can inhibit SV-T helicase function, assays can also be performed to determine the effect of SV-T mutations on replication.

4. Expression Assays

Expression of genes contained within any of the constructs provided herein can be assessed by standard procedures known to one of skill in the art. Such assays are well known in the art and include, but are not limited to, enzyme-linked immunosorbent assays (ELISA), sodium dodecyl sulfate-polyacrylamide gel electrophoresis (SDS-PAGE) and detection of electrophoresed products by Western Blotting or coomassie blue staining, and other similar methods. In addition, expression can be assessed by assaying for the expression of a reporter gene, such as a fluorescent protein, e.g., GFP, or other detectable protein. The expression of the reporter gene can be correlated with expression as determined by any one or more other methods to assess expression. In performing such assays transfected cells can be lysed and cell lysates processed for analysis.

5. Immunogenicity Assays

The constructs provided herein can be modified to optimize their immunostimulatory effect. For example, the constructs can be modified to reduce acute inflammatory responses activated by the innate immune system in response to foreign pathogens. The effects of construct modifications on immunogenicity can be assayed by any method known to one of skill in the art. For example, reporter genes can be used to detect activation of signal transducers for inflammatory stimuli, including nuclear factor κB (NF-κB), Jun N-terminal kinase (JNK) and p38 mitogen-activated protein kinase (MAPK).

One component of the innate immune system are toll-like receptors (TLRs), transmembrane receptor proteins encoded to recognize patterns of pathogen-derived ligands and activate cells via a conserved Toll/IL-1R signal pathway that leads to activation of NF-κB and other transcriptional regulators. Toll-like receptor 9 (TLR9) binds bacterial DNA due to a greater frequency of unmethylated CpG bases than in vertebrates (Bauer et al. (2001), Proc. Natl. Acad. Sci. USA 98(16):9237-9242); Medzhitov (2001), Nature Immunol. 2(1):15-16; Hemmi et al. (2000), Nature 408:740-745), resulting in activation of downstream mediators such as NF-κB, and activation of proinflammatory cytokine production.

As one example of the modifications to optimize immunostimulatory effects of the constructs provided herein, CpG motifs can be removed from coding regions by silent mutations, and removed from non-coding regions when there is no deleterious effect on function. The effects of CpG mutation on immunostimulatory effects of the constructs can be assayed by monitoring downstream mediators activated by TLR9. For example, cells can contain an NF-κB reporter, such as, for example, an NF-κB luciferase reporter. Luciferase activity can be monitored using a luminometer, and measurements can be qualitative or quantitative. Alternatively, as another example, production of cytokines, such as IL-8, can be monitored by any assay known to one of skill in the art, such as, for example ELISA, as an indication of immunostimulatory activation.

Control experiments can be used in assays that detect immunostimulatory activation induced by CpG motifs within constructs. For example, activation can be abrogated by blocking cellular uptake with synthetic oligonucleotides lacking CpG, or by Bafilomycin A, which blocks endosomal maturation (Yoshimori et al. (1991), J. Biol. Chem. 266:17707-17712). Furthermore, activation can also be abrogated by methylation of CpG motifs. For example, constructs can be methylated in vitro by incubation with SssI methylase prior to assay (Kroft et al. (2001), Biology of Reproduction 65:1522-1527). Any suitable cell type, such as, for example, 293 cells, can be used in the assay.

6. Animal Models

Non-human animal models can be used to assess the activity of any of the constructs provided herein. For example, non-human animals can be used as models of a disease or condition. Exemplary animal models include animal models of cancer. In one example, animal models of cancer can be developed by injection of tumor cell lines into nude mice. Nude mice can be utilized in human cancer models because the human cells will not be rejected by the mice and will form solid tumors when the appropriate cells lines are used. For example, the osteosarcoma cell lines SAOS-2 (ATCC #HTB-85) and U-2OS (ATCC #HTB-96) can be used.

### G. PREPARATION, FORMULATION AND ADMINISTRATION OF ONCOVECTOR CONSTRUCTS AND ONCOVECTOR CONSTRUCT COMPOSITIONS

Oncovector constructs and oncovector construct compositions can be formulated for administration by any route known to those of skill in the art including intramuscular, intravenous, intradermal, intraperitoneal injection, subcutaneous, epidural, nasal, oral, rectal, topical, inhalational, buccal (e.g., sublingual), and transdermal administration or any route. Oncovector constructs can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition which is used.

In some embodiments, i.e. those involving subcutaneous injection, oncovector constructs can be co-administered with interstitial delivery enhancing agents. These agents act by degrading components of the interstitial matrix to increase the dispersion and bioavailability of locally injected drugs. The interstitial matrix is a complex three-dimensional structure made of various structural macromolecules including collagens, elastin, and fibronectins. Glycosaminoglycans (i.e. hyaluronan) and proteoglycans form a hydrated gel-like substance in the interstitial matrix, which acts as a filter controlling the rate of drug flow. Glycosaminoglycanases such as for example, hyaluronidase enzyme (rHuPH20) degrade this interstitial matrix filter through depolymerization of the viscoelastic component (Bookbinder et al., J Controlled Release 114:230-241 (2006)). Thus, co-administration of an interstitial delivery enhancing agent such as a hyaluronidase, for example rHuPH20, can improve delivery of the oncovector constructs provided herein.

Various delivery systems are known and can be used to administer oncovector constructs, such that the constructs are taken up by cells and incorporated into the nucleus (see e.g., Patil et al., AAPS Journal 7(1):E61-E77 (2005)). These include, but are not limited to, receptor- and non-receptor-mediated endocytosis; encapsulation in liposomes (e.g., cationic liposomes, anionic liposomes, pH sensitive liposomes, immunoliposomes, pegylated stealth liposomes), polymeric micelles, lipoplexes, polyplexes, microparticles (e.g., stabilized plasmid-lipid particles), and microcapsules; microinjection; and particle bombardment (i.e. gene gun). Generally, oncovector constructs are delivered to the nuclear compartment of the cell where the various components of the construct described herein are expressed. Uptake of oncovector constructs into the nucleus can be enhanced by binding to nuclear localization signal peptides, transcription factors (e.g., GAL4, SV-40, and SMGA), histones, and by the use of peptide nucleic acid constructs and cationic delivery vectors (see e.g., Uherek, Adv Drug Deliv Rev 44:153-166 (2000); Kamiya et al., Adv Drug Deliv Rev 52:153-164 (2001); Jaaskelainen et al., Biochim Biophys Acta 1195:115-123 (1994); Jaaskelainen et al., Int J Pharm 167:191-203 (1998); Monnard et al., Biochim Biophys Acta 1329:39-50 (1997)).

Pharmaceutical compositions containing oncovector constructs can be prepared. Generally, pharmaceutically acceptable compositions are prepared in view of approvals for a regulatory agency or otherwise prepared in accordance with generally recognized pharmacopoeia for use in animals and in humans. Pharmaceutical compositions can include carriers such as a diluent, adjuvant, excipient, or vehicle with which an oncovector construct is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil:water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Unit dose forms as used herein refer to physically discrete units suitable for human and animal subjects and packaged individually as is known in the art. Each unit dose contains a predetermined quantity of a therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly(alkylene glycols), polyhydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state also can be present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of oncovector constructs can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations of oncovector constructs suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes also can be employed with oncovector construct administration. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time.

For oncovector construct delivery methods that involve exposure to the endosomal compartment of the cell, administration methods can be employed to decrease the exposure of oncovector constructs to degradative processes within the endosome and facilitate delivery to the cellular cytoplasm. These methods include use of agents that promote rapid release from the endosomal compartment and/or disrupt the endosomal membrane. Such agents include, but are not limited to, fusogenic lipids (i.e. 1,2-dioleoyl-sn-glycero-3-phosphoethanolamine (DOPE)); lysosomatotropic agents such as monensin and chloroquine; viral peptides such as hemagglutinin HA2; fusogenic peptides such as poly(L-lysine) (PLL); and cationic polymers such as polyethyleneimine (PEI) and dendrimers (see e.g., Akhtar and Juliano, Trends Cell Bio 2:139-144 (1992); Wu-Pong, Adv Drug Deliv Rev 44:59-70 (2000); Kamiya et al., Adv Drug Deliv Rev 52:153-164 (2001); Brown et al., Int J Pharm 229:1-21 (2001); Luo and Saltzman, Nat Biotechnol 18:33-37 (2000); Kunisawa et al., Adv Drug Deliv Rev 52:177-186 (2001); Hope et al., Mol Membr Biol 15:1-14 (1998)).

Additional methods can be employed to decrease the exposure of oncovector constructs to degradative processes, such as exposure to nucleases and immunological intervention via antigenic and immunogenic responses. Examples of such methods include assembly of the oncovector construct as a stable plasmid, as described herein. Oncovector constructs also can be modified to modulate serum stability and half-life as well as reduce immunogenicity. Such modifications can be effected by any means known in the art and include modification of the primary DNA sequence such as, for example, removal or reduction of CpG motifs, or methylation of CpG motifs, as described herein.

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects), administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

An oncovector construct is included in the pharmaceutically acceptable carrier in an amount sufficient to exert a therapeutically useful effect in the absence of undesirable side effects on the patient treated. Therapeutically effective concentration can be determined empirically by testing the compounds in known in vitro and in vivo systems, such as the assays provided herein.

The concentration of an oncovector construct in the composition will depend on absorption, inactivation and excretion rates of the complex, the physicochemical characteristics of the complex, the dosage schedule, and amount administered as well as other factors known to those of skill in the art.

The amount of an oncovector construct to be administered for the treatment of a disease or condition, such as for example, cancer, can be determined by standard clinical techniques. In addition, in vitro assays and animal models can be employed to help identify optimal dosage ranges. The precise dosage, which can be determined empirically, can depend on the route of administration and the seriousness of the disease.

An oncovector construct can be administered at once, or can be divided into a number of smaller doses to be administered at intervals of time. Oncovector constructs can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them.

Exemplary Delivery Methods

The oncovector constructs provided herein can be administered as naked DNA, or within delivery vehicles. Hence, the nucleic acid molecule constructs provided herein can be packaged and delivered using any drug delivery system known to one of skill in the art. In particular, the delivery system is one that is capable of efficient delivery directed to tumor cells. For example, the nucleic acid molecules are delivered systemically. In some examples, the oncovector construct also can be administered in the presence of a hyaluronidase, such as PH20.

Drug delivery systems can include particulate carriers, such as those composed primarily of lipids and/or polymers. Such drug delivery systems can alter the pharmacokinetics and the biodistribution of their associated drug or to function as a reservoir. For example, a drug delivery system that includes a particulate carrier can ameliorate problems of poor solubility, can prevent rapid breakdown of the drug in vivo (e.g. digestion of plasmid by DNAses), reduce rapid clearance by the liver and enhance the selectivity of the drug for its target tissue.

Exemplary of drug delivery systems for delivery of the nucleic acid molecules provided herein are plasmid/nanoparticle complex that can include polyethylenimine (PEI) polymers (Genesee Scientific), polypropylenimine dendrimers PPIG3 polymers, B-amino-ester polymers, liposome formulations (Invitrogen) or sugar molecules such as cyclodextrin polymers.

In particular examples, the plasmid/nanoparticle complex can be delivered to the tumor site through a mechanism known as the enhanced permeability and retention effect (EPR), sometimes called passive targeting. In certain pathological conditions, such as in inflamed tissues and solid tumors, the permeability of the tissue vasculature increases to the point that particulate carriers, which are normally excluded from tissues, can extravasate and localize in the tissue intrastitial space. In contrast to tight blood vessels in most normal tissues, angiogenic blood vessels as they occur in tumors have gaps as large as 600 to 800 nm between adjacent endothelial cells. Carriers can extravasate through these gaps into the tumor interstitial space, in a size-dependent manner. As tumors have impaired lymphatic drainage, the carriers concentrate in the tumor, and large increases in tumor drug concentrations (10-fold, or more, higher) can be achieved relative to administration of the same dose of free drug.

In some examples, the constructs can be conjugated to a targeting protein to enhance targeting to tumor cells. For example, active targeting to the tumor cells can achieve significant increased vector expression at the tumor site. Exemplary of a targeting protein is transferrin. The transferrin receptor is found overexpressed in at least 40% of human tumors and cell lines and thus can provide a suitable receptor for targeted delivery of transferrin-linked DNA/nanoparticle complexes.

### H. EXEMPLARY METHODS OF TREATMENT

The oncovector constructs provided herein can be used for treatment of any disease or condition for which a particular oncovector construct is designed. Typically, such treatments include those where selective cell death, i.e. via cell fusion resulting in apoptosis, is desired. The oncovector constructs provided herein are designed to self replicate and promote fusogenic activity in a target cell population. Oncovector constructs have therapeutic activity alone or in combination with other agents. This section provides exemplary therapeutic uses of oncovector constructs. These described therapies are exemplary and do not limit the applications of oncovector constructs.

The oncovector constructs provided herein can be rationally designed by one of skill in the art to treat any disease or condition where selective cell death in a population of cells is desired. For example, an oncovector construct can be designed to target a particular cell population by inclusion of a cell type specific promoter. Such oncovector constructs would be active in cell types with particular transcriptional regulator expression profiles. In particular, the absence or presence of transcriptional activators and/or repressors in a particular cell type can determine whether an oncovector construct containing a particular promoter will be active in that cell. Thus, one of skill in the art can select a promoter for the oncovector construct based on the transcriptional regulator profiles of potential target cell populations known to be active in a particular disease. In some embodiments, the etiology of the disease is characterized by an aberrant transcriptional regulation profile, which facilitates the selective activity of the oncovector construct designed to treat that disease.

Oncovector constructs provided herein are intended for use in therapeutic methods in which selective cell death is desired. Such methods include, but are not limited to, methods of treatment of diseases, disorders and conditions, such as, but not limited to, inflammatory disorders; neurodegenerative disorders; heart disease; angiogenesis-mediated disease; and cancers such as leukemias (e.g., lymphoblastic, myeloid, hairy cell), lymphomas (e.g., AIDS-related, Burkitt, cutaneous T-cell, Hodgkin, non-Hodgkin, primary central nervous system (CNS)), carcinomas (e.g., adrenocortical, basal cell, islet cell, Merkel cell, squamous cell), astrocytomas (e.g., cerebellar, cerebral), gastrointestinal tumors (e.g., carcinoid, stromal), germ cell tumors (e.g., extracranial, extragonadal, ovarian), Kaposi sarcoma, osteosarcoma, malignant fibrous histiocytoma, brain stem glioma, malignant glioma, ependymoma, medulloblastoma, Dermatofibrosarcoma protuberans, supratentorial primitive neuroectodermal tumors, visual pathway and hypothalamic glioma, bronchial adenomas, carcinoid tumors, chronic myeloproliferative disorders, colorectal cancers, retinoblastoma, intraocular melanoma, Ewing family of tumors, gestational trophoblastic tumor, gliomas, macroglobulinemia, melanoma, mesothelioma, metastatic squamous neck cancer, multiple endocrine neoplasia syndrome, multiple myeloma, mycosis fungoides, myelodysplastic syndromes, myeloproliferative diseases, neuroblastoma, pheochromocytoma, pineoblastoma, plasma cell neoplasm, pleuropulmonary blastoma, rhabdomyosarcoma, soft tissue sarcomas, uterine sarcoma, Sézary syndrome, Waldenström macroglobulinemia, Wilms tumor, and cancers of the anus, appendix, bile duct, bladder, bone, brain, breast, cervix, colon, endometrium, esophagus, eye, gallbladder, stomach, head and neck, liver, pharynx, pancreas, kidney, larynx, lung, lip and oral cavity, mouth, nasal cavity and paranasal sinus, ovary, pancreas, parathyroid, penis, pituitary, prostate, rectum, renal pelvis and ureter, salivary gland, skin, small intestine, testicles, throat, thymus, thyroid, urethra, uterus, vagina, vulva and hematological system.

The following are some exemplary conditions for which oncovector constructs or nanoparticles thereof can be used as a treatment agent alone or in combination with other agents.

1. Cancers

The oncovector constructs provided herein can be used to treat various forms of cancer. Oncovector constructs can be designed to target cancer cell populations characterized by particular transcriptional regulation profiles. In some embodiments, the cancer cells to be treated can be characterized by lack of or reduced expression of tumor suppressor genes such as, for example, p53 and retinoblastoma (Rb). Cancer cells characterized by loss of p53 or Rb expression can be treated, for example, with oncovector constructs containing a promoter that is normally repressed by p53 and/or Rb. In some embodiments, the promoter is E2F. Exemplary cancers that can be treated with an oncovector construct containing the E2F promoter include, but are not limited to, sarcomas, mesothelioma, carcinoids, melanoma, neuroblastoma, retinoblastoma, osteosarcoma, and cancers of the lung, colon, esophagus, ovary, pancreas, skin, stomach, head and neck, bladder, prostate, liver, brain, adrenal gland, breast, endometrium, kidney, thyroid, parathyroid, cervix, bone, eye and hematological system. A subset of these exemplary cancers is discussed below.

a) Lung Cancer

Lung cancer, also known as carcinoma of the lung, is a disease where epithelial tissue in the lung grows uncontrollably. This leads to metastasis, invasion of adjacent tissue and infiltration beyond the lungs. The main types of lung cancer are small cell lung carcinoma and non-small cell lung carcinoma, which are discussed below. Typically, lung cancer is initiated by activation of oncogenes or inactivation of tumor suppressor genes. The oncovector constructs provided herein can be used to treat forms of lung cancer. The oncovector constructs can be used as a treatment agent alone or in combination with other forms of treatment.

i) Non-Small Cell Lung Carcinoma (NSCLC)

There are three main sub-types of non-small lung carcinomas: squamous cell lung carcinoma, adenocarcinoma and large cell lung carcinoma. Squamous cell lung carcinoma usually starts near a central bronchus and is often characterized by cavitation and necrosis within the center of the cancer. Well-differentiated squamous cell lung cancers often grow more slowly than other cancer types. Adenocarcinoma usually originates in peripheral lung tissue. Most cases of adenocarcinoma are associated with smoking. However, among non-smokers, adenocarcinoma is the most common form of lung cancer. A subtype of adenocarcinoma, the bronchioloalveolar carcinoma, is more common in female non-smokers. Large cell lung carcinoma is a fast-growing form that grows near the surface of the lung. It is often poorly differentiated and tends to metastasize early.

ii) Small Cell Lung Carcinoma (SCLC)

Small cell lung carcinoma is less common than non-small lung carcinoma. SCLC tends to arise in the larger breathing tubes and grows rapidly, becoming quite large. The SCLC cell contains dense neurosecretory granules which give this an endocrine/paraneoplastic syndrome association. While initially more sensitive to chemotherapy, SCLC ultimately carries a worse prognosis and is often metastatic at presentation. This type of lung cancer is strongly associated with smoking.

iii) Pathophysiology

Similar to other cancers described herein, lung cancer is initiated by activation of oncogenes or inactivation of tumor suppressor genes. Mutations in the Rb gene which lead to loss of Rb function have been identified in small cell lung carcinomas (Nevins, Human Molecular Genetics 10(7):699-703 (2001)). Mutations in the K-ras proto-oncogene are responsible for 20-30% of non-small cell lung cancers. Chromosomal damage can lead to loss of heterozygosity. This can cause inactivation of tumor suppressor genes. Damage to chromosomes 3p, 5q, 13q and 17p are particularly common in small cell lung carcinoma. The p53 tumor suppressor gene, located on chromosome 17p, is often affected.

Several genetic polymorphisms are associated with lung cancer. These include polymorphisms in genes coding for interleukin-1, cytochrome P450, apoptosis promoters such as caspase-8, and DNA repair molecules such as XRCC1. People with these polymorphisms are more likely to develop lung cancer after exposure to carcinogens.

iv) Treatment

The oncovector constructs provided herein can be used to treat forms of lung cancer described above. The oncovector constructs can be used as a treatment agent alone or in combination with other forms of treatment including, but not limited to, targeted therapy, chemotherapy, radiotherapy, and surgery. Forms of targeted therapy for lung cancer include, but are not limited to Gefitinib (Iressa), which targets the tyrosine kinase domain of the epidermal growth factor receptor (EGF-R); Erlotinib (Tarceva), another tyrosine kinase inhibitor; the angiogenesis inhibitor bevacizumab; cyclo-oxygenase-2 inhibitors; the apoptosis promoter exisulind; proteasome inhibitors; bexarotene; and vaccines. Chemotherapeutic agents often used in the treatment of lung cancer include, but are not limited to, cisplatin, carboplatin, gemcitabine, paclitaxel, docetaxel, etoposide, vinorelbine, topotecan and irinotecan. Radiotherapy methods for lung cancer include, but are not limited to, chest radiation, prophylactic cranial irradiation (PCI), and radiofrequency ablation.

b) Colorectal Cancer

Colorectal cancer, also known as colon cancer or bowel cancer, includes cancerous growths in the colon, rectum and appendix. It is the third most common form of cancer and the second leading cause of cancer-related death in the Western world. Many colorectal cancers are thought to arise from adenomatous polyps in the colon. These mushroom-like growths are usually benign, but some may develop into cancer over time. The most common colon cancer cell type is adenocarcinoma. Other, rarer types include lymphoma and squamous cell carcinoma. Typically, colorectal cancer is caused by mutations in DNA replication or DNA repair genes. The oncovector constructs provided herein can be used to treat colorectal cancer. The oncovector constructs can be used as a treatment agent alone or in combination with other forms of treatment.

i) Pathophysiology

Colorectal cancer is a disease originating from the epithelial cells lining the gastrointestinal tract. Hereditary or somatic mutations in DNA replication or DNA repair genes, including APC, K-Ras, NOD2 and p53 genes, can lead to unrestricted cell division. Chronic inflammation, such as in inflammatory bowel disease, can predispose patients to malignancy.

ii) Treatment

The oncovector constructs provided herein can be used to treat colorectal cancer. The oncovector constructs can be used as a treatment agent alone or in combination with other forms of treatment including, but not limited to, chemotherapy, radiotherapy, immunotherapy (i.e. Bacille Calmette-Guérin (BCG)), vaccine and surgery. Chemotherapeutic agents for the treatment of colorectal cancer can include, but are not limited to, 5-fluorouracil (5-FU), Capecitabine (Xeloda®), Leucovorin (LV, Folinic Acid), Oxaliplatin (Eloxatin®), Irinotecan (Camptosar®), Bevacizumab (Avastin®), Cetuximab (Erbitux®), Panitumumab (Vectibix), Bortezomib (Velcade®), Oblimersen (Genasense®, G3139), Gefitinib and Erlotinib (Tarceva®), and Topotecan (Hycamtin®).

c) Bladder Cancer

Bladder cancer refers to any of several types of malignant growths of the urinary bladder. Bladder cancer is characterized by the uncontrolled division of abnormal cells in the bladder. The bladder, located in the pelvis, is a hollow, muscular organ that stores urine. The most common type of bladder cancer begins in cells lining the inside of the bladder and is called urothelial cell or transitional cell carcinoma (UCC or TCC). Other types of bladder cancer include tumors arising from squamous cell carcinoma, adenocarcinoma, sarcoma, small cell carcinoma and secondary deposits from cancers elsewhere in the body. Bladder cancer development is often characterized by mutations in various oncogenes and tumor suppressor genes. The oncovector constructs provided herein can be used to treat bladder cancer. The oncovector constructs can be used as a treatment agent alone or in combination with other forms of treatment.

i) Pathophysiology

Bladder cancer development typically involves the acquisition of mutations in various oncogenes and tumor suppressor genes. Genes which tend to be altered in bladder cancer include FGFR3, HRAS, Rb and p53. A family history of bladder cancer also is a risk factor for the disease. In some cases, people appear to inherit reduced ability to break down certain chemicals, which makes them more sensitive to carcinogens.

ii) Treatment

The oncovector constructs provided herein can be used to treat bladder cancer. The oncovector constructs can be used as a treatment agent alone or in combination with other forms of treatment including, but not limited to, chemotherapy, radiotherapy, immunotherapy (i.e. Bacille Calmette-Guérin (BCG)), and surgery.

d) Ovarian Cancer

Ovarian cancer is a malignant tumor on or within an ovary. Ovarian cancer is classified according to the histology of the tumor, obtained in a pathology report. Surface epithelial-stromal tumor, including serous and mucinous cystadenocarcinoma, is the most common type of ovarian cancer. Other forms of ovarian cancer include sex cord-stromal tumor (i.e. estrogen-producing granulosa cell tumor and virilizing Sertoli-Leydig or arrhenoblastoma), and germ cell tumor. Genetic factors often play a role in the development of ovarian cancer. The oncovector constructs provided herein can be used to treat ovarian cancer. The oncovector constructs can be used as a treatment agent alone or in combination with other forms of treatment.

i) Pathophysiology

In many cases, genetic factors play a role in the development of ovarian cancer. Carriers of certain mutations of the BRCA1 or the BRCA2 gene are at a higher risk of developing both breast cancer and ovarian cancer. Mutations in the tumor suppressor gene p53 also have been observed in ovarian tumors (Greenblatt et al., Cancer Research 54:4855-4878 (1994)).

ii) Treatment

The oncovector constructs provided herein can be used to treat ovarian cancer. The oncovector constructs can be used as a treatment agent alone or in combination with other forms of treatment including, but not limited to, chemotherapy and surgery.

e) Skin Cancer

Skin cancer is a malignant growth on the skin and generally develops in the epidermis. The most common types of skin cancer are basal cell carcinoma (BCC) and squamous cell carcinoma (SCC) which can be locally disfiguring but are unlikely to metastasize. The most dangerous type of skin cancer is malignant melanoma. This form of skin cancer can be fatal if not treated early but contain only a small proportion of all skin cancers. More rare types of skin cancer include Dermatofibrosarcoma protuberans, Merkel cell carcinoma, and Kaposi's sarcoma. The oncovector constructs provided herein can be used to treat skin cancer. The oncovector constructs can be used as a treatment agent alone or in combination with other forms of treatment including, but not limited to, chemotherapy, surgery, radiotherapy, and cryotherapy.

f) Prostate Cancer

Prostate cancer occurs when cells of the prostate, a male reproductive organ which helps make and store seminal fluid, mutate and begin to multiply uncontrollably. These cells can spread from the prostate to other parts of the body, including the bones and lymph nodes. Prostate cancer can cause pain, difficulty in urinating, erectile dysfunction and other symptoms. Many factors, including genetics and diet, have been implicated in the development of prostate cancer. The oncovector constructs provided herein can be used to treat prostate cancer. The oncovector constructs can be used as a treatment agent alone or in combination with other forms of treatment.

i) Pathophysiology

A man's genetic profile contributes to his risk of developing prostate cancer. For example, BRCA1 and BRCA2 genes that are known risk factors for ovarian cancer and breast cancer in women also have been implicated in prostate cancer. Other genes which tend to be altered in prostate cancer include Rb and p53 (Banerjee et al., Cancer Research 52:6297-6304 (1992); Greenblatt et al., Cancer Research 54:4855-4878 (1994)).

ii) Treatment

The oncovector constructs provided herein can be used to treat prostate cancer. The oncovector constructs can be used as a treatment agent alone or in combination with other forms of treatment including, but not limited to, surgery, radiation therapy, hormonal therapy, chemotherapy, cryosurgery, and high intensity focused ultrasound (HIFU). Radiotherapy techniques include, but are not limited to, external beam radiation therapy, tomotherapy, and permanent implant brachytherapy. Hormonal therapies (i.e. medications or surgery to prevent prostate cancer cells from receiving dihydrotestosterone (DHT)) include, but are not limited to, orchiectomy (a surgery to remove the testicles); antiandrogens such as flutamide, bicalutamide, nilutamide, and cyproterone acetate which directly block the actions of testosterone and DHT within prostate cancer cells; ketoconazole and aminoglutethimide, which block the production of adrenal androgens such as DHEA; Abarelix (a GnRH antagonist); and leuprolide, goserelin, triptorelin, and buserelin (GnRH agonists).

g) Breast Cancer

Breast cancer is a cancer of the glandular breast tissue. Breast cancer can be divided into groups based on the tissue of origin, e.g. epithelial (carcinoma) versus stromal (sarcoma). The majority of breast cancers are carcinomas, which can be divided further into subclassifications such as ductal carcinoma (DCIS), lobular carcinoma (LCIS), papillary carcinoma, tubular/cribriform carcinoma, mucinous carcinoma, medullary carcinoma, and metaplastic carcinoma. In some cases, breast cancer is caused by a combination of environmental and hereditary factors. However, in most cases of breast cancer, the cause is unknown. The oncovector constructs provided herein can be used to treat breast cancer. The oncovector constructs can be used as a treatment agent alone or in combination with other forms of treatment.

i) Pathophysiology

In some breast cancer cases, there is a strong inherited familial risk. Two autosomal dominant genes, BRCA1 and BRCA2, account for most of the cases of familial breast cancer. Other genes which tend to be altered in breast cancer include Rb and p53 (Nevins, Human Molecular Genetics 10(7):699-703 (2001); Greenblatt et al., Cancer Research 54:4855-4878 (1994)). Other risk factors include age, diet, alcohol and tobacco use, obesity, hormones, and various environmental factors.

ii) Treatment

The oncovector constructs provided herein can be used to treat breast cancer. The oncovector constructs can be used as a treatment agent alone or in combination with other forms of treatment including, but not limited to, surgery (i.e. lumpectomy or mastectomy), hormonal therapy, chemotherapy, radiotherapy and targeted therapy. Hormonal therapies include, but are not limited to, tamoxifen, aromatase inhibitors, GnRH analogs, and ovarian ablation/suppression. Chemotherapeutic agents include, but are not limited to, cyclophosphamide, methotrexate, 5-fluorouracil, Adriamycin (doxorubicin), paclitaxel, Taxotere (docetaxel), and epirubicin. Radiotherapy techniques include, but are not limited to, IMRT (intensity modulated radiation therapy), brachytherapy (i.e. Mammosite), and targeted intraoperative radiotherapy (TARGIT). Targeted therapies include, but are not limited to, trastuzumab (Herceptin®), which is a monoclonal antibody that blocks the activity of the HER2 protein; and bevacizumab, which is a monoclonal antibody that blocks the activation of the VEGF receptor.

2. Selection of the Components of an Oncovector Construct for Treatment

Determination of the components of an oncovector construct is a consideration when determining which oncovector construct molecule to use in treating a selected disease. Several factors can be empirically determined to rationally design an oncovector construct for the treatment of a disease or disorder. First, the disease to be treated should be identified. Typically, such a disease is one which exhibits a unique transcriptional regulation profile in a population of cells that contribute to the etiology of the disease. Second, one or more promoters that is active under such transcriptional regulation can be identified. Such promoters would preferentially drive expression of the oncovector construct in disease cells, and likewise, would be inactive in normal or healthy cells. One of skill in the art knows or could identify transcriptional regulators that are involved in or characteristic of the etiology of the selected diseases. For example, the contribution of oncogenes and tumor repressors to some exemplary cancers are described above. One of skill in the art could then determine the appropriate promoter that would function as desired under the cell-specific transcriptional profile. The resultant oncovector construct is then a candidate therapeutic for treating the selected disease.

For example, loss of tumor suppressors such as Rb and p53, often triggers the onset of variety of cancers, including but not limited to, the exemplary cancers described above. Thus, an oncovector construct can be designed, that has as a component a promoter that is active in the absence of Rb and/or p53, to selectively target tumor cells as a mechanism of treating cancer. Exemplary of such a promoter is the E2F promoter, which is suppressed by Rb and p53 in normal cells and activated in cancer cells. Thus, a candidate oncovector construct for the treatment of a variety of cancers would be one that is driven by the E2F promoter.

3. Combination Therapies

Oncovector constructs can be used in combination with each other and with other existing drugs and therapeutics to treat diseases and conditions. For example, as described herein oncovector constructs can be used to treat diseases, such as cancer, and/or control tumor proliferation. Such treatments can be performed in conjunction with anti-tumorigenic drugs and/or therapeutics. Examples of antitumorigenic drugs and therapies useful for combination therapies include tyrosine kinase inhibitors and molecules capable of modulating tyrosine kinase signal transduction including, but not limited to, 4-aminopyrrolo[2,3-d]pyrimidines (see for example, U.S. Pat. No. 5,639,757), and quinazoline compounds and compositions (e.g., U.S. Pat. No. 5,792,771). Other compounds useful in combination therapies include steroids such as the angiostatic 4,9(11)-steroids and C21-oxygenated steroids, angiostatin, endostatin, vasculostatin, canstatin and maspin, angiopoietins, bacterial polysaccharide CM101 and the antibody LM609 (U.S. Pat. No. 5,753,230), thrombospondin (TSP-1), platelet factor 4 (PF4), interferons, metalloproteinase inhibitors, pharmacological agents including AGM-1470/TNP-470, thalidomide, and carboxyamidotriazole (CAI), cortisone such as in the presence of heparin or heparin fragments, anti-Invasive Factor, retinoic acids and paclitaxel (U.S. Pat. No. 5,716,981), shark cartilage extract, anionic polyamide or polyurea oligomers, oxindole derivatives, estradiol derivatives and thiazolopyrimidine derivatives.

Treatment of cancers can include combination therapy with anti-cancer agents such as antibodies, small molecule tyrosine kinase inhibitors, antisense oligonucleotides, vaccines, or immunoconjugates (i.e. antibodies coupled to radioactive isotope or cytotoxin). Exemplary of such anti-cancer agents include Gefitinib, Tykerb, Panitumumab, Erlotinib, Cetuximab, Trastuzumab, Imatinib, a platinum complex or a nucleoside analog. Other anticancer agents, include radiation therapy or a chemotherapeutic agent and/or growth inhibitory agent, including coadministration of cocktails of different chemotherapeutic agents. Examples of cytotoxic agents or chemotherapeutic agents include, for example, taxanes (such as paclitaxel and docetaxel) and anthracycline antibiotics, doxorubicin/adriamycin, carminomycin, daunorubicin, aminopterin, methotrexate, chlorambucil, methopterin, dichloro-methotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, maytansinol, epothilone A or B, taxotere, taxol, and the like. Other such therapeutic agents include estramustine, cisplatin, combretastatin and analogs, and cyclophosphamide. Preparation and dosing schedules for such chemotherapeutic agents can be used according to manufacturers' instructions or as determined empirically by the skilled practitioner. Preparation and dosing schedules for such chemotherapy also are described in Chemotherapy Service Ed., M. C. Perry, Williams & Wilkins, Baltimore, Md. (1992).

Additional compounds can be used in combination therapy with oncovector constructs. Anti-hormonal compounds can be used in combination therapies, such as with oncovector constructs, to treat certain tumors. Examples of such compounds include an anti-estrogen compound such as tamoxifen; an anti-progesterone such as onapristone and an anti-androgen such as flutamide, in dosages known for such molecules. It also can be beneficial to coadminister a cardioprotectant (to prevent or reduce myocardial dysfunction that can be associated with therapy) or one or more cytokines. In addition to the above therapeutic regimes, the patient can be subjected to surgical removal of cancer cells and/or radiation therapy.

Adjuvants and other immune modulators can be used in combination with oncovector constructs in treating cancers, for example to increase immune response to tumor cells. Combination therapy can increase the effectiveness of treatments and in some cases, create synergistic effects such that the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF.

### I. EXAMPLES

The following examples are put forth so as to provide those of ordinary skill in the art with a disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly) s.c., subcutaneous(ly); and the like.

### Example 1

### Optimization of Construct Components

In this example, components of constructs provided herein were modified to optimize human codon usage (human optimization), and also were modified to remove CpG motifs to reduce immunogenicity. The following considerations were followed during the optimization of all components.

1) Any codon that contains a CpG motif was not used in the optimized sequence. In analyzing the human codon optimized usage for a particular gene sequence, the number and fraction for these codons were listed as zero (see below for optimization of SV40 T antigen (TAg).

2) Any two codons placed next to each other can form a CpG motif. Thus, the choice of any codon was made to avoid these combinations.

3) The sequences were optimized based on the human codon usage frequency table (Table 15) to balance the percentages of codons selected with the published abundance of human transfer RNAs so that no codon is overloaded or limiting. In order to do this in a CpG-free manner, the first two considerations above were followed while balancing the codon frequencies.

4) New restriction sites can be generated during codon modifications. Thus, to ensure that unique restriction sites are still unique after modification, the modified sequences were checked with the Gene Construction Kit® sequence analysis program by Textco. If an unwanted restriction site was generated, the sequence was further modified by silent mutations to disrupt the unwanted sequences.

In performing the analysis above for modification of the component sequences, the Gene Construction Kit® was used to design sequences, to analyze restriction sites, and to analyze CpG motifs. Also, the analysis program was programmed to recognize specific unique restriction sites, and was programmed to search for “CG” as a general CpG motif and also “TCG” as a screen for the core optimal human CpG.

For example, using the methods outlined above, the wild-type TAg was synthesized using the reference amino acid sequence of the SV40 polyoma virus, with the introns removed (GenBank accession No. NC_{—}001669; the nucleotide sequence is set forth in SEQ ID NO: 561, encoding a sequence of amino acids set forth in SEQ ID NO: 564). The TAg gene sequence was then modified to optimize codon usage within human cells and to eliminate the presence of CpG dinucleotides (CpG-free gene coding sequence) as described below.

TAg has a sequence of nucleotides that is fairly devoid of CpG dinucleotides (see e.g., SEQ ID NO: 561). There are two CG dinucleotides in the sequence. The first corresponds to nucleotide positions 1045-7 (CGG) and codes for an arginine. It was modified to AGA, which also codes for arginine but does not contain a CpG motif. The second corresponds to nucleotide positions 1675-7 (CGC) and also codes for arginine. This was changed to AGG, which also codes for arginine but does not contain a CpG motif. The sequence of the CpG modified TAg is set forth in SEQ ID NO: 562.

Following CpG modification of TAg, the sequence was modified for human codon optimization. In the process of human codon optimization, new CpG motifs were generated. These were eliminated using the same process as outlined above. The sequence was again modified for human optimization. The process was repeated a few times before the final sequence CpG-free, human codon optimized sequence was determined. The human codon optimized usage of CpG-free TAg is depicted below.

TABLE 15
Human Codon Optimized Usage
of TAg synthesized CpG-free

| Amino | | | |
|---|---|---|---|
| Acid | Codon | Number | Fraction |
| ALA | GCA | 13 | 0.34 |
| ALA | GCC | 7 | 0.18 |
| ALA | GCG | 0 | 0 |
| ALA | GCT | 18 | 0.47 |
| ARG | AGA | 13 | 0.48 |
| ARG | AGG | 14 | 0.51 |
| ARG | CGA | 0 | 0 |
| ARG | CGC | 0 | 0 |
| ARG | CGG | 0 | 0 |
| ARG | CGT | 0 | 0 |
| ASN | AAC | 14 | 0.41 |
| ASN | AAT | 20 | 0.58 |
| ASP | GAC | 17 | 0.35 |
| ASP | GAT | 31 | 0.64 |
| CYS | TGC | 3 | 0.2 |
| CYS | TGT | 12 | 0.8 |
| END | TAA | 0 | 0 |
| END | TAG | 0 | 0 |
| END | TGA | 1 | 1 |
| GLN | CAA | 8 | 0.27 |
| GLN | CAG | 21 | 0.72 |
| GLU | GAA | 30 | 0.52 |
| GLU | GAG | 27 | 0.47 |
| GLY | GGA | 13 | 0.36 |
| GLY | GGC | 4 | 0.11 |
| GLY | GGG | 7 | 0.19 |
| GLY | GGT | 12 | 0.33 |
| HIS | CAC | 6 | 0.31 |
| HIS | CAT | 13 | 0.68 |
| ILE | ATA | 10 | 0.33 |
| ILE | ATC | 7 | 0.23 |
| ILE | ATT | 13 | 0.43 |
| LEU | CTA | 0 | 0 |
| LEU | CTC | 17 | 0.23 |
| LEU | CTG | 25 | 0.35 |
| LEU | CTT | 16 | 0.22 |
| LEU | TTA | 0 | 0 |
| LEU | TTG | 13 | 0.18 |
| LYS | AAA | 27 | 0.42 |
| LYS | AAG | 36 | 0.57 |
| MET | ATG | 23 | 1 |
| PHE | TTC | 16 | 0.44 |
| PHE | TTT | 20 | 0.55 |
| PRO | CCA | 10 | 0.31 |
| PRO | CCC | 8 | 0.25 |
| PRO | CCG | 0 | 0 |
| PRO | CCT | 14 | 0.43 |
| SER | AGC | 5 | 0.1 |
| SER | AGT | 13 | 0.27 |
| SER | TCA | 7 | 0.14 |
| SER | TCC | 8 | 0.17 |
| SER | TCG | 0 | 0 |
| SER | TCT | 14 | 0.29 |
| THR | ACA | 11 | 0.35 |
| THR | ACC | 7 | 0.22 |
| THR | ACG | 0 | 0 |
| THR | ACT | 13 | 0.41 |
| TRP | TGG | 12 | 1 |
| TYR | TAC | 7 | 0.26 |
| TYR | TAT | 19 | 0.73 |
| VAL | GTA | 6 | 0.17 |
| VAL | GTC | 9 | 0.26 |
| VAL | GTG | 16 | 0.47 |
| VAL | GTT | 3 | 0.08 |

The sequence also was checked for restriction sites, and any restriction sites that were in common with desired unique restriction sites within the final vector (described in Example 5) were disrupted. For example, within the sequence of wild-type TAg is an AseI site (ATTAAT set forth in SEQ ID NO: 550), corresponding to nucleotides 622-628 of the sequence for wild-type TAg (SEQ ID NO: 561). The final construct also contains an AseI restriction site, which was designed to be a unique restriction site at the 5′ end of the human cytomegalovirus (CMV) immediate early promoter. Therefore, a silent mutation was created in ATTAAT to include the following change: ATTAAc (the lower case nucleotide being the changed nucleotide). In the optimized sequence of TAg set forth in SEQ ID NO: 563 these nucleotides, corresponding to positions 622-628, were changed from ATTAAT to ATTAAc.

The final optimized CpG-free, human codon optimized sequence is set forth in SEQ ID NO: 563. The resulting CpG-free, human codon optimized modified sequence was synthesized by using hybridizing nucleotides and performing overlapping PCR with the proof reading DNA polymerase Pfu1 as described in Example 2 below. Oligonucleotides were generated and synthesis was performed by Integrated DNA Technologies (Coralville, Iowa). The CpG-free, human codon optimized sequence encodes a sequence of amino acids that is the same sequence encoded by wild-type TAg, i.e. an amino acid sequence set forth in SEQ ID NO: 564.

Similar methods were used to modify other components. Exemplary of constructs that were modified are set forth below in Table 16.

TABLE 16
Modified construct components

| | | | | CpG Modified and Hu- | | |
|---|---|---|---|---|---|---|
| | Unmodified | | CpG Modified | man Codon Optimized | | |
| | SEQ ID NO | | SEQ ID NO | SEQ ID NO | | |
| | (SEQ ID NO | | (SEQ ID NO | (SEQ ID NO | | Protein |
| | with terminal | | with terminal | with terminal | | SEQ ID |
| Component | restriction sites | | restriction sites | restriction sites | | NO |
| Internal Ribosomal | 103, 104 | | 102 | n/a | | n/a |
| Entry Site (IRES) | | | | | | |
| Enhanced Green | 543 | | n/a | 54 | | 544, 546 |
| Fluorescent Protein | | | | | | |
| (EGFP) | | | | | | |
| SV40 T antigen | 561 | | 562 | 563 | | 564 |
| SV40 early | 113, 114 | | 115, 116 | n/a | | n/a |
| promoter/ori | | | | | | |
| Kanomycin/Neomycin | 105 | | 106, 107 | 108, 109 | | 110 |
| resistance gene | | | | | | |
| (Kan/NeoR) | | | | | | |
| Kan/NeoR bacterial | 503 | | 107 | n/a | | n/a |
| promoter | | | | | | |
| HSV-TK gene | 498 | | n/a | 499 | | 501 |
| E2F1 Promoter | 534, 535 | | 536, 537 | n/a | | n/a |
| Avian Reovirus p10 | 8, 10 | (72, 74) | n/a | 9, 11 | (73, 75) | 39, 40 |
| Reptile Reovirus p14 | 12 | (76) | n/a | 13 | (77) | 41 |
| (RRVp14) | | | | | | |
| HERV3 | 35 | (100) | n/a | 36 | (101) | 58 |
| Baboon Reovirus p15 | 14 | (78) | n/a | n/a | | 42 |
| (BRVp15) | | | | | | |
| Gibbon Ape Leukemia | 15 | (80) | n/a | 16 | (81) | 43 |
| Virus Envelope | | | | | | |
| Protein (GALV) | | | | | | |
| Simian Virus5-F | 17 | (83) | n/a | 18 | (82) | 44 |
| Protein (SV5F) | | | | | | |
| Vesicular Stomatitis | 6 | (70) | n/a | 7 | (71) | 38 |
| Virus Protein G | | | | | | |
| (VSVG) | | | | | | |

### Example 2

### Generation of Synthetic Genes

Gene synthesis of construct components was performed by hybridizing oligonucleotides and performing overlap extension polymerase chain reaction with the proofreading DNA polymerase Pfu1. Most of the synthesized cDNAs were created by one of the following contract research organizations: Eton Bioscience Inc. (San Diego, Calif.), Blue Heron Biotechnology Inc. (Bothell, Wash.), or GenScript (Piscataway, N.J.). In each case, the synthetic genes were generated with NheI/BamHI or BstXI/NotI flanking restriction sites to facilitate insertion of the genes into expression vectors (described in Example 5). Primer oligonucleotides used for synthesizing genes of interest were synthesized by Integrated DNA Technologies, Inc. (San Diego, Calif.). The oligonucleotides used in the synthesis of exemplary genes are set forth in Table 17 below.

TABLE 17
Oligonucleotides used in the synthesis of exemplary genes

| | | Primer |
|---|---|---|
| | | SEQ ID |
| Gene | Oligonucleotide | NO |
| Avian Reovirus (ARV) p10 | AF1 - forward primer | 274 |
| | AF2 - forward primer | 275 |
| | AF3 - forward primer | 276 |
| | AF4 - forward primer | 277 |
| | AF5 - forward primer | 278 |
| | AR1 - reverse primer | 279 |
| | AR2 - reverse primer | 280 |
| | AR3 - reverse primer | 281 |
| | AR4 - reverse primer | 282 |
| Reptilian Reovirus | RRF1 - forward primer | 283 |
| (RRV) p14 | RRF2 - forward primer | 284 |
| | RRF3 - forward primer | 285 |
| | RRF4 - forward primer | 286 |
| | RRF5 - forward primer | 287 |
| | RRR1 - reverse primer | 288 |
| | RRR2 - reverse primer | 289 |
| | RRR3 - reverse primer | 290 |
| | RRR4 - reverse primer | 291 |
| | RRR5 - reverse primer | 292 |
| Baboon Reovirus (BRV) p15 | BRF1 - forward primer | 293 |
| | BRF2 - forward primer | 294 |
| | BRF3 - forward primer | 295 |
| | BRF4- forward primer | 296 |
| | BRF5 - forward primer | 297 |
| | BRF6 - forward primer | 298 |
| | BRR1 - reverse primer | 299 |
| | BRR2 - reverse primer | 300 |
| | BRR3 - reverse primer | 301 |
| | BRR4 - reverse primer | 302 |
| | BRR5 - reverse primer | 303 |
| | BRR6 - reverse primer | 304 |
| zGFP (cpG-free) | 1F1 - forward primer | 305 |
| | 1F2 - forward primer | 306 |
| | 1F3 - forward primer | 307 |
| | 1F4 - forward primer | 308 |
| | 1F5 - forward primer | 309 |
| | 1F6 - forward primer | 310 |
| | 2F1 - forward primer | 311 |
| | 2F2 - forward primer | 312 |
| | 2F3 - forward primer | 313 |
| | 1R1 - reverse primer | 314 |
| | 1R2 - reverse primer | 315 |
| | 1R3 - reverse primer | 316 |
| | 1R4 - reverse primer | 317 |
| | 1R5 - reverse primer | 318 |
| | 2R1 - reverse primer | 319 |
| | 2R2 - reverse primer | 320 |
| | Prev1 - reverse primer | 321 |
| | Prev2 - reverse primer | 322 |
| | Prev3 - reverse primer | 323 |
| Simian virus 5 F (SV5F) | SV1F1 - forward primer | 324 |
| | SV1F2 - forward primer | 325 |
| | SV1F3 - forward primer | 326 |
| | SV1F4 - forward primer | 327 |
| | SV1F5 - forward primer | 328 |
| | SV1F6 - forward primer | 329 |
| | SV1F7 - forward primer | 330 |
| | SV1F8 - forward primer | 331 |
| | SV1F9 - forward primer | 332 |
| | SV1R1 - reverse primer | 333 |
| | SV1R2 - reverse primer | 334 |
| | SV1R3 - reverse primer | 335 |
| | SV1R4 - reverse primer | 336 |
| | SV1R5 - reverse primer | 337 |
| | SV1R6 - reverse primer | 338 |
| | SV1R7 - reverse primer | 339 |
| | SV2F1 - forward primer | 340 |
| | SV2F2 - forward primer | 341 |
| | SV2F3 - forward primer | 342 |
| | SV2F4 - forward primer | 343 |
| | SV2F5 - forward primer | 344 |
| | SV2F6 - forward primer | 345 |
| | SV2F7 - forward primer | 346 |
| | SV2R1 - reverse primer | 347 |
| | SV2R2 - reverse primer | 348 |
| | SV2R3 - reverse primer | 349 |
| | SV2R4 - reverse primer | 350 |
| | SV2R5 - reverse primer | 351 |
| | SV2R6 - reverse primer | 352 |
| | SV2R7 - reverse primer | 353 |
| | SV3F2 - forward primer | 355 |
| | SV3F3 - forward primer | 356 |
| | SV3F4 - forward primer | 357 |
| | SV3F5 - forward primer | 358 |
| | SV3R1 - reverse primer | 359 |
| | SV3R2 - reverse primer | 360 |
| | SV3R3 - reverse primer | 361 |
| | SV3R4 - reverse primer | 362 |
| | SV3R5 - reverse primer | 363 |
| | SV3R6 - reverse primer | 364 |
| SV40Tag (TAg) | 1F1 - forward primer | 365 |
| | 1F2 - forward primer | 366 |
| | 1F3 - forward primer | 367 |
| | 1F4 - forward primer | 368 |
| | 1F5 - forward primer | 369 |
| | 1F6 - forward primer | 370 |
| | 1F7 - forward primer | 371 |
| | 1R1 - reverse primer | 372 |
| | 1R2 - reverse primer | 373 |
| | 1R3 - reverse primer | 374 |
| | 1R4 - reverse primer | 375 |
| | 1R5 - reverse primer | 376 |
| | 1R6 - reverse primer | 377 |
| | 1R7 - reverse primer | 378 |
| | 2F1 - forward primer | 379 |
| | 2F2 - forward primer | 380 |
| | 2F3 - forward primer | 381 |
| | 2F4 - forward primer | 382 |
| | 2F5 - forward primer | 383 |
| | 2F6 - forward primer | 384 |
| | 2F7 - forward primer | 385 |
| | 2R1 - reverse primer | 386 |
| | 2R2 - reverse primer | 387 |
| | 2R3 - reverse primer | 388 |
| | 2R4 - reverse primer | 389 |
| | 2R5 - reverse primer | 390 |
| | 2R6 - reverse primer | 391 |
| | 2R7 - reverse primer | 392 |
| | 3F1 - forward primer | 393 |
| | 3F2 -forward primer | 394 |
| | 3F3 -forward primer | 395 |
| | 3F4 - forward primer | 396 |
| | 3F5 - forward primer | 397 |
| | 3F6 - forward primer | 398 |
| | 3F7 - forward primer | 399 |
| | 3R1 - reverse primer | 400 |
| | 3R2 - reverse primer | 401 |
| | 3R3 - reverse primer | 402 |
| | 3R4 - reverse primer | 403 |
| | 3R5 - reverse primer | 404 |
| | 3R6 - reverse primer | 405 |
| | 3R7 - reverse primer | 406 |
| | 4F1 - forward primer | 407 |
| | 4F2 - forward primer | 408 |
| | 4F3 - forward primer | 409 |
| | 4F4 - forward primer | 410 |
| | 4F5 - forward primer | 411 |
| | 4F6 - forward primer | 412 |
| | 4R1 - reverse primer | 413 |
| | 4R2 - reverse primer | 414 |
| | 4R3 - reverse primer | 415 |
| | 4R4 - reverse primer | 416 |
| | 4R5 - reverse primer | 417 |
| | 4R6 - reverse primer | 418 |
| Vesicular stomatitis | 1F1 - forward primer | 419 |
| virus G protein (VSVG) | 1F2 - forward primer | 420 |
| | 1F3 - forward primer | 421 |
| | 1F4 - forward primer | 422 |
| | 1F5 - forward primer | 423 |
| | 1F6 - forward primer | 424 |
| | 1F7 - forward primer | 425 |
| | 1F8 - forward primer | 426 |
| | 1R1 - reverse primer | 427 |
| | 1R2 - reverse primer | 428 |
| | 1R3 - reverse primer | 429 |
| | 1R4 - reverse primer | 430 |
| | 1R5 - reverse primer | 431 |
| | 1R6 - reverse primer | 432 |
| | 2F1 - forward primer | 433 |
| | 2F2 - forward primer | 434 |
| | 2F3 - forward primer | 435 |
| | 2F4 - forward primer | 436 |
| | 2F5 - forward primer | 437 |
| | 2F6 - forward primer | 438 |
| | 2R1 - reverse primer | 439 |
| | 2R2 - reverse primer | 440 |
| | 2R3 - reverse primer | 441 |
| | 2R4 - reverse primer | 442 |
| | 2R5 - reverse primer | 443 |
| | 2R6 - reverse primer | 444 |
| | 2R7 - reverse primer | 445 |
| | 3F1 - forward primer | 446 |
| | 3F2 - forward primer | 447 |
| | 3F3 - forward primer | 448 |
| | 3F4 - forward primer | 449 |
| | 3F5 - forward primer | 450 |
| | 3R1 - reverse primer | 451 |
| | 3R2 - reverse primer | 452 |
| | 3R3 - reverse primer | 453 |
| | 3R4 - reverse primer | 454 |
| | 3R5 - reverse primer | 455 |
| | 3R6 - reverse primer | 456 |

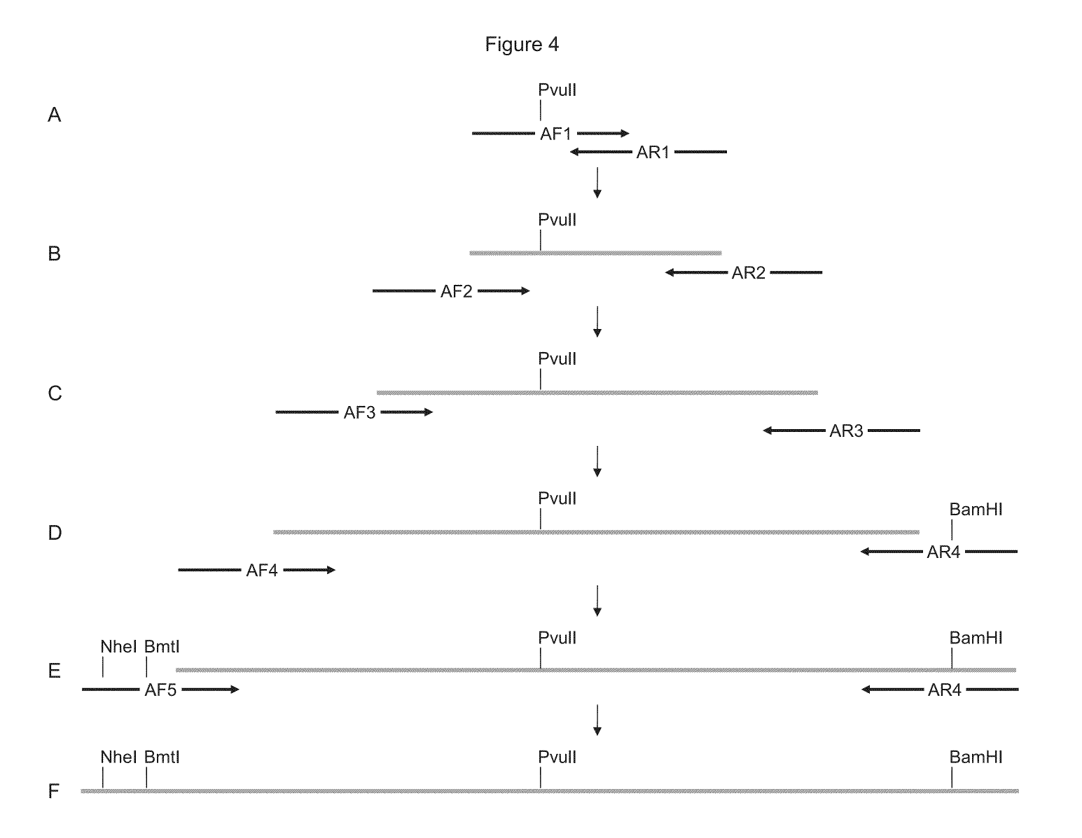

An exemplary illustration for the gene synthesis of Avian Reovirus (ARV) ARVp10 using the above-noted oligonucleotides is set forth in FIG. 4. The synthesized genes were inserted into pCR2.1-Topo-TA (Invitrogen; SEQ ID NO: 470). The topo-TA kit allowed for instant ligation of a gene fragment containing an adenine nucleotide overhang, which was ligated with a topoisomerase enzyme that was linked to the provided vector. Using similar methods, exemplary resulting synthesized genes are set forth in Table 18 below.

TABLE 18
Nucleotide and amino acid sequences of synthesized genes

| | Gene | Protein |
|---|---|---|
| | Product | Product |
| Gene | SEQ ID NO | SEQ ID NO |
| Avian Reovirus (ARV) p10 | 73 | 39 |
| Reptilian Reovirus (RRV) p14 | 77 | 41 |
| Baboon Reovirus (BRV) p15 | 78 | 42 |
| zGFP (cpG-free, human | 545 | 546 |
| codon-optimized EGFP) | | |
| Simian virus 5 F (SV5F) | 82 | 44 |
| SV40Tag (TAg) | 563 | 564 |
| Vesicular stomatitis virus G | 71 | 38 |
| protein (VSVG) | | |

### Example 3

### Modification of SV40 T Antigen (TAg)

In this example, TAg was modified to disrupt the binding sites for P53, Rb and HSP70. TAg mutations were rationally designed based on binding sites known in the art for p53 (Schmieg and Simmons, (1988) Virology. 164(1):132-40; Kierstead and Tevethia, (1993) J Virol. 67(4):1817-1829), Rb (DeCaprio et al., (1988) Cell. 54(2):275-283), and HSP70 (Campbell et al., (1997) Genes Dev. 11(9):1098-1110).

The TAg mutations were introduced by site-directed mutagenesis using the Quickchange Site-Directed mutagenesis kit (Stratagene), according to the manufacturer's instructions, with specifically-designed complementary oligonucleotides which served as primers that incorporated one or more codons encoding one or more amino acids that differ from the wild-type amino acid at that position into the newly synthesized DNA. The complementary oligonucleotide primers are set forth in Table 19 below. The codons encoding the substitute amino acids are set forth in bold and underlined.

TABLE 19
Mutagenesis Primers for TAg Mutants

| TAg | | | SEQ |
|---|---|---|---|
| Mutant | Primer | Mutagenesis Primer Sequence | ID NO |
| L19F | forward | 5′ CAACTGATGGATCTGTTGGGATTTGAGAGGTCTGCATG | 202 |
| | | GGGTAATATC 3′ | |
| | reverse | 5′ GATATTACCCCATGCAGACCTCTCAAATCCCAACAGAT | 203 |
| | | CCATCAGTTG 3′ | |
| P28S | forward | 5′ GGTCTGCATGGGGTAATATCTCCCTTATGAGGAAAGCA | 204 |
| | | TACCTG 3′ | |
| | reverse | 5′ CAGGTATGCTTTCCTCATAAGGGAGATATTACCCCATG | 205 |
| | | CAGACC 3′ | |
| L103P | forward | 5′ CATTTAATGAAGAGAATCCATTTTGCTCTGAGG 3′ | 206 |
| | reverse | 5′ CCTCAGAGCAAAATGGATTCTCTTCATTAAATG 3′ | 207 |
| C105A | forward | 5′ GAAGAGAATTTGTTTGCTTCTGAGGAAATGCC 3′ | 208 |
| | reverse | 5′ GGCATTTCCTCAGAAGCAAACAAATTCTCTTC 3′ | 209 |
| E107L | forward | 5′ GAGAATTTGTTTTGCTCTCTGGAAATGCCTAGTTCTG 3′ | 210 |
| | reverse | 5′ CAGAACTAGGCATTTCCAGAGAGCAAAACAAATCTC 3′ | 211 |
| E107K | forward | 5′ GAGAATTTGTTTTGCTCTAAGGAAATGCCTAGTTCTG 3′ | 212 |
| | reverse | 5′ CAGAACTAGGCATTTCCTTAGAGCAAAACAAATTCTC 3′ | 213 |
| E108L | forward | 5′ GAATTTGTTTTGCTCTGAGCTCATGCCTAGTTCTG 3′ | 214 |
| | reverse | 5′ CAGAACTAGGCATGAGCTCAGAGCAAAACAAATTC 3′ | 215 |
| S112N | forward | 5′ GGAAATGCCTAGTAATGATGATGAGG 3′ | 216 |
| | reverse | 5′ CCTCATCATCATTACTAGGCATTTCC 3′ | 217 |
| S189N | forward | 5′ CCAGGCACAACAACTATAATCATAATATTC 3′ | 218 |
| | reverse | 5′ GAATATTATGATTATAGTTGTTGTGCCTGG 3′ | 219 |
| D402R | forward | 5′ CACTGTCTCCTCCCTAAAATGAGGAGTGTAGTCTATG | 220 |
| | | ACTTCCT 3′ | |
| | reverse | 5′ AGGAAGTCATAGACTACACTCCTCATTTTAGGGAGGA | 221 |
| | | GACAGTG 3′ | |
| P453S | forward | 5′ GGAAAGCTCTCAATGTCAACCTGTCCCTTGATAGACTC | 222 |
| | | AACTTTG 3′ | |
| | reverse | 5′ CAAAGTTGAGTCTATCAAGGGACAGGTTGACATTGAG | 223 |
| | | AGCTTTCC 3′ | |
| V585R | forward | 5′ TATGCTGATATGGTATAGACCAAGGGCTGAGTTTGCC | 224 |
| | | AAAGCATTC 3′ | |
| | reverse | 5′ GAATGCTTTGGGCAAACTCAGCCCTTGGTCTATACCAA | 225 |
| | | TCAGCATA 3′ | |
| D604R | forward | 5′ TGAGTGGAAAGAAAGGCTTAGAAAGGAGTTTTCTCTT | 226 |
| | | TCAGT 3′ | |
| | reverse | 5′ ACTGAAAGAGAAAACTCCTTTCTAAGCCTTTCTTTCCA | 227 |
| | | CTCA 3′ | |
| Δ366- | forward | 5′ GGAGCAGATGTTGACAAATAGGTTCAGAATGGACATA | 228 |
| 370 | | ATGTTTGGC 3′ | |
| | reverse | 5′ GCCAAACATTATGTCCATTCTGAACCTATTTGTCAACA | 229 |
| | | TCTGCTCC 3′ | |
| Δ434- | forward | 5′ GGACCCATTGATTCAGGGAAAACTGGAAAGCTCTCAA | 230 |
| 444 | | TGTCAACC 3′ | |
| | reverse | 5′ GGTTGACATTGAGAGCTTTCCAGTTTTCCCTGAATCAA | 231 |
| | | TGGGTCC 3′ | |
| L103P/ | forward | 5′ CATTTAATGAAGAGAATCCATTTGCTTCTGAGGAAAT | 232 |
| C105A | | GC 3′ | |
| | reverse | 5′ GCATTTCCTCAGAAGCAAATGGATTCTCTTCATTAAA | 233 |
| | | TG 3′ | |
| L103P/ | forward | 5′ GCATTTAATGAAGAGAATCCATTTTGCTCTCTGGAAA | 234 |
| E107L | | TGCCTAG 3′ | |
| | reverse | 5′ CTAGGCATTTCCAGAGAGCAAAATGGATTCTCTTCAT | 235 |
| | | TAAATGC 3′ | |
| L103P/ | forward | 5′ GCATTTAATGAAGAGAATCCATTTTGCTCTGAGCTCA | 236 |
| E108L | | TGCCTAGTTC 3′ | |
| | reverse | 5′ GAACTAGGCATGAGCTCAGAGCAAAATGGATTCTCTT | 237 |
| | | CATTAAATGC 3′ | |
| C105A/ | forward | 5′ GAAGAGAATTTGTTTGCTTCTCTGGAAATGCCTAGTT | 238 |
| E107L | | CTGATGATGAG 3′ | |
| | reverse | 5′ CTCATCATCAGAACTAGGCATTTCCAGAGAAGCAAAC | 239 |
| | | AAATTCTCTTC 3′ | |
| E107L/ | forward | 5′ GAAGAGAATTTGTTTTGCTCTCTGCTCATGCCTAGTTC | 242 |
| E108L | | TGATGATGAG 3′ | |
| | reverse | 5′ CTCATCATCAGAACTAGGCATGAGCAGAGAGCAAAA | 243 |
| | | CAAATTCTCTTC 3′ | |
| C105A/ | forward | 5′ GAAGAGAATTTGTTTGCTTCTCTGCTCATGCCTAGTTC | 244 |
| E107L/ | | TGATGATG 3′ | |
| E108L | reverse | 5′ CATCATCAGAACTAGGCATGAGCAGAGAAGCAAACA | 245 |
| | | AATTCTCTTC 3′ | |
| L103P/ | forward | 5′ GGAATGCATTTAATGAAGAGAATCCATTTTGCTCTCTG | 246 |
| E107L/ | | CTCATGCCTAGTTCTGATGATG 3′ | |
| E108L | reverse | 5′ CATCATCAGAACTAGGCATGAGCAGAGAGCAAAATGG | 247 |
| | | ATTCTCTTCATTAAATGCATTCC 3′ | |
| L103P/ | forward | 5′ GGAATGCATTTAATGAAGAGAATCCATTTGCTTCTCTG | 248 |
| C105A/ | | CTCATGCCTAGTTCTGATGATG 3′ | |
| E107L/ | reverse | 5′ CATCATCAGAACTAGGCATGAGCAGAGAAGCAAATG | 249 |
| E108L | | GATTCTCTTCATTAAATGCATTCC 3′ | |
| E107L/ | forward | 5′ GAATTTGTTTTGCTCTCTGCTCATGCCTAGTAATGATG | 250 |
| E108L/ | | ATGAGG 3′ | |
| S112N | reverse | 5′ CCTCATCATCATTACTAGGCATGAGCAGAGAGCAAAA | 251 |
| | | CAAATTC 3′ | |
| C105A/ | forward | 5′ GAAGAGAATTTGTTTGCTTCTCTGCTCATGCCTAGTAA | 252 |
| E107L/ | | TGATGATGAGG 3′ | |
| E108L/ | reverse | 5′ CCTCATCATCATTACTAGGCATGAGCAGAGAAGCAA | 253 |
| S112N | | ACAAATTCTCTTC 3′ | |
| C105A/ | forward | 5′ GAAGAGAATTTGTTTGCTTCTGAGCTCATGCCTAGTA | 254 |
| E108L/ | | ATGATGATGAGG 3′ | |
| S112N | reverse | 5′ CCTCATCATCATTACTAGGCATGAGCTCAGAAGCAAA | 255 |
| | | CAAATTCTCTTC 3′ | |
| C105A/ | forward | 5′ GAAGAGAATTTGTTTGCTTCTCTGGAAATGCCTAGTA | 256 |
| E107L/ | | ATGATGATGAGG 3′ | |
| S112N | reverse | 5′ CCTCATCATCATTACTAGGCATTTCCAGAGAAGCAAA | 257 |
| | | CAAATTCTCTTC 3′ | |
| C105A/ | forward | 5′ GAAGAGAATTTGTTTGCTTCTGAGCTCATGCCTAGTTC | 240 |
| E108L | | TGATGATGAG 3′ | |
| | reverse | 5′ CTCATCATCAGAACTAGGCATGAGCTCAGAAGCAAAC | 241 |
| | | AAATTCTCTTC 3′ | |
| C105A/ | forward | 5′ GAATTTGTTTGCTTCTGAGGAAATGCCTAGTAATGAT | 258 |
| S112N | | GATGAGG 3′ | |
| | reverse | 5′ CCTCATCATCATTACTAGGCATTTCCTCAGAAGCAAA | 259 |
| | | CAAATTC 3′ | |

The QuikChange method involves linear amplification of template DNA by the Pfu 1 Ultra high-fidelity DNA polymerase. The forward and reverse primer pairs, both containing one or more codons for substitute amino acids, were extended during cycling using the pCMV-SV40Tag (WT)-I-GFP (SEQ ID NO: 695) plasmid as a template (see Example 6A). Extension of the primers resulted in incorporation of the new codons into the newly synthesized strands, and resulted in a mutated plasmid with staggered nicks. Following amplification, the nucleic acid was treated with DpnI, which digests the dam-methylated parental strands of the pCMV-SV40TAg-WT-I-GFP plasmid. This resulted in “selection” of the newly-synthesized mutated plasmids, which were not methylated. The vector DNA containing the new codons was transformed into XL1-Blue supercompetent E. coli cells (Invitrogen), where bacterial ligase repaired the nicks and allowed normal replication to occur. Wild-type and mutant TAg genes were sequenced using the forward sequencing primers set forth in SEQ ID NOS: 467, 468, and 461 which begin within the TAg gene at 494, 1125, 1786 bp, respectively. The protein sequences of TAg mutants are set forth in Table 20 below. Exemplary generated constructs containing SV40-TAg mutants are described in Example 6.

TABLE 20
CpG-free SV40-TAg mutant protein sequences

| | SEQ ID |
|---|---|
| SV40-TAg Mutant | NO |
| L19F | 565 |
| E107L | 566 |
| E107K | 567 |
| E108L | 568 |
| D402R | 569 |
| D402E | 570 |
| P453S | 571 |
| V585R | 572 |
| D604R | 573 |
| P28S | 574 |
| L103P | 575 |
| C105A | 576 |
| S112N | 577 |
| S189N | 578 |
| E107L/E108L | 579 |
| L103P/C105A | 580 |
| L103P/E107K | 581 |
| C105A/E107K | 582 |
| C105A/D402E | 583 |
| C105A/V585R | 584 |
| E107K/V585R | 585 |
| E107K/D402E | 586 |
| L103P/D402E | 587 |
| L103P/V585R | 588 |
| E107L/E108L/D402R | 589 |
| E107L/E108L/P453S | 590 |
| E107L/E108L/V585R | 591 |
| E107L/E108L/D604R | 592 |
| E107L/E108L/L19F/D402R | 593 |
| E107L/E108L/L19F/P453S | 594 |
| E107L/E108L/L19F/V585R | 595 |
| E107L/E108L/L19F/D604R | 596 |
| E107L/E108L/P28S/D402R | 597 |
| E107L/E108L/P28S/P453S | 598 |
| E107L/E108L/P28S/V585R | 599 |
| E107L/E108L/P28S/D604R | 600 |
| E107L/E108L/L19F/P28S/L103P/C105A/V585R | 601 |
| E107L/E108L/L19F/P28S/L103P/C105A/D604R | 602 |
| delta 366-370 (deletion) | 603 |
| delta 434-444 (deletion) | 604 |

### Example 4

### Modification of SV5F Protein

In this example, the SV5F gene was modified in order to enhance the fusogenic capacity of the encoded SV5F protein. The mutations were rationally designed based on amino acid changes known in the art to improve the alphα-helix and hydrophobicity of the fusogenic peptide (Bagai and Lamb, (1997) Virology 238(2):283-90; Russell et al., (2004) J Virology. 78(24):13727-42). The synthesized WT SV5F cDNA was subcloned into the pIRES-EGFP backbone construct designated pCMV-I-zGFP plasmid (SEQ ID NO: 694; see Example 5) using NheI-BamHI restriction sites generated during PCR in order to make pCMV-SV5F-WT-I-zGFP plasmid (SEQ ID NO: 718). The SV5F mutations were introduced by site-directed mutagenesis using the Quickchange Site-Directed mutagenesis kit (Stratagene), as described above, using the pCMV-SV5F-WT-I-zGFP plasmid (SEQ ID NO: 718) as a template. Specifically-designed complementary oligonucleotides served as primers that incorporated one or more codons encoding one or more amino acids that differed from the wild-type amino acid at that position into the newly synthesized DNA. The complementary oligonucleotide primers for the SV5F mutants are set forth in Table 21 below. The codons encoding the substitute amino acids are set forth in bold and underlined.

After sequencing to verify the mutant SV5F sequence, the SV5F-containing fragment was excised by NheI/BamHI digestion and inserted into the NheI/BamHI-digested original starting plasmid (pCMV-SV5F-WT-I-zGFP; SEQ ID NO: 718) to eliminate the risk of any inadvertent changes to the remainder of the plasmid during the mutagenesis procedure. Exemplary generated constructs containing SV5F mutants are described in Example 6.

TABLE 21
Mutagenesis Primers for SV5F Mutants

| | Gene | | | | |
|---|---|---|---|---|---|
| SV5F | Product | Protein | | Mutagenesis Primer | Primer |
| Mutant | SEQ ID NO | SEQ ID NO | Primer | Sequence | SEQ ID NO |
| G105A | 84 | 45 | forward | 5′ GGAGATTTGCTGCAGT | 260 |
| | | | | TGTGATTGGG 3′ | |
| | | | reverse | 5′ CCCAATCACAACTGCA | 261 |
| | | | | G CAAATCTCC 3′ | |
| G109A | 85 | 46 | forward | 5′ CTGGAGTTGTGATTGC | 262 |
| | | | | CCTGGCTGCCCTG 3′ | |
| | | | reverse | 5′ AGGGCAGCCAGGGCAA | 263 |
| | | | | TCACAACTCCAGC 3′ | |
| G114A | 86 | 47 | forward | 5′ GGGCTGGCTGCCCTGG | 264 |
| | | | | CTGTGGCTACAGCAGCA | |
| | | | | C 3′ | |
| | | | reverse | 5′ GTGCTGCTGTAGCCACA | 265 |
| | | | | GCCAGGGCAGCCAGCCC | |
| | | | | 3′ | |
| G105A/ | 87 | 48 | forward | 5′ GAGAAGGAGATTTGCT | 266 |
| G109A | | | | GCAGTTGTGATTGCCCTG | |
| | | | | GCTGCCCTGG 3′ | |
| | | | reverse | 5′ CCAGGGCAGCCAGGGC | 267 |
| | | | | AATCACAACTGCAGCAA | |
| | | | | ATCTCCTTCTC 3′ | |
| G105A/ | 88 | 49 | forward | 5′ GAGAAGGAGATTTGCT | 268 |
| G114A | | | | GCAGTTGTGATTGGGCTG | |
| | | | | GCTGCCCTGGCTGTGGCT | |
| | | | | ACAGCAGC 3′ | |
| | | | reverse | 5′ GCTGCTGTAGCCACAG | 269 |
| | | | | CCAGGGCAGCCAGCCCA | |
| | | | | ATCACAACTGCAGCAAA | |
| | | | | TCTCCTTCTC 3′ | |
| G109A/ | 89 | 50 | forward | 5′ GCTGGAGTTGTGATTG | 270 |
| G114A | | | | CCCTGGCTGCCCTGGCTG | |
| | | | | TGGCTACAGCAGC 3′ | |
| | | | reverse | 5′ GCTGCTGTAGCCACAG | 271 |
| | | | | CCAGGGCAGCCAGGGCA | |
| | | | | ATCACAACTCCAGC 3′ | |
| G105A/ | 90 | 51 | forward | 5′ GAGAAGGAGATTTGCT | 272 |
| G109A/ | | | | GCAGTTGTGATTGCCCTG | |
| G114A | | | | GCTGCCCTGGCTGTGGCT | |
| | | | | ACAGCAGC 3′ | |
| | | | reverse | 5′ GCTGCTGTAGCCACAG | 273 |
| | | | | CCAGGGCAGCCAGGGCA | |
| | | | | ATCACAACTGCAGCAAA | |
| | | | | TCTCCTTCTC 3′ | |

### Example 5

### Synthesis and Generation of Backbone Constructs

A. pIRES-EGFP Backbone

The commercially available pIRES2-EGFP vector construct (Clontech; SEQ ID NO: 1) was used as a primary backbone scaffold for the construction of various further backbone constructs. For use, the pIRES-EGFP construct was synthetically generated. Genes and DNA fragments, flanked by endonuclease recognition sequences, were synthesized by performing PCR with overlapping synthetic oligonucleotides as described above. The scaffold vector and synthesized fragments were then digested with the appropriate restriction endonuclease enzymes, and the fragments were ligated into the recipient vector with T4 DNA ligase. The recognition sites of the restriction endonucleases used to generate the various backbone constructs are set forth in Table 22 below.

TABLE 22
Restriction Endonuclease Recognition Sequences

| Restriction | | Recognition Site | SEQ ID |
|---|---|---|---|
| Enzyme | Recognition Site | After Cut | NO: |
| AseI | ...ATTAAT... | ...AT TAAT... | 550 |
| | ...TAATTA... | ... TAAT TA ... | |
| BamHI | ...GGATCC... | ...G GATCC... | 551 |
| | ...CCTAGG... | ...CCTAG G | |
| BglII | ...AGATCT... | ...A GATCT... | 552 |
| | ...TCTAGA... | ...TCTAG A... | |
| BspHI | ...TCATGA... | ...T CATGA... | 553 |
| | ...AGTACT... | ...AGTAC T... | |
| BstXI | ...CCANNNNNNTGG... | ...CCANNNNN NTGG... | 554 |
| | ...GGTNNNNNNACC... | ...GGTN NNNNNACC... | |
| NheI | ...GCTAGC... | ...G CTAGC... | 555 |
| | ...CGATCG... | ...CGATC G... | |
| NotI | ...GCGGCCGC... | ...GC GGCCGC... | 556 |
| | ...CGCCGGCG... | ...CGCCGG CO... | |
| PacI | ...TTAATTAA... | ...TTAAT TAA... | 557 |
| | ...AATTAATT... | ...AAT TAATT... | |
| PflFI | ...GACNNNGTC... | ...GACN NNGTC... | 558 |
| | ...CTGNNNCAG... | ...CTGNN NCAG... | |
| SexAI | ...ACCWGGT... | ...A CCWGGT... | 559 |
| | ...TGGWCCA... | ...TGGWCC A... | |
| XbaI | ...TCTAGA... | ...T CTAGA... | 560 |
| | ...TCTAGA... | ...TCTAG A... | |

Exemplary primary backbone constructs that were generated are set forth in Table 23 below.

TABLE 23

| | | | | | | | SEQ |
|---|---|---|---|---|---|---|---|
| Construct | | | | | polyA | SV40 | ID |
| name (s) | Promoter | 1^{st} gene | IRES | 2^{nd} gene | signal | ori^{a} | NO. |
| pIRES2-zGFP | CMV | — | + | zGFP | SV40 | o | 694 |
| pC-T-I-EGFP | CMV | TAg | + | EGFP | SV40 | o | 695 |
| pC-T(ss)-I-EGFP | CMV | TAg(ss) | + | EGFP | SV40 | o | 696 |
| (ss = redundant | | | | | | | |
| NotI and BstXI | | | | | | | |
| sites removed) | | | | | | | |
| pC-T(ss)-I-zGFP | CMV | TAg(ss) | + | zGFP | SV40 | o | 697 |
| pIRES2-SVT(wt) | CMV | — | + | TAg | SV40 | o | 698 |
| (pC-I-T) | | | | | | | |
| pCzGFP-I-T | CMV | zGFP | + | TAg | SV40 | o | 699 |

### B. Intermediate 1 Backbone Construct

To generate the Intermediate 1 backbone construct, the original Kan/NeoR gene (SEQ ID NO: 105) in the pIRES2-EGFP vector was replaced with a synthesized, CpG free, human codon optimized Kan/NeoR transcription unit. To this end, a DNA fragment was generated containing a CpG-free, human optimized Kan/NeoR transcription unit set forth in SEQ ID NO: 106, containing 1) a bacterial promoter set forth in SEQ ID NO: 107, corresponding to nucleotides 28-56 of SEQ ID NO:106, 2) a cDNA encoding the Kan/NeoR gene (SEQ ID NO: 109), corresponding to nucleotides 190-984 of the transcription unit of SEQ ID NO: 106, and 3) a synthetic polyadenylation (pA) signal (SEQ ID NO: 190), corresponding to nucleotides 985-1134 of SEQ ID NO: 106. Restriction recognition sequences for NotI, SexAI, and PacI were included at the 5′ end of the fragment, and the restriction sites for Pf1FI, Bg1II, and BspH1 were included at the 3′ end of the fragment. A variant fragment was also generated, substituting a variant of the Kan/NeoR gene set forth in SEQ ID NO: 108 for that encoded by SEQ ID NO: 109. The Kan/NeoR protein encoded by SEQ ID NO: 108 and 109 is the same as that encoded by the original, unmodified Kan/NeoR gene (SEQ ID NO: 105) and is set forth in SEQ ID NO: 110. Following digestion of the recipient pIRES2-EGFP vector and the synthesized fragment with NotI/BspHI, the digested fragment was inserted into the vector by standard procedures. The nucleotide sequence of the intermediate 1 backbone construct is set forth in SEQ ID NO: 2. The features of the Intermediate 1 backbone construct is set forth in FIG. 3A.

C. Intermediate 2 Backbone Construct

The Intermediate 2 backbone construct was generated by inserting a synthetic DNA fragment containing a synthetic pA signal for EGFP and the SV40 early promoter and origin into the Intermediate 1 backbone construct between the unique NotI and PacI restriction sites present in the synthetic fragment introduced into the pIRES-EGFP vector described in part A above. The synthetic PA signal for EGFP is set forth in SEQ ID NO: 196, and was designed to contain a 5′ NotI sequence (corresponding to nucleotides 1-8), an XbaI sequence (corresponding to nucleotides 12-17), and a 3′ SexAI sequence (corresponding to nucleotides 259-266). The synthetic SV40 early promoter/origin of replication is set forth in SEQ ID NO: 120, and was designed to contain a 5′ SexAI sequence (corresponding to nucleotides 1-8) and a 3′ PacI sequence (corresponding to nucleotides 149-158). The two synthetic constructs were joined by cutting and ligation at the SexAI sequence using standard procedures. The sequence of the resulting synthesized fragment is set forth in SEQ ID NO: 195, which contains a 5′ NotI sequence (corresponding to nucleotides 1-8) and a 3′ PacI sequence (corresponding to nucleotides 408-416.) Following digestion of the recipient backbone construct, Intermediate 1 vector, and the synthesized fragment with NotI/PacI, the digested fragment was inserted into the vector by standard procedures. The nucleotide sequence of the Intermediate 2 backbone construct is set forth in SEQ ID NO: 3. The features of the intermediate 2 backbone construct are set forth in FIG. 3B.

D. Intermediate 3 Backbone Construct (BB2)

The Intermediate 3 backbone construct was designed to contain a newly synthesized pUC origin, and also to temporarily remove the CMV promoter and a redundant Bg1II site. This was accomplished by generating a synthetic pUC origin with flanking Pf1FI-BamHI restriction sites for ligation into the Intermediate 2 backbone construct. The synthetic pUC origin region is set forth in SEQ ID NO: 121, and was designed to contain a 5′ Pf1FI sequence (corresponding to nucleotides 1-9), a 5′ Bg1II restriction site (corresponding to nucleotides 12-17), a 3′ AseI restriction site (corresponding to nucleotides 662-667), a 3′ NheI restriction site (corresponding to nucleotides 671-676), and a 3′ BamHI restriction site (corresponding to nucleotides 680-685). Following digestion of the recipient backbone construct Intermediate 2 vector and the synthesized fragment with Pf1FI and BamHI, the digested fragment was inserted into the vector by standard procedures. Digesting the Intermediate 2 vector with Pf1FI/BamHI removed the CMV promoter and the multiple cloning site, which included a unique Bg1II restriction site (corresponding to nucleotides 609-615 of SEQ ID NO: 3). Inserting the synthesized fragment introduced a new Bg1II restriction site and added back NheI and BamHI cloning sites. The nucleotide sequence of the Intermediate 3 backbone construct, which was designated BB2, is set forth in SEQ ID NO: 4. The features of the intermediate 3 backbone construct are set forth in FIG. 3C.

E. Intermediate 4 Backbone Construct

The Intermediate 4 backbone construct was designed to contain an additional transcription unit using the unique Pf1FI-Bg1II sites in the Intermediate 3 backbone construct. The transcription unit (SEQ ID NO: 497) was designed to contain 1) a synthetic cell-cycle dependent promoter that is a 110 base pair version of the E2F1 promoter (set forth in SEQ ID NO: 505, including a 5′ Pf1FI sequence corresponding to nucleotides 1-109), 2) a synthetic CpG-free, human condon-optimized HSV1-TK gene (SEQ ID NO: 499), and 3) a synthetic pA sequence (SEQ ID NO: 193, followed by a 3′ Bg1II sequence corresponding to nucleotides 151-156). The sequence of the resulting synthesized fragment is set forth in SEQ ID NO: 497, which contains a 5′ Pf1FI sequence (corresponding to nucleotides 1-9) and a 3′ Bg1II sequence (corresponding to nucleotides 1399-1404). Following digestion of the recipient backbone construct, Intermediate 3 vector, and the synthesized fragment with Pf1FI and Bg1II, the digested fragment was inserted into the vector by standard procedures. The nucleotide sequence of the Intermediate 4 backbone construct is set forth in SEQ ID NO: 5. The features of the Intermediate 4 backbone construct are set forth in FIG. 3D.

The intermediate 4 backbone construct contains AseI and NheI restriction sites for the insertion of a promoter of choice, NheI and BamHI restriction sites for the insertion of a gene of interest (first gene position), an EMCV IRES, followed by a gene sequence encoding EGFP between the BstXI and NotI restriction sites (second gene position), a synthetic pA signal 3′ of the second gene position, an SV40 early promoter and ori, a bacterial promoter, a human optimized CpG-free Kan-NeoR gene, a synthetic pA for the Kan-NeoR gene, a synthetic cell cycle-dependent conditional promoter controlling the expression of a synthetic human optimized CpG-free HSV1-TK gene, a synthetic pA sequence, and an unmodified pUC ori. Exemplary customized constructs using the Intermediate backbone are set forth in FIGS. 3E and 3F and are further described in Example 6.

F. BB3 Backbone Constructs

The BB3 backbone construct was generated by 1) excising the EGFP gene between the BstXI and NotI restriction sites and 2) inserting a transcription unit (SEQ ID NO: 734), encoding a reporter protein, red fluorescent protein (RFP) (SEQ: 732), between the Pf1FI and Bg1II restriction sites of Intermediate 3. The transcription unit contained, a promoter nested between Pf1FI and StuI restriction site (nucleotides 1-95 of SEQ ID NO: 734), a CpG-free, human codon-optimized RFP coding sequence (zRFP) (SEQ ID NO: 731) nested between flanking StuI and EcoRI restriction sites (SEQ ID NO: 733 or nucleotides 90-982 of SEQ ID NO: 731) and a polyadenylation sequence between EcoRI and Bg1II restriction sites (nucleotides 977-1138 of SEQ ID NO: 734). The BB3 backbone construct contains AseI and NheI restriction sites for the insertion of a promoter of choice, NheI and BamHI restriction sites for the insertion of a gene of interest (first gene position), an EMCV IRES, followed BstXI and NotI restriction sites for the insertion of a second gene of interest (second gene position), a synthetic pA signal 3′ of the second gene position, an SV40 early promoter and ori, a bacterial promoter, a human optimized CpG-free Kan-NeoR gene, a synthetic pA for the Kan-NeoR gene, a promoter for controlling the expression of a synthetic human optimized CpG-free RFP gene (zRFP), a synthetic pA sequence, and an unmodified pUC ori.

Functional BB3 constructs were generated by inserting a promoter, and two genes of interest in the specified locations (see Example 6). An exemplary BB3 construct pCzGFP-I-T-BB3 (SEQ ID NO: 607) contained CpG-free, human codon-optimized EGFP in the first position, and SV40-Tag in the second position, under the regulation of a CMV promoter. These and other features of pCzGFP-I-T-BB3 (SEQ ID NO: 607) are depicted in FIG. 3G.

Non-replicating dSV control constructs were also generated by inserting a synthetic plasmid, set forth in SEQ ID NO: 111, into the SexAI/PacI restriction site of the BB3 backbone. These dSV vectors contained the SV40pA, but no SV40 core ori. The features of an exemplary non-replicating dSV vector, pCzGFP-I-T-dSV (SEQ ID NO: 608), corresponding to pCzGFP-I-T-BB3 (SEQ ID NO: 607) described above, are depicted in FIG. 3H.

G. BB4 Backbone Constructs

BB4 backbone construct was generated from the BB3 construct pCzGFP-I-T-BB3 (SEQ ID NO: 607; see Example 6), which contained human codon optimized GFP in the first gene position and SV40-TAg in the second position. To generate BB4 constructs, the synthetic pA signal immediately 3′ of the second gene position and SV40 promoter/ori were replaced by a synthetic fragment, set forth in SEQ ID NO: 201, generated by Blue Heron Biotechnology Inc. (Bothell, Wash.). The new synthetic fragment contained a 5′ NotI restriction site followed by an SV40 pA signal (SV40pA), a BB3 format modified SV40 ori with a wild-type core SV40 ori, and a 3′ PacI restriction site. This fragment was ligated into a BB3 vector following NotI/PacI digestion to remove the synthetic polyadenylation sequence, and SV40 ori. The resultant plasmid was designated pCzGFP-I-T-BB4, and the sequence is set forth in SEQ ID NO: 719. Non-replicating dSV4-1 and dSV4-2 control constructs were also generated by inserting synthetic plasmids (Blue Heron Biotechnology Inc.), set forth in SEQ ID NOS: 199 and 198, respectively, into the NotI/PacI restriction sites of pCzGFP-I-T-BB3 (SEQ ID NO: 607). These dSV vectors contained the SV40pA, but no SV40 core on and were designated pCzGFP-I-T-dSV4-1 (SEQ ID NO: 720) and pCzGFP-I-T-dSV4-2 (SEQ ID NO: 721). The features of pCzGFP-I-T-BB4 and pCzGFP-I-T-dSV4-2 are set forth in FIGS. 31 and J, respectively. Generated BB4 constructs are set forth in the Table below:

TABLE 24

| | | | | | | | |
|---|---|---|---|---|---|---|---|
| | | | | | | | SEQ |
| Construct | | | | | polyA | SV40 | ID |
| name (s) | Promoter | 1^{st} gene | IRES | 2^{nd} gene | signal | ori^{a} | NO. |

Backbone 4 (BB4) Constructs

| | | | | | | | |
|---|---|---|---|---|---|---|---|
| pCzGFP-I-T-BB4 | CMV | zGFP | + | TAg | SV40 | + | 719 |
| pCzGFP-I-T-dSV4-1 | CMV | zGFP | + | TAg | SV40 | − | 720 |
| pCzGFP-I-T-dSV4-2 | CMV | zGFP | + | TAg | SV40 | − | 721 |

H. BB5 Constructs

BB5 backbone construct was generated from the BB3 construct pCzGFP-I-T-BB3 (SEQ ID NO: 607; see Example 6). The BB5 backbone construct was created by inserting a synthetic fragment, set forth in SEQ ID NO: 200, containing 5′ NotI and XbaI restriction sites followed by the bovine growth hormone polyadenylation signal (BGHpA) followed by a BB3 format modified SV40 ori and 3′ PacI restriction site, into the NotI/PacI-digested pCzGFP-I-T-BB3 plasmid. The resultant vector was designated pCzGFP-I-T-BB5; the sequence is set forth in SEQ ID NO: 726, and the features are set forth in FIG. 3K and the Table below.

TABLE 25

| | | | | | | | |
|---|---|---|---|---|---|---|---|
| | | | | | | | SEQ |
| Construct | | | | | polyA | SV40 | ID |
| name (s) | Promoter | 1^{st} gene | IRES | 2^{nd} gene | signal | ori^{a} | NO. |

Backbone 5 (BB5) Constructs

| | | | | | | | |
|---|---|---|---|---|---|---|---|
| pCzG-I-T-BB5 | CMV | zGFP | + | TAg | BGH | + | 726 |

### Example 6

### Summary of Generated Experimental Constructs

Using the backbone construct generated in Example 5, and the genes synthesized in Examples 1-4, several constructs were generated that were used in subsequent examples. The vectors were autonomously replicating plasmids (ARPs) containing an SV40 TAg and an SV40 ori. In some cases, plasmids lacking the SV40 promoter/ori, and thus incapable of SV40-TAg-mediated replication, were also generated. Variable components of the constructs included the promoter, inserted between the AseI and NheI restriction sites of the backbone construct; the first expressed gene, inserted between the NheI and BamHI restriction sites; the internal entry ribosomal site (IRES); the second expressed gene, inserted between the BstXI and NotI restriction sites; and the SV40 origin of replication (ori). Tables 26 and 27 summarize the generated vector. The nomenclature used for referring to the vectors is designated plasmid (p), promoter type (e.g. CMV or C), first gene of interest (e.g. GFP or G), IRES (I) and second gene of interest (e.g. SV40 TAg or T). If a component or element of the construct was synthesized to be human codon-optimized and CpG free it is represented by a preceding “z”.

A. Experimental Constructs Derived from pIRES2-GFP

pIRES2-GFP served as the backbone construct for the generation of initial fusogenic constructs (SEQ ID NOS:713-718) which were used to evaluate the fusogenic activities of various fusogenic protein candidates. These initial fusogenic constructs were generated by the insertion of NheI/BamHI-flanked synthetic fusogenic protein cDNAs into the NheI-BamHI sites of pIRES2-zGFP (SEQ ID NO: 694). These constructs expressed both the fusogenic cDNA and zGFP under the regulation of the CMV promoter. These vectors were designated pC-F-I-zG, where “F” is a fusogenic cDNA (e.g., GALV, ARVp10, RRVp14, BRVp15 etc). Resultant vectors were: pCzARVp10-IzG (SEQ ID NO: 715), pCzRRVp14-I-zG (SEQ ID NO: 716), pCzBRVp15-I-zG (SEQ ID NO: 717), pCzGALV-I-zG (SEQ ID NO: 713), pCzVSVG-I-zG (SEQ ID NO: 714), pCzSV5F-I-zG (SEQ ID NO: 718). These constructs were used in the initial evaluation of fusogenic activity of fusogenic protein candidates, as the GFP co-expression facilitated detection and assessment of multinucleated syncytia.

pIRES2-GFP also served as the backbone construct for generation of experimental constructs containing an SV40 Large T Antigen (Tag). A fragment containing CpG-free, human codon-optimized SV-40 Large T Antigen (TAg), set forth in SEQ ID NO: 563, was generated by overlapping PCR and oligonucleotide hybridization as described in Example 2 above. The fragment contained the synthetic TAg cDNA nested between 5′ NheI and BstXI restrictions sites and 3′ NotI and BamHI restriction sites. Flanking the TAg cDNA with multiple sets of restriction sites allowed for TAg insertion into either the first or second gene positions of any of the backbone vectors described above. The TAg synthetic fragment was subcloned into the NheI/BamHI sites of pIRES2-EGFP (SEQ ID NO: 1) to generate the backbone and reporter construct pCMV-SV40TAg-I-EGFP (pC-T-I-EGFP), set forth in SEQ ID NO: 695. The SV40 TAg cDNA was also digested with BstXI/NotI and subcloned into pIRES2-EGFP (SEQ ID NO: 1), replacing the coding sequence for EGFP. This procedure resulted in the pIRES2-SV40TAg (pC-I-T) backbone vector set forth in SEQ ID NO: 698. A reporter construct, pCzGFP-I-T (SEQ ID NO: 699), was generated by inserting a synthetic CpG-free, human codon-optimized EGFP (zGFP; SEQ ID NO: 545) into the first gene position of the pC-I-T vector, following NheI/BamHI digestion of the synthesized zGFP construct and pC-I-T vector.

B. Experimental Constructs Derived from Intermediate 4 Backbone Construct

To generate specific constructs derived from the Intermediate 4 backbone, the backbone construct was digested with the appropriate restriction endonucleases followed by ligation of the digested backbone with the new features flanked by the same restriction sites. For example, an Intermediate 4-derived construct was generated to contain a CMV promoter, a gene encoding a fusogenic protein (SV5F protein) in the first position and SV40-TAg in the second position (see FIG. 3E). The CMV promoter was inserted between the AseI and NheI restriction sites; the first gene of interest, SV5F, was inserted into the first gene position between the NheI and BamHI restriction sites; and SV40 TAg was inserted into the second gene position between the BstXI and NotI restriction sites. A further exemplary vector derived from Intermediate 4 was generated to confer cell cycle-dependent gene expression by replacing the CMV promoter with a cell cycle-dependent promoter (CCD) between the AseI and NheI restriction sites using standard procedures. An exemplary plasmid map, depicting a such a bicistronic construct with the gene for a fusogenic protein (SV5F protein) in the first gene position and SV40-TAg in the second position, under the regulation of a CCD promoter is set forth in FIG. 3F.

C. Experimental Constructs Derived from BB3 Backbone Construct

Customized BB3 vectors were generated by digesting the backbone construct with the appropriate restriction endonucleases followed by ligation of the digested backbone with the new features containing the same restriction sites. Specifically, a promoter sequence of interest was inserted between the AseI/NheI restriction sites and any two genes of interest were inserted into the first and second gene positions, in either orientation, between the NheI/BamHI and BstXI/NotI restriction sites, respectively. Retention of the restriction sites permitted replacement of the feature at a later time point if so desired. Prior to use, the sequences of the genes of interest located in the first and second positions were confirmed by sequencing. For bicistronic constructs containing a CMV promoter, the gene of interest located in the first position, 3′ of the CMV promoter, was sequenced using a forward sequencing primer which anneals to a sequence within the CMV, set forth in SEQ ID NO: 462 (5′-GTAGGCGTGTACGGTGGGAGG-3′) and a reverse primer which anneals to a sequence within the IRES element, set forth in SEQ ID NO: 464 (5′-CATATAGACAAACGCACACC-3′). The gene in the second location was sequenced using a forward primer which recognized a sequence within the IRES element, set forth in SEQ ID NO: 463 (5′-GAGGTTAAAAAAACGTCTAGG-3′) and a reverse primer which recognizes a sequence within the SV40 pA sequence, set forth in SEQ ID NO: 465 (5′-TTTCAGGTTCAGGGGGAGGTG-3′).

For example, a BB3-derived vector was generated that contained human codon optimized GFP in the first gene position and SV40-TAg in the second position, which was designated pCzGFP-I-T-BB3 (SEQ ID NO: 607; also see FIG. 3G). The pCzGFP-I-T-BB3 construct was used to subclone in SV40 TAg mutant cDNAs into the second gene position between the BstXI and NotI restriction sites, to create test vectors, set forth in SEQ ID NOS: 616-646 (see Example 5). FIG. 3L depicts a vector map of an exemplary BB3-derived construct containing a CMV promoter, a fusogenic gene encoding GALV in the first position and SV40 Tag in the second position (SEQ ID NO: 653).

Restriction of expression and replication of bicistronic constructs to tumor cells was achieved by inserting a cell cycle-dependent promoter in place of the CMV promoter in a AseI/NheI-digested BB3 construct. Examples of constructs with cell cycle dependent expression are set forth in SEQ ID NOS: 666-676.

A summary of exemplary generated BB3-derived vectors is set forth in Table 26.

D. Experimental Constructs Derived from Intermediate BB4 Construct

Customized BB4 vectors were generated by digesting the backbone construct with the appropriate restriction endonucleases followed by ligation of the digested backbone with the new features containing the same restriction sites. BB4-derived constructs were generated from the backbone construct set forth in SEQ ID NO:719. The constructs were generated by replacing zGFP sequence in the first gene of interest site separated by NheI and BamHI restriction sites with another gene of interest, such as genes encoding fusogenic proteins. A summary of exemplary generated BB4-derived vectors is set forth in Table 26.

E. Delta-SV40 Ori (dSV) Constructs

As a negative control, plasmids lacking the SV40 promoter/ori, and thus incapable of SV40-TAg-mediated replication, were also generated (designated dSV; also see Example 5). These non-replicating plasmids (nRPs) were generated by replacing the SV40 ori of any backbone construct described above or other construct with a synthetic linker (ACCTGGTTAGGAGGGGAGGAGGATTAATTAA; SEQ ID NO:111) containing flanking SexAI and PacI restriction sites, and were designated dSV (delta SV40 ori). The presence or lack of the SV40 ori for the BB3 and dSV constructs was confirmed by sequencing using a reverse sequencing primer that binds within the synthetic KanR cDNA. A summary of exemplary generated dSV-derived vectors is set forth in Table 26.

TABLE 26
Summary of Constructs

| | | | | | | | |
|---|---|---|---|---|---|---|---|
| | | | | | | | SEQ |
| Construct | | | | | polyA | SV40 | ID |
| name (s) | Promoter | 1^{st} gene | IRES | 2^{nd} gene | signal | ori^{a} | NO. |

BB5 BACKBONE CONSTRUCTS
Reporter Constructs

| | | | | | | | |
|---|---|---|---|---|---|---|---|
| pCMV-GFP-IRES-LTAg- | CMV | zGFP | + | TAg | syn | + | 607 |
| WT BB3 | | | | | | | |
| (pCzGFP-I-T-BB3) | | | | | | | |
| (pCzGFP-I-T(WT)-BB3) | | | | | | | |
| pCMV-GFP-IRES-LTAg- | CMV | zGFP | + | TAg | syn | − | 608 |
| WT BB3 | | | | | | | |
| (pCzGFP-I-T-dSV) | | | | | | | |
| (pCzGFP-I-T(WT)-dSV) | | | | | | | |
| pCzGFP-delta-I-T-BB3 | CMV | zGFP | − | — | syn | − | 609 |
| pCzGFP-I-nT-BB3 | CMV | zGFP | + | native TAg^{b} | syn | + | 610 |
| pC-mKate2-I-T-BB3 | CMV | mKate2 | + | TAg | syn | + | 611 |
| pC-Luc-I-T-BB3 | CMV | Luciferase | + | TAg | syn | + | 612 |
| pC-Luc-I-T-dSV | CMV | Luciferase | + | TAg | syn | − | 613 |
| pC-Bgal-I-T-BB3 | CMV | Beta- | + | TAg | syn | + | 614 |
| | | galactosidase | | | | | |
| pC-Bgal-I-T-dSV | CMV | Beta- | + | TAg | syn | − | 615 |
| | | galactosidase | | | | | |
| pC-SEAP-I-T-BB3 | CMV | SEAP | + | TAg | syn | + | 677 |

TAg Mutant Constructs

| | | | | | | | |
|---|---|---|---|---|---|---|---|
| pCzGFP-I-T(L19F)-BB3 | CMV | zGFP | + | TAg (L19F) | syn | + | 616 |
| pCzGFP-I- | CMV | zGFP | + | TAg (P28S) | syn | + | 617 |
| T(P28S)-BB3 | | | | | | | |
| pCzGFP-I- | CMV | zGFP | + | TAg (L103P) | syn | + | 618 |
| T(L103P)-BB3 | | | | | | | |
| pCzGFP-I- | CMV | zGFP | + | TAg (C105A) | syn | + | 619 |
| T(C105A)-BB3 | | | | | | | |
| pCzGFP-I- | CMV | zGFP | + | TAg (E107L) | syn | + | 620 |
| T(E107L)-BB3 | | | | | | | |
| pCzGFP-I- | CMV | zGFP | + | TAg (E107K) | syn | + | 621 |
| T(E107K)-BB3 | | | | | | | |
| pCzGFP-I- | CMV | zGFP | + | TAg (E108L) | syn | + | 622 |
| T(E108L)-BB3 | | | | | | | |
| pCzGFP-I- | CMV | zGFP | + | TAg (S112N) | syn | + | 623 |
| T(S112N)-BB3 | | | | | | | |
| pCzGFP-I- | CMV | zGFP | + | TAg (S189N) | syn | + | 624 |
| T(S189N)-BB3 | | | | | | | |
| pCzGFP-I- | CMV | zGFP | + | TAg (D402R) | syn | + | 625 |
| T(D402R)-BB3 | | | | | | | |
| pCzGFP-I- | CMV | zGFP | + | TAg (P453S) | syn | + | 626 |
| T(P453S)-BB3 | | | | | | | |
| pCzGFP-I- | CMV | zGFP | + | TAg (V585R) | syn | + | 627 |
| T(V585R)-BB3 | | | | | | | |
| pCzGFP-I- | CMV | zGFP | + | TAg (D604R) | syn | + | 628 |
| T(D604R)-BB3 | | | | | | | |
| pCzGFP-I- | CMV | zGFP | + | TAg | syn | + | 629 |
| T(L103P/C105A)- | | | | (L103P/C105A) | | | |
| BB3 | | | | | | | |
| pCzGFP-I- | CMV | zGFP | + | TAg | syn | + | 630 |
| T(L103P/E107L)- | | | | (L103P/E107L) | | | |
| BB3 | | | | | | | |
| pCzGFP-I- | CMV | zGFP | + | TAg | syn | + | 631 |
| T(L103P/E108L)- | | | | (L103P/E108L) | | | |
| BB3 | | | | | | | |
| pCzGFP-I- | CMV | zGFP | + | TAg | syn | + | 632 |
| T(C105A/E107L)- | | | | (C105A/E107L) | | | |
| BB3 | | | | | | | |
| pCzGFP-I- | CMV | zGFP | + | TAg | syn | + | 633 |
| T(C105A/E108L)- | | | | (C105A/E108L) | | | |
| BB3 | | | | | | | |
| pCzGFP-I- | CMV | zGFP | + | TAg | syn | + | 634 |
| T(E107L/E108L)- | | | | (E107L/E108L) | | | |
| BB3 | | | | | | | |
| pCzGFP-I- | CMV | zGFP | + | TAg | syn | + | 635 |
| T(C105A/E107L/ | | | | (C105A/E107L/ | | | |
| E108L)-BB3 | | | | E108L) | | | |
| pCzGFP-I- | CMV | zGFP | + | TAg | syn | + | 636 |
| T(L103P/E107L/ | | | | (L103P/E107L/ | | | |
| E108L)-BB3 | | | | E108L) | | | |
| pCzGFP-I- | CMV | zGFP | + | TAg | syn | + | 637 |
| T(L103P/C105A/ | | | | (L103P/CA105A/ | | | |
| E107L/E108L)- | | | | E107L/E108L) | | | |
| BB3 | | | | | | | |
| pCzG-I- | CMV | zGFP | + | TAg | syn | + | 638 |
| T(E107L/E108L/ | | | | (E107L/E108L/ | | | |
| S112N)-BB3 | | | | S112N) | | | |
| pCzG-I- | CMV | zGFP | + | TAg | syn | + | 639 |
| T(C105A/E107L/ | | | | (C105A/E107L/ | | | |
| E108L/S112N)-BB3 | | | | E108L/S112N) | | | |
| pCzG-I- | CMV | zGFP | + | TAg | syn | + | 640 |
| T(C105A/E108L/ | | | | (C105A/E108L/ | | | |
| S112N)-BB3 | | | | S112N) | | | |
| pCzG-I-T(C105A/ | CMV | zGFP | + | TAg | syn | + | 641 |
| E107L/S112N)-BB3 | | | | (C105A/E107L/ | | | |
| | | | | S112N) | | | |
| pCzG-I-T(C105A/ | CMV | zGFP | + | TAg | syn | + | 642 |
| S112N)-BB3 | | | | (C105A/S112N) | | | |
| pCzG-I-T(L103P/ | CMV | zGFP | + | TAg | syn | + | 643 |
| E107K)-BB3 | | | | (L103P/E107K) | | | |
| pCzG-I- | CMV | zGFP | + | TAg | syn | + | 644 |
| T(L105A/E107K)- | | | | (L105A/E107K) | | | |
| BB3 | | | | | | | |
| pCzGFP-I- | CMV | zGFP | + | TAg | syn | + | 645 |
| T(Δ366-370)- | | | | (Δ366-370) | | | |
| BB3 | | | | | | | |
| pCzGFP-I- | CMV | zGFP | + | TAg | syn | + | 646 |
| T (Δ434-444)- | | | | (Δ434-444) | | | |
| BB3 | | | | | | | |

Fusogenic Constructs

| | | | | | | | |
|---|---|---|---|---|---|---|---|
| pCzARVp10-I-T-BB3 | CMV | zARVp10 | + | TAg | syn | + | 647 |
| pCzARVp10-I-T-dSV | CMV | zARVp10 | + | TAg | syn | − | 648 |
| pCzRRVp14-I-T-BB3 | CMV | zRRVp14 | + | TAg | syn | + | 649 |
| pCzRRVp14-I-T-dSV | CMV | zRRVp14 | + | TAg | syn | − | 650 |
| pCzBRVp15-I-T-BB3 | CMV | zBRVp15 | + | TAg | syn | + | 651 |
| pCzBRVp15-I-T-dSV | CMV | zBRVp15 | + | TAg | syn | − | 652 |
| pCzGALV-I-T-BB3 | CMV | GALV | + | TAg | syn | + | 653 |
| pCzGALV-I-T-dSV | CMV | GALV | + | TAg | syn | − | 654 |
| pCzSV5F-I-T-BB3 | CMV | SV5F | + | TAg | syn | + | 655 |
| pCzSV5F-I-T-dSV | CMV | SV5F | + | TAg | syn | − | 656 |
| pCzVSVG-I-T-BB3 | CMV | VSVG | + | TAg | syn | + | 657 |
| pCzVSVG-I-T-dSV | CMV | VSVG | + | TAg | syn | − | 658 |

Enhanced Fusogenic Constructs

| | | | | | | | |
|---|---|---|---|---|---|---|---|
| pCzSV5F-I- | CMV | SV5F | + | TAg | syn | + | 659 |
| T(G105A)-BB3 | | (G105A) | | | | | |
| pCzSV5F-I- | CMV | SV5F | + | TAg | syn | + | 660 |
| T(G109A)-BB3 | | (G109A) | | | | | |
| pCzSV5F-I- | CMV | SV5F | + | TAg | syn | + | 661 |
| T(G114A)-BB3 | | (G114A) | | | | | |
| pCzSV5F-I- | CMV | SV5F | + | TAg | syn | + | 662 |
| T(G105A/G109A)- | | (G105A/G109A) | | | | | |
| BB3 | | | | | | | |
| pCzSV5F-I- | CMV | SV5F | + | TAg | syn | + | 663 |
| T(G105A/G109A/ | | (G105A/G109A/ | | | | | |
| G114A)-BB3 | | G114A) | | | | | |

Pro-Drug Converting Enzyme Constructs

| | | | | | | | |
|---|---|---|---|---|---|---|---|
| pC-zCDase-I-T-BB3 | CMV | zCDase | + | TAg | syn | + | 664 |
| pC-zCDase-I-T-dSV | CMV | zCDase | + | TAg | syn | − | 665 |

Cell Cycle-Dependent Promoter Constructs

| | | | | | | | |
|---|---|---|---|---|---|---|---|
| pCMV/EF1-zGFP- | CMV | zGFP | + | TAg | syn | + | 666 |
| I-T-BB3 | enhancers/ | | | | | | |
| | EF1 | | | | | | |
| | promoter | | | | | | |
| pCMV/EF2-zGFP- | CMV | zGFP | + | TAg | syn | + | 667 |
| I-T-BB3 | enhancers/ | | | | | | |
| | EF2 tata- | | | | | | |
| | less | | | | | | |
| | promoter | | | | | | |
| pCMV/EF2 (ss)- | CMV | zGFP | + | TAg | syn | + | 668 |
| zGFP-I-T-BB3 | enhancers/ | | | | | | |
| | EF2 tata- | | | | | | |
| | less | | | | | | |
| | promoter | | | | | | |
| | (redundant | | | | | | |
| | BamHI site | | | | | | |
| | removed) | | | | | | |
| pCMV/EF2-zGFP-I-T | CMV | zGFP | + | TAg | syn | + | 669 |
| (L103P)-BB3 | enhancers/ | | | (L103P) | | | |
| | EF2 tata- | | | | | | |
| | less | | | | | | |
| | promoter | | | | | | |
| pCMV/EF2-zGFP-I-T | CMV | zGFP | + | TAg | syn | + | 670 |
| (C105A)-BB3 | enhancers/ | | | (C103A) | | | |
| | EF2 tata- | | | | | | |
| | less | | | | | | |
| | promoter | | | | | | |
| pCMV/EF2-zGFP-I-T | CMV | zGFP | + | TAg | syn | + | 671 |
| (E107K)-BB3 | enhancers/ | | | (E107K) | | | |
| | EF2 tata- | | | | | | |
| | less | | | | | | |
| | promoter | | | | | | |
| pCMV/EF2-zGFP-I-T | CMV | zGFP | + | TAg | syn | + | 672 |
| (L103P/C105A)-BB3 | enhancers/ | | | (L103P/C105A) | | | |
| | EF2 tata- | | | | | | |
| | less | | | | | | |
| | promoter | | | | | | |
| pCMV/EF2-zGFP-I-T | CMV | zGFP | + | TAg | syn | + | 673 |
| (L103P/E107K)-BB3 | enhancers/ | | | (L103P/E107K) | | | |
| | EF2 tata- | | | | | | |
| | less | | | | | | |
| | promoter | | | | | | |
| pCMV/EF2-zGFP-I-T | CMV | zGFP | + | TAg | syn | + | 674 |
| (C105A/E107K)-BB3 | enhancers/ | | | (C105A/E107K) | | | |
| | EF2 tata- | | | | | | |
| | less | | | | | | |
| | promoter | | | | | | |
| pCMV/EF1/E2F-zGFP- | CMV | zGFP | + | TAg | syn | + | 675 |
| I-T-BB3 | enhancers/ | | | | | | |
| | EF1 and | | | | | | |
| | tata-less | | | | | | |
| | E2F | | | | | | |
| | promoter | | | | | | |
| pCMV/int-zGFP-I- | CMV plus | zGFP | + | TAg | syn | + | 676 |
| T-BB3 | pCI intron | | | | | | |

Reverse Orientation Constructs

| | | | | | | | |
|---|---|---|---|---|---|---|---|
| pC-T-I-zGFP-BB3 | CMV | TAg | + | zGFP | syn | + | 689 |
| pC-T-I-zGFP-dSV | CMV | TAg | + | zGFP | syn | − | 690 |
| pC-T-I-Luc-BB3 | CMV | TAg | + | Luciferase | syn | + | 691 |
| pC-T-I-Luc-dSV | CMV | TAg | + | Luciferase | syn | − | 692 |
| pC-T-I-zGALV-BB3 | CMV | TAg | + | zGALV | syn | + | 693 |

pIRES2-EGFP-BASED CONSTRUCTS

| | | | | | | | |
|---|---|---|---|---|---|---|---|
| pCzSV5F-I-T | CMV | zSV5F | + | TAg | SV40 | o | 700 |
| pCzVSVG-I-T | CMV | zVSVG | + | TAg | SV40 | o | 701 |
| pCz ARVp10-I-T | CMV | zARVp10 | + | TAg | SV40 | o | 702 |
| pCz RRVp14-I-T | CMV | zRRVp14 | + | TAg | SV40 | o | 703 |
| pCz BRVp15-I-T | CMV | zBRVp15 | + | TAg | SV40 | o | 704 |
| pCzGALV-I-T | CMV | zGALV | + | TAg | SV40 | o | 705 |
| pCzGFP-I-T(E107L) | CMV | zGFP | + | TAg (E107L) | SV40 | o | 706 |
| pCzGFP-I- | CMV | zGFP | + | TAg | SV40 | o | 707 |
| T(E107L/D402R) | | | | (E107L/D402R) | | | |
| pCzGFP-I- | CMV | zGFP | + | TAg | SV40 | o | 708 |
| T(E107L/E108L) | | | | (E107L/E108L) | | | |
| pCzGFP-I- | CMV | zGFP | + | TAg | SV40 | o | 709 |
| T(E107L/E108L/ | | | | (E107L/E108L/ | | | |
| D402R) | | | | D402R) | | | |
| pCzGFP-I- | CMV | zGFP | + | TAg | SV40 | o | 710 |
| T(E107L/E108L/ | | | | (E107L/E108L/ | | | |
| D453S) | | | | D453S) | | | |
| pCzGFP-I- | CMV | zGFP | + | TAg | SV40 | o | 711 |
| T(E107L/E108L/ | | | | (E107L/E108L/ | | | |
| V585R) | | | | V585R) | | | |
| pCzGFP-I- | CMV | zGFP | + | TAg | SV40 | o | 712 |
| T(E107L/E108L/ | | | | (E107L/E108L/ | | | |
| D604R) | | | | D604R) | | | |
| pCzGALV-I-zG | CMV | zGALV | + | zGFP | SV40 | o | 713 |
| pCzVSVG-I-zG | CMV | zVSVG | + | zGFP | SV40 | o | 714 |
| pCz ARVp10-I-zG | CMV | zARVp10 | + | zGFP | SV40 | o | 715 |
| pCz RRVp14-I-zG | CMV | zRRVp14 | + | zGFP | SV40 | o | 716 |
| pCz BRVp15-I-zG | CMV | zBRVp15 | + | zGFP | SV40 | o | 717 |
| pCz SV5F-I-zG | CMV | zSV5F | + | zGFP | SV40 | o | 718 |

BACKBONE 4 (BB4) CONSTRUCTS

| | | | | | | | |
|---|---|---|---|---|---|---|---|
| pCzGALV-I-T-BB4 | CMV | zGALV | + | TAg | SV40 | + | 722 |
| pCzGALV-I-T-dSV4-1 | CMV | zGALV | + | TAg | SV40 | − | 723 |
| pCzCDase-I-T-BB4 | CMV | zCDase | + | TAg | SV40 | + | 724 |
| pCzCDase-I-T-dSV4 | CMV | zCDase | + | TAg | SV40 | − | 725 |

^{a}SV40 ori o: original, unmodified SV40 ori from pIRES2-EGFP (contains CpG)
^{b}native TAg is the unmodified SV40-T Ag gene sequence (not modified to remove CpG or optimized for human codon frequency)

F. Other Constructs

Other constructs were generated that contain additional internal promoters in addition to the IRES in order to regulate expression of the second gene independent of the first gene. Also, constructs were generated that contain a modified IRES sequence whereby a potentially redundant ATG start site for the gene in the 2^{nd} positions was removed. These constructs are set forth in Table 27.

TABLE 27
Other Constructs

| | | | | | | | | SEQ |
|---|---|---|---|---|---|---|---|---|
| Construct | | | Internal | | Internal | | SV40 | ID |
| name | Promoter | 1^{st} gene | Promoter | IRES | Promoter | 2^{nd} gene | ori | NO. |
| pC-T-I-RSV- | CMV | TAg | — | + | Reos | GALV | + | 727 |
| GALV-BB3 | | | | | sarcoma | | | |
| | | | | | virus | | | |
| | | | | | (RSV) | | | |
| pC-Luc-RSV-I-T-BB3 | CMV | Luciferase | Reos | + | — | TAg | + | 728 |
| | | | sarcoma | | | | | |
| | | | virus | | | | | |
| | | | (RSV) | | | | | |
| pCzG-1I-T-BB3 | CMV | zGFP | — | 1I^{a} | — | TAg | + | 729 |
| pCzG-1I-T-dSV | CMV | zGFP | — | 1I^{a} | — | TAg | − | 688 |

^{a}IRES mutant 1I: ATGG at 3′ end mutated to ATCC to remove potentially redundant ATG start site for gene in the 2^{nd} position

### Example 7

### Replication and Protein Expression in Human Embryonic Kidney (HEK) 293 Cells

In this example, gene expression and replication in HEK 293 cells was examined in cells transfected with the autonomous replicating vector (ARP) pCMV-GFP-IRES-LTAg-WT-BB3 (also called pCzGFP-I-T-BB3; set forth in SEQ ID NO: 607). Cells transfected with a control non-replicating vector (nRP) containing a deletion in the SV40 origin, designated pCMV-GFP-IRES-LT Ag-dSV (also called pCzGFP-I-T-dSV; set forth in SEQ ID NO: 608), also was examined. Reverse orientation plasmids also were tested.

### A. Gene Expression in HEK 293 Cells

HEK293 cells (American Type Culture Collection (ATCC), Manassas, Va.) were grown in DMEM media with 10% heat deactivated (55° C. for 1 hr) fetal bovine serum (Omega Scientific, Tarzana, Calif.). Cells were split 1:4 twice per week in order to maintain sub-confluent cultures. Cells were removed by brief (1 minute) trypsinization and a lack of SV40 LTAg expression was confirmed by Western blot.

5×10⁵ HEK 293 cells/well were plated in a 6-well flat bottom tissue culture plate and transfected with 100 ng of pCzGFP-I-T-BB3 (SEQ ID NO: 607) or pCzGFP-I-T-dSV; SEQ ID NO: 608 using Lipofectamine 2000 (L2K), according to the manufacturer's instructions. To examine the effect of gene position, within the bicistronic vector, on gene expression, ARP and nRP plasmids, containing LTAg in the first gene position and zGFP in the second gene position, were also transfected at 100 ng. These reverse orientation plasmids were designated pC-T-I-zGFP-BB3 (SEQ ID NO: 689) and pC-T-I-zGFP-dSV (SEQ ID NO: 690), respectively.

After 2-3 hrs of transfection time, the transfection solution was removed, and the cells were harvested into a 50 ml tube containing a total of 20 ml medium. 1-ml aliquots of the cell suspension were dispensed into well plates of various size. Cells to be analyzed on Days 1 or 2 were plated in 24-well plates; cells to be analyzed on Days 3 or 4 were plated in 12-well plates, and cells to be analyzed on Day 5 were plated in 6-well plates.

Gene expression from the vectors was examined using the GFP reporter construct contained within the vectors. Four days post-transfection, ARP (pCzGFP-I-T-BB3 or pCTIzG-BB3) and nRP (pCzGFP-I-T-dSV or pC-T-I-zGFP-dSV) transfected cells, were assessed semi-quantitatively for GFP expression, by comparing relative fluorescent micrographs taken using identical exposure settings. 50 msec, gain 1 exposures demonstrated dramatically increased GFP expression from the ARP (pCzGFP-I-T-BB3 and pCTIzG-BB3 transfected cells compared to their nRP counterparts (pCzGFP-I-T-dSV and pCTIzG-dSV). GFP expression was undetectable in cells expressing either of the nRP plasmids, under the exposure settings used. The micrographs also revealed that cells transfected with the ARP with GFP in the first position (pCzGFP-I-T-BB3) exhibited higher levels of GFP expression than cells transfected with the ARP with GFP in the second position (pCTIzG-BB3).

GFP expression was also measured quantitatively using a GFP assay kit (BIO 101® Systems). At 1, 2, and 3 days post-transfection, fluorescent cell lysates were diluted into sample buffer according to kit instructions and read in a plate reader (excitation A488, emission 436) in flat-bottomed fluorescent-permissive plastic plates with black opaque sidewalls. GFP fluorescence for untransfected cells was also measured at each time point as a negative control. GFP expression (ng/ml) was determined using a standard curve generated from purified GFP protein. The GFP signal, for each condition, at one day post-transfection defined the basal expression level, and the fold increase of GFP fluorescence was determined by normalizing the GFP fluorescence at two and three days post-transfection to the GFP signal to that detected at 1 day post-transfection.

The results showed that the levels of GFP expression was dramatically less in cells transfected with either control nRP vector (pCzGFP-I-T-dSV or pC-T-I-zGFP-dSV) than those of the ARP vectors (pCzGFP-I-T-BB3 and pC-T-I-zGFP-BB3), and the fold increases of GFP expression for the nRP vectors were indistinguishable from non-transfected cells. Throughout the study, the untransfected cells exhibited a low GFP signal that increased very slightly over time. GFP signals, detected in cells transfected with pCzGFP-I-T-dSV or pC-T-I-zG-dSV nRP vectors, were approximately 2-fold higher than the signal for untransfected cells at all time points. Thus, the GFP expression fold increase from GFP expression at 1 day post-transfection, for the nRP vectors was indistinguishable from that of the untransfected cells, confirming that the nRP vectors did not replicate.

Cells transfected with the ARP with GFP in the second position, pC-T-I-zGFP-BB3, expressed twice the amount of GFP as the nRP vectors, or about 4 times the signal from untransfected cells at 2 days post-transfection. At 3 days post-transfection pC-T-I-zGFP-BB3-transfected cells expressed approximately 4.5 times more GFP than the nRP vectors.

Cells transfected with the ARP with GFP in the first position expressed approximately 3.5 times more GFP than those transfected with the nRP vectors and almost 2 times more than cells transfected with replicating constructs with GFP in the second position. At 3 days post-transfection, GFP signal from cells transfected with pCzGFP-I-T-BB3 increased to over 5 times more than cells transfected with the nRP vectors and nearly 2 times more than cells transfected with the ARP with GFP in the second position.

In summary, these results confirmed that both of the non-replicating plasmids express GFP at similar levels, which did not substantially increase over the three days of the study. The replicating plasmids, however, both demonstrated substantially more GFP expression than the non-replicating plasmids, and the GFP expression continued to increase over the course of the study. Additionally, the replicating plasmid with GFP in the first position expressed more GFP than the replicating plasmid with GFP in the second position at each time point after the first day, indicating there is a higher level of expression of the gene located in the first position than the gene located in the second position of the bicistronic plasmid.

### B. Replication of the ARP Vector in HEK 293 Cells

In this example, replication of the ARP vector was determined using TaqMan® quantitative PCR (qPCR). Primers for TaqMan® qPCR are set forth in Table 28 below. HEK 293 cells were transfected with the ARP vector pCzGFP-I-T-BB3 (SEQ ID NO: 607), or with the control nRP counterpart vector pCzGFP-I-T-dSV (SEQ ID NO: 608), as described in part A above. HEK 293 cells were also transfected with the ARP or nRP vectors containing GFP in the second position, pC-T-I-zG-BB3 (SEQ ID NO: 689) or pC-T-I-zG-dSV (SEQ ID NO: 690), respectively. Untransfected cells also were used as a negative control.

Cells were harvested at 0, 1, 2, 3, and 4 days post transfection. For each time point, DNA was extracted with Invitrogen HQ Purelink kit, according to manufacturer's instructions, and eluted with 100 μl of DNase-free H₂O. Mock or Dpn1 digestion was then performed using 10 μl of the 100 μl DNA solution and 10 μl of reaction mix to eliminate the originally transfected, bacterially-derived plasmids. The remaining newly synthesized plasmids were subjected to quantitative PCR (qPCR), using 1 μl reaction mix per well, a zGFP-specific forward primer (5′-CAAGATTAGACACAACATAGAGGATGGA-3′; SEQ ID NO: 476), zGFP-specific reverse primer (5′-TGTGATCTCTCTTTTCATTAGGATCTTT-3′; SEQ ID NO: 477), and TaqMan® probe, (SEQ ID NO: 478: 5′-CTGTGCAGCTGGC-3′, with a 6-carboxyfluorescein (FAM) fluorophore covalently attached to the 5′ end and the dihydrocyclopyrroloindole tripeptide minor groove binder (MGB) quencher covalently attached to the 3′ end). The relative plasmid copy number was then quantified by TaqMan® quantification of PCR products versus a standard curve, for each time point. The same DNA extraction and qPCR procedures were also conducted with untransfected cells to determine the background signal from contaminating chromosomal DNA. The relative copy numbers were determined for each condition at each time point.

The untransfected cells yielded a low background relative plasmid copy number, which remained more or less the same at each time point. Cells transfected with the non-replicating vectors, pCzGFP-I-T-dSV or pC-T-I-zGFP-dSV, produced similar relative plasmid copy numbers at each time point, that were about 100 times background levels. The relative copy numbers for both non-replicating plasmids remained constant over the time course of the study. The self-replicating plasmids, pCzGFP-I-T-BB3 and pC-T-I-zG-BB3, demonstrated a steady increase in relative plasmid copy number over the course of the study. The rates at which the ARP vectors replicated, were indistinguishable from each other. In this study, the replicative ability of the ARP vectors, and the non-replicative nature of the nRP vectors, were confirmed, and it was further determined that the ARP vectors were able to replicate at similar levels, irrespective of the location of the TAg gene.

In further experiments, replication of the ARP vector, pCzGFP-I-T-BB3, in HEK293 cells was tested using gene-specific primers capable of amplifying other regions of the vector, using the protocol described above. In addition to the primers used for zGFP cDNA above, forward and reverse primers specific for wild type, CpG-free TAg (forward primer: SEQ ID NO: 491 and reverse primer: SEQ ID NO: 492) also were tested. These further experiments yielded results similar to those described above, confirming the ability of pCzGFP-I-T-BB3 to self-replication.

TABLE 28
TaqMan ® quantitative PCR (qPCR) primers

| Target | Primer | Sequence | SEQ ID NO |
|---|---|---|---|
| IRES | forward | 5′-TCTCGCCAAAGGAATGCAA-3′ | 471 |
| | reverse | 5′-CCTGCAAAGGGTCGCTACAG-3′ | 472 |
| | probe | 5′-FAM-TCTGTTGAATGTCGTGAAGG-MGB-3′ | 473 |
| zEGFP | forward | 5′-CAAGATTAGACACAACATAGAGGATGGA-3′ | 476 |
| | reverse | 5′-TGTGATCTCTCTTTTCATTAGGATCTTT-3′ | 477 |
| | probe | 5′-FAM- CTGTGCAGCTGGC-MGB-3′ | 478 |
| pUC ori | forward | 5′-GCGTAATCTGCTGCTTGCAA-3′ | 479 |
| | reverse | 5′-AAGCCAGTTACCTTCGGAAAAA-3′ | 480 |
| | probe | 5′-FAM-TTTGCCGGATCAAGAG-MGB-3′ | 481 |
| zLacZ | forward | 5′-AGAGAGGAGGCTATGCTGACAGA-3′ | 484 |
| | reverse | 5′-GAATCCAACATCACAGGCTTCA-3′ | 485 |
| | probe | 5′-VIC3-CTGCTGAGATCCCCA-MGB-3′ | 486 |

### Example 8

### Replication of ARP Vectors in Human Tumor Cell Lines

In this example, the replicative ability of autonomous replicating plasmids (ARP) was examined in a variety of cell lines.

### A. ARP Replication in a Panel of Human Tumor Cell Lines

A comprehensive panel of more than 45 human tumor cell lines was tested for replication of ARP vectors or non-replicating (nRP) control constructs (e.g. pancreatic cancer cell line, BxPC-3 (Caliper Lie Sciences); prostate carcinoma cell line, PC-3-Luc (Caliper Life Sciences); Burkitt's lymphoma cell line, Ramos (ATCC); mesothelioma cell line, REN (Smythe et al., (1994) Cancer Res. 54(8):2055-2059); osteosarcoma cell line, Saos2 (ATCC); osteosarcoma cell line, U2OS (ATCC). Plasmid replication was determined by SYBR® Green (BioRad) quantitative PCR (qPCR) using primer pairs set forth in Table 29 below.

TABLE 29
SYBR ® Green qPCR primers

| Target | Primer | Sequence | SEQ ID NO |
|---|---|---|---|
| IRES | forward | 5′- ATAGTTGTGGAAAGAGTCAA-3′ | 474 |
| | reverse | 5′- TTAACCTCGACTAAACACAT-3′ | 475 |
| zEGFP | forward | 5′- CAAGATTAGACACAACATAGAGGATGGA-3′ | 476 |
| | reverse | 5′- TGTGATCTCTCTTTTCATTAGGATCTTT-3′ | 477 |
| EGFP | forward | 5′- AAGCTGGAGTACAACTACAA-3′ | 487 |
| | reverse | 5′- ATCTTGAAGTTCACCTTGAT-3′ | 488 |
| pUC ori | forward | 5′- GCGTAATCTGCTGCTTGCAA-3′ | 479 |
| | reverse | 5′AAGCCAGTTACCTTCGGAAAAA-3′ | 480 |
| zLacZ | forward | 5′-AGAGAGGAGGCTATGCTGACAGA-3′ | 484 |
| | reverse | 5′- GAATCCAACATCACAGGCTTCA-3′ | 485 |
| zSV40-T Ag | forward | 5′- CCATAGGCATAGGGTGAGTGCAA-3′ | 491 |
| | reverse | 5′- GTCTCTGGTCAGGGCTGAATACAT-3′ | 492 |
| zGALV | forward | 5′-ACTGCAAAGAGTGGGACTGTGAGA | 493 |
| | reverse | 5′-GGCATTGCTGAAATTTCTGGGTCC | 494 |

Each cell line was transfected, using an electroporation-based method using a Nucleofector II (Amaxa), with replication competent pCzGFP-I-T-BB3 (SEQ ID NO: 607) or non-replicating pCzGFP-I-T-dSV (SEQ ID NO: 608) vectors, at a ratio of 1 μg plasmid per 4×10⁶ cells. Other replication-competent constructs were also tested, including SV40-Tag mutant vector pCzGFP-I-T (S677A S679A)-BB3 (SEQ ID NO: 730), and control vectors pSVB3 (Peden et al. (1980 Science, 290:1392-1396) and pEBNA DEST (Life Technologies, Catalog No. A10989). Cells were co-transfected with a plasmid coding for a more intensely fluorescent GFP, pmaxGFP (SEQ ID NO: 469), as a control measure for transfection efficiency. After different time points post-transfection (0-5 days), plasmid DNA was extracted from the cells via a simplified Hirt method (Hirt, B, (1967) J Mol Biol. 26(2):365-369) and the plasmid copy number was determined using SYBR® Green qPCR, using primers specific for the pUC ori (see above). Because GFP expression should increase concomitantly with plasmid copy number, GFP fluorescence was also monitored visually, to confirm the results observed by qPCR.

A wide range of replication competence among the cell lines tested was observed, ranging from barely detectable to >1000 fold amplification of plasmid (e.g. Ramos vs. U2OS cells). There was no obvious correlation between the ability of cell lines to replicate plasmid and transfection efficiency, tissue type, cell cycle speed or mutational status.

Levels of TAg expression were measured in these cell lines to determine if reduced TAg expression contributed to the reduced replication and GFP expression observed. The levels of TAg expression in weekly replicating lines were significantly lower than those in better replicating lines, suggesting that TAg expression and or/stability could be an underlying cause for the differences in replication observed among the different cell lines.

To determine if runaway replication of the plasmid itself was cytotoxic, cell viability assays (MTS) were performed at each time point following DNA delivery. Even in cell lines which were highly transfectable, and exhibited high levels of plasmid replication, such as the U2OS cells, the impact on cell viability due to replication itself was minimal (10% reduction in viability compared to the dsv-transfected cells), indicating that the vector alone is not sufficient to induce cell death.

Examples of replication results in specific transfected cell lines are provided in Table 30 below.

TABLE 30
Summary of ARP Replication in Human Tumor Cell Lines

| | | | pCzGFP- | | |
|---|---|---|---|---|---|
| | | | I-T(S677A | | |
| Tissue/ | | pCzGFP- | S679A)- | | |
| Tumor Type | Cell Line | I-T-BB3 | BB3? | pSVB3 | pEBNA |
| Kidney | Caki1 | 0 | n.d. | 0 | 0 |
| (RCC) | Caki2 | 0 | n.d. | 0 | 0 |
| | 786O | + | n.d. | n.d. | n.d. |
| Osteosarcoma | U2OS | +++ | n.d. | +++ | 0 |
| | Saos2 | +++ | n.d. | + | 0 |
| | MG63 | 0 | 0 | 0 | 0 |
| | KHOS/NP | 0 | n.d | n.d. | n.d. |
| | SJSA-1 | 0 | n.d. | n.d. | n.d. |
| | 143B | 0 | n.d. | n.d. | n.d. |
| | TI-73 | 0 | n.d. | n.d. | n.d. |
| Colon | HCT116 WT | 0 | n.d. | n.d. | n.d. |
| | HCT116 | 0 | n.d. | n.d. | n.d. |
| | p53^{-/-} | | | | |
| | HCT116 | 0 | n.d. | n.d. | n.d. |
| | p21^{-/-} | | | | |
| Breast | MCF10a | 0 | n.d. | n.d. | n.d. |
| | MCF7 | 0, +* | n.d. | n.d. | n.d. |
| | MDA-MB- | 0^{†} | n.d. | n.d. | n.d. |
| | 231 | | | | |
| Lung | A549 | 0 | n.d. | + | 0 |
| | H1299 | +, +++* | + | + | n.d. |
| Pancreas | MiaPaCa2 | + | + | n.d. | n.d. |
| | Panc2.13 | + | n.d. | n.d. | n.d. |
| | Capan1 | 0 | n.d. | n.d. | n.d. |
| | Capan2 | 0 | n.d. | n.d. | n.d. |
| Lymphoma | HL60 | 0 | n.d. | n.d. | n.d. |
| | Raji | + | n.d. | n.d. | n.d. |
| | Ramos | + | n.d. | n.d. | n.d. |
| | EB3 | 0 | n.d. | n.d. | n.d. |
| | HS-Sulta | 0 | n.d. | n.d. | n.d. |
| | NB4 | 0 | n.d. | + | n.d. |
| Fibroblasts | MRC5- | 0 | n.d. | n.d. | n.d. |
| (lung) | TERT | | | | |
| Mesothelioma | REN | +++ | n.d. | 0 | n.d. |
| | H2452 | 0 | n.d. | n.d. | n.d. |
| | H28 | 0 | n.d. | n.d. | n.d. |
| | H2052 | 0 | n.d. | n.d. | n.d. |
| | H226 | 0 | n.d. | n.d. | n.d. |
| | MSTO- | 0 | + | n.d. | n.d. |
| | 211H | | | | |
| Cervix | GH354 | 0^{†} | n.d. | n.d. | n.d. |
| Adeno- | GH329 | 0^{†} | n.d. | + | n.d. |
| carcinoma | | | | | |
| (E1A+, E1B+) | | | | | |
| Kidney | 293 | + | n.d. | n.d. | n.d. |
| | 293T | + | n.d. | n.d. | n.d. |
| Merkel cell | MKL-1 | 0 | n.d. | n.d. | n.d. |
| carcinoma | | | | | |

0 = no replication (<10 fold)
0^{†} = no replication (<10 fold), but a few bright green cells were observed by fluorescent microscopy
+ = replication up to 100 fold
+++ = replication greater than 100 fold
*= results fell into two categories from different experiments

### B. Replication of ARP in a Prostate Cancer (PC-3-Luc) Cell Line

Cells from the PC-3-Luc cell line (Caliper Life Sciences), a subclone of the PC-3 human prostate cell line that has been stably transfected with the firefly luciferase gene (Luc), were maintained as adherent monolayer cultures in F12-K medium (Mediatech, Manassas, Va.) supplemented with 10% fetal bovine serum (FBS) (Omega Scientific, Tarzana, Calif.) and incubated in 5% CO₂ with a balance of air at 37° C. Cells were split and the medium was changed biweekly. PC-3-Luc cells were transiently transfected with pCz-GFP-I-T-BB3 (SEQ ID NO: 607) using Lipofectamine 2000 (Invitrogen, Carlsbad, Calif.) as described in Example 7A above. Cells were harvested at 0, 1, 2, 3, and 4 days post transfection. Plasmid DNA was extracted from the harvested cells, Dpn1-digested (Example 7B), to eliminate the originally transfected, methylated, bacterially derived, plasmids. Plasmid copy numbers were determined using SYBR® Green quantitative PCR, as described in part A above, to determine the fold increase of copy number at each time point. The copy number of the replication plasmid (pCz-GFP-I-T-BB3) was detectable beginning at day 1 post-transfection and by day 2 post-transfection was about 75-fold that observed at day 0. By Day 4, the copy number of the replicating plasmid, ARP (pCzGFP-I-T-BB3), was approximately 200 fold that observed at day 0, whereas the non-replicating plasmid demonstrated a negligible increase in plasmid copy number at all time points.

### C. Replication of ARP in a Pancreatic Cancer (BxPC-3) Cell Line

Pancreatic cells were also analyzed as described in part A above. Transfected BxPC-3-Luc cells (Caliper Life Sciences) were harvested at 0, 1, 2, 3, and 4 days post transfection, and plasmid DNA was extracted, Dpn1-digested, to eliminate the originally transfected, methylated, bacterially derived, plasmids, and analyzed by SYBR Green quantitative PCR as discussed in part B above. At Day 4, the copy number of the replicating plasmid, ARP (pCzGFP-I-T-BB3), was approximately 40 fold that observed at day 0, whereas the non-replicating plasmid demonstrated a negligible increase in plasmid copy number at all time points.

### D. Replication of ARP in REN Mesothelioma Cells

Human mesothelioma REN cells (Smythe et al., (1994) Cancer Res. 54(8):2055-2059) were maintained in RPMI 1640 medium (Mediatech, Manassas, Va.) supplemented with 10% fetal bovine serum (FBS) (Omega Scientific, Tarzana, Calif.), incubated in 5% CO₂ with a balance of air at 37° C. Cells were harvested and the medium was changed biweekly. As another example of ARP replication capability, REN cells were transfected using either the replication-incompetent plasmid expressing GFP (pCzGFP-I-T-dSV; SEQ ID NO: 608), or the replication-competent ARP expressing the GFP gene (pCzGFP-I-T-BB3; SEQ ID NO:607). The cells were imaged by fluorescence microscopy seventy-two hrs post-transfection. A significant enhancement of GFP expression was observed in the cells that received the replication-competent ARP.

### Example 9

### Plasmid Replication with SV40 Large T Antigen Provided in Trans

In this example, expression of a gene contained within an autonomous replicating plasmid (ARP) was monitored when provided with increasing amounts of an additional source of SV40 Large T Antigen (TAg). PC-3-Luc cells were maintained as described in Example 8B above. 2×10⁵ PC-3-Luc cells were plated in 6 well plates in 3 ml media. The following day, the cells were transiently transfected, according to the protocol described in Example 7A, with the autonomous replicating plasmid expressing the gene for Gibbon Ape Leukemia Virus envelope protein (GALV), pCzGALV-I-T-BB3 (SEQ ID NO:653). In replicate wells, cells were co-transfected with 0, 10, 20, 50, 100, or 200% of pCz-GFP-I-T-BB3 (SEQ ID NO: 607), as an additional source of TAg. Replication of the pCzGALV-I-T-BB3 vector was measured by SYBR® Green quantitative PCR, as described in Example 8A above, using the GALV-specific qPCR primers set forth in SEQ ID NOS: 493 and 494, at 0, 1, 2, 3, and 4 days post-transfection. The fold increase in copy number was determined for each condition at each time point by normalizing the signal to that observed at day 0 for the same condition.

Increased co-transfection of a plasmid, supplying additional TAg in trans, corresponded to increased pCzGALV-I-T-BB3 plasmid replication, in a dose dependent manner. At day 3, PC-3-Luc cells expressing the pCzGALV-I-T-BB3 plasmid alone generated about a 2.5-fold increase in GALV-containing plasmids; co-transfection with 200% more TAg yielded a greater than 50-fold increase in GALV-containing plasmids; co-transfection with 100% more TAg resulted in a greater than 25-fold increase in GALV-containing plasmids; co-transfection with 50% or 20% additional TAg-expressing plasmid resulted in about a 10-fold increase in GALV plasmid copy number; 10% TAg supplementation yielded approximately a 5-fold increase in GALV-plasmid. The results from this experiment indicate that ARP replication is not solely governed by TAg intrinsic to the ARP. Instead, ARP replication and expression also can be regulated by TAg produced from a separate plasmid, and this suggests that other sources of TAg within a host cell also may control ARP expression.

### Example 10

### Replication of Plasmids Containing TAg Mutants that Fail to bind Rb and p53

In this example, TAg, in the autonomous replicating plasmids (ARPs), was mutated to prevent TAg binding to Rb and p53 (see Example 3). The TAg mutants were then tested for their abilities to confer selective ARP replication to p53 and Rb-deficient cells.

### A. Replication of TAg Mutants

Derivatives of pCzGFP-I-T-BB3 were generated expressing various Rb- or p53-binding mutants of TAg (L103P (SEQ ID NO: 618), C105A (SEQ ID NO: 619), E107L (SEQ ID NO: 620), E107K (SEQ ID NO: 621), E107L/E108L (SEQ ID NO: 634), and P453S (SEQ ID NO: 626), V585R (SEQ ID NO: 627), D604R (SEQ ID NO: 628)) by replacing the TAg sequence as described in Example 5 and 6. The TAg wild-type (WT) and mutant plasmids were electroporated into Rb and p53 deficient cells such as the osteosarcoma line Saos2 (ATCC), the p53-dysfunctional cell line U2.9927 (U2OS with dominant negative p53), or U2OS cells at 0.25 μg DNA/10⁶ cells (ATCC), and lack of binding was confirmed by pull-down assays. GFP expression, as a surrogate for replication, was assessed 3 days post-electroporation. The pCzGFP-I-T-dSV plasmid (SEQ ID NO:608), containing wild-type TAg, but lacking the SV40 ori was used as a negative control. Each of the replication competent mutant plasmids were capable of replication in the Rb and p53-deficient cell lines. Plasmids encoding the Rb binding mutant E107K and the p53 binding mutant V585R performed best and displayed levels of replication similar to those expressing wild-type TAg

B. Selectivity of Replication in Tumor vs. Normal Cells

In this example, the derivatives of pCzGFP-I-T-BB3 containing the p53-binding mutant of TAg V585R (designated pCzGFP-I-T(V585R)-BB3; set forth in SEQ ID NO: 627) was further tested for selective replication in p53-deficient over p53-normal cells. U2.9927 cells express a dominant negative form of p53 (DNp53), and were thus used as the p53 deficient cell line. U2OS cells (ATCC), from which U2.9927 cells were derived express wild-type p53, and served as the p53-normal cells. U2.9927 and U2OS cells were transiently transfected with pCzGFP-I-T(WT)-BB3 (SEQ ID NO: 607) or pCzGFP-I-T(V585R)-BB3 (SEQ ID NO: 627) by electroporation as described in part A above, and the replication efficacy was measured by GFP expression 3 days post-electroporation and by qPCR. The cells transfected with pCzGFP-I-T(V585R)-BB3 containing the V585R TAg mutant replicated just as well as WT in U2OS cells. Similar studies were also performed using Saos2 cells, which do not express p53.

In a further experiment, U2OS cells were treated with either nutlin-3 for 12-24 hr (a compound that leads to accumulation of high levels of active p53 through inhibition of Mdm2 or an IPTG-inducible ARF (Stott et al., EMBO, 1998) for 16-24 hr, a known activator of P53 (Lowe and Sherr, (2003) Curr Opin Genet Dev. 13(1):77-83), followed by transfection by pCzGFP-I-T(WT)-BB3 (SEQ ID NO: 607) or pCzGFP-I-T(V585R)-BB3 (SEQ ID NO: 627) and assessment of replication by way of GFP fluorescence and qPCR. Under conditions of enhanced p53 activity, pCzGFP-I-T(V585R)-BB3 replicated at the level of pCzGFP-I-T(WT)-BB3 or better. These experiments demonstrate that p53 binding mutants of TAg do not provide selectivity of replication for tumor over normal cells.

### Example 11

### Fusogenic Activity of Autonomously Replicating Plasmids (ARPs) Constructs

### A. Indentifying Candidate Fusogenic Proteins

Six fusogenic proteins capable of cell-cell fusogenic activity without the help or association with other genes were tested for independent fusogenic activity. These proteins, Avian Reovirus P10 protein (ARVp10), Reptile Reovirus P14 (RRVp14), Baboon Reovirus P15 (BRVp15), Simian Virus 5 F protein (SV5F), Vesicular Stomatitis Virus G protein (VSVG), Gibbon Ape Leukemia Virus envelope protein (GALV), and their corresponding GenBank Accession Nos. and Protein and DNA SEQ ID NOS are set forth in Table 31 below.

Initial fusogenic constructs were made by the insertion of NheI-BamHI flanked synthetic fusogenic protein cDNAs into the NheI-BamHI sites of pIRES2-zGFP (SEQ ID NO: 694) as described in Example 6, generating constructs with the formula name pC-“zF”-I-zG, where “zF” is a CpG free, human codon-optimized cDNA for a fusogenic protein, I is the internal ribosomal entry site (IRES), and zG is zGFP. When transfected into cells, these constructs express both the fusogenic cDNA and zGFP under the regulation of the CMV promoter. SEQ ID NOS for the pC-“zF”-I-G constructs are set forth in Table 31 below.

TABLE 31
Independent Fusogenic Protein Candidates

| | | SEQ ID NO | | | |
|---|---|---|---|---|---|
| Fusogenic | GenBank | | | | pC-“zF”- |
| Protein | Accession | Wild-Type | Optimized | | I-zG |
| (“F”) | No. | cDNA | cDNA | Protein | vector |
| ARVp10 | AY395797 | 8 | 9 | 39 | 715 |
| RRVp14 | DD038189 | 12 | 13 | 41 | 716 |
| BRVp15 | AF06787 | 14 | n/a | 42 | 717 |
| SV5F | NC006430 | 17 | 18 | 44 | 718 |
| VSVG | AJ318514 | 6 | 7 | 38 | 714 |
| GALV | NC001885 | 15 | 16 | 43 | 713 |

Fifty (50) ng of each pC-“zF”-I-zG construct were transiently transfected into HEK293 cells, using Lipofectamine 2000 (BioRad), according to the manufacturer's instructions. The cells were then observed by GFP fluorescence microscopy, 24 hr after transfection, for fusogenic activity: cell-cell fusion and multinucleated syncytia formation. Fusogenic proteins RRVp14, BRVp15, and GALV exhibited the most fusogenic activity and were selected for further study.

### B. Generation of Replicating and Non-Replicating Control Fusogenic Constructs

Autonomous replicating constructs containing, SV40 TAg and cDNA encoding a fusogenic protein, were constructed by digesting the pCzGFP-I-T-BB3 (SEQ ID NO: 607) or vector with NheI/BamHI, to remove zGFP, and subcloning the CpG-free, human codon-optimized fusogenic cDNAs, containing 5′ NheI and 3′ BamHI restriction sites, into the vacated zGFP position, to give rise to constructs with the formula name pC-“zF”-I-T-BB3, where “zF” is a human codon-optimized fusogenic cDNA, set forth in Table 31 above. As set forth in Example 6, the following replicating fusogenic constructs were generated by this method: pCzARVp10-I-T-BB3 (SEQ ID NO: 647), pCzRRVp14-I-T-BB3 (SEQ ID NO: 649), pCzBRVp15-I-T-BB3 (SEQ ID NO: 651), pCzGALV-I-T-BB3 (SEQ ID NO: 653), pCzSV5F-I-T-BB3 (SEQ ID NO: 655), and pCzVSVG-I-T-BB3 (SEQ ID NO: 657).

Non-replicating constructs (nRPs), containing the above-mentioned fusogenic cDNAs, were also generated, using the procedure above, except the fusogenic cDNA was subcloned into the pCzGFP-I-T-dSV construct (SEQ ID NO: 608), which lacks the SV40 ori. As set forth in Example 6, the non-replicating fusogenic constructs generated are as follows: (pCzARVp10-I-T-dSV (SEQ ID NO: 648), pCzRRVp14-I-T-dSV (SEQ ID NO: 650), pCzBRVp15-I-T-dSV (SEQ ID NO: 652), pCzGALV-I-T-dSV (SEQ ID NO: 654), pCzSV5F-I-T-dSV (SEQ ID NO: 656), and pCzVSVG-I-T-dSV (SEQ ID NO: 658).

### C. Fusogenic Activity of RRVp14 ARPs in HEK293 Cells

In this example, the ability of stand-alone fusogenic cDNA-containing construct designated pCzRRVp14-I-T-BB3 (SEQ ID NO: 649) containing the fusogenic peptide RRVp14 to form syncytia in transfected HEK 293 cells was examined. 5×10⁵ HEK293 cells were plated in triplicate in wells of a 6-well plate and transfected with 20 ng of replicating or non-replicating RRVp14 fusogenic plasmids (pCzRRVp14-I-T-BB3 (SEQ ID NO: 649) or pC-RRVp14-I-T-dSV (SEQ ID NO: 650)) by electroporation. Three days post-transfection, the cells were fixed with ice cold 100% methanol, stained with 0.05%% crystal violet (please confirm) for 30 minutes at room temperature, and then washed with 100% methanol to destain. After washing, the cells were assessed for syncytia formation. HEK293 cells expressing the nRP vectors formed a relatively low level of syncytia, whereas expression of the ARP resulted in the formation of several large multinucleated syncytia. Thus, the results show that ARPs express fusogenic peptides as demonstrated by syncytia formation in ARP-transfected HEK293.

### D. Fusogenic Activity of GALV ARPs in HEK293 Cells

In this example, the replicating and non-replicating fusogenic vectors, pC-GALV-I-T-BB3 (SEQ ID NO: 653) and pC-GALV-I-T-dSV (SEQ ID NO: 654), were compared in a fusion assay with HEK293 cells. Confluent plates (˜5×10⁵ cells) in 6-well plates were transfected with 500 ng plasmid using JetPEI™ (Polyplus-Transfection) at a 3:1 ratio. Cells were analyzed for cell fusion by phase contrast microscopy, using a 10× objective, at 10, 22, and 36 hr post-transfection, and compared with untransfected HEK293 cells.

By 10 hr post-transfection, the replication-competent pC-GALV-I-T-BB3 vector displayed visible cell-cell fusion, whereas the non-replicating dSV vector did not lead to cell-cell fusion at this early time-point. Similar cell-cell fusion was evident for both vectors by approximately 22 hr post transfection, and maximal cell fusion was observed by approximately 36 hr post transfection for both vectors. Untransfected cells did not exhibit cell-cell fusion at any time point. The fusion observed, at 10 hr post-transfection, for cells expressing the replicating vector exhibit fusion, but not for cells transfected with the non-replicating vector, is consistent with the hypothesis that the replicating vector is capable of expressing higher levels of GALV as a consequence of autonomous replication.

In a further experiment, to estimate the number of cells that might be fused in a single syncytium, HEK293 cells were transfected with low amounts of the replication-competent pC-GALV-I-T-BB3 (SEQ ID NO: 653) vector (10 ng/10⁵ cells) in order to produce a minimal amount of fusogenic foci. After allowing for cell-cell fusion to occur for six days, the number of cells that make up a fusogenic focus was estimated to be approximately 174 cells.

### E. Induction of Syncytia Formation and Cell Death by Fusogenic ARPs

In this example, fusogenic peptides were tested for their abilities to induce syncytia formation and cell death in human tumor cell lines. Fusogenic activity was assessed by microscopy and cell viability was measured by MTS assay.

PC-3-Luc cells (Caliper Life Sciences) were co-transfected with ARP pCzGALV-I-T-BB3 (SEQ ID NO: 653) and pCzGFP-I-T-BB3 (SEQ ID NO: 607), or non-replicating pCzGALV-I-T-dSV (SEQ ID NO: 654) and pCzGFP-I-T-dSV (SEQ ID NO: 608) at a ratio of 80:20 using electroporation as described above. Syncytia formation was assessed daily for 3 days post-transfection. Cells expressing the replication-competent plasmid exhibited enhanced syncytia formation, while minor syncytia were observed in cells expressing the non-replicating plasmid.

In a further experiment, U2OS cells (ATCC) were co-transfected with ARP pCzRRVp14-I-T-BB3 (SEQ ID NO: 649), or nRP pCzRRVp14-I-T-dSV (SEQ ID NO: 650), and pCzGFP-I-T-BB3 (SEQ ID NO: 607) at an 80:20 ratio, to better visualize syncytia formation. On day 3 post-transfection, the cell nuclei were stained with DAPI and examined for syncytia formation using bright field and fluorescent microscopy. Micrographs of transfected U2OS cells illustrated replication-dependent syncytia formation, as cells expressing the ARP fusogenic vector were positive for syncytia formation, while the U2OS cells expressing the nRP fusogenic vector neglected to form syncytia. The same cells were also monitored for viability using an MTS assay. U2OS cells expressing the non-replicating fusogenic plasmid exhibited about 3% cell death, compared to cells expressing the pCzGFP-I-T-BB3 control plasmid (see Example 8). In contrast, cells expressing the ARP fusogenic vector, pCzRRVp14-I-T-BB3, exhibited about a 25% reduction in viability, indicating that the cell death observed for the ARP-expressing cells was replication-dependent.

### Example 12

### Effect of ARP on Tumor Incidence and Growth in Xenograft Tumor Model

In this example, a human xenograft model was generated to assess subcutaneous tumor formation of tumor-causing cells that had been pre-transfected with auto-replicating or non-replicating plasmids expressing a fusogenic protein. Tumor take rate and size were measured to validate the model and to determine replication-dependent tumor inhibition by ARPs.

### A. Xenotransplantation of PC-3-Luc Cells

To generate a tumor mouse model, 2×10⁶ PC-3-Luc cells (Caliper Life Sciences), in 100 μl serum-free Opti-MEM medium, were injected subcutaneously into both hind flanks of male immunocompromised, athymic nude mice. On a weekly basis, for 7 weeks, the tumor growth was monitored by external caliper measurement. In order to determine the volume by external caliper, the greatest longitudinal diameter (length) and the greatest transverse diameter (width) were determined Tumor volume based on caliper measurements were calculated using a modified ellipsoid formula (Euhus et al., (1986) J Surg Oncol. 31:229-234; Tomayko and Reynolds, (1989) Cancer Chemother Pharmacol. 24:148-154):

Tumor volume=π/6(length×width²).

Sites of injection yielding tumor volumes of at least 63 mm³ were considered to be tumor-positive, as defined by the National Cancer Institute (NCI). At 7 weeks, 15 out of 16 (94%) PC-3 injection sites presented tumors by NCI standards.

### B. Effect of ARP Pre-Transfection on Tumor Incidence and Growth

To test the effect of ARPs that express fusogenic proteins on tumor incidence and growth, PC-3-Luc (Caliper Life Sciences) or REN cells (Smythe et al., (1994) Cancer Res. 54(8):2055-2059) were pre-transfected with the replication competent ARP, or replication incompetent nRP, vector containing the cDNA for GALV or RRVp14 fusogenic peptides, under the regulation of a CMV promoter, and injected into immunocompromised mice to analyze the effect of ARPs expressing fusogenic proteins on tumor incidence and growth.

i. REN Cells

REN human mesothelioma tumor cells (Smythe et al., (1994) Cancer Res. 54(8):2055-2059) were grown in vitro and transfected, according to standard protocol using Lipofectamine L2K transfection reagent, with plasmid expressing the GALV gene product in either the replication competent vector (pC-GALV-I-T-BB3; SEQ ID NO: 653) or the vector incapable of replication (pC-GALV-I-T-dSV; SEQ ID NO: 654). As a control, untransfected REN cells were also used. In one study (Study 1 in Table 32), 800 ng of plasmid DNA was transfected into 1×10⁶ REN cells. Tumor cells were transfected for approximately 4 hrs, whereupon they were harvested into serum-free media and injected into the subcutaneous flank of athymic nude (NCr nu/nu) male mice. All animals received a 100 μL subcutaneous injection of three million tumor cells (3×10⁷ cells/mL) into each rear flank of the animal (2 injections/mouse). For a positive control, untransfected REN cells were also injected into nude mice. All animals received two subcutaneous injections of tumor cells. Tumors were then allowed to initiate and grow over time, and their sizes were monitored by weekly external caliper measurements, as described in part A above. Tumor incidence was measured as the percentage of the number of tumors with volumes greater than 63 mm³ divided by the number of injection sites. Mean tumor volumes were calculated using the volumes of all visible, caliper-measurable tumors.

Nine weeks after tumor cell implantation, 94% of the injection sites that received untransfected REN cells had developed flank tumors (by NCI standards). In contrast, mice that received the REN cells pre-transfected with 800 ng of the pC-GALV-I-T-dSV construct had an incidence of 43.8%, a 53% reduction in the incidence of tumor formation compared to untransfected control (p<0.05). The lowest incidence of tumors was observed in the incidence of tumors from REN cells pre-transfected with 800 ng of the replicating pC-GALV-I-T-BB3 construct. The incidence of tumors in this group was 12.5%, an 81% reduction in incidence compared to untransfected control (p<0.05).

Mean tumor volume derived from untransfected cells, increased steadily from 0 to 218±39 mm³ (mean±SEM) in 9 weeks. In contrast, tumors derived from cells transfected with 800 ng of the non-replicating GALV-expressing construct steadily grew in volume from 0 to 68±17 mm³ in the span of 9 weeks, and tumors derived from cells transfected with 800 ng of replicating GALV-expressing construct steadily grew in volume from 0 to 18±9 mm³ in 9 weeks. Comparison of tumor volumes among treatment groups demonstrated that pre-transfection of the REN cells with 800 ng of the replication incompetent pC-GALV-dSV and the pC-GALV-BB3 vectors significantly reduced mean tumor volume by 69% and 92%, respectively, compared to untreated mean tumor volume (p<0.05) by week 9. When only volumes of tumors >63 mm³ were considered, pre-transfection of the REN cells with 800 ng of non-replicating pC-GALV-dSV and replication-competent pC-GALV-I-T-BB3 reduced tumor volume by 66% and 43%, respectively (p<0.05).

In additional studies, the amount of DNA transfected into REN cells was varied. In all experiments, pre-transfection with non-replicating fusogenic constructs reduced the tumor incidence from REN cells in immunocompromised mice, and pre-transfection with replicating fusogenic constructs eliminated tumor incidence.

TABLE 32
Tumor incidence and mean tumor volume following subcutaneous
injection of pre-transfected REN mesothelioma cells.

| | | | | % | Mean | |
|---|---|---|---|---|---|---|
| | Amount | | | Reduction | Tumor | |
| | DNA | Tumor | % Tumor | in | Volume^{b} | |
| Plasmid | Transfected | Incidence | Incidence | Incidence^{a} | (mm³) | p-value^{c} |
| Untransfected | N/A | 15/16 | 93.8% | — | 218 ± 39 | N/A |
| pC-GALV-I-T-dSV | 800 ng | 7/16 | 43.8% | 53.3% | 68 ± 17 | p < 0.05 |
| (SEQ ID NO: 654) | | | | | | |
| pC-GALV-I-T-BB3 | 800 ng | 2/16 | 12.5% | 86.7% | 18 ± 9 | p < 0.05 |
| (SEQ ID NO: 653) | | | | | | |

^{a}Compared to tumor incidence for untransfected cells
^{b}Mean ± SEM; at 9 weeks
^{c}p-value based upon 2-way ANOVA: Comparison of tumor incidence of transfected cells vs. untransfected cells

Together, the studies using pre-transfected REN and PC-3-Luc cells to generate Xenotransplant tumor models demonstrated that the expression of the GALV protein reduced the tumorigenesis of cancer cells, especially when the expression of GALV was enhanced by the use of a replication-competent plasmid vector.

ii. PC-3-Luc Cells

Human PC-3-Luc prostate cells were grown in vitro, as described previously, and transfected with a plasmid expressing the GALV gene product in either the replication competent vector (pC-zGALV-I-T-BB3; SEQ ID NO: 653) or the replication incompetent vector (pC-zGALV-I-T-dSV; SEQ ID NO: 654) according to standard protocol using Lipofectamine L2K transfection reagent. Two different quantities of each DNA construct were transfected into the PC-3-Luc cells in this study: 200 ng of plasmid DNA/1×10⁶ PC-3-Luc cells or 400 ng of plasmid DNA/1×10⁶ PC-3-Luc cells.

Transfected cells were harvested, 4 hr post-injection, into serum-free media and injected subcutaneously into both flanks of athymic nude (NCr nu/nu) mice (2 injections/mouse). Untransfected PC-3-Luc tumor cells were also injected into nude mice as a positive control. Each injection contained two million tumor cells in 100 μL (2×10⁷ cells/mL suspension). Tumor incidence and growth, were monitored over time by weekly external caliper measurement, as described in part A above. Tumor incidence was measured as the percentage of the number of tumors with volumes greater than 63 mm³ divided by the number of injection sites. Mean tumor volumes were calculated using the volumes of all visible, caliper-measurable tumors.

Five weeks after tumor cell implantation, 94% (17/18) of the injection sites receiving untransfected PC-3-Luc cells formed subcutaneous flank tumors. In contrast, the mice that received the PC-3-Luc cells pre-transfected with the pC-GALV-I-T-dSV had a significantly reduced incidence of tumor formation (31% and 87% of animals that received cells transfected with 200 and 400 ng of pC-GALV-I-T-dSV plasmid respectively, p<0.5). When these tumor cells were pre-transfected with the replication competent pC-GALV-I-T-BB3 vector, tumor incidence was further reduced compared to untransfected controls (19% and 56% respectively when transfected with 200 and 400 ng respectively). These results are summarized in Table 33. Over the five-week course of the study, the mean tumor volume progressed nearly linearly for all conditions.

Although higher amounts of plasmid DNA used for transfection did not result a higher reduction in tumor incidence, there was a similar trend in reduction of tumor incidence with both amounts of DNA transfected (Δ=20% at 200 ng; 46% at 400 ng). Notably, the reduction in tumor incidence was consistently greatest when tumor cells were transfected with the replication-competent vector compared to the replication incompetent vector, suggesting that the ability for the plasmid to replicate enhances the potency of the fusogenic plasmid vector. In addition to a reduction in tumor incidence, the mean volume of the tumors that grew in the study was significantly smaller in the group that received cells pre-transfected with the fusogenic ARP DNA compared to the untransfected PC-3 tumor cells.

In summary, the pre-transfection of tumor cells with the autologous replicating DNA plasmids demonstrated significant reduction in tumor incidence and growth in mice, indicating that the expression of the fusogenic plasmid DNA in tumor cells can result in an anti-proliferative response which is enhanced by use of a replication-competent construct.

Several additional studies were performed, in which the amount of DNA transfected was varied as presented in Table 33, and tumor growth was monitored for 4-7 weeks. In some experiments, tumors from cells transfected with the ARP and nRP fusogenic vectors were compared to those generated by untransfected cells. In other experiments, the tumors from cells expressing fusogenic replicating and non-replicating vectors were compared with tumors from cells transfected with replicating control (GFP) DNA (pCzGFP-I-T-BB3; SEQ ID NO: 607) to distinguish the fusogenic property of the vectors from the effects of vector replication. In some experiments, tumors from both untransfected and pCzGFP-I-T-BB3-transfected cells were compared to those caused by the fusogenic vectors. The percent tumor incidence and mean tumor volumes for tumors generated are set forth below in Table 34. In all cases, a decrease in the tumor take rate and a decrease of mean tumor volumes was observed for tumors derived from cells expressing the fusogenic proteins compared to those derived from untransfected or control-transfected cells. The tumor incidence and mean volumes were further reduced following injection of cells transfected with auto-replicating fusogenic proteins.

TABLE 33
Summary of tumor incidence of PC-3 tumors after pre-
transfection with GALV-expressing fusogenic plasmids

| | | | | | Mean | |
|---|---|---|---|---|---|---|
| | | | | % | Tumor | |
| | Amount of | | | Reduction | Volume ± | |
| | DNA | Tumor | % Tumor | in | SEM^{b} | |
| Plasmid | Transfected | Incidence | Incidence | Incidence^{a} | (mm3) | p-value^{c} |
| Untransfected | — | 17/18 | 94.4% | — | 459 ± 69 | |
| pC-GALV-I-T-dSV | 200 ng | 4/16 | 25% | 73.4% | 69 ± 30 | p < 0.05 |
| (SEQ ID NO: 654) | | | | | | |
| pC-GALV-I-T-BB3 | 200 ng | 1/16 | 6.3% | 93.6% | 15 ± 11 | p < 0.05 |
| (SEQ ID NO: 653) | | | | | | |
| pC-GALV-I-T-dSV | 400 ng | 14/16 | 87.5% | 7.5% | 332 ± 72 | p > 0.05 |
| (SEQ ID NO: 654) | | | | | | |
| pC-GALV-I-T-BB3 | 400 ng | 7/16 | 43.8% | 53.2% | 155 ± 70 | p < 0.05 |
| (SEQ ID NO: 653) | | | | | | |

^{a}Compared to Tumor Incidence for Untransfected
^{b}Mean ± SEM measured Week 5 post tumor cell injection
^{c}p-value based upon 2-way ANOVA: Comparison of tumor incidence of transfected cells vs. untransfected cells

TABLE 34
Tumor incidence and mean tumor volume following subcutaneous
injection of pre-transfected PC-3-Luc cells.

| | | Tumor | Total | | | Mean |
|---|---|---|---|---|---|---|
| | | Incidence/ | Vector | | | Tumor |
| Study | | Total | DNA/ | | Tumor | Volume ± |
| No. | Vector | Animals | 10⁶ cells | | Incidence | SEM^{(mm3)} |
| 1^{a} | Untransfected | 17/18 | NA | | 94.4% | 481 ± 72 |
| | pC-GALV-I-T-dSV | 14/16 | 400 | ng | 87.5% | 347 ± 76 |
| | (SEQ ID NO: 654) | | | | | |
| | pC-GALV-I-T-BB3 | 7/16 | 400 | ng | 43.8% | 162 ± 73 |
| | (SEQ ID NO: 653) | | | | | |
| | pC-GALV-I-T-dSV | 4/16 | 200 | ng | 25.0% | 73 ± 32 |
| | (SEQ ID NO: 654) | | | | | |
| | pC-GALV-I-T-BB3 | 1/16 | 200 | ng | 6.3% | 16 ± 12 |
| | (SEQ ID NO: 653) | | | | | |
| 2^{a} | Untransfected | 12/13 | NA | | 92% | 403 ± 87 |
| | Control DNA: CpG-BB | 12/15 | 1 | μg | 80% | 461 ± 11 |
| | (SEQ ID NO: 466) | | | | | |
| | pC-GALV-I-T-dSV | 12/14 | 400 | ng | 86% | 345 ± 55 |
| | (SEQ ID NO: 654) | | | | | |
| | pC-GALV-I-T-BB3 | 11/15 | 400 | ng | 73% | 276 ± 60 |
| | (SEQ ID NO: 653) | | | | | |
| 3^{b} | Untransfected | 14/15 | NA | | 93% | 814 ± 126 |
| | Control DNA: CpG-BB | 9/15 | 1 | μg | 60% | 302 ± 108 |
| | pC-GALV-I-T-dSV | 5/14 | 400 | ng | 36% | 44 ± 18 |
| | (SEQ ID NO: 654) | | | | | |
| | pC-GALV-I-T-BB3 | 4/15 | 400 | ng | 27% | 79 ± 40 |
| | (SEQ ID NO: 653) | | | | | |

^{a}Tumor measurements recorded 5 weeks post tumor cell injection
^{b}Tumor measurements recorded 6 weeks post tumor cell injection

### Example 13

### ARP Gene Delivery Via Hydrodynamic Tail-Vein (HTV) Injection

In this example, hydrodynamic tail (HTV) injection of naked plasmid DNA was evaluated as a form of gene delivery to the liver. Investigators have shown that HTV delivery of naked plasmid DNA results in high levels of transgene expression in the parenchyma of the liver (Sebestyén et al., (2006) J Gene Med. 8(7):852-73) which is capable of persistent expression in immunocompromised, athymic mice (Zhang et al., (1999) Hum Gene Ther. 10(10):1735-1737). Liver targeting and distribution of gene expression within the liver following HTV injection were determined.

### A. Targeting DNA to the Liver Via HTV Injection

In this example, the targeting of naked plasmid DNA to the liver via hydrodynamic tail vein injection (HTV) was confirmed. 10 μg of a naked luciferase-expressing reporter plasmid, pLLRNL (Xu et al. (1989) Virology 171(2):331-41) was injected by HTV in 2.5 ml volume of isotonic saline in a 5-7 second interval into sedated homozygous nude male mice. 24 hrs following injection, mice were injected with 200 μl D-luciferin (15 mg/ml). After 10 minutes, the location(s) of the vectors, were determined by detecting luciferase activity in vivo using the non-invasive IVIS bioluminescence imaging system, where the total flux of light emitted over time was measured throughout the mouse bodies. Image analysis was performed using Living Image software v4.0 (Caliper Life Sciences). Results showed high expression levels of the luciferase-expressing reporter plasmid in the liver 24 hrs after HTV injection.

In a further experiment, luciferase-containing autonomous replicating plasmids and non-replicating plasmids were cloned by replacing the GFP genes in pCzGFP-I-T-BB3 (SEQ ID NO:607) and pCzGFP-I-T-dSV (SEQ ID NO:608), with cDNA encoding luciferase (Luc), using the NheI and BamHI restriction sites, to generate pC-Luc-I-T-BB3 (SEQ ID NO:612) and pC-Luc-I-T-dSV (SEQ ID NO:613). pLLRNL, pC-Luc-I-T-BB3, and pC-Luc-I-T-dSV were each injected into the tail vein of 3 male homozygous nude mice at 0.4 mg/kg body weight in 0.1 ml isotonic saline/g body weight. The luminescence emitted from the mouse livers was measured using the IVIS bioluminescence imaging system, as described in the previous experiment, at 1, 3, 7, 10, 14, and 17 days post injection. The results are set forth in Table 35. The expression of the luciferase-containing vectors persisted until the last day of the study (17 days).

TABLE 35
Luminescence as measured by Flux (photons/sec)
in Mouse Liver Using pC-Luc Expression Vectors.

| | | Days Post Injection | | | | | |
|---|---|---|---|---|---|---|---|
| Plasmid | | 1 | 3 | 7 | 10 | 14 | 17 |
| pLLRNL | Mean | 4.07E+07 | 3.02E+05 | 1.73E+05 | 1.19E+05 | 1.24E+05 | 1.74E+05 |
| | SD | 4.09E+07 | 1.16E+05 | 9.74E+04 | 7.15E+03 | 1.55E+04 | 6.59E+03 |
| pC-Luc-I-T-dSV | Mean | 2.03E+09 | 6.73E+07 | 1.05E+07 | 5.65E+06 | 6.47E+06 | 4.60E+06 |
| (SEQ ID NO: 613) | SD | 9.86E+08 | 4.80E+07 | 5.73E+06 | 1.96E+06 | 2.45E+06 | 3.38E+06 |
| pC-Luc-I-T-BB3 | Mean | 4.15E+09 | 2.23E+07 | 5.74E+06 | 2.68E+06 | 3.37E+06 | 2.61E+06 |
| (SEQ ID NO: 612) | SD | 1.72E+09 | 9.29E+06 | 3.40E+06 | 1.83E+06 | 1.97E+06 | 1.23E+06 |

### Example 14

### Liver Toxicity Following Hydrodynamic Tail Vein (HTV) Injection of Autonomously Replicating Plasmids (ARPs)

In this example, the tolerability of HTV injection of ARPs was assessed. Nu/Nu mice were injected with vehicle only, or ARP pCzGFP-I-T-BB3 (SEQ ID NO:607) or pC-GALV-I-T-BB3 (SEQ ID NO:653) via HTV on day 0 at 0.044-0.4 mg/kg body weight. For some mice, a second dose of 0.8-1.6 mg/kg was administered at day 14. The experimental dosage details are set forth in Table 36 below.

TABLE 36
Experimental Setup for HTV Injection of ARP Vectors

| | | | Dose #1 | Dose #1 | | Dose #2 | Dose #2 |
|---|---|---|---|---|---|---|---|
| Group | Vector | n | (mg/kg) | (total μg) | n | (mg/kg) | (total μg) |
| 1 | Untreated | 7 | — | — | — | — | — |
| 2 | pCzGFP-I-T-BB3 | 14 | 0.4 | ~10 | 4 | 0.8 | ~20 |
| | (SEQ ID NO: 607) | | | | | | |
| 3 | pC-GALV-I-T-BB3 | 14 | 0.4 | ~10 | 9 | 0.8 | ~20 |
| 4 | (SEQ ID NO: 653) | 14 | 0.13 | ~3 | — | — | — |
| 5 | | 14 | 0.044 | ~1 | 4 | 0.8 | ~20 |
| | | | | | 5 | 1.6 | ~40 |

Liver toxicity was assessed by drawing ˜200 μl blood from each mouse on days 2, 4, 7, and 22 and measuring the liver enzymes, alanine aminotransferase (ALT) and aspartate aminotransferase (AST) by using a Liasys Chemistry Analyzer.

Single and repeat HTV administration of GALV ARP resulted in transient elevation of both AST and ALT liver enzymes, by day 2, returning to basal levels by day 4. All doses of GALV ARP resulted in similar responses. Administration of GFP ARP 2 days post HTV injection, mice injected with GFP demonstrated a similar increase in ALT levels as was observed for the GALV ARP, but an increase in AST levels was not observed. Injection of vehicle alone GFP ARP and GALV ARPs resulted in a transient rise in the liver enzyme levels on day 2 post injection that resolved for all groups by day 4 post injection.

Because the elevation in liver enzymes on day 2 was observed in all treatment groups, the increase was determined to be an expected cardiovascular response as a result of the transient increase in blood pressure and blood volume that results from HTV injection. The increase in serum transaminases for the mice treated with the GALV-expressing vectors was consistent with reports by other investigators (Liu F. and Liu D., (1999) Hydrodynamics-based transfection in animals by systemic administration of plasmid DNA. Gene Ther. 6: 1258-1266). For mice injected with the GALV ARP, no adverse events (AEs) were observed at the highest level tested (1.6 mg/kg (˜40 μg/mouse)), confirming that HTV delivery of oncovector plasmids was well-tolerated under the conditions tested.

### Example 15

### Characterization of Liver-Localized Tumor Models in Nude Mice

In this example, intrasplenic and intralobular injections of tumor-causing cells into male nude mice were evaluated as liver-localized tumor models. To non-invasively assess tumor growth over time, human prostate cancer PC-3 cells stably transfected with the firefly luciferase gene (PC-3-Luc; described in Example 8), were used. PC-3-Luc cells emit light, produced by luciferase enzyme activity upon administration of the luciferin substrate. The luminescence produced is approximately equivalent among individual PC-3-Luc cells (Feng et al., (2011) J Cancer. 2:123-131), allowing the tumor burden for each animal to be approximated by the light emission following substrate administration (Jenkins et al., (2003) Clin Exp Metastasis. 20(8):745-56).

### A. Intralobular Liver-Localized Tumor Model

In this example, liver-localized tumors were generated by direct intralobular injection of PC-3-Luc cells. Male (Nu/Nu) mice were anesthetized, and an incision was made on the abdomen to expose the liver. 1.5×10⁵ PC-3-Luc cells, in 15 μl sterile HBSS with 50% Matrigel, were injected directly into the liver. The abdominal musculature and skin were sutured and the animals recovered on a warming pad to restore body temperature and returned to their home cage. Tumor formation, for a total of 38 animals, was monitored in vivo using the IVIS Caliper imaging system (Caliper Life Sciences; details to follow), at 1, 7, 14, 21, 28, 35, 42, and 56 days post inoculation. For luciferase detection imaging, 200 ml of 15 mg/ml D-Luciferin (Caliper Life Sciences) in PBS was injected intraperitoneally eight to twelve minutes before imaging. Emitted light was detected using a CCD camera (IVIS/Caliper imaging system). The photometry of developed tumors was calculated by Living Image 4.0 (Caliper Life Sciences) software, and the results were used to generate a tumor growth curve.

Tumor growth or regression was calculated for each animal relative to its initial tumor burden at the beginning of treatment to account for differences of overall signal due to differing tumor establishment and localization in individual animals.

In vivo imaging indicated that the tumors continued to grow over the course of the study. At the early time points (days 1, 7, 14), the tumor growth was slow. The rate of growth increased by day 21 and the rate of growth (slope of the curve) continued to increase between each time point until completion of the study.

Tumors were also visually confirmed by examining the tissue structure of liver sections following hematoxylin and eosin (H&E) staining. The tumor take rate, following intralobular injection of PC-3-Luc cells was >95%, with a single focal tumor per animal.

### B. Intrasplenic Liver-Localized Tumor Model

To establish liver tumors in which tumor causing cells infiltrate the liver in addition to generating bulk sub-cutaneous tumors, PC-3-Luc cells were delivered to the liver after injection into the spleen, using a modified protocol developed by Anderson et al. (Anderson et al., (1998) Clin. Cancer Res. 4:1649-1659). Briefly, male Nude mice were anesthetized, and an incision was made on the left side of the abdomen, such that the spleen could be retracted and exteriorized. 2×10⁶ PC-3-Luc cells were injected into the lumen of the spleen, followed by splenic squeeze. After allowing the cells to distribute for approximately one minute, the blood vessels connected to the spleen were ligated and the spleen was removed. The abdominal musculature and skin were then sutured. Recovering mice were warmed to restore body temperature and returned to their home cages. Intrasplenically-delivered tumor cells were expected to migrate via the portal vein from the spleen into the liver, resulting in the formation of multiple tumor foci over time

Tumor formation was monitored in vivo by the IVIS bioluminescence system as described in part A above at several time points from 1 up to 45 days post cell injection. A total of 30 animals were monitored for tumor formation over 4 independent studies. 53% (16/30) of the mice developed liver tumors (<63 mm³, NCI criteria) following intrasplenic injection of PC-3-Luc cells. In one experiment, animals were sacrificed at day 35 post intrasplenic injection, and the tissue structure of liver sections was examined following H&E staining (described in part A above). In these animals, the presence of liver-localized tumors was confirmed. Intrasplenic delivery of PC-3-Luc cells to the liver resulted in the formation of multiple tumor foci over time, and although each animal received the same dose of tumor cells, the number and size of the tumors that initiated were variable between animals.

### Example 16

### Efficacy of Hydrodynamic Tail Vein (HTV)-Delivered Oncovectors in Mouse Liver Tumor Models

In this example, liver localized tumor models were used as a readout of anti-proliferative activity of autonomously replicating plasmids (ARPs) expressing fusogenic proteins or a pro-drug converting enzyme. Naked ARPs were injected by HTV delivery into PC-3-Luc mouse liver models (described in Example 15) to assess the efficacy of the oncovectors with respect to tumor size. Tumor burden was assessed via bioluminescence as described in Example 15. To reduce variability between each animal, due to attenuation of the overall signal because of variations in the precise localization of the tumors, tumor growth or regression was calculated for each animal relative to its initial tumor burden at the beginning of treatment.

### A. Efficacy of Fusogenic Oncovectors

To evaluate the effect of ARP delivery into an intrasplenic liver tumor model, 2×10⁶ PC-3-Luc cells were delivered to the livers, by intrasplenic injection (as described in Example 15B), of 50 mice on day 0. The tumors derived from the PC-3-Luc cells were imaged in vivo using the IVIS/Caliper imaging system described in Example 15 on days 1, 7, and 14 post-injection, eight to twelve minutes post injection of luciferin in saline (15 mg/kg). Tumor burden was quantified by measuring the total flux of light emitted over time. Image analysis was performed using Living Image software v4.0 (Caliper Life Sciences).

27 animals were found to bear liver-localized tumors. These animals were randomized into three treatment groups of approximately equal mean tumor burden (˜7×10⁷ photons/sec) on Day 14 post injection. On day 17 post-injection, the mice received an HTV injection of the plasmid DNA: 0.4 mg plasmid/kg mouse body weight. One group of mice received administration of the plasmid containing the GALV gene in the replication-competent backbone, pC-GALV-I-T-BB3 (SEQ ID NO: 653). To assess the baseline anti-proliferative activity of the fusogenic GALV gene in the non-replicating backbone, another group of mice received an HTV injection of pC-GALV-I-T-dSV (SEQ ID NO: 654). A third group of mice received HTV injection of a replication-competent plasmid containing a reporter Green Fluorescent Protein (GFP) gene instead of the fusogenic GALV gene (pCzGFP-I-T-BB3 (SEQ ID NO: 607)), to resolve the anti-proliferative effects due to the expression of the fusogenic GALV protein from effects resulting from plasmid replication. Tumor growth was monitored on days 20, 23, 27, and 30 post tumor cell injection. On day 34, the mice were administered a second dose (0.4 mg/kg) of plasmid, and the tumor growth was assessed on days 35, 37, 41, 44, 49, 52, and 55. Tumor growth was monitored by non-invasive luciferase imaging throughout the study, and tumor burden was normalized to the luciferase expression measured on Day 14 post tumor cell injection. The growth of tumors was expressed as the fold-increase in luciferase expression compared to the original baseline value recorded on day 14 post cell injection. The relative increase in luciferase expression was calculated for each treatment group over time. Tumor Growth Inhibition (% TGI) for treatment groups was determined on Day 52 post tumor cell injection, and was calculated using the following formula:

[1−(T_{B}−T_{A})÷(C_{B}−C_{A})]×100

where T_{B} is the average tumor luciferase expression (photons/sec) in the treatment (pC-GALV-I-T-dSV or pC-GALV-I-T-BB3) groups at 38 days after initiation of treatment (52 days post tumor cell implantation); T_{A} is the average luciferase expression (photons/sec) in the treatment groups at day −3 before treatment (14 days post tumor cell implantation); C_{B} is the average luciferase expression (photons/sec) in the control group (pCzGFP-I-T-BB3) at 38 days after initiation of treatment (52 days post tumor cell implantation); and C_{A} is the average luciferase expression (photons/sec) in the control group at day −3 before treatment. Results from this study are recounted below.

Following the first plasmid dose, a minimal increase in luciferase expression (tumor size) was observed in all experimental groups. Following the second dose, the experimental group injected with the replicating GFP plasmid exhibited a rapid and sustained increase in luciferase expression, indicating increased tumor size. The non-replicating GALV-expressing plasmid also exhibited increased luciferase expression following the second plasmid dose. However, the rate of increase was less than that observed for the replicating GFP plasmid (control group). By day 55, the average fold increase in luciferase expression in mice administered the non-replicating GALV plasmid was reduced by 41% compared to the control group. Tumor growth in mice administered the replicating GALV plasmid was reduced by 76% compared to the control group and the difference was statistically significant (p<0.05).

The inhibition of tumor growth observed is consistent with an anti-proliferative effect due to expression of the GALV gene, with the anti-tumor activity of the non-replicating vector (pC-GALV-I-T-dSV) being less than the anti-tumor activity of the replicating vector (pC-GALV-I-T-BB3). These results suggest that the GALV-expressing plasmids have anti-proliferative activity, and that the replication competent GALV-expressing vector, pC-GALV-I-T-BB3, has greater potency than the non-replicating vector, presumably due to an increase in the cytotoxicity that results from the higher expression of GALV.

In a subsequent experiment, the anti-tumor activities of the same three plasmids, and including non-replicating GFP-expressing plasmid (pCzGFP-I-T-dSV (SEQ ID NO: 608)), were evaluated. Mice bearing intrasplenic liver localized PC-3-Luc tumors received five HTV injections of either the oncovector ARPs or a GFP-expressing reporter construct (dosed on days 19, 22, 27, 30 and 34 post cell injection). While the greatest tumor inhibition was observed in the group of mice receiving the replication competent oncovector ARP (pC-GALV-I-T-BB3), this tumor inhibition was not statistically significant compared to the other treatment groups. It is possible that the increase in frequency of dosing may have affected the expression of the fusogenic proteins, due to the continual transient effects on liver parenchymal cells that occur as a result of HTV injection (as evidenced by the transient elevation in serum transaminases that was observed after HTV injection.)

### B. Efficacy of a Pro-Drug Converting Enzyme

In this example, the efficacy of an oncovector ARP expressing a pro-drug converting enzyme was evaluated in an intralobular liver-localized tumor mouse model. The experimental setup for this experiment was similar to that described in part A above, using an liver-localized tumor model generated by intralobular injection of PC-3-Luc cells as described in Example 15A. Additionally, rather than expressing a fusogenic peptide, the experimental constructs used in this study expressed the cytosine deaminase (CDase) gene, which converts non-toxic 5-fluorocytosine (5-FC) to 5-fluorouracil (5-FU), a potent chemotherapy compound. To clone these constructs, the GFP genes in replicating pCzGFP-I-T-BB3 (SEQ ID NO: 607) and non-replicating pCzGFP-I-T-dSV (SEQ ID NO: 608) were replaced with CDase gene (SEQ ID NO: 500) as described in Example 6 above, to generate the replicating pC-CDase-I-T-BB3 (SEQ ID NO: 664) and non-replicating pC-CDase-I-T-dSV plasmids (SEQ ID NO: 665).

Mice receiving intralobular injection of PC-3-Luc cells (1.5×10⁵ in 15 μL of serum-free HBSS), as described in Example 15A, were administered one of the replicating or non-replicating CDase-expressing constructs or the non-replicating GFP-expressing control construct (pCzGFP-I-T-dSV (SEQ ID NO: 608)), at 0.4 mg plasmid/kg body weight, via HTV injection (refer to Example 13 for experimental details of HTV injection). Tumors were allowed to grow over a period of three weeks, whereupon they received an initial HTV injection of either the oncovector ARP or the non-replicating GFP-expressing control construct (Study day 22). Forty eight hours after HTV injection, the animals received their initial administration of 5-FC via an initial intraperitoneal loading dose of 5-FC (100 mg/kg; 5 mg/mL in saline). 5-FC was subsequently provided in their drinking water (5 mg/mL) throughout the remainder of the study. The animals received two subsequent HTV re-administrations of the same vector on Study days 36 and 53. Tumor growth was monitored in vivo using the IVIS bioluminescence system (see Example 15) over a period of 60 days. Upon completion of the study, there were no statistically significant differences between the different treatment groups with respect to tumor size.

While it is well established that HTV injection will provide high levels of gene transfer and expression to hepatocytes, it was not established whether HTV injection would facilitate gene transfer to the intralobular tumors. To address this issue, a satellite group of mice were included in this study that received a single HTV injection of a reporter plasmid expressing the beta-galactosidase gene driven by the CMV promoter (pC-β-gal; 0.4 mg/kg) on day 22 post tumor cell injection. Forty eight hours post HTV injection, the mice were sacrificed and their livers collected frozen in OCT. Sections of the liver were prepared (5 μM) and subsequently fixed (0.2% glutaraldehyde) and stained for lacZ was injected into the HTV of three mice. The livers were harvested 48 hr after HTV injection and frozen in OCT. 5 μm cryosections were fixed with 0.2% glutaraldehyde and then incubated with a staining solution containing 1 mg/ml 5-bromo-4-chloro-3-indolyl-β-D-galactopyranoside (X-gal), 35 mM potassium ferrocyanide, 2 mM MgCl₂, 0.02% NP-40, and 0.01% sodium deoxycholate for 2 h at 37° C. Lac Z staining was visualized by light microscopy (original image obtained using 40× objective). Micrographs taken of representative liver sections demonstrated uniform expression of HTV-injected pC-β-gal throughout the liver. However, no gene expression was observed in the tumor tissue. This inability to demonstrate gene transfer and expression may be a primary factor responsible for the lack of tumor inhibition observed in the intralobular efficacy study using the CDase expressing ARPs.

### Example 17

### In Vivo Replication and Tumor Selectivity of Autonomous Replicating Vectors (ARPs)

In this example the replication of the autonomous replicating vectors (ARPs) is demonstrated in vivo in an intralobular liver-localized tumor model and compared to non-replicating vectors. The selectivity of replication and expression in tumor cells, as compared to healthy tissue, is also assessed by examining the biodistribution of ARP plasmids and gene expression.

A liver-localized tumor model is generated by intralobular injection of PC-3 (no Luc) cells as described in Example 15A. About 14 days post-injection, mice are administered pC-Luc-I-T-BB3 (SEQ ID NO: 612) or pC-Luc-I-T-dSV (SEQ ID NO: 613) by HTV injection (experimental details provided in Example 13). At multiple time points following plasmid administration, luciferase expression is monitored at regular intervals in vivo using bioluminescence (detailed in Example 15) and quantified to confirm in vivo replication via increased luciferase expression over time. Because intralobular injection of PC-3 cells was shown to generate single focal tumors, luciferase signal is monitored at the foci as well as surrounding tissue to determine the specificity of the replication. To further confirm replication, at 2-3 time points post plasmid administration, a minimum of 3 animals from each experimental group are sacrificed, the livers are harvested and dissected to separate tumors from healthy tissue. The respective tissues are then pulverized, plasmid DNA is extracted using a modified Hirt method, and the plasmid number is determined by qPCR as described in Example 7B, providing a quantitative readout for the tissue specificity of plasmid replication.

In a further experiment, the luciferase gene in replicating and non-replicating plasmids used above is replaced with a gene encoding β-galactosidase, generating pC-βgal-I-T-BB3 (SEQ ID NO: 614) and pC-βgal-I-T-dSV (SEQ ID NO: 615). Mice containing a liver-localized tumor following intralobular injection of PC-3 cells are injected with replicating and non-replicating plasmids as described above. At multiple time points, a minimum of 3 animals are sacrificed, the liver is extracted, cryosectioned, and stained for lac Z as described in Example 16B. Sections containing healthy tissue and the tumor are assessed for lac Z staining to determine the tissue selectivity of transgene expression.

### Example 18

### Bystander Effect of Autonomous Replicating Oncovectors

In this example, the autonomous replicating plasmids (ARPs), expressing fusogenic peptides or pro-drug converting enzymes are tested for their abilities to induce cell death in neighboring cells, not expressing ARP, known as the bystander effect, in xenotransplant mouse liver tumor models.

### A. Bystander Effect of Fusogenic Oncovectors

PC-3 cells are transfected with the following replication competent (ARP) or non-replicating (nRP) vectors containing the cDNA for the fusogenic peptides RRVp14; BRVp15; or GALV, under the regulation of a CMV promoter via electroporation: pC-RRVp14-I-T-BB3 (SEQ ID NO: 649), pC-RRVp14-I-T-dSV (SEQ ID NO: 650), pC-BRVp15-I-T-BB3 (SEQ ID NO: 651), pC-BRVp15-I-T-dSV (SEQ ID NO: 652), pC-GALV-I-T-BB3 (SEQ ID NO: 653), pC-GALV-I-T-dSV (SEQ ID NO: 654). Cells are also co-transfected, at a plasmid ratio of 1:1, with a non-replicating GFP-expressing plasmid which lacks the IRES sequence (pCzGFP-deltaI-T-BB3; SEQ ID NO: 609). Luciferase-expressing PC-3 cells (PC-3-Luc) are also transfected with pCzGFP-deltaI-T-BB3. Post-transfection, the ARP-transfected cells are mixed at different ratios with the control, transfected PC-3-Luc cells. The cell mixtures are then injected subcutaneously into nude mice, and luciferase activity is monitored, and tumor size is directly measured in vivo as described in Example 12 for 4-7 weeks. In the case of mice injected with CDase-expressing construct, the mice are provided drinking water with or without 5-FC. The reduction in luciferase-containing cells is quantified and used to calculate the number of neighboring, ARP non-expressing cells can be killed by a single cell targeted with an ARP, and serves as measurement of bystander activity. On the last day of the study, the tumors are extracted, cryo-sectioned, and analyzed for syncytia formation. GFP expression facilitates visualization of the syncytia. ARPs best able to propagate fusogenic/anticancer activity beyond the initially targeted cancer cells are then chosen for further study and optimization.

### B. Combinatorial Oncovector Treatment and Bystander Effect

In a further experiment, the combination of a fusogenic peptide and an adjunct therapy gene is tested to assess whether combinatorial treatment can enhance the bystander effect. For this experiment, a construct is generated to introduce the pro-drug, adjunct therapy gene CDase (SEQ ID NO: 500; encoding CDase protein set forth as SEQ ID NO: 502), between the Pf1FI and Bg1II restriction sites of the fusogenic ARP construct exhibiting the greatest bystander effect in part A above. The newly generated construct is co-transfected, at a plasmid ratio of 1:1, with a non-replicating GFP-expressing plasmid which lacks the IRES sequence (pCzGFP-deltaI-T-BB3; SEQ ID NO: 609) into PC-3 cells. Transfected PC-3 cells and untransfected PC-3-Luc cells are mixed at different ratios, and the cell mixtures are injected subcutaneously into nude mice. The injected animals are divided into two groups. One group is provided with 5-FC in the drinking water while the control group is not. Reporter expression (e.g. bioluminescence or fluorescence) is monitored weekly to directly measure tumor size in vivo over a period of 4-7 weeks. The reporter expression is compared between the 5-FC-treated and untreated animals to determine the combined bystander effect.

### Example 19

### Tumor-Specific Gene Expression and Replication of Autonomous Replicating Plasmids (ARPs) Using Cell Cycle-Dependent Promoters

In this example, a panel of cell cycle-dependent (CCD) promoters are used to assess if gene expression and replication of ARPs are restricted to tumor cells. The efficacy of tumor-specific gene regulation of CCD promoters is examined in cell culture and in vivo.

### A. Tumor Selectivity of Cell Cycle-Dependent Promoters

To test the tumor selectivity of various promoters which have been described as tumor-specific, constructs are cloned using pCzGFP-I-T-dSV (SEQ ID NO: 608). GFP is replaced with the gene for the reporter protein, secreted embryonic alkaline phosphatase (SEAP) between the NheI and BamHI restriction sites, and the CMV promoter is replaced with promoters listed in Table 37 between the AseI and NheI restriction sites. A variety of normal and cancer cell lines from different cell lineages are transfected with the reporter constructs. 48-72 hrs after cell transfection, cell culture media are removed and transferred to a microcentrifuge tube. Any detached cells present in the culture medium are pelleted by centrifugation. Supernatant is removed and analyzed for SEAP activity. The cells are also harvested, homogenized, and the level of TAg expression is determined by Western blot. These two assays provide the extent of tumor selectivity and TAg expression.

TABLE 37
Tumor Specific Promoters

| | SEQ ID NO |
|---|---|
| | (SEQ ID NO w/ |
| Cell Cycle-Dependent (CCD) Promoter | restriction sites) |
| CMV promoter | 504 |
| Telomerase promoter | 530 (679) |
| E2F-1 promoter | 534 (680), 535 (681) |
| Modified E2F-1 promoter | 536 (682), 537 (683) |
| Synthetic E2F-like promoter | 538 (684), 539 (685), |
| | 540 (686), 541 (687) |
| Antigen 33 (A33) promoter | 532 |
| Cyclo-oxygenase-2 (COX-2) promoter | 533 |
| Human carcinoembryonic antigen (CEA) | 531 |
| promoter | |
| Cyclin A (CycA) promoter | 519 |
| Cell division cycle 2 (Cdc2) promoter | 520 |
| Cell division cycle 25 (Cdc25) promoter | 521 |
| B-myb promoter | 522 |
| p107 promoter | 523 |
| Tyrosine Kinase (TK) promoter | 526 |
| DNA polymerase alpha promoter | 527 |
| Histone 2A (H2A) promoter | 528 |
| C-myc promoter | 529 |
| Synthetic cell cycle-dependent promoter | 505 |

### B. Cell Cycle-Dependent Expression and Replication of ARPs in Cell Culture

The CMV promoters in pCzGFP-I-T-BB3 (SEQ ID NO: 607) and pCzGFP-I-T-dSV (SEQ ID NO: 608) constructs are replaced with each of the CCD promoters, listed in Table 37, between AseI and NheI restriction sites as described in Example 5, to generate pCCDzGFP-I-T-BB3 and pCCDzGFP-I-T-dSV. Plasmids containing CCD promoters are also combined with TAg mutants, described in Example 10, to test for further selectivity of tumor-specific expression via limiting replication of leaky plasmid expression in cells containing functional Rb and/or p53.

Normal cells, represented by low passage cells, such as small airway epithelial cells (SAEC), and tumor cells, such as PC-3 cells, are transfected with replication competent pCCDzGFP-I-T-BB3, non-replicating pCCDzGFP-I-T-dSV, or non-discriminately replicating pCzGFP-I-T-BB3 (SEQ ID NO: 607). GFP expression and plasmid replication are measured in cells for each condition, as described in Example 8. The selectivity of ARP expression is determined by comparing the GFP expression and plasmid copy levels of the cells transfected with the replicating, cell cycle-dependent construct, pCCD-GFP-I-T-BB3, with the non-replicating negative control, pCCD-GFP-I-T-dSV, and the universal replicating positive control vector, pCzGFP-I-T-BB3 (SEQ ID NO: 607).

### C. Cell Cycle-Dependent Expression and Replication of ARPs In Vivo

To determine the specificity of the cell cycle-dependent promoter in controlling ARP gene expression in vivo, the constructs in part B above demonstrating selective tumor expression are used to confirm tumor-specific expression and replication in vivo. The GFP genes in the pCCDzGFP-I-T-BB3 and pCCDzGFP-I-T-dSV constructs used in part B are replaced with luciferase or β-galactosidase (β-gal) reporter constructs, generating pCCD-Luc-I-T-BB3, pCCD-Luc-I-T-dSV, pCCD-βgal-I-T-BB3, and pCCD-βgal-I-T-dSV. The replication and selectivity of ARP expression in an intralobular liver-localized tumor model, as described in Example 17.

### Example 20

### Systemic Delivery of Autonomous Replicating Oncovectors into Xenograft Mouse Tumor Model

### A. Nanoparticle Delivery System Optimization

To develop a method to efficiently deliver ARPs to tumor cells, several drug delivery systems (DDSs) are tested. Various plasmid/nanoparticle complexes are generated containing pC-mKate2-I-T-BB3 (SEQ ID NO: 611) and one of the following: polyethylenimine (PEI) polymer (Genesee Scientific), polypropylenimine dendrimer PPIG3 polymer (Chisholm, 2009), B-amino-ester polymer (Stemgent; Huang, 2009), liposome formulation (Invitrogen), or sugar molecule such as cyclodextrin polymers (Calando; Bellocq, 2003). The plasmid/nanoparticle complexes are then administered systemically to an intralobular-injected liver-localized tumor model by HTV injection (see Examples 13 and 15A), at various concentrations. Animals are monitored for reporter expression, e.g. fluorescence and bioluminescence imaging, in vivo, as described in the Examples above to ascertain the extent and location of plasmid delivery, and determine if tumor localization occurs through enhanced permeability and retention effect (EPR). Optimized conditions for plasmid/nanoparticle delivery are then used to administer ARPs expressing fusogenic peptides to test for efficacy in the treatment of human carcinoma xenografts in part B below.

### B. Nanoparticle Delivery of Fusogenic ARPs

In this example, autonomous replicating constructs containing DNA encoding fusogenic peptides are packaged into nanoparticles, prepared in part A, and administered to an intralobular liver-localized tumor model via systemic delivery to determine if the systemic delivery of these complexes can, in a replication dependent manner, reduce growth of established PC-3-Luc subcutaneous and liver tumors.

The zRFP genes in ARP and nRP constructs are replaced with the mKate2 reporter construct (SEQ ID NO: 549). Nanoparticle alone, or nanoparticle/plasmid complexes containing the mKate2-containing ARPs or nRPs are injected into nude mice containing intralobular-injected liver-localized tumors (described in Example 15A) via the hydrodynamic tail vein (HTV). Tumor size and plasmid replication and localization are monitored by reporter expression in vivo, e.g. bioluminescence and fluorescence imaging, respectively.

Since modifications will be apparent to those of skill in this art, it is intended that this invention be limited only by the scope of the appended claims.

What is claimed is:

1. A non-viral nucleic acid vector, comprising:
a) an origin of replication;
b) a first open reading frame coding for a fusogenic protein; and
c) a second open reading frame coding for a replication initiator protein that activates the origin of replication for episomal replication of the vector in a cell in which it is expressed.

2. The non-viral nucleic acid vector of claim 1 that is a non-viral oncovector.

3. The non-viral nucleic acid vector of claim 1 that comprises at least one promoter that is operatively linked to control expression of a first and/or second open reading frame.

4. The non-viral nucleic acid vector of claim 3, wherein:
the first open reading frame and second open reading frame are separated by an internal ribosome entry site (IRES); and
the first and second open reading frame are expressed under control of the same promoter.

5. The non-viral nucleic acid vector of claim 4, wherein the nucleic acid comprises in reading frame order 5′ to 3′:
a) a promoter operatively linked to control expression of the first and second open reading frame, a first open reading frame coding for a fusogenic protein, an IRES, a second open reading frame coding for the replication initiator and an origin of replication; or
b) a promoter operatively linked to control expression of the first and second open reading frame, a second open reading frame coding for the replication initiator, an IRES, a first open reading frame coding for a fusogenic protein and an origin of replication.

6. The non-viral nucleic acid vector of claim 3, wherein the nucleic acid molecule comprises:
a first promoter that is operatively linked to control expression of the first open reading frame coding for the fusogenic protein; and
a second promoter that is operatively linked to control expression of the second open reading frame coding for the replication initiator.

7. The non-viral nucleic acid vector of claim 6, wherein the nucleic acid comprises in reading frame order 5′ to 3′:
a first promoter operatively linked to control expression of the first open reading frame, a first open reading frame coding for a fusogenic protein, a second promoter that is operatively linked to control expression of the second open reading frame, a second open reading frame coding for a replication initiator and an origin of replication; or
a second promoter that is operatively linked to control expression of the second open reading frame, a second open reading frame coding for a replication initiator, a first promoter operatively linked to control expression of the first open reading frame, a first open reading frame coding for a fusogenic protein.

8. The non-viral nucleic acid vector of claim 6, wherein the first and second promoter are the same or different.

9. A non-viral nucleic acid vector composition or combination, comprising:
a) a first nucleic acid molecule comprising an origin of replication and a first open reading frame coding for a fusogenic protein; and
b) a second nucleic acid molecule comprising a second open reading frame coding for a replication initiator that activates the origin of replication in the first nucleic acid molecule when the first and second nucleic acid molecule are delivered into the same host cell for episomal replication of the first nucleic acid molecule.

10. The non-viral nucleic acid vector composition or combination of claim 9, wherein the first and second nucleic acid molecule are part of the same molecule or are on separate nucleic acid molecules.

11. The non-viral nucleic acid vector composition or combination of claim 9, wherein the first and second nucleic acid molecule each comprise at least one promoter that is operatively linked to control expression of the first and second open reading frame.

12. The non-viral nucleic acid vector composition or combination of claim 11, wherein the promoter is the same or different.

13. The non-viral nucleic acid vector of claim 1, wherein the origin of replication and replication initiator are selected from among:
a) an SV40 origin and an SV40 T antigen;
b) a BKV origin and BKV large T antigen;
c) a BKV origin and SV40 T antigen; and
c) an EBV origin and Epstein Barr virus Nuclear Antigen (EBNA).

14. The non-viral nucleic acid vector of claim 13, wherein the origin of replication is an SV40 origin and the replication initiator is an SV40 T antigen.

15. The non-viral nucleic acid vector of claim 14, wherein the SV40 origin is selected from among:
an SV40 origin that comprises an SV40 large T antigen core binding site set forth in SEQ ID NO:123;
a variant thereof having the formula set forth in SEQ ID NO:124 that exhibits at least 85% sequence identity to SEQ ID NO:123; or
an SV40 origin of replication that comprises a variant SV40 T antigen core binding site set forth in any of SEQ ID NOS: 125-189 and that exhibits at least 85% sequence identity to SEQ ID NO:123.

16. The non-viral nucleic acid vector of claim 14, wherein the origin of replication is a modified SV40 origin that is modified to remove upstream enhancers or that is modified to remove CpG motifs and/or is human codon-optimized.

17. The non-viral nucleic acid vector of claim 16, wherein the origin of replication is selected from among an SV40 origin of replication that comprises the sequence set forth in SEQ ID NO:113, 114, 115, or 116 or that comprises a sequence that exhibits at least 85% sequence identity to any of SEQ ID NOS: 113, 114, 115 or 116.

18. The non-viral nucleic acid vector of claim 14, wherein the second open reading frame codes a replication initiator that is an SV40 large T antigen.

19. The non-viral nucleic acid vector of claim 18, wherein the second open reading frame coding for a replication initiator protein is a modified SV40 large T antigen that is modified to remove CpG motifs, is human-codon optimized and/or is modified to reduce its cellular transforming activity.

20. The non-viral nucleic acid vector of claim 18, wherein:
the second open reading frame coding for an SV40 large T antigen comprises the sequence set forth in any of SEQ ID NOS: 561, 562 or 563, degenerates thereof or a sequence that exhibits at least 85% sequence identity to any of SEQ ID NOS: 561, 562 or 563 or degenerates thereof; or
the second open reading frame encodes an SV40 large T antigen comprising the sequence of amino acids set forth in SEQ ID NO:564, or a variant thereof that exhibits at least 85% sequence identity to SEQ ID NO:564.

21. The non-viral nucleic acid vector of claim 19, wherein the encoded SV40 large T antigen is modified to reduce its cellular transforming activity to reduce or eliminate binding to p53, HSP70 or Rb.

22. The non-viral nucleic acid vector of claim 21, wherein the encoded modified SV40 large T antigen comprises an amino acid replacement at an amino acid residue selected from among L17, G18, L19, E20, R21, S22, A23, W24, G25, N26, I27, P28, L29, M30, R31, K32, L103, C105, E107, E108, S112, S189, N366, D367, L368, L369, D370, D402, T434, L435, A436, A437, A438, L439, L440, E441, L442, C443, G444, P453, V585, D604, S677 or S679 corresponding to positions set forth in SEQ ID NO:564.

23. The non-viral nucleic acid vector of claim 22, wherein the encoded modified SV40 large T antigen comprises an amino acid replacement selected from among L19F, P28S, L103P, C105A, E107K, E107L, E108L, S112N, S189N, D402R, D402E, P453S, V585R, D604R, S677A and S679A.

24. The non-viral nucleic acid vector of claim 23, wherein the encoded modified SV40 large T antigen comprises an amino acid replacement selected from among E107L/E108L; E107L/E108L/D402R; E107L/E108L/P453S; E107L/E108L/V585R; E107L/E108L/D604R; L19F/E107L/E108L/D402R; L19F/E107L/E108L/P453S; L19F/E107L/E108L/V585R; L19F/E107L/E108L/D604R; P28S/E107L/E108L/D402R; P28S/E107L/E108L/P453S; P28S/E107L/E108L/V585R; P28S/E107L/E108L/V604R; L19F/P28S/L103P/C105A/E107L/E108L/V585R; L19F/P28S/L103P/C105A/E107L/E108L/D604R; L103P/C105A; L103P/E107K; C105A/E107K; C105A/D402E; C105A/V585R; E107K/V585R; E107K/D402E; L103P/D402E and L103P/V585R.

25. The non-viral nucleic acid vector of claim 24, wherein the second open reading frame codes for a replication initiator protein that is an SV40 large T antigen comprising the sequence of amino acids set forth in any of SEQ ID NOS: 565-604, or a sequence of amino acids that exhibits at least 85% sequence identity to any of SEQ ID NOS: 565-604.

26. The non-viral nucleic acid vector of claim 1, wherein the encoded fusogenic protein is a fusogenic protein that is a viral or eukaryotic fusogenic protein.

27. The non-viral nucleic acid vector of claim 26, wherein the fusogenic protein is selected from among VSV-G (Vesicular stomatitis virus G protein), MV (Measles virus) F protein, SIV (Simian immunodeficiency virus) F protein, HIV (Human immunodeficiency virus) 1+2 F protein, MuLV (Murine leukemia virus) F protein, Chicken LV Env Protein, SER virus F protein, NDV (Newcastle disease virus) F protein, GALV (Gibbon ape leukemia virus) F protein, SV5 (Simian virus 5) F protein, PPRV-F protein, Mumps F protein, Sendai virus F protein, Human parainfluenza virus types 1 (HPIV 1) F protein, HPIV 2 F protein, HPIV 3 F protein, CDV (Canine distemper virus) F protein, R'Pest F protein, SV41 (Simian virus 41) F protein, HRSV (Human respiratory syncytial virus) F protein, Human endogenous retroviral-3 (HERV-3), Reovirus FAST proteins, Avian Reovirus p10, Avian Reovirus p10 (S1133 variant V68I), Reptilian Reovirus p14, Baboon Reovirus p15, Eukaryotic Membrane Fusion Proteins, EFF-1, AFF-1, Tetraspanin Proteins, Yeast G Protein, Syncytin 1, Syncytin 2, Syntaxin (SNARE) and SNAP25 (SNARE), Synaptobrevin (SNARE) and variants thereof that exhibit fusogenic activity.

28. The non-viral nucleic acid vector of claim 27, wherein:
the first open reading frame coding for a fusogenic protein comprises the sequence of nucleotides set forth in SEQ ID NO: 6, 8, 10, 12, 14, 15, 17 or 27 or a sequence that exhibits at least 80% sequence identity thereto; or
the first open reading frame encodes a fusogenic protein comprising the sequence of amino acids set forth in SEQ ID NO: 38, 39, 40, 41, 42, 43, 44 or 53 or a sequence of amino acids that exhibits at least 85% sequence identity thereto.

29. The non-viral nucleic acid vector of claim 1, wherein the open reading frame coding for a fusogenic protein is modified to remove CpG motifs, is human codon-optimized and/or is modified in the N-terminal fusogenic peptide of the F1 subunit to increase the fusogenic activity of the encoded fusogenic protein.

30. The non-viral nucleic acid vector of claim 29, wherein the first open reading frame coding for a fusogenic protein comprises the sequence of nucleotides set forth in SEQ ID NO:7, 9, 11, 13, 16, 18 or a sequence that exhibits at least 85% sequence identity thereto.

31. The non-viral nucleic acid vector of claim 29, wherein the fusogenic protein is modified in the N-terminal fusogenic peptide of the F1 subunit and the modification is an amino acid replacement of at least one Glycine residue with an Alanine.

32. The non-viral nucleic acid vector of claim 31, wherein the encoded fusogenic protein is a modified SV5F protein that comprises an amino acid replacement in the F1 subunit at an amino acid residue selected from among 105, 109 and 115 corresponding to positions set forth in SEQ ID NO:44.

33. The non-viral nucleic acid vector of claim 32, wherein the encoded modified SV5F fusogenic protein comprises an amino acid replacement selected from among G105A, G109A and G114A.

34. The non-viral nucleic acid vector of claim 33, wherein the encoded modified SV5F fusogenic protein comprises an amino acid replacement selected from among G105A/G109A, G105A/G114A, G109A/G114A and G105A/G109A/G114A.

35. The non-viral nucleic acid vector of claim 33, wherein:
the first open reading frame coding for a fusogenic protein comprises the sequence of nucleotides set forth in SEQ ID NO:19-25 or a sequence that exhibits at least 85% sequence identity thereto; or
the first open reading frame encodes a fusogenic protein that has the sequence of amino acids set forth in SEQ ID NO: 45-51 or a sequence of amino acids that exhibits at least 85% sequence identity thereto.

36. The non-viral nucleic acid vector of claim 27, wherein the encoded fusogenic protein is a modified Ser virus F protein comprising an amino acid replacement selected from among L539A, L548A, L548V and L548G corresponding to positions set forth in SEQ ID NO:53.

37. The non-viral nucleic acid vector of claim 3, wherein the promoter is a constitutive promoter, a tissue-specific promoter or a cell-specific promoter.

38. The non-viral nucleic acid vector of claim 37, wherein the promoter is a CMV promoter.

39. The non-viral nucleic acid vector of claim 37, wherein the promoter is a cell-specific promoter selected from among endothelial nitric oxide synthase (eNOS) promoter; vascular endothelial growth factor (VEGF) receptor (flk1) promoter; insulin promoter; promoter of gonadotropin-releasing hormone receptor gene; matrix metalloproteinase 9 promoter; promoter of parathyroid hormone receptor; and dopamine beta-hydroxylase promoter.

40. The non-viral nucleic acid vector of claim 37, wherein the promoter is a tumor-specific promoter.

41. The non-viral nucleic acid vector of claim 40, wherein the tumor-specific promoter is a cell-cycle dependent promoter.

42. The non-viral nucleic acid vector of claim 41, wherein the promoter is selected from among cycA, cdc2, cdc25, B-myb, E2F-1, p107, HsOrc1, adenoE1A, cyclin B1, cyclin B2, Cdc2, and topoisomerase IIα.

43. The non-viral nucleic acid vector of claim 42, wherein the promoter is an E2F-1 promoter comprising the sequence of nucleotides set forth as nucleotides 37 to 303 of SEQ ID NO:506, as nucleotides 1194 to 1460 of SEQ ID NO: 483 or as nucleotides set forth in SEQ ID NO:534 or 535 or a variant sequence thereof that exhibits at least 85% sequence identity thereto.

44. The non-viral nucleic acid vector of claim 43, wherein the promoter is modified to remove CpG motifs, to reduce the promoter strength, to enhance the promoter strength, to reduce expression levels of the encoded protein or to increase expression levels of the encoded protein.

45. The non-viral nucleic acid vector of claim 44, wherein the promoter is modified by addition or insertion of an enhancer element selected from among SP-1, CAT box or CHR element.

46. The non-viral nucleic acid vector of claim 44, wherein the promoter is an E2F-1 promoter comprising the sequence of nucleotides set forth in one of SEQ ID NO:536-541 or a variant sequence thereof that exhibits at least 85% sequence identity thereto.

47. The non-viral nucleic acid vector of claim 1, wherein episomal replication occurs specifically in a tumor cell and not in a normal cell.

48. The non-viral nucleic acid vector of claim 1, comprising an open reading frame coding for an adjunct therapy protein or second bystander product.

49. The non-viral nucleic acid vector of claim 48, wherein the adjunct therapy protein or second bystander product is selected from among a protein that induces apoptosis, a toxin, a prodrug modifying protein, a protein that interferes with a signal transduction cascade involved with cellular survival or proliferation, an immunomodulatory protein and an angiogenesis inhibitor.

50. The non-viral nucleic acid vector of claim 49, wherein the adjunct therapy protein or second bystander protide is a prodrug modifying protein that is herpes simplex 1 thymidine kinase gene (HSV-TK), cytosine deaminase (CD) or cytochrome p450.

51. The non-viral nucleic acid vector of 50, wherein:
the open reading frame coding for a prodrug modifying protein encodes a prodrug modifying protein comprising the sequence of amino acids set forth in SEQ ID NO:501 or SEQ ID NO:502 or a variant sequence thereof that exhibits at least 85% sequence identity thereto; or
the open reading frame coding for a prodrug modifying protein comprises the sequence of nucleotides set forth in SEQ ID NO: 498 or 500 or a variant sequence thereof that exhibits at least 85% sequence identity thereto.

52. The non-viral nucleic acid vector of claim 50, wherein the open reading frame coding for a prodrug modifying protein is modified to remove CpG motifs and/or is humanized.

53. The non-viral nucleic acid vector of claim 52, wherein the open reading frame coding for a prodrug modifying protein comprises the sequence of nucleotides set forth in SEQ ID NO:499 or a variant sequence thereof that exhibits at least 85% sequence identity thereto.

54. The non-viral nucleic acid vector of claim 48, wherein the adjunct therapy gene is selected from among a cytokine and a chemokine.

55. The non-viral nucleic acid vector of claim 48, wherein the nucleic acid molecule comprises at least one promoter that is operatively linked to control expression of the first open reading frame, second open reading frame and/or the open reading frame coding for a second bystander product or adjunct therapy gene.

56. The non-viral nucleic acid vector of claim 1, comprising an open reading frame coding for a reporter protein.

57. The non-viral nucleic acid vector of claim 56, wherein the reporter protein is selected from among chloramphenicol acetyl transferase (CAT), β-galactosidase, luciferase, alkaline phosphatase, a fluorescent protein, and horse radish peroxidase, and an antibiotic resistance marker.

58. The non-viral nucleic acid vector of claim 56, wherein the nucleic acid molecule comprises at least one promoter that is operatively linked to control expression of the first open reading frame, second open reading frame and/or the open reading frame coding for a reporter protein.

59. The non-viral nucleic acid vector of claim 1, wherein the first and/or second open reading frame is operatively linked to one or more regulatory elements to control expression of the gene.

60. The non-viral nucleic acid vector of claim 59, wherein the regulatory element is a polyadenylation signal or an internal promoter.

61. The non-viral nucleic acid vector of claim 1, comprising:
a) a promoter that controls expression of the first and second open-reading frame;
b) a first open reading frame coding for a fusogenic protein or variant thereof that exhibits fusogenic activity;
c) an IRES;
d) a second open reading frame coding for a replication initiator or variant thereof that binds the origin of replication; and
e) an origin of replication or variant thereof that is compatible with the replication initiator to initiate replication of DNA from the replication initiator or variant thereof, whereby the replication initiator activates the origin of replication for episomal replication, wherein:
the first open reading frame is positioned before the second open reading frame in the nucleic acid molecule; or
the second open reading frame is positioned before the first open reading frame in the nucleic acid molecule.

62. The non-viral nucleic acid vector of claim 61, comprising the sequence of nucleotides set forth in SEQ ID NO: 647, 649, 651, 653, 655, 657, 659-663, 693, 700-705, 722 or 727 or a sequence of nucleotides that exhibits at least 85% sequence identity to any of SEQ ID NO: 647, 649, 651, 653, 655, 657, 659-663, 693, 700-705, 722 or 727.

63. A non-viral nucleic acid vector, comprising:
a) an origin of replication;
b) a first open reading frame coding for a prodrug converting enzyme; and
c) a second open reading frame coding for a replication initiator protein that activates the origin of replication for episomal replication of the vector in a cell in which it is expressed.

64. The non-viral nucleic acid vector of claim 63, comprising:
a) a promoter that controls expression of the first and second open-reading frame;
b) a first open reading frame coding for a prodrug converting enzyme or variant thereof;
c) an IRES;
d) a second open reading frame coding for a replication initiator or variant thereof that binds the origin of replication; and
e) an origin of replication or variant thereof that is compatible with the replication initiator to initiate replication of DNA from the replication initiator or variant thereof, whereby the replication initiator activates the origin of replication for episomal replication, wherein:
the first open reading frame is positioned before the second open reading frame in the nucleic acid molecule; or
the second open reading frame is positioned before the first open reading frame in the nucleic acid molecule.

65. The non-viral nucleic acid vector of claim 63, wherein the prodrug modifying protein is herpes simplex 1 thymidine kinase gene (HSV-TK), cytosine deaminase (CD) or cytochrome p450.

66. The non-viral nucleic acid vector of claim 63, wherein the origin of replication and replication initiator are selected from among:
a) an SV40 origin and an SV40 T antigen;
b) a BKV origin and BKV large T antigen;
c) a BKV origin and SV40 T antigen; and
c) an EBV origin and Epstein Barr virus Nuclear Antigen (EBNA).

67. The non-viral nucleic acid vector of claim 66, wherein the origin of replication is an SV40 origin or variant thereof and the replication initiator is an SV40 T antigen or variant thereof.

68. The non-viral nucleic acid vector of claim 63, comprising the sequence of nucleotides set forth in SEQ ID NO: 664 or 724 or a sequence of nucleotides that exhibits at least 80% sequence identity to SEQ ID NO: 664 or 724.

69. The non-viral nucleic acid vector of claim 1 that is a naked DNA.

70. A nanoparticle, comprising the non-viral nucleic acid vector of claim 1.

71. A nanoparticle, comprising the non-viral nucleic acid vector of claim 63.

72. The nanoparticle of claim 70 that is conjugated to a protein that targets a tumor.

73. The nanoparticle of claim 71 that is conjugated to a protein that targets a tumor.

74. A method of treating cancer, comprising administering a non-viral nucleic acid vector of claim 1 to a subject that has a cancer.

75. The method of treatment of claim 74, wherein the cancer is selected from among a sarcomas, mesothelioma, carcinoids, melanoma, neuroblastoma, retinoblastoma, osteosarcoma, and cancers of the lung, colon, esophagus, ovary, pancreas, skin, stomach, head and neck, bladder, prostate, liver, brain, adrenal gland, breast, endometrium, kidney, thyroid, parathyroid, cervix, bone, eye and hematological system.

76. The method of treatment of claim 74, further comprising treating the subject by a targeted therapy, chemotherapy, radiotherapy, immunotherapy, hormonal therapy, cryotherapy or surgery.

77. A method of treating cancer, comprising administering a non-viral nucleic acid vector of claim 63 to a subject that has a cancer.

78. The method of treatment of claim 77, wherein the cancer is selected from among a sarcomas, mesothelioma, carcinoids, melanoma, neuroblastoma, retinoblastoma, osteosarcoma, and cancers of the lung, colon, esophagus, ovary, pancreas, skin, stomach, head and neck, bladder, prostate, liver, brain, adrenal gland, breast, endometrium, kidney, thyroid, parathyroid, cervix, bone, eye and hematological system.

79. The method of treatment of claim 77, further comprising treating the subject by a targeted therapy, chemotherapy, radiotherapy, immunotherapy, hormonal therapy, cryotherapy or surgery.
